(19) United States
(12) Patent Application Publication
(10) Pub. No.: US 2016/0194299 A1
(43) Pub. Date: Jul. 7, 2016

(54) COMPOUNDS FOR TREATMENT OF DRUG RESISTANT AND PERSISTENT TUBERCULOSIS

(71) Applicants: THE CALIFORNIA INSTITUTE FOR BIOMEDICAL RESEARCH, La Jolla, CA (US); THE GLOBAL ALLIANCE FOR TB DRUG DEVELOPMENT, INC., New York, NY (US); THE SCRIPPS RESEARCH INSTITUTE, La Jolla, CA (US)

(72) Inventors: Arnab K. CHATTERJEE, San Diego, CA (US); Feng WANG, Carlsbad, CA (US); Peter G. SCHULTZ, La Jolla, CA (US); Chunping XU, San Diego, CA (US); Kehinde AJAYI, San Diego, CA (US); Jianing WANG, San Diego, CA (US); Rajkumar HALDER, Kolkata, West Bengal (IN); Puneet KUMAR, Woodbridge, NJ (US); Baiyuan YANG, San Diego, CA (US); Renhe LIU, San Diego, CA (US); Bo CHENG, San Diego, CA (US); Takushi KANEKO, Guilford, CT (US)

(21) Appl. No.: 14/893,465

(22) PCT Filed: May 22, 2014
(86) PCT No.: PCT/US2014/039227

§ 371 Date: Nov. 23, 2015

Related U.S. Application Data
(60) Provisional application No. 61/827,539, filed on May 24, 2013.
(60) Provisional application No. 61/950,752, filed on Mar. 10, 2014.

(51) Int. Cl. C07D 333/38; C07D 417/14; C07D 413/12; C07D 471/04; C07D 409/14; C07D 417/12; C07D 409/12

(52) U.S. Cl. CPC C07D 333/38; C07D 417/12; C07D 417/14; C07D 409/12; C07D 471/04; C07D 409/14; C07D 413/12

(57) ABSTRACT

Described herein are compounds and compositions for treating drug resistant and persistent tuberculosis. Also described herein is a method of screening for identifying biofilm formation inhibitors.

### CROSS-REFERENCE TO RELATED APPLICATIONS

This application claims the benefit of U.S. Application No. 61/827,539, filed May 24, 2013, and U.S. Application No. 61/950,752, filed Mar. 10, 2014, both of which are hereby incorporated by reference in their entirety.

### BACKGROUND OF THE INVENTION

One of the greatest needs in global health is the development of new drugs against tuberculosis (TB) that shorten the duration of TB chemotherapy, and that are potent against drug resistant strains of Mtb for which current therapies are no longer effective [Gandhi N R, et al. (2010) HIV coinfection in multidrug- and extensively drug-resistant tuberculosis results in high early mortality. Am J Respir Crit Care Med 181:80-86]. TB is exceptional among bacterial infections in that even drug-susceptible strains are difficult to treat rapidly and effectively. This is in part due to the phenomenon of Mtb persistence, a state of phenotypic drug-tolerance that is attributed to a quiescent or non-replicating population of bacilli. Long treatment regimes make compliance problematic, and lead to the emergence of drug resistant mutants.

### SUMMARY OF THE INVENTION

In one aspect, provided herein are compounds of Formula (I), a pharmaceutically acceptable salt, solvate, polymorph, prodrug, metabolite, N-oxide, stereoisomer, or isomer thereof:

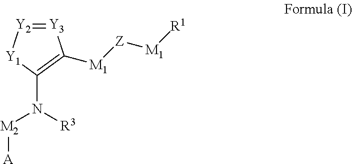

wherein:
M₁ are each independently a bond, —C(═O)— or —S(═O)₂—;
M₂ is —(CH₂)—, —C(═O)— or —S(═O)₂—;
Z is a bond or NR²;
Y₁ is S, O or NR²;
Y₂ is CR⁴ or N;
Y₃ is CR⁵ or N;
R¹ is —O-(optionally substituted alkyl), —O-(alkenyl), —O-(alkynyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), —O-(alkyl)-(NR⁶R⁷), —NR⁶R⁷ or R⁸;
R² and R³ are each independently selected from H, optionally substituted alkyl, and optionally substituted aryl; or R¹ and R² taken together form a heterocycle;
R⁴ is H, halogen, —CN, optionally substituted alkyl, optionally substituted aryl, —R^{b}COOR^{a} or —R^{b}CH(COOR^{a})₂;
R⁵ is H, halogen, optionally substituted alkyl or cycloalkyl; or R⁴ and R⁵ taken together form a carbocycle or an optionally substituted heterocycle;
R⁶ and R⁷ are each independently selected from H and optionally substituted alkyl; or R⁶ and R⁷ taken together form an optionally substituted heterocycle with the nitrogen to which they are attached;
R⁸ is optionally substituted alkyl, optionally substituted aryl, carbocyclyl, optionally substituted aralkyl, optionally substituted heteroaryl, optionally substituted heterocyclyl, —R^{b}COOR^{a} or —R^{b}CONR^{a}R^{a};
each R^{a} is independently selected from H and alkyl;
R^{b} is a bond or alkylenyl;
R^{c} is a bond or alkenylenyl; and
A is optionally substituted aryl, optionally substituted heterocyclyl, optionally substituted heteroaryl, optionally substituted carbocyclyl, optionally substituted aralkyl, optionally substituted heteroaralkyl or —R^{c}-(optionally substituted heteroaryl).

In some embodiments of a compound of Formula (I), Y₃ is CR⁵. In some embodiments of a compound of Formula (I), Y₁ is S. In some embodiments of a compound of Formula (I), Y₂ is CR⁴. In some embodiments of a compound of Formula (I), each M₁ is —C(═O)—. In some embodiments of a compound of Formula (I), Z is NR². In some embodiments of a compound of Formula (I), M₂ is —C(═O)—. In some embodiments of a compound of Formula (I), R¹ is —O-(optionally substituted alkyl); and R² and R³ are both H.

In another aspect, provided herein are compounds of Formula (Ia), a pharmaceutically acceptable salt, solvate, polymorph, prodrug, metabolite, N-oxide, stereoisomer, or isomer thereof:

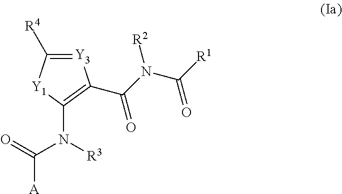

wherein:
Y₁ is S or O or NR²;
Y₃ is CR⁵ or N;
R¹ is —O-(optionally substituted alkyl), —O-(alkenyl), —O-(alkynyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), —O-(alkyl)-(NR⁶R⁷), —NR⁶R⁷ or R⁸;
R² and R³ are each independently selected from H, optionally substituted alkyl, and optionally substituted aryl; or R¹ and R² taken together form a heterocycle;
R⁴ is H, halogen, —CN, alkyl, aryl, —R^{b}COOR^{a} or —R^{b}CH(COOR^{a})₂;
R⁵ is H, halogen, optionally substituted alkyl, or cycloalkyl; or R⁴ and R⁵ taken together form a carbocycle or an optionally substituted heterocycle;
R⁶ and R⁷ are each independently selected from H and optionally substituted alkyl; wherein the optional substituent is halogen; or R⁶ and R⁷ taken together form an optionally substituted heterocycle with the nitrogen to which they are attached; wherein the optional substituent is halogen;
R⁸ is optionally substituted alkyl;
each R^{a} is independently selected from H and alkyl;
R^{b} is a bond or alkylenyl;
R^{c} is a bond or alkenylenyl; and
A is optionally substituted aryl, optionally substituted heterocyclyl, optionally substituted heteroaryl, optionally substituted carbocyclyl, optionally substituted aralkyl, optionally substituted heteroaralkyl or —R^{c}-(optionally substituted heteroaryl).

In some embodiments of a compound of Formula (Ia), R¹ is —O-(optionally substituted alkyl); wherein the optionally substituted alkyl is substituted with halogen. In some embodiments of a compound of Formula (Ia), R¹ is —O-(cycloalkyl). In some embodiments of a compound of Formula (Ia), R¹ is —O-(heterocyclyl). In some embodiments of a compound of Formula (Ia), R¹ is —O-(optionally substituted aralkyl) or —O-(optionally substituted heteroaralkyl). In some embodiments of a compound of Formula (Ia), R¹ is —O-(alkyl)-(heterocyclyl). In some embodiments of a compound of Formula (Ia), R⁴ and R⁵ taken together form an optionally substituted heterocycle; wherein the optionally substituted heterocycle is substituted with a group selected from alkyl, aralkyl and —SO₂Me. In some embodiments of a compound of Formula (Ia), R⁶ and R⁷ taken together form a heterocycle with the nitrogen to which they are attached; wherein the heterocycle is selected from piperidinyl and morpholinyl. In some embodiments of a compound of Formula (Ia), n Y₃ is CR⁵. In some embodiments of a compound of Formula (Ia), Y₁ is S. In some embodiments of a compound of Formula (Ia), Y₁ is S and Y₃ is CH. In some embodiments of a compound of Formula (Ia), R⁴ is H. In some embodiments of a compound of Formula (Ia), R¹ is —O-(optionally substituted alkyl); and R² and R³ are both H.

In some embodiments of a compound of Formulas (Ia), A is optionally substituted aryl, optionally substituted heterocyclyl, optionally substituted heteroaryl, optionally substituted carbocyclyl, optionally substituted aralkyl, optionally substituted heteroaralkyl or —R^{c}-(optionally substituted heteroaryl); and the optionally substituted aryl, the optionally substituted heterocyclyl, the optionally substituted heteroaryl, the optionally substituted carbocyclyl, the optionally substituted aralkyl and the optionally substituted heteroaralkyl are substituted with 1-6 R¹⁰; wherein each R¹⁰ is independently selected from H, halogen, —CN, —NO₂, —CF₃, alkyl, —SR⁶, —OR⁶, —NR⁶R⁷, —NR⁶C(═O)(alkyl), —NR⁶C(═O)(cycloalkyl), —NR⁶C(═O)(heterocyclyl), —NR⁶C(═O)(aryl), —NR⁶C(═O)(heteroaryl), —C(═O)NR⁶R⁷, —C(═O)NR⁶(cycloalkyl), —C(═O)NR⁶(heterocycloalkyl), —C(═O)NR⁶(aryl), —C(═O)NR⁶(heteroaryl), —NR⁶C(═O)NR⁶R⁷, —NR⁶C(═O)NR⁷(cycloalkyl), —NR⁶C(═O)NR⁷(heterocycloalkyl), —NR⁶C(═O)NR⁷(aryl), —NR⁶C(═O)NR⁷(heteroaryl), —NR⁶C(═O)O(alkyl), —NR⁶C(═O)O(cycloalkyl), —NR⁶C(═O)O(heterocycloalkyl), —NR⁶C(═O)O(aryl), —NR⁶C(═O)O(heteroaryl), —NR⁶SO₂(alkyl), —NR⁶SO₂(cycloalkyl), —NR⁶SO₂(heterocycloalkyl), —NR⁶SO₂(aryl), —NR⁶SO₂(heteroaryl), —SO₂NR⁶R⁷, —SO₂NR⁶(cycloalkyl), —SO₂NR⁶(heterocycloalkyl), —SR⁶, —SO₂R⁶, —SO₂NR⁶(aryl), —SO₂NR⁶(heteroaryl), haloalkyl, aryl, heteroaryl, heterocyclyl and tetrazoyl.

In some embodiments of a compound of Formula (Ia), A is optionally substituted aryl. In some embodiments of a compound of Formula (Ia), A is optionally substituted heteroaryl. In some embodiments of a compound of Formula (Ia), A is selected from:

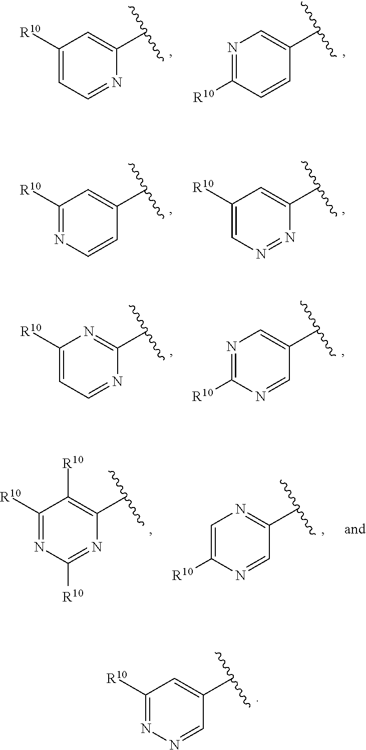

In some embodiments of a compound of Formula (Ia), A is:

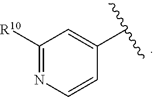

In some embodiments of a compound of Formula (Ia), A is selected from:

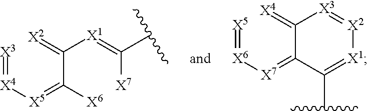

wherein X¹, X², X³, X⁴, X⁵, X⁶, and X⁷ are independently selected from N and CR¹⁰; and at least one of X¹-X⁷ is N. In some embodiments of a compound of Formula (Ia), A is selected from:

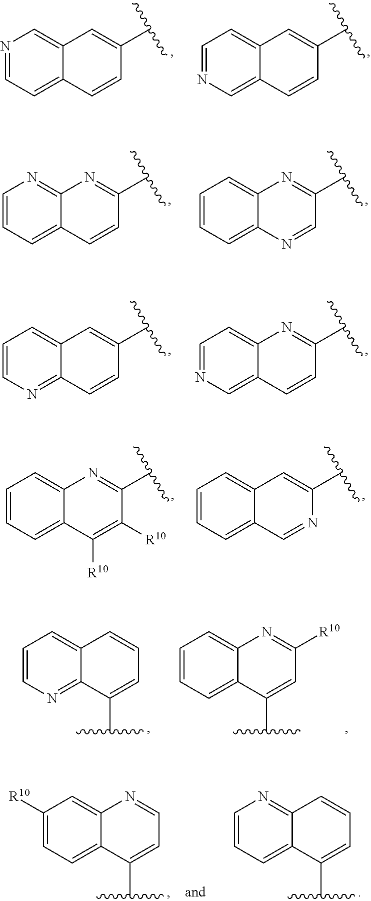

In some embodiments of a compound of Formula (Ia), A is selected from:

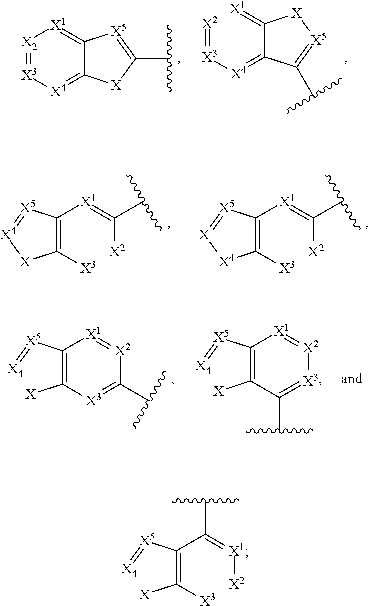

wherein X¹, X², X³, X⁴, X⁵, X⁶, and X⁷ are independently selected from N and CR¹⁰; and X is O, S, or NR². In some embodiments of a compound of Formula (Ia), A is selected from:

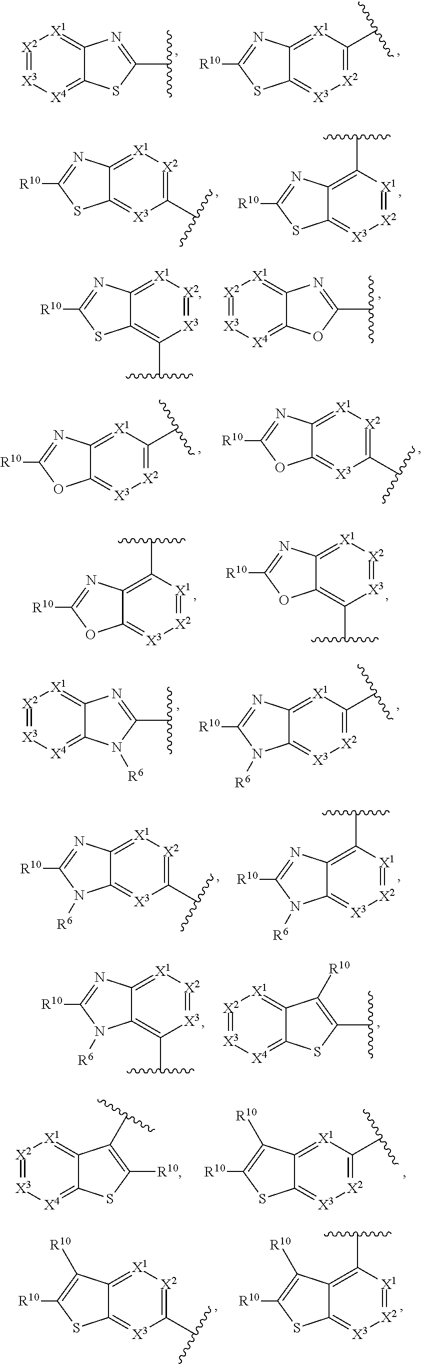

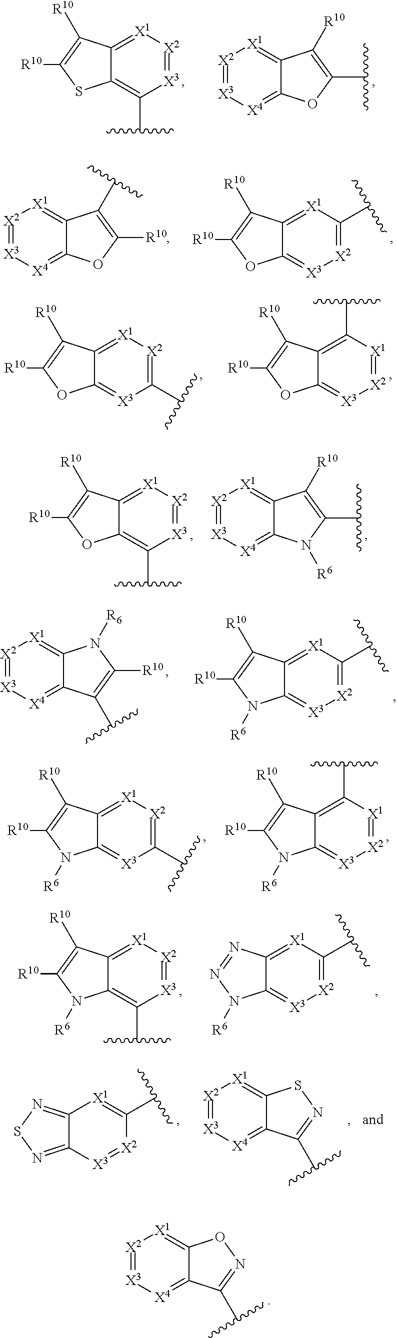

In some embodiments of a compound of Formula (Ia), A is selected from:

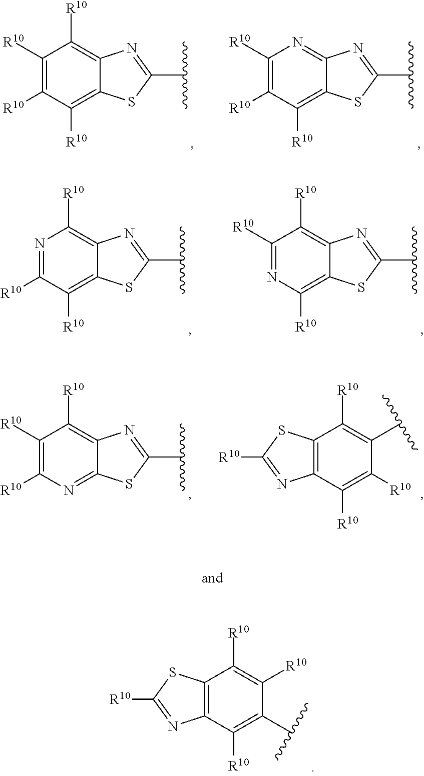

In some embodiments of a compound of Formula (Ia), A is selected from:

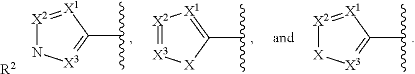

In some embodiments of a compound of Formula (Ia), A is selected from:

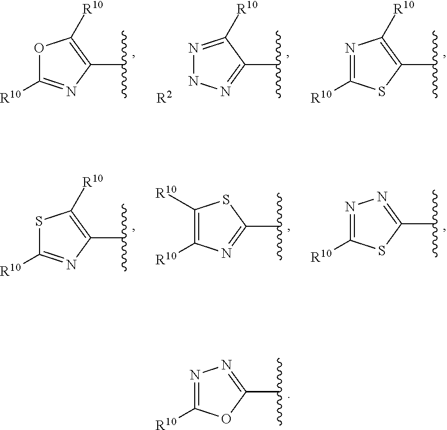

In some embodiments of a compound of Formula (Ia), A is selected from:

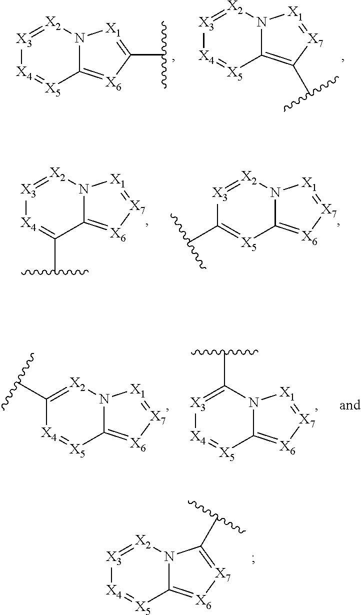

wherein X¹, X², X³, X⁴, X⁵, X⁶, and X⁷ are independently selected from N and CR¹⁰. In some embodiments of a compound of Formula (Ia), A is selected from:

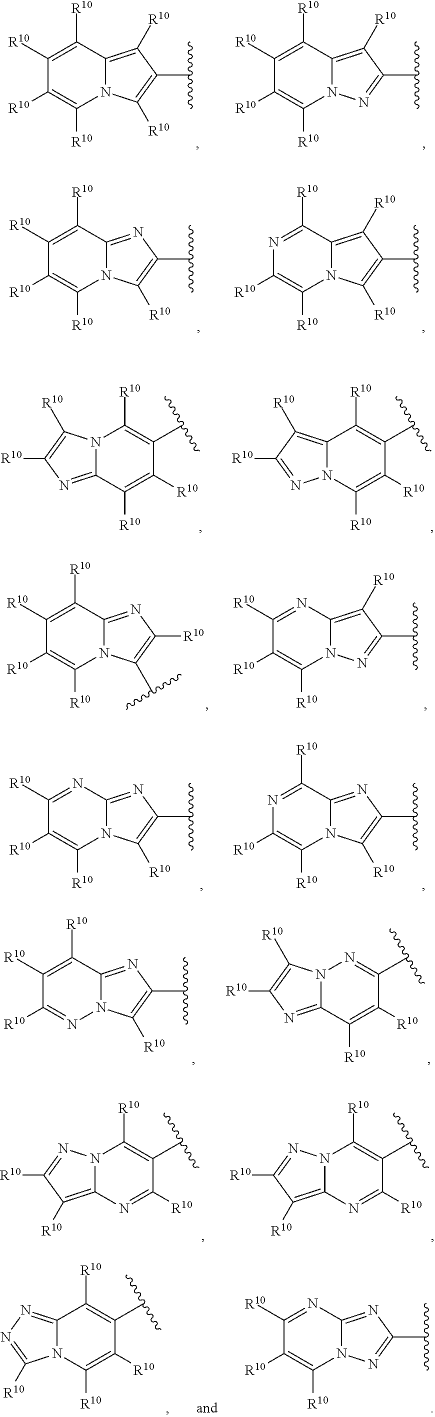

In some embodiments of a compound of Formula (Ia), A is selected from:

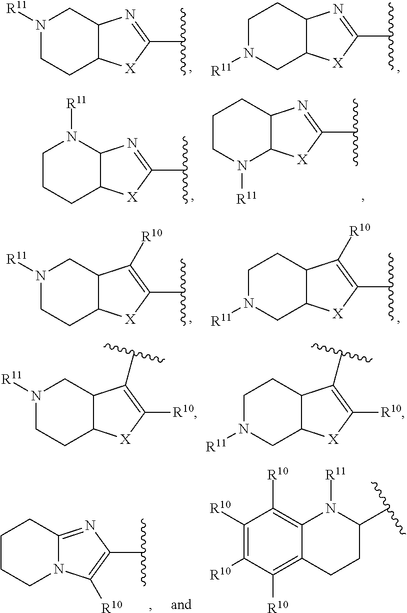

wherein: X is O, S, or NR²; and R¹¹ is H, alkyl, aryl, heteroaryl, —SO₂-(alkyl), —SO₂-(cycloalkyl), —SO₂-(aryl), —SO₂-(heteroaryl), —SO₂-(heterocycloalkyl), —C(═O)O(alkyl), —C(═O)O(cycloalkyl), —C(═O)O(heterocycloalkyl), —C(═O)O(aryl), —C(═O)O(heteroaryl), —C(═O)NR⁶R⁷, —C(═O)NR⁶(cycloalkyl), —C(═O)NR⁶(heterocycloalkyl), —C(═O)NR⁶(aryl), —C(═O)NR⁶(heteroaryl), —C(═O)(alkyl), —C(═O)(cycloalkyl), —C(═O)(heterocycloalkyl), —C(═O)(aryl), or —C(═O)(heteroaryl). In some embodiments of a compound of Formula (Ia), A is selected from:

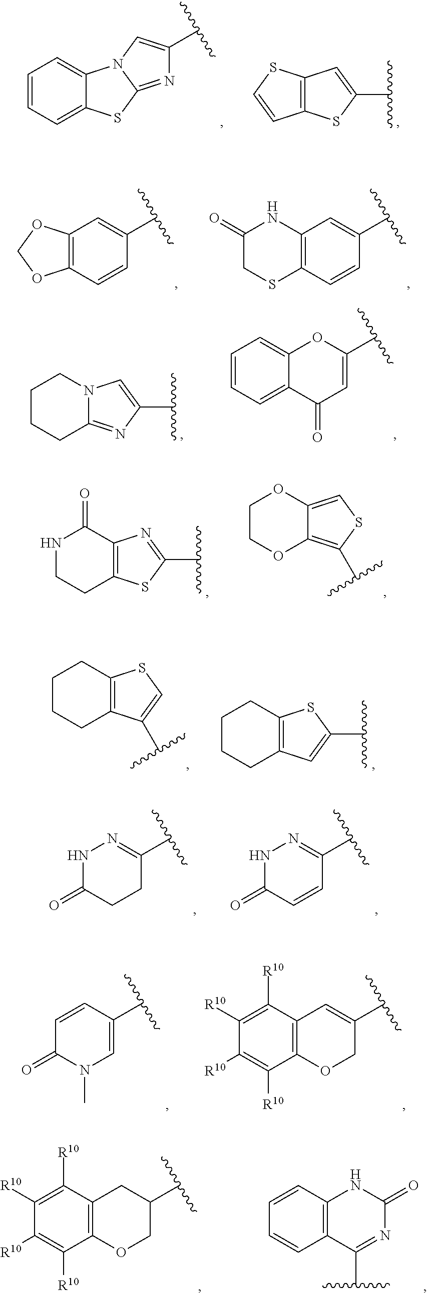

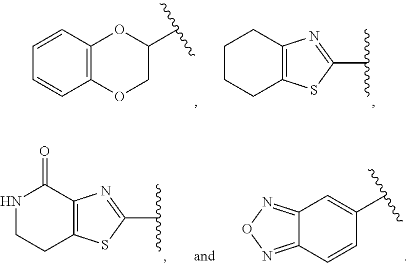 [SMILES: *C1COc2ccccc2O1 ; *c1nc2c(s1)CCCC2 ; *c1nc2c(s1)CCNC2=O ; *c1ccc2nonc2c1]

In another aspect, provided herein are compounds of Formula (Ic), a pharmaceutically acceptable salt, solvate, polymorph, prodrug, metabolite, N-oxide, stereoisomer, or isomer thereof:

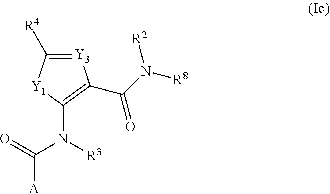

wherein:
Y₁ is S or O;
Y₃ is CR⁵ or N;
R² and R³ are each independently selected from H and optionally substituted alkyl;
R⁴ is H, halogen, —CN, optionally substituted alkyl, or optionally substituted aryl;
R⁵ is H, optionally substituted alkyl, or halogen;
R⁸ is optionally substituted alkyl, optionally substituted aryl, optionally substituted aralkyl, carbocyclyl, optionally substituted carbocyclylalkyl, optionally substituted heteroaryl, optionally substituted heteroarylalkyl, optionally substituted heterocyclyl, optionally substituted heterocyclylalkyl, —R^{b}COOR^{a} or —R^{b}CONR^{a}R^{a}; or R² and R⁸ taken together form an optionally substituted heterocycle with the nitrogen to which they are attached;
each R^{a} is independently selected from H and alkyl;
R^{b} is a bond or alkylenyl; and
A is optionally substituted heteroaryl, optionally substituted aryl or optionally substituted heterocyclyl.

In some embodiments of a compound of Formula (Ic), R⁸ is optionally substituted aryl; wherein the optionally substituted aryl is substituted with halogen. In some embodiments of a compound of Formula (Ic), R⁸ is optionally substituted heteroaryl; wherein the optionally substituted heteroaryl is substituted with a group selected from alkyl, —O-(alkyl) and —NR⁶R⁷. In some embodiments of a compound of Formula (Ic), R⁸ is optionally substituted heterocyclyl; wherein the optionally substituted heterocyclyl is substituted with alkyl. In some embodiments of a compound of Formula (Ic), R² and R⁸ taken together form an optionally substituted heterocycle with the nitrogen to which they are attached.

In some embodiments of a compound of Formula (Ic), Y₃ is CR⁵. In some embodiments of a compound of Formula (Ic), R⁵ is H. In some embodiments of a compound of Formula (Ic), Y₁ is S. In some embodiments of a compound of Formula (Ic), R⁴ is H.

In some embodiments of a compound of Formula (Ic), wherein A is optionally substituted heteroaryl, optionally substituted aryl or optionally substituted heterocyclyl; and the optionally substituted aryl, the optionally substituted heterocyclyl, the optionally substituted heteroaryl are substituted with 1-6 R¹⁰; wherein each R¹⁰ is independently selected from H, halogen, —CN, —NO₂, —CF₃, alkyl, —SR⁶, —OR⁶, —NR⁶R⁷, —NR⁶C(═O)(alkyl), —NR⁶C(═O)(cycloalkyl), —NR⁶C(═O)(heterocyclyl), —NR⁶C(═O)(aryl), —NR⁶C(═O)(heteroaryl), —C(═O)NR⁶R⁷, —C(═O)NR⁶(cycloalkyl), —C(═O)NR⁶(heterocycloalkyl), —C(═O)NR⁶(aryl), —C(═O)NR⁶(heteroaryl), —NR⁶C(═O)NR⁶R⁷, —NR⁶C(═O)NR⁷(cycloalkyl), —NR⁶C(═O)NR⁷(heterocycloalkyl), —NR⁶C(═O)NR⁷(aryl), —NR⁶C(═O)NR⁷(heteroaryl), —NR⁶C(═O)O(alkyl), —NR⁶C(═O)O(cycloalkyl), —NR⁶C(═O)O(heterocycloalkyl), —NR⁶C(═O)O(aryl), —NR⁶C(═O)O(heteroaryl), —NR⁶SO₂(alkyl), —NR⁶SO₂(cycloalkyl), —NR⁶SO₂(heterocycloalkyl), —NR⁶SO₂(aryl), —NR⁶SO₂(heteroaryl), —SO₂NR⁶R⁷, —SO₂NR⁶(cycloalkyl), —SO₂NR⁶(heterocycloalkyl), —SR⁶, —SO₂R⁶, —SO₂NR⁶(aryl), —SO₂NR⁶(heteroaryl), haloalkyl, aryl, heteroaryl, heterocyclyl and tetrazoyl.

In some embodiments of a compound of Formula (Ic), A is selected from:

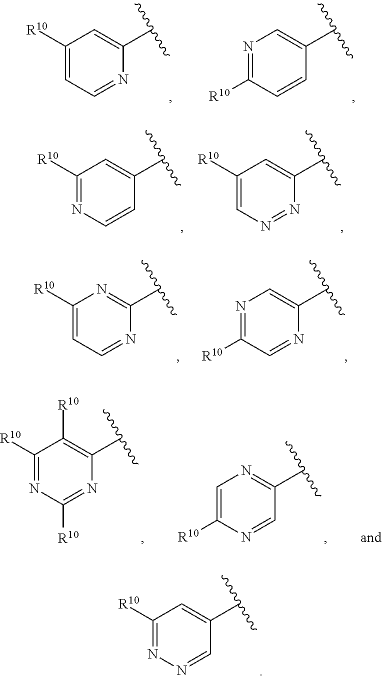

In some embodiments of a compound of Formula (Ic), A is

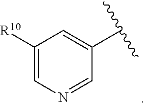

In some embodiments of a compound of Formula (Ic), A is selected from:

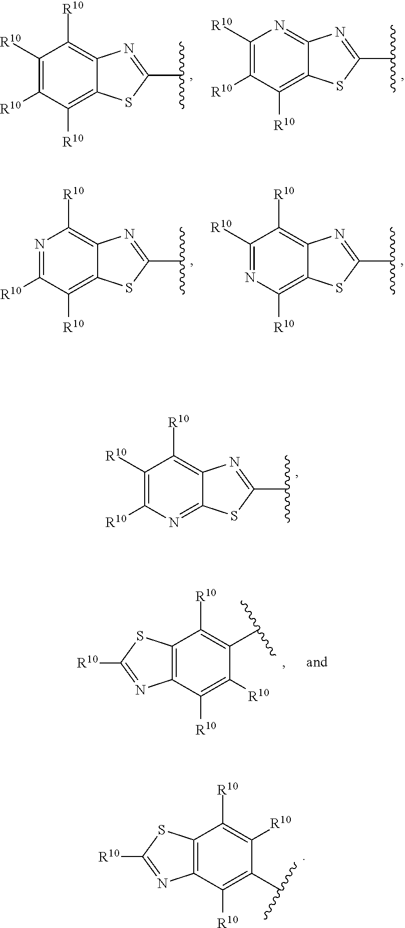

In some embodiments of a compound of Formula (Ic), A is

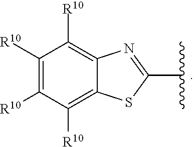

In another aspect, provided herein are compounds of Formula (Id), a pharmaceutically acceptable salt, solvate, polymorph, prodrug, metabolite, N-oxide, stereoisomer, or isomer thereof:

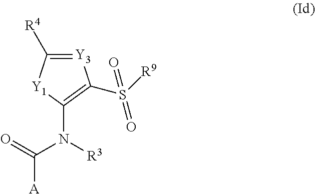

wherein:
Y₁ 1 S or O;
Y₂ is CR⁵ or N;
R³ is H, haloalkyl or alkyl;
R⁴ is H, halogen, CN or optionally substituted alkyl;
R⁵ is H, optionally substituted alkyl or halogen;
R⁹ is optionally substituted alkyl, optionally substituted heteroaryl or optionally substituted aryl; and
A is optionally substituted heteroaryl, optionally substituted aryl, or optionally substituted heterocyclyl.

In another aspect, provided herein are compounds, or pharmaceutically acceptable salts, solvates, polymorphs, prodrugs, metabolites, N-oxides, stereoisomers, or isomers thereof, selected from the compounds of Table A.

In another aspect, provided herein are compounds selected from:

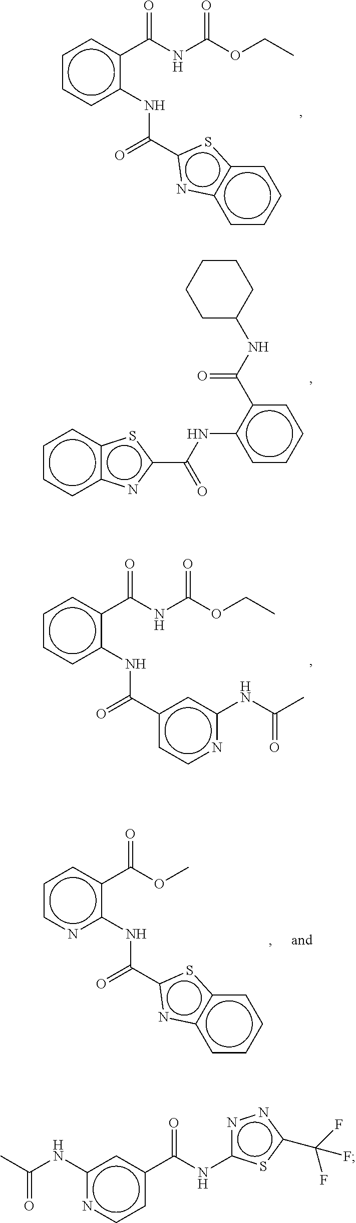

or pharmaceutically acceptable salts, solvates, polymorphs, prodrugs, metabolites, N-oxides, stereoisomers, or isomers thereof.

In another aspect, provided herein are compounds of Formula (IIb), a pharmaceutically acceptable salt, solvate, polymorph, prodrug, metabolite, N-oxide, stereoisomer, or isomer thereof:

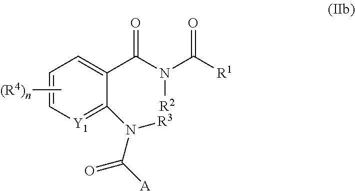

wherein:
Y₁ is N, CH, or CR⁴;
R¹ is —O-(optionally substituted alkyl), —O-(alkenyl), —O-(alkynyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), —O-(alkyl)-(NR⁶R⁷), —NR⁶R⁷ or R⁸;
R² and R³ are each independently selected from H, optionally substituted alkyl, and optionally substituted aryl; or R¹ and R² taken together form a heterocycle;
each R⁴ is independently selected from halogen, —CN, optionally substituted alkyl, optionally substituted alkoxy, optionally substituted aryl, —R^{b}COOR^{a}, and —R^{b}CH(COOR^{a})₂;
R⁶ and R⁷ are each independently selected from H and optionally substituted alkyl; or R⁶ and R⁷ taken together form an optionally substituted heterocycle with the nitrogen to which they are attached;
R⁸ is optionally substituted alkyl, optionally substituted aryl, carbocyclyl, optionally substituted aralkyl, optionally substituted heteroaryl, or optionally substituted heterocyclyl;
each R^{a} is independently selected from H and alkyl;
R^{b} is a bond or alkylenyl;
R^{c} is a bond or alkenylenyl;
n is 0, 1, 2, or 3; and
A is optionally substituted aryl, optionally substituted heterocyclyl, optionally substituted heteroaryl, optionally substituted carbocyclyl, optionally substituted aralkyl, optionally substituted heteroaralkyl or —R^{c}-(optionally substituted heteroaryl).

In some embodiments of a compound of Formula (IIb), Y₁ is N or CH. In some embodiments of a compound of Formula (IIb), Y₁ is N. In some embodiments of a compound of Formula (IIb), R¹ is —O-(optionally substituted alkyl); and R² and R³ are both H. In some embodiments of a compound of Formula (IIb), A is optionally substituted aryl, optionally substituted heterocyclyl, optionally substituted heteroaryl, optionally substituted carbocyclyl, optionally substituted aralkyl, optionally substituted heteroaralkyl or —R^{c}-(optionally substituted heteroaryl); and the optionally substituted aryl, the optionally substituted heterocyclyl, the optionally substituted heteroaryl, the optionally substituted carbocyclyl, the optionally substituted aralkyl and the optionally substituted heteroaralkyl are substituted with 1-6 R¹⁰; wherein each R¹⁰ is independently selected from H, halogen, —CN, —NO₂, —CF₃, alkyl, —SR⁶, —OR⁶, —NR⁶R⁷, —NR⁶C(═O)(alkyl), —NR⁶C(═O)(cycloalkyl), —NR⁶C(═O)(heterocyclyl), —NR⁶C(═O)(aryl), —NR⁶C(═O)(heteroaryl), —C(═O)NR⁶R⁷, —C(═O)NR⁶(cycloalkyl), —C(═O)NR⁶(heterocycloalkyl), —C(═O)NR⁶(aryl), —C(═O)NR⁶(heteroaryl), —NR⁶C(═O)NR⁶R⁷, —NR⁶C(═O)NR⁷(cycloalkyl), —NR⁶C(═O)NR⁷(heterocycloalkyl), —NR⁶C(═O)NR⁷(aryl), —NR⁶C(═O)NR⁷(heteroaryl), —NR⁶C(═O)O(alkyl), —NR⁶C(═O)O(cycloalkyl), —NR⁶C(═O)O(heterocycloalkyl), —NR⁶C(═O)O(aryl), —NR⁶C(═O)O(heteroaryl), —NR⁶SO₂(alkyl), —NR⁶SO₂(cycloalkyl), —NR⁶SO₂(heterocycloalkyl), —NR⁶SO₂(aryl), —NR⁶SO₂(heteroaryl), —SO₂NR⁶R⁷, —SO₂NR⁶(cycloalkyl), —SO₂NR⁶(heterocycloalkyl), —SR⁶, —SO₂R⁶, —SO₂NR⁶(aryl), —SO₂NR⁶(heteroaryl), haloalkyl, aryl, heteroaryl, heterocyclyl and tetrazoyl.

In another aspect, provided herein are compounds of Formula (IIc), a pharmaceutically acceptable salt, solvate, polymorph, prodrug, metabolite, N-oxide, stereoisomer, or isomer thereof:

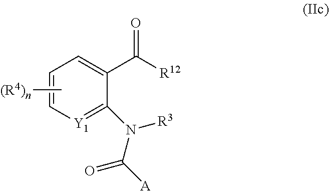

wherein:
Y₁ is N, CH, or CR⁴;
R¹² is —NR²R⁸ or —OR²;
R² and R³ are each independently selected from H and optionally substituted alkyl;
each R⁴ is independently selected from halogen, —CN, optionally substituted alkyl, optionally substituted alkoxy, and optionally substituted aryl;
R⁸ is optionally substituted alkyl, optionally substituted aryl, optionally substituted aralkyl, carbocyclyl, optionally substituted carbocyclylalkyl, optionally substituted heteroaryl, optionally substituted heteroarylalkyl, optionally substituted heterocyclyl, optionally substituted heterocyclylalkyl, —R^{b}COOR^{a} or —R^{b}CONR^{a}R^{a}; or R² and R⁸ taken together form an optionally substituted heterocycle with the nitrogen to which they are attached;
each R^{a} is independently selected from H and alkyl;
R^{b} is a bond or alkylenyl;
n is 0, 1, 2, or 3; and
A is optionally substituted heteroaryl, optionally substituted aryl or optionally substituted heterocyclyl.

In some embodiments of a compound of Formula (IIc), Y₁ is N or CH. In some embodiments of a compound of Formula (IIc), Y₁ is N. In some embodiments of a compound of Formula (IIc), A is optionally substituted heteroaryl, optionally substituted aryl or optionally substituted heterocyclyl; and the optionally substituted aryl, the optionally substituted heterocyclyl, the optionally substituted heteroaryl are substituted with 1-6 R¹⁰; wherein each R¹° is independently selected from H, halogen, —CN, —NO₂, —CF₃, alkyl, —SR⁶, —OR⁶, —NR⁶R⁷, —NR⁶C(═O)(alkyl), —NR⁶C(═O)(cycloalkyl), —NR⁶C(═O)(heterocyclyl), —NR⁶C(═O)(aryl), —NR⁶C(═O)(heteroaryl), —C(═O)NR⁶R⁷, —C(═O)NR⁶(cycloalkyl), —C(═O)NR⁶(heterocycloalkyl), —C(═O)NR⁶(aryl), —C(═O)NR⁶(heteroaryl), —NR⁶C(═O)NR⁶R⁷, —NR⁶C(═O)NR⁷(cycloalkyl), —NR⁶C(═O)NR⁷(heterocycloalkyl), —NR⁶C(═O)NR⁷(aryl), —NR⁶C(═O)NR⁷(heteroaryl), —NR⁶C(═O)O(alkyl), —NR⁶C(═O)O(cycloalkyl), —NR⁶C(═O)O(heterocycloalkyl), —NR⁶C(═O)O(aryl), —NR⁶C(═O)O(heteroaryl), —NR⁶SO₂(alkyl), —NR⁶SO₂(cycloalkyl), —NR⁶SO₂(heterocycloalkyl), —NR⁶SO₂(aryl), —NR⁶SO₂(heteroaryl), —SO₂NR⁶R⁷, —SO₂NR⁶(cycloalkyl), —SO₂NR⁶(heterocycloalkyl), —SR⁶, —SO₂R⁶, —SO₂NR⁶(aryl), —SO₂NR⁶(heteroaryl), haloalkyl, aryl, heteroaryl, heterocyclyl and tetrazoyl.

In some embodiments of a compound of Formula (IIc), R⁸ is optionally substituted alkyl, optionally substituted aralkyl, carbocyclyl, optionally substituted carbocyclylalkyl, optionally substituted heteroarylalkyl, optionally substituted heterocyclyl, or optionally substituted heterocyclylalkyl.

Also provided herein is a pharmaceutical composition comprising a compound of Formulas (I), (Ia), (Ib), (Ic), (Id), (II), (IIa), (IIb), or (IIc) or as described above and below, or a pharmaceutically acceptable salt, solvate, polymorph, prodrug, metabolite, N-oxide, stereoisomer, or isomer thereof, and a pharmaceutically acceptable excipient.

Further provided herein is a method to treat drug resistant and persistent tuberculosis in a mammal, the method comprising administering to the mammal a compound of Formulas (I), (Ia), (Ib), (Ic), (Id), (II), (IIa), (IIb), or (IIc) or as described above and below.

Also provided herein is a kit comprising: a biofilm formation media; and instructions for conducting a biofilm formation assay. The biofilm formation media may comprise M63 salts minimal medium. Also the biofilm formation media may comprise glucose, casamino acid, magnesium sulfate, calcium chloride, or any combination thereof. The kit may further comprise one or more agents. The one or more agents may comprise rifampicin (RIF), TMC207, isoniazid (INH), DMSO, or any combination thereof. The one or more agents may be a chemical compound, protein, nucleic acid, or any combination thereof. The protein may be an antibody, enzyme, receptor, kinase, and/or proteinase. The one or more agents may further be a bactericide. The kit may further comprise one or more cells. The one or more cells may be a bacterial cell. The one or more cells may be a escherichia, staphylococcus, and/or pseudomonas. The one or more cells may also be a mycobacterium. The one or more cells may be a Mycobacterium smegmatis cell. The instructions for conducting the biofilm formation assay comprise (i) instructions for culturing one or more cells; (ii) instructions for contacting the one or more cells with one or more agents; and (iii) instructions for assaying the biofilm formation of the one or more cells. The kit may further comprise one or more plate readers. The one or more plate readers may be a multilabel reader. The one or more plate readers comprises one or more detectors. The one or more detectors can enable wavelength reading, emission reading, barcode reading, or any combination thereof. The one or more plate readers may be an EnVision® Multilabel Reader.

Also provided here is a method comprising: a) culturing one or more cells in a biofilm formation media; b) contacting the one or more cells with one or more agents; and c) assaying a biofilm formation of the one or more cells. The method may further comprise identifying the one or more agents as a biofilm formation inhibitor based on the biofilm formation assay. Also, the method may further comprise identifying the one or more agents as a growth inhibitor based on the biofilm formation assay. The biofilm formation media may comprise M63 salts minimal medium. The biofilm formation media may comprise glucose, casamino acid, magnesium sulfate, calcium chloride, or any combination thereof. The one or more agents may comprise rifampicin (RIF), TMC207, isoniazid (INH), DMSO, or any combination thereof. The one or more agents may be a chemical compound, protein, nucleic acid, or any combination thereof. The protein may be an antibody, enzyme, receptor, kinase, and/or proteinase. The one or more agents may also a bactericide. The one or more cells may be a bacterial cell. The one or more cells may be a escherichia, staphylococcus, and/or pseudomonas. The one or more cells may be a mycobacterium. The one or more cells may be a Mycobacterium smegmatis cell. The assaying of the biofilm formation may comprise one or more plate readers. The one or more plate readers may be a multilabel reader. The one or more plate readers may comprise one or more detectors. The one or more detectors may enable wavelength reading, emission reading, barcode reading, or any combination thereof. The one or more plate readers may be an EnVision® Multilabel Reader. The assaying of the biofilm formation may comprise one or more detectors for detecting a wavelength, emission, barcode, or any combination thereof.

Further provided here is a method of treating a disease or condition in a subject in need thereof, the method comprising administering to the subject one or more agents, wherein the one or more agents are identified by the method for identifying the one or more agents as a biofilm formation inhibitor based on the biofilm formation assay. The disease or condition may be a pathogenic infection. The pathogenic infection may be a bacterial infection. The bacterial infection may be tuberculosis.

Also provided herein is a composition comprising one or more agents identified by the method identified by the method for identifying the one or more agents as a biofilm formation inhibitor based on the biofilm formation assay. The composition may further comprise an excipient, adjuvant, buffer, oil, gel, solution, or any combination thereof.

Further provided herein is a system for identifying one or more agents for treating a bacterial infection in a subject in need thereof, the system comprising: a) a biofilm formation media; and b) a plate reader. The biofilm formation media may comprise M63 salts minimal medium. The biofilm formation media comprises glucose, casamino acid, magnesium sulfate, calcium chloride, or any combination thereof. The system may further comprise one or more agents. The one or more agents may comprise rifampicin (RIF), TMC207, isoniazid (INH), DMSO, or any combination thereof. The one or more agents may be a chemical compound, protein, nucleic acid, or any combination thereof. The protein may be an antibody, enzyme, receptor, kinase, and/or proteinase. The one or more agents may be a bactericide. The system may further comprise one or more cells. The one or more cells may be a bacterial cell. The one or more cells may be a escherichia, staphylococcus, and/or pseudomonas. The one or more cells may be a mycobacterium. The one or more cells may be a Mycobacterium smegmatis cell. The one or more plate readers is a multilabel reader. The one or more plate readers may comprise one or more detectors. The one or more detectors may enable wavelength reading, emission reading, barcode reading, or any combination thereof. The one or more plate readers may be an EnVision® Multilabel Reader.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1 describes hit compounds from screen under biofilm culture condition: (A) chemical structures of active compounds; (B) TCA1 has selective activity against mycobacteria; (C) chemical structures of the affinity resin and the photo-affinity probe used in pull-down experiments.

FIG. 2 describes in vitro activity of TCA1: (A) kill-kinetics of Mtb by TCA1 (3.75 μg/ml) alone or in combination with INH (1 μg/ml) or RIF (2 μg/ml) in comparison to RIF (2 μg/ml) and INH (1 μg/ml) alone in 7H9 medium; (B) activity of TCA1 (3.75 μg/ml) against a RIF resistant Mtb strain and (C) INH resistant Mtb strain in 7H9 medium; (D) kill-kinetics of a XDR-TB strain by TCA1 (7.5 μg/ml) in 7H9 medium; (E) activity of TCA1 against non-replicating Mtb under nutrient starvation conditions; (F) qPCR analysis of expression ratios of selected genes from TCA1 (3.75 μg/ml) treated and untreated (0.1% DMSO) Mtb.

FIG. 3 describes in vivo efficacy of TCA1 in mouse models: (A) in an acute Mtb infection mouse model (2 week infection), followed by 4 weeks of drug treatment, TCA1 showed significant bactericidal activity in lungs and (B) spleen both alone (100 mg/kg) and in combination (40 mg/kg) with INH (25 mg/kg) or RIF (10 mg/kg); The low activity of RIF as a mono-therapy in this model is consistent with what has been observed in a previous study [Makarov V, et al. (2009) Benzothiazinones kill Mycobacterium tuberculosis by blocking arabinan synthesis. Science 324:801-804]; (C) in a chronic TB infection mouse model (4 week low dose infection), followed by 4 weeks of drug treatment, TCA1 showed activity in lungs and (D) spleen both alone (100 mg/kg) and in combination (40 mg/kg) with INH (25 mg/kg) or RIF (10 mg/kg) (p-value <0.05). Mice were gavaged with TCA1 once per day and 5 days/week. RIF and INH were administered in drinking water.

FIG. 4 describes TCA1 is a DprE1 inhibitor: (A) sequence alignment of DprE1 of M. smegatis and Mtb. A Y321C (Y314C in Mtb) mutation was identified in both M. smegatis and Mtb strains resistant to TCA1; (B) inhibition of DprE1 by TCA1 in the cell-free assay for DPA production analyzed by TLC and autoradiography; M. smegmatis membrane or cell envelope fractions were incubated with phospho-[14C]-ribose diphosphate and with 25 μg/ml TCA1, or BTZ043 (left) or TCA1 in a dose-response fashion (right). Both TCA1 and BTZ043 potently inhibit conversion of the substrate, decaprenylphosphoryl ribose (DPR) to the product, decaprenylphosphoryl arabinose (DPA) by DprE1/DprE2 epimerase; (C) molecular surface of Mtb DprE1 with the FAD domain in light blue and the substrate binding domain in beige; the surface areas in pale green and magenta indicate the positions of Cys387 and Tyr314 (left); non-covalent contacts between TCA1 and DprE1 (right); residues within a 4 Å-radius of the inhibitor (violet) are shown as sticks with FAD in yellow; dashed lines indicate shortest contacts (yellow=polar, orange=hydrophobic/van der Waals) between the residues and the inhibitor; Trp230, located within 4 Å of the carbamate moiety of TCA1, has been omitted for clarity.

FIG. 5 describes TCA1 inhibits MoCo biosynthesis: (A) Mtb overexpressing dprE1 is sensitive to TCA1 (7.5 μg/ml) under nutrient starvation conditions; (B) Mtb overexpressing dprE1 conferred resistance to TCA1 (3.75 μg/ml) in 7H9 medium; in the meantime, TCA1 acts synergistically with INH (1 μg/ml) or RIF (2 μg/ml) against the same strain; (C)-(F) MoCo inhibition assay; HPLC profiles of MoCo form “A” dephospho standard and sample extracted from Mtb. Arrow indicates the position of MoCo form “A” dephospho; (C) MoCo form “A” dephospho standard from synthetic source; (D) extracted sample from Mtb in the absence of TCA1; (E) extracted sample from Mtb after treatment with TCA1 (7.5 μg/ml, 18 h); (F) extracted sample from Mtb in the absence of TCA1, spiked with a MoCo form “A” dephospho standard; (G) kill-kinetics of wild-type Mtb and Mtb overexpressing moeW by 7.5 μg/ml TCA1 in media using nitrate as the only nitrogen source; (H) kill-kinetics of wild-type Mtb and Mtb overexpressing moeW by 7.5 μg/ml TCA1 under nutrient starvation conditions.

FIG. 6: In an acute TB infection mouse model (2 week infection), followed by 4 weeks of gavage (once per day and 5 days/week), TCA1 showed bactericidal activity both in lungs (A) and spleen (B). The dose of TCA1, INH and RIF is 40 mg/kg, 25 mg/kg and 10 mg/kg, respectively. RIF and INH were administered in drinking water.

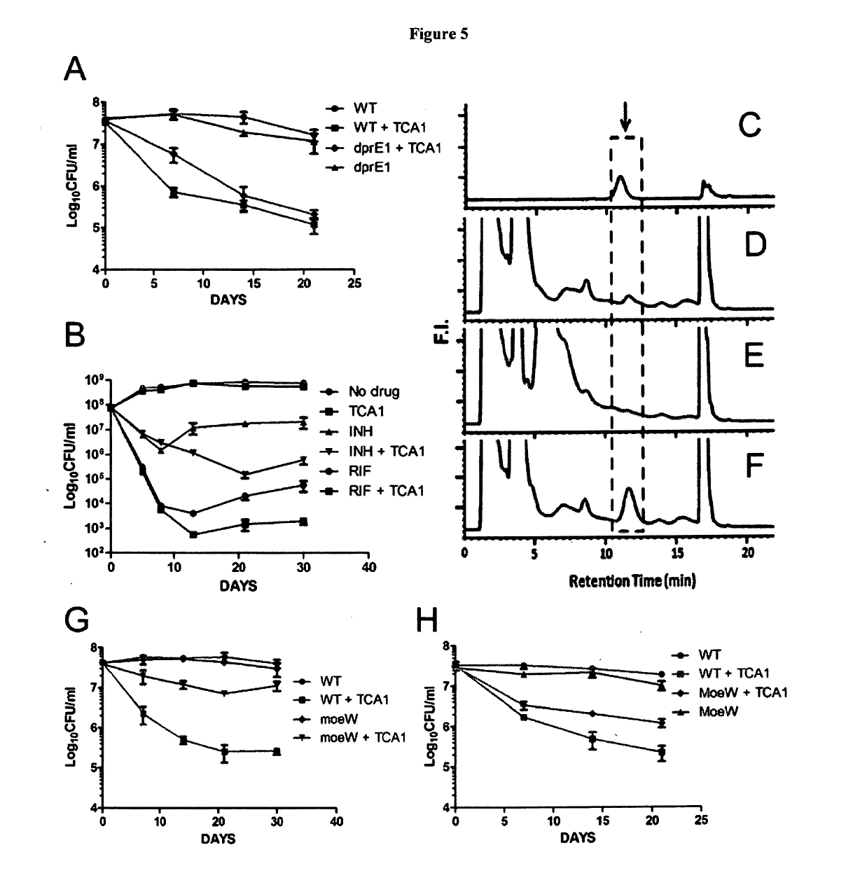

FIG. 7: DprE1 is incubated with the drug of interest (different concentration of TCA1) for 15 min. BTZ-BODIPY is added and the sample is incubated for 1 h at 37° C. Samples are then analyzed by SDS-PAGE [Coomassie staining (top) and fluorescence scan (bottom)]. Lane 1: 9 μM DprE1, 20 μM FAD, 20 μM BTZ-BODIPY; Lane 2-8: 9 μM DprE1, 20 μM FAD, 20 μM BTZ-BODIPY, plus TCA1 (0, 50, 25, 12.5, 6.3, 3.1, 1.6 μM).

FIG. 8 describes chemical structure of Molybdenum cofactor.

FIG. 9: In-gel fluorescence scanning Probe-labeled MoeW was detected by click conjugation to a rohodmine-azide reporter tag, followed by SDS PAGE and in-gel fluorescence scanning Lane 1&2, 3&4, 5&6, 7&8 are series of 2-fold dilution of the E. coli lysate. Lane 1, 3, 5, and 7 are lysate of E. coli cells without IPTG induction. Lane 2, 4, 6, and 8 are lysate of E. coli cells with IPTG induction. In lane 4, 6, and 8, a band with the size of MoeW is present, while it is absent in non-induced samples (pointed by red arrows). The band is not very strong because most of MoeW was found in inclusion body, not in soluble fraction, when overexpressed in E. coli.

### DETAILED DESCRIPTION OF THE INVENTION

MDR (Multi-Drug-Resistant) and XDR (eXtensively Drug-Resistant) Mtb strains are becoming widespread resulting in high failure rates despite the use of second and third line antibiotics and longer treatment times (up to 2 years). A new drug in drug regimen should shorten chemotherapy and overcome the emergence of resistance to have a real impact on TB.

Although numerous cell-based screens against Mtb have been performed, to date most screens are designed to identify molecules that are active against rapidly growing mycobacteria under growth-optimal laboratory conditions, and are inherently biased toward identifying bactericidal or bacteriostatic compounds against replicating Mtb [Pethe K, et al. (2010) A chemical genetic screen in Mycobacterium tuberculosis identifies carbon-source-dependent growth inhibitors devoid of in vivo efficacy. Nat Commun 1:57]. However, it is becoming apparent that the culture conditions used in a screen very much affect our ability to identify inhibitors that will be active in vivo [(a) Pethe K, et al. (2010) A chemical genetic screen in Mycobacterium tuberculosis identifies carbon-source-dependent growth inhibitors devoid of in vivo efficacy. Nat Commun 1:57; (b) Stanley S A, et al. (2012) Identification of novel inhibitors of M. tuberculosis growth using whole cell based high-throughput screening. ACS Chem Biol 7:1377-1384]. This issue is a particular concern in the development of drugs targeting persistent Mtb. Both target- and cell-based screens have been carried out under conditions that are thought to simulate those Mtb encounters during a chronic infection [(a) Mak P A, et al. (2012) A high-throughput screen to identify inhibitors of ATP homeostasis in non-replicating Mycobacterium tuberculosis. ACS Chem Biol 7:1190-1197; (b) Gold B, et al. (2012) Nonsteroidal anti-inflammatory drug sensitizes Mycobacterium tuberculosis to endogenous and exogenous antimicrobials. Proc Natl Acad Sci USA 109:16004-16011]. For example, it has been shown that oxygen deprivation or nutrient starvation in Mtb cultures triggers metabolic changes resulting in non-replicating, phenotypically drug resistant bacilli in vitro [(a) Wayne L G & Hayes L G (1996) An in vitro model for sequential study of shiftdown of Mycobacterium tuberculosis through two stages of nonreplicating persistence. Infect. Immun. 64:2062-2069; (b) Wayne L G & Sohaskey C D (2001) Nonreplicating persistence of Mycobacterium tuberculosis. Annu Rev Microbiol 55:139-163]. Indeed, anaerobic Mtb cultures are resistant to isoniazid (INH) and partly resistant to rifampicin (RIF), but highly sensitive to pyrazinamide (PZA) [Mitchison D A & Coates A R (2004) Predictive in vitro models of the sterilizing activity of anti-tuberculosis drugs. Curr Pharm Des 10:3285-3295], underscoring the differing drug sensitivities of Mtb in different metabolic states. Given the lack of clear consensus on cell culture conditions that best reflect the in vivo biology of Mtb, a screen was carried out based on in vitro biofilm formation in the hope of identifying compounds with new mechanism(s) of action that may be effective against drug resistant and persistent Mtb. The molecule TCA1, which was identified through this screen, not only showed bactericidal activity against both replicating (wild type and drug-resistant) and non-replicating Mtb, but also was efficacious in acute and chronic Mtb infection mouse models, both alone and in combination with INH or RIF. Moreover, genetic and biochemical studies showed that TCA1 functions by inhibiting two distinct biosynthetic pathways with concomitant down-regulation of genes known to be involved in mycobacterial persistence.

### A High Throughput Screen Under Biofilm Culture Conditions

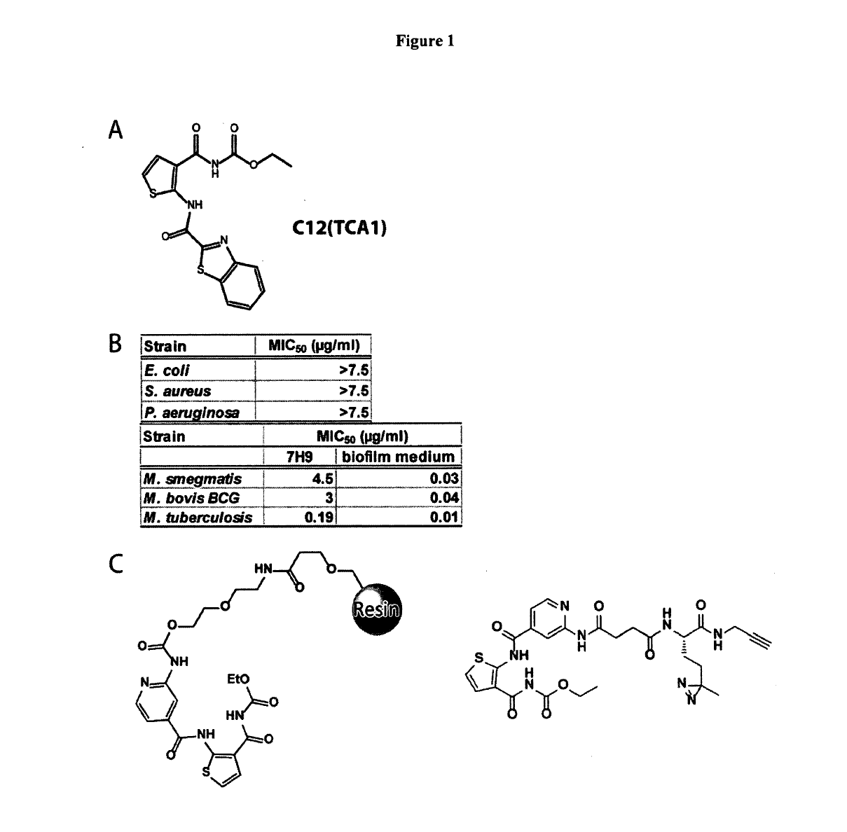

Pathogenic Mtb is not conducive to high-throughput screens involving automation as these experiments would need to be carried out in a biosafety level-3 facility. However, Mycobacterium smegmatis (M. smegmatis), a saprophytic, non-pathogenic mycobacteria that also forms in vitro biofilms [Ojha A, et al. (2005) GroEL1: a dedicated chaperone involved in mycolic acid biosynthesis during biofilm formation in mycobacteria. Cell 123:861-873] which induce drug-tolerance [Teng R & Dick T (2003) Isoniazid resistance of exponentially growing Mycobacterium smegmatis biofilm culture. FEMS Microbiol Lett 227:171-174], is amenable to high-throughput screening. Therefore, the primary cell-based screen was based upon the inhibition of biofilm formation in M. smegmatis. It was found that the in vitro biofilm, visualized as a pellicle that grows at the air-liquid interface, covered the whole surface of the well in a 384 well plate once formed, affording a high signal to noise ratio for positive hits. A diverse library of 70,000 heterocycles was screened (Supplemental information) which afforded 17 compounds with minimum inhibitory concentrations (MIC₅₀s) of less than 10 μM in a biofilm inhibition assay. Two classes of compounds were identified: one class inhibited the growth of mycobacteria under biofilm culture conditions, while the second class inhibited the formation of biofilms without significant growth inhibition. These hit compounds from the primary screen were then tested for their ability to inhibit in vitro biofilm growth in virulent Mtb H37Rv using a scaled-up 24-well assay as previously described [Ojha A K, et al. (2008) Growth of Mycobacterium tuberculosis biofilms containing free mycolic acids and harbouring drug-tolerant bacteria. Mol Microbiol 69:164-174]. Two compounds, C7 and TCA1 were found to also inhibit biofilm formation by Mtb H37Rv (FIG. 1A). TCA1, which displayed potent inhibitory activity against Mtb under both biofilm and planktonic culture conditions, was selected for further studies.

### In Vitro Bactericidal Activity

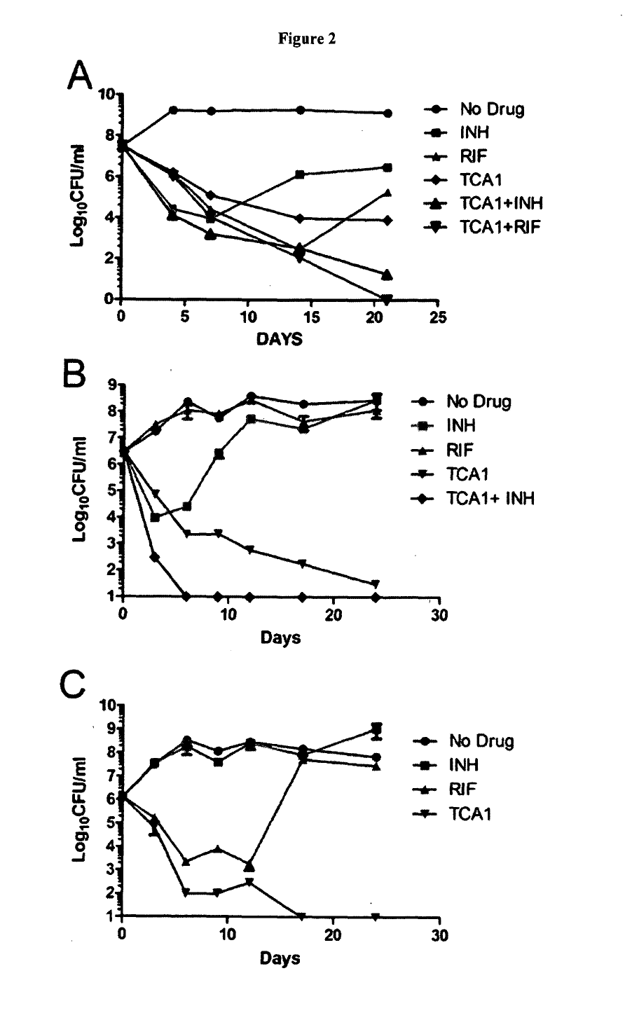

TCA1 showed selective inhibitory activity against bacterial growth—it is inactive against E. coli, S. aureus, and P. aeruginosa. The target for its bactericidal activity may be specific to the genus Mycobacterium (FIG. 1B). Interestingly, the activities of TCA1 against M. smegmatis, M. bovis BCG, and Mtb were 20-150 fold higher in biofilm medium (MIC₅₀ 0.03 μg/ml, 0.04 μg/ml and 0.01 μg/ml, respectively) than in 7H9 medium (MIC₅₀ 4.5 μg/ml, 3 μg/ml and 0.19 μg/ml, respectively). This observation underscored the variable efficacy of a drug in different growth media [Stanley S A, et al. (2012) Identification of novel inhibitors of M. tuberculosis growth using whole cell based high-throughput screening. ACS Chem Biol 7:1377-1384], which in part may result from the expression of distinct target genes and metabolic pathways. TCA1 was bactericidal with a MIC₉₉ of 2.1 μg/ml in solid medium. To evaluate the bactericidal activity of TCA1 against Mtb in comparison to the two frontline TB drugs, INH and RIF, a 21-day kinetic killing assay was performed using comparable levels of each of the three drugs (20×MIC₅₀ of each of the three drugs). TCA1 was active by itself against exponentially growing virulent Mtb in 7H9 media, with a more than 3 log reduction in the number of bacilli over a treatment period of 21 days. Treatment with INH or RIF resulted in a comparable drop in CFU over the first seven days of treatment, but the subsequent outgrowth of bacilli detected in INH and RIF treated cultures was absent in TCA1 treated cultures. Furthermore, TCA1 in combination with either RIF or INH was able to sterilize a Mtb culture in ˜3 weeks (FIG. 2A); removal of drug after 3 weeks of combination drug treatment did not lead to Mtb outgrowth.

The activity of TCA1 on drug resistant Mtb was also tested. RIF resistance is a marker for MDR-TB (90% of RIF resistant strains are also MDR) and typically requires 18-24 months of treatment. TCA1 by itself was active against a clinical strain that is resistant to RIF (due to a mutation in rpoB) and, more importantly, in combination with INH, sterilized the cultures within one week (FIG. 2B). Removal of both drugs after 3 weeks of treatment did not result in outgrowth. TCA1 was also found to be bactericidal against a strain with a mutation in katG (resulting in resistance to INH) (FIG. 2C). Finally, TCA1 was tested against an XDR-TB strain, mc28013, which is resistant to 10 TB drugs, including all front line drugs. TCA1 showed potent bactericidal activity against the XDR-TB strain (5 log CFU reduction in 3 weeks) (FIG. 2D). Given the lack of cross resistance to TCA1 in any of these drug-resistant strains, TCA1 may function by a distinct mechanism.

The activity of TCA1 was tested against non-replicating Mtb in a nutrient starvation assay, a widely used in vitro model of the Mtb dormancy phenotype [(a) Gengenbacher M, Rao S P, Pethe K, & Dick T (2010) Nutrient-starved, non-replicating Mycobacterium tuberculosis requires respiration, ATP synthase and isocitrate lyase for maintenance of ATP homeostasis and viability. Microbiology 156:81-87; (b) Betts J C, Lukey P T, Robb L C, McAdam R A, & Duncan K (2002) Evaluation of a nutrient starvation model of Mycobacterium tuberculosis persistence by gene and protein expression profiling. Mol Microbiol 43:717-731]. Under these conditions, Mtb enters a non-replicating state and has been shown to become tolerant to drugs, without acquiring heritable drug-resistance inducing mutations [Betts J C, Lukey P T, Robb L C, McAdam R A, & Duncan K (2002) Evaluation of a nutrient starvation model of Mycobacterium tuberculosis persistence by gene and protein expression profiling. Mol Microbiol 43:717-731]. TCA1 showed bactericidal activity against non-replicating Mtb at a concentration of 7.5 μg/ml (40×MIC₅₀ in 7H9 medium), reducing CFU by 3 logs in three weeks (FIG. 2E). Under the same assay conditions, RIF (40×MIC₅₀ in 7H9 medium) showed less bactericidal activity than TCA1. The activity of TCA1 was also tested in an intra-macrophage cell culture system to determine whether it is active against intracellular mycobacteria, since in the mouse model of infection and in humans, Mtb is believed to reside mainly in macrophages. TCA1 was found to be quite potent in an intracellular CFU assay with a MIC₅₀ of 0.6 μg/ml [MIC₅₀ (RIF)=2.7 μg/ml, MIC₅₀ (INH)=0.2 μg/ml]. Finally, TCA1 showed no cytotoxicity against five mammalian cell lines (Huh7, 293T, K562, HepG2, and Vero cells) at the highest concentration tested (100 μM for Vero cells and 25 μM for the others); and a hERG assay indicated that TCA1 had no activity at 30 μM).

### TCA1 is Efficacious in Acute and Chronic Mtb Infection Mouse Models

The activity of TCA1 was examined in a mouse model of Mtb infection. The physical and pharmacokinetic (PK) characteristics of TCA1 were determined. It was stable to proteolytic activity in human or mouse plasma for up to 4 hours. Moreover, a GSH trapping assay indicated no GSH adduct was formed, and TCA1 had no inhibitory activity against four CYP enzymes. After intra-venous (IV) administration, TCA1 exhibited a low clearance (CL) and steady-state volume of distribution (Vss) with an elimination half life of 0.73 hour. Following oral administration of 20 and 50 mg/kg in solution formulation, TCA1 showed a high Cₘₐₓ (2122 and 5653 nM, respectively), moderate exposure with oral bioavailability ranging from 19%-46% and a half life of 1.8 hours.

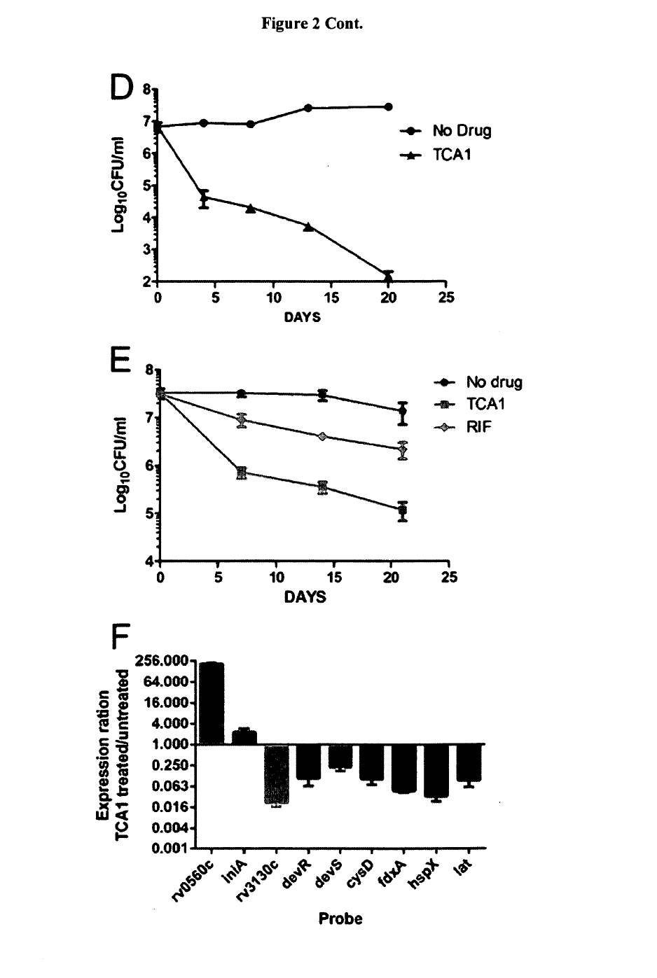

The in vivo efficacy experiments were initially performed in an acute infection model with a low dose of TCA1. BALB/c mice were infected with a low dose of Mtb H37Rv (˜200 bacilli). Two weeks after infection, mice were treated with TCA1 (40 mg/kg), INH (25 mg/kg) or RIF (10 mg/kg) for 4 weeks [dosed once per day, 5 days per week;]. The doses of INH and RIF were consistent with those published in the literature [Makarov V, et al. (2009) Benzothiazinones kill Mycobacterium tuberculosis by blocking arabinan synthesis. Science 324:801-804]. After 4 weeks of treatment with TCA1—the CFU dropped 0.5 log in lung and 1.5 logs in spleen, which was comparable to the potency of RIF, but less than that of INH. The gross pathology and histopathology also showed significant improvement in both tissues. The in vivo efficacy of TCA1 (40 mg/kg) was also tested in combination with INH (25 mg/kg) or RIF (10 mg/kg). In the acute infection model, TCA1+INH and TCA1+RIF showed nearly a 2 and 3 log CFU reduction in lung, respectively, and a more than 3 log CFU reduction in spleen (FIGS. 3A and 3B). There is a greater CFU drop in the lungs of mice treated by the combination of TCA1 and INH relative to the combination of INH and RIF.

The compound was also tested in a mouse model of chronic TB infection. Mice were challenged with a low dose aerosol infection and treatment was initiated 4 weeks after infection. Similar combination treatments were efficacious in the chronic infection model as well (FIGS. 3C and 3D). Because the mice were able to tolerate 40 mg/kg TCA1, the dose was increased to 100 mg/kg using a similar protocol as in the acute infection model. After 4 weeks, the CFU dropped nearly 2 log units in lungs and more than 3 log units in spleen, demonstrating that the in vivo bactericidal activity of TCA1 is dose-dependent (FIGS. 3A and 3B). The mice again showed no obvious adverse effects or weight loss after 4 weeks of treatment. The compound was tested in the chronic infection model at 100 mg/kg. Again, TCA1 demonstrated efficacy in both lung (1 log CFU reduction) and spleen (1.4 log CFU reduction) (FIGS. 3C and 3D). These results demonstrate that the in vitro efficacy of TCA1 is recapitulated in vivo. Thus, in vitro mycobacterial biofilm may be a useful phenotype to identify novel compounds effective against Mtb in vivo either alone or in combination with existing TB drugs.

### Mechanism of Action Studies of TCA

To gain insight into the mechanism of action of TCA1, Mtb H37Rv was treated with TCA1 (3.75 μg/ml) in 7H9 media and carried out genome-wide transcriptional analysis. Similar to INH and ethambutol [Boshoff H I, et al. (2004) The transcriptional responses of Mycobacterium tuberculosis to inhibitors of metabolism: novel insights into drug mechanisms of action. J Biol Chem 279:40174-40184], cell wall and fatty acid biosynthetic genes were affected by TCA1 treatment, perhaps because TCA1 may interfere with these pathways. Unlike other known TB drugs, 10 of the 86 genes differentially downregulated compared to the DMSO control are genes previously implicated in TB dormancy, stress response, and RIF susceptibility. These include rv3130c-rv3134c, fdxA, and hspX (members of the dos regulon), cysD, and rv3288c-rv3290c (members of the sigF regulon). The microarray results were confirmed by qPCR (FIG. 2F). Most of these genes are part of the dormancy regulon controlled by dosR [Voskuil M I, et al. (2003) Inhibition of respiration by nitric oxide induces a Mycobacterium tuberculosis dormancy program. J Exp Med 198:705-713], and are upregulated under hypoxic conditions or by nitric oxide exposure. For example, fdxA, a low-redox-potential electron carrier, was highly upregulated in Mtb under hypoxic conditions [Muttucumaru D G, Roberts G, Hinds J, Stabler R A, & Parish T (2004) Gene expression profile of Mycobacterium tuberculosis in a non-replicating state. Tuberculosis (Edinb) 84:239-246], but significantly downregulated (>20 fold) in response to TCA1 treatment. Likewise, rv3130c was induced (>300 fold) under multiple-stress conditions [Deb C, et al. (2009) A novel in vitro multiple-stress dormancy model for Mycobacterium tuberculosis generates a lipid-loaded, drug-tolerant, dormant pathogen. PLoS One 4:e6077], but was downregulated (>30 fold) by TCA1. This downregulation of genes involved in dormancy and drug tolerance appears to be unique to TCA1. As such, TCA1 may potentially sensitize Mtb to killing by antibiotics.

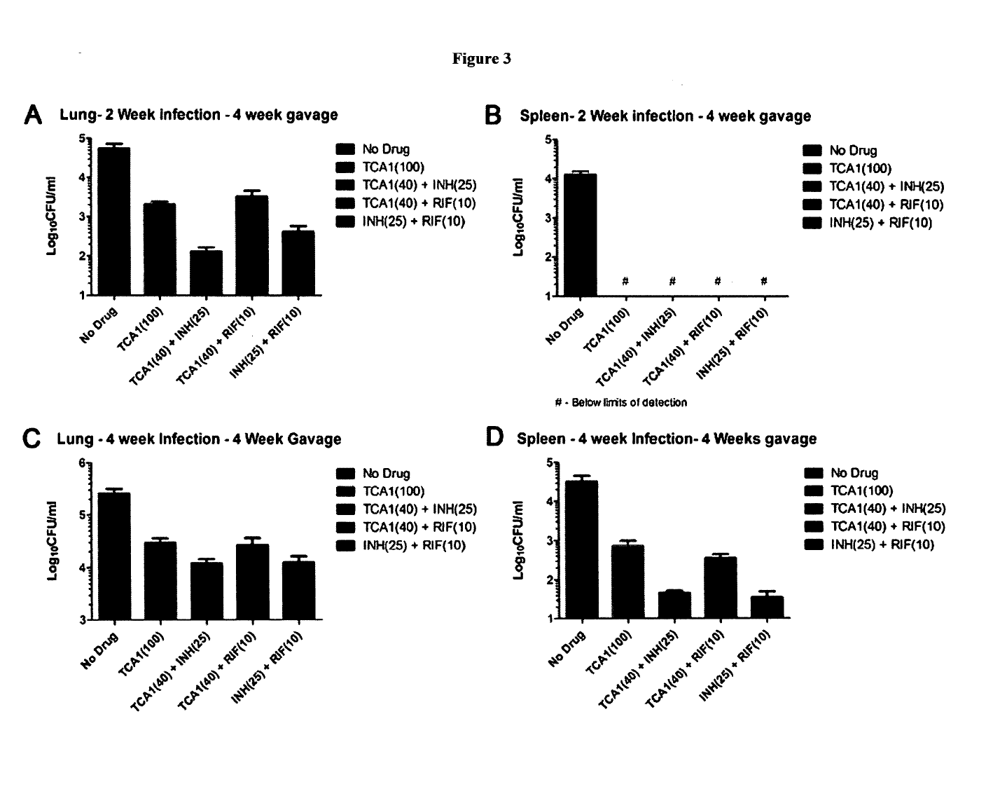

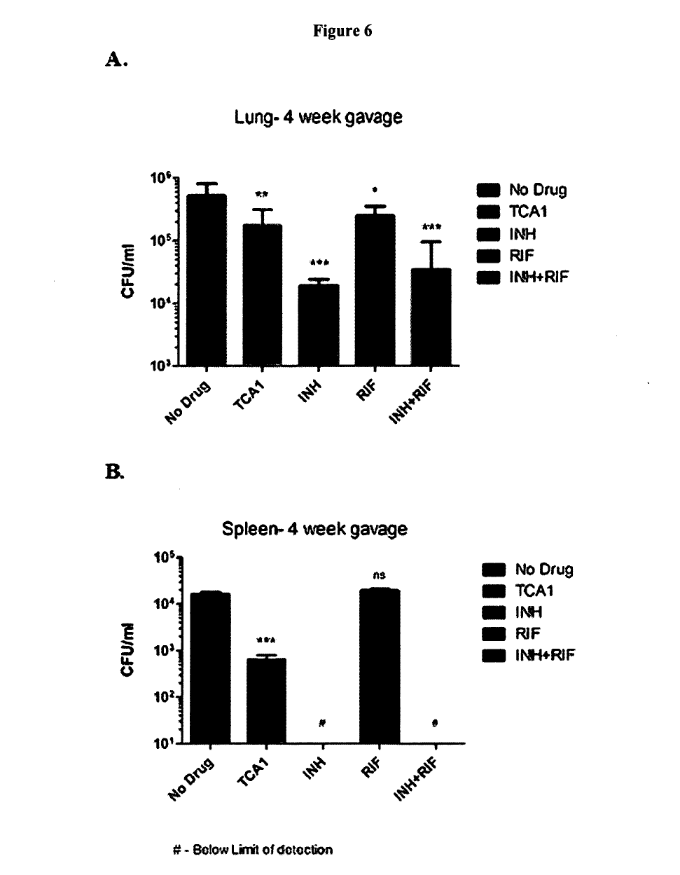

To further explore the mechanism of action of TCA1, a TCA1 resistant mutant that carries the cosmid (MSMEG_6379-MSMEG_6384) was isolated by selection of M. smegmatis, transformed with a genomic cosmid library and grown in biofilm formation medium. Overexpression of each gene in this cosmid revealed that MSMEG 6382, which is homologous to rv3790 in the Mtb genome, conferred high-level resistance to TCA1 (>20×MIC₅₀) in both M. smegmatis and Mtb. Spontaneous resistant mutants of M. smegmatis and Mtb were isolated, even though the spontaneous mutation rate to TCA1 resistance was extremely low (10⁻⁸-10⁻⁹). Whole genome sequencing of the genomic DNA of the resistant mutants revealed that they all have a single point mutation resulting in the amino acid replacement Tyr321Cys in MSMEG_6382 and Tyr314Cys in rv3790 (FIG. 4A). rv3790, encodes DprE1, a component of the essential decaprenylphosphoryl-β-D-ribofuranose 2′-epimerase (DprE1/DprE2) required for cell wall arabinan biosynthesis. Indeed, TCA1 suppressed the activity of M. smegmatis DprE1 in membrane and cell envelope enzymatic fractions in a dose-dependent manner (FIG. 4B). DprE1 was previously identified as the target of the benzothiazinones (BTZ) and a nitro-triazole molecule [(a) Stanley S A, et al. (2012) Identification of novel inhibitors of M. tuberculosis growth using whole cell based high-throughput screening. ACS Chem Biol 7:1377-1384; (b) Makarov V, et al. (2009) Benzothiazinones kill Mycobacterium tuberculosis by blocking arabinan synthesis. Science 324:801-804]. Both scaffolds contain active nitro-moieties and are believed to covalently modify Cys387 upon activation. A competitive binding assay was performed using a fluorescently labeled BTZ analog. TCA1 potently competed with BTZ in binding to DprE1, indicating that the binding site of TCA1 may overlap with BTZ (FIG. 7). However, TCA1 does not have an active nitro-moiety and the Tyr314Cys mutant strain that is resistant to TCA1 is sensitive to BTZ. As such, the binding mechanism of TCA1 may be different from these nitro-heterocycles.

To determine the molecular basis by which TCA1 inhibits DprE1, the crystal structure of the enzyme bound to TCA1 was determined. The overall structure of the DprE1-TCA1 complex was largely unaltered compared to that of the ligand-free protein with the same crystal symmetry [Batt S M, et al. (2012) Structural basis of inhibition of Mycobacterium tuberculosis DprE1 by benzothiazinone inhibitors. Proc Natl Acad Sci USA 109:11354-11359]. The enzyme, which is structurally related to the vanillyl-alcohol oxidase family of flavoproteins [Mattevi A, Fraaije M W, Coda A, & van Berkel W J (1997) Crystallization and preliminary X-ray analysis of the flavoenzyme vanillyl-alcohol oxidase from Penicillium simplicissimum. Proteins 27:601-603], consists of an FAD-binding and a substrate binding domain with the flavin moiety of FAD positioned at the interface between the two domains (FIG. 4C, left). The substrate binding domain includes two disordered loop regions (residues 269-283, 316-330) that leave the active site open and accessible for inhibitors. TCA1 was bound in the central cavity of the enzyme, adjacent to the isoalloxazine ring of FAD, in a boomerang-like conformation with the thiophene moiety inserted deeply into the bottom of the active site (FIG. 4C, right). The benzothiazole ring was oriented roughly parallel to the isoalloxazine of FAD. Non-covalent interactions between TCA1 and the enzyme were dominated by hydrophobic and van der Waals interactions (FIG. 4C), with the flavin contributing a large fraction of the total contact surface. Polar contacts were sparse, but include the H-bonds between the carboxamido group and thiazole nitrogen of TCA1 and Nζ of Lys418 (3.0 Å and 3.1 Å, respectively). The carbamate moiety made van der Waals interactions with the phenyl ring of Tyr314, consistent with the observation that substituting this tyrosine with cysteine renders DprE1 insensitive to TCA1. Superimposition of the structures of DprE1 bound to the BTZ analog (CT325) [Batt S M, et al. (2012) Structural basis of inhibition of Mycobacterium tuberculosis DprE1 by benzothiazinone inhibitors. Proc Natl Acad Sci USA 109:11354-11359] and TCA1 showed that the binding sites of these two inhibitors overlap significantly.

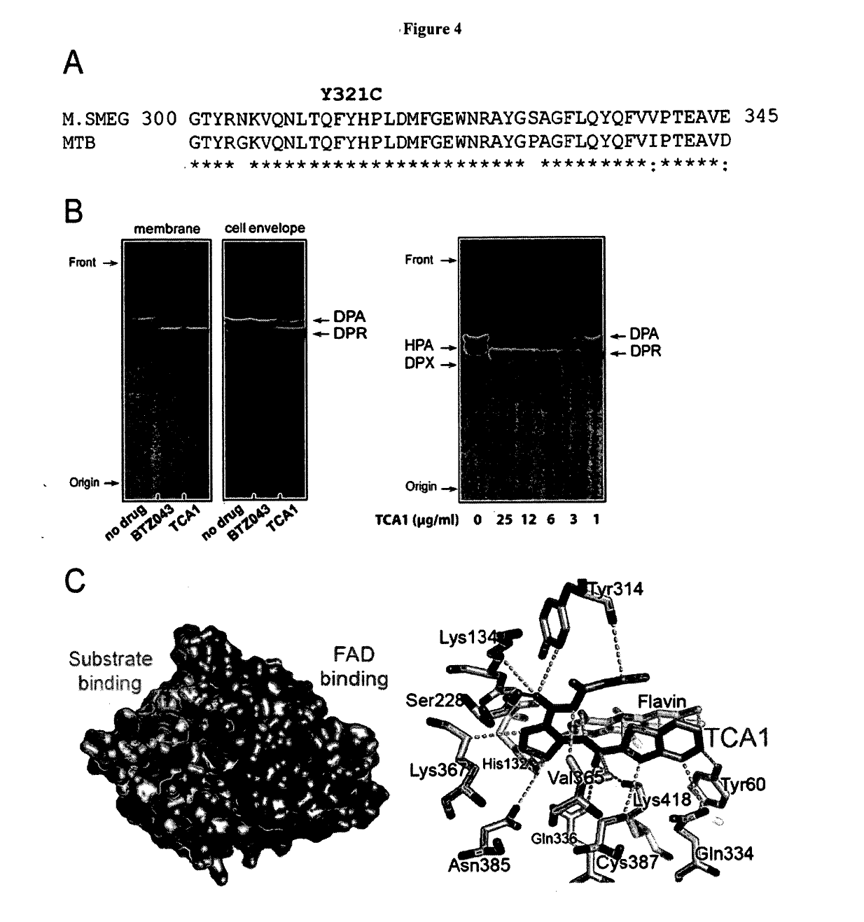

Given the above results, DprE1 may be a relevant target for the bactericidal activity of TCA1 against replicating bacteria, similar to BTZ. However, there are some clear distinctions between TCA1 and BTZ. First, BTZ is not active against non-replicating Mtb [Makarov V, et al. (2009) Benzothiazinones kill Mycobacterium tuberculosis by blocking arabinan synthesis. Science 324:801-804], while TCA1 is active against replicating and non-replicating Mtb. Secondly, the gene expression profiles of Mtb treated by two compounds are also very different—TCA1 downregulates persistence genes that are usually upregulated in Mtb dormant models, whereas BTZ does not [Makarov V, et al. (2009) Benzothiazinones kill Mycobacterium tuberculosis by blocking arabinan synthesis. Science 324:801-804]. The Mtb strain overexpressing DprE1 is resistant to TCA1 in 7H9 medium, but still sensitive to TCA1 in the nutrient starvation model (FIG. 5A). Moreover, TCA1 still potentiates INH or RIF on this DprE1 overexpressing strain (FIG. 5B). Thus, TCA1 could potentially act on an additional mycobacterial target.

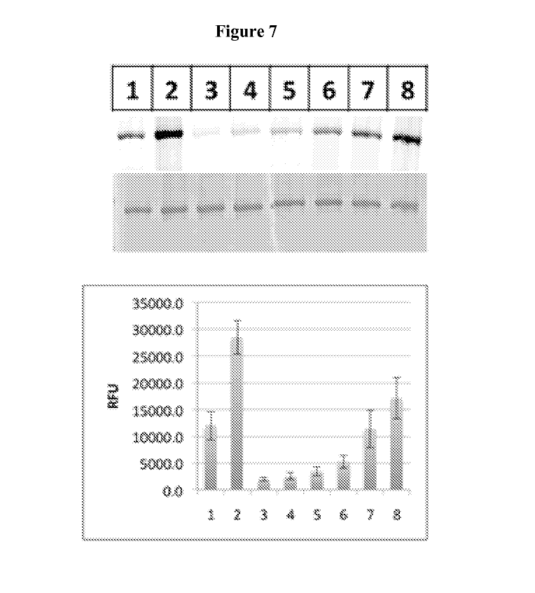

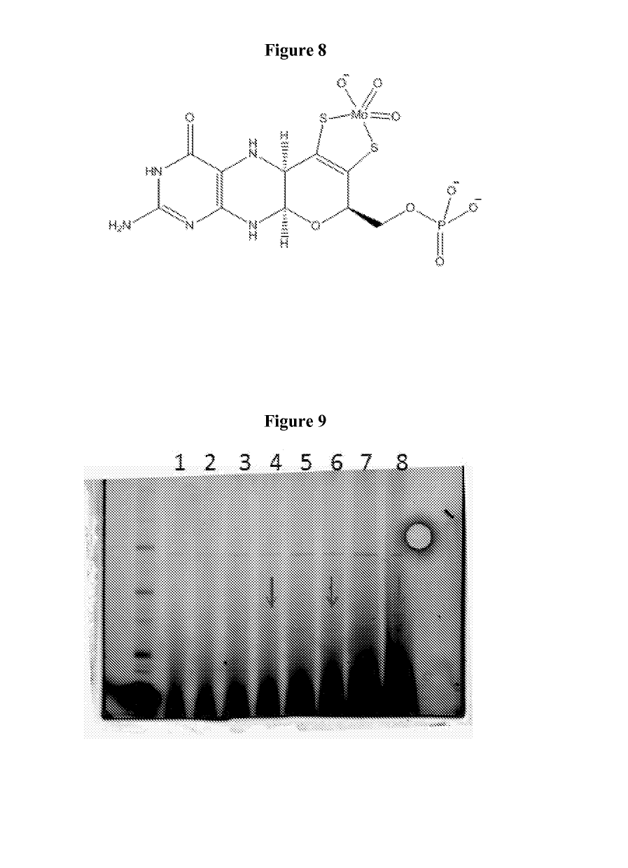

Because TCA1 had diminished activity against the DprE1(Y314C) mutant in normal growth medium, it is possible that a second TCA1 target is not essential for Mtb growth under conditions of optimal growth. This makes the selection of relevant mutants more difficult, and therefore affinity-based methods were used to identify additional potential targets. Among a group of analogs of TCA1, it was found that a pyridyl analog, TCA17, had very similar in vitro activity to that of TCA1. TCA17 was immobilized on a resin through a linker moiety (TCAP1; FIG. 1C) and used in a pull-down experiment with cell lysates from Mtb. A 35 kDa band was identified on an SDS-polyacrylamide gel electrophoresis gel (SDS-PAGE) after silver staining which disappeared in the presence of 50 μM TCA1 as competitor. Mass spectrometry identified the band as MoeW, a protein involved in the biosynthesis of the molybdenum cofactor (MoCo) (FIG. 8) with homologs in only a few bacterial genomes. To confirm the binding of TCA1 to MoeW, moeW was overexpressed in E. coli (which lacks a moeW gene homolog in its genome) and treated this strain with a photoaffinity probe analogous to TCA1 (TCAP2; FIG. 1C) followed by UV irradiation and cell lysis. As shown in FIG. 9, a band with the size of MoeW was present on a SDS-PAGE gel for the sample from the moeW-induced strain and absent in the un-induced control sample. These results demonstrate that TCA1 scaffold directly binds to MoeW.

MoeW is predicted to contain a FAD/NAD binding domain by protein sequence analysis, but its function has yet to be determined. The gene encoding MoeW is only conserved in Mtb and BCG, not in M. smegmatis or other mycobacterial species, although it is homologous to moeB, another gene involved in MoCo biosynthesis pathway and conserved in all mycobacteria species and many other bacteria [Williams M J, Kana B D, & Mizrahi V (2011) Functional analysis of molybdopterin biosynthesis in mycobacteria identifies a fused molybdopterin synthase in Mycobacterium tuberculosis. J Bacteriol 193:98-106]. All molybdenum-utilizing enzymes identified to date contain MoCo. MoCo is essential for the nitrate respiratory and assimilatory function of Mtb nitro-reductase. Some of these nitro-reductases have been found to be involved in the response of Mtb to hypoxia and nitric oxide [(a) Williams M J, Kana B D, & Mizrahi V (2011) Functional analysis of molybdopterin biosynthesis in mycobacteria identifies a fused molybdopterin synthase in Mycobacterium tuberculosis. J Bacteriol 193:98-106; (b) Malm S, et al. (2009) The roles of the nitrate reductase NarGHJI, the nitrite reductase NirBD and the response regulator GlnR in nitrate assimilation of Mycobacterium tuberculosis. Microbiology 155:1332-1339]. To determine if TCA1 can block the biosynthesis of MoCo, cell extracts were analyzed from Mtb treated with TCA1 by detection of dephosphorylated MoCo form “A” using HPLC with fluorescence detection. Indeed, TCA1 (7.5 μg/ml) completely abolished the formation of MoCo in Mtb (FIG. 5C-5F). It is known that MoCo is indispensable for nitrate assimilation by Mtb and thus essential for Mtb to survive in media which uses nitrate as the only nitrogen source (designated as nitrate media). An Mtb strain overexpressing moeW was generated, and it was found to confer resistance to TCA1 in nitrate media over 30 days of treatment (FIG. 5G). These results clearly demonstrate that TCA1 asserts its activity against Mtb by inhibition of MoCo biosynthesis through interaction with MoeW. The Mtb strain overexpressing moeW conferred resistance to TCA1 in nutrient starvation model over 21 days of treatment as well (FIG. 5H), but the lower level of resistance to TCA1 in the nutrient starvation model than in nitrate media indicates that the mechanism of action of TCA1 may be more complicated under the former condition. Nevertheless, the biochemical and genetic results clearly demonstrate MoeW is a relevant target of TCA1.

A novel cell-based screen was developed involving the growth of mycobacteria as an in vitro biofilm (a pellicle). The natural mode of growth of Mtb in liquid culture in the absence of detergent is as a pellicle at the liquid-air interface. Indeed, BCG is grown as a pellicle for vaccine production. This assay allowed for the identification of a potent inhibitor TCA1 against both replicating and non-replicating Mtb as well as drug-resistant Mtb. TCA1 functions by a unique mechanism involving downregulation of persistence genes and inhibition of both cell wall and MoCo biosynthesis. Moreover, TCA1 showed excellent in vivo efficacy in both acute and chronic TB infection mouse models. This compound served as a lead for the development of a new class of drugs against persistent and drug resistant Mtb. Indeed, compounds presented herein were identified with activities under both aerobic and anaerobic conditions. This work underscores the power of cell-based phenotypic screens to uncover molecules with novel mechanisms of action that provide new approaches to the treatment of human disease.

### DEFINITIONS

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“Amino” refers to the —NH₂ radical.

“Cyano” or “nitrile” refers to the —CN radical.

“Hydroxy” or “hydroxyl” refers to the —OH radical.

“Nitro” refers to the —NO₂ radical.

“Oxo” refers to the ═O substituent.

“Oxime” refers to the ═N—OH substituent.

“Thioxo” refers to the ═S substituent.

“Alkyl” refers to a straight or branched hydrocarbon chain radical, has from one to thirty carbon atoms, and is attached to the rest of the molecule by a single bond. Alkyls comprising any number of carbon atoms from 1 to 30 are included. An alkyl comprising up to 30 carbon atoms is referred to as a C₁-C₃₀ alkyl, likewise, for example, an alkyl comprising up to 12 carbon atoms is a C₁-C₁₂ alkyl. Alkyls (and other moieties defined herein) comprising other numbers of carbon atoms are represented similarly. Alkyl groups include, but are not limited to, C₁-C₃₀ alkyl, C₁-C₂₀ alkyl, C₁-C₁₅ alkyl, C₁-C₁₀ alkyl, C₁-C₈ alkyl, C₁-C₆ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (iso-propyl), n-butyl, i-butyl, s-butyl, n-pentyl, 1,1-dimethylethyl (t-butyl), 3-methylhexyl, 2-methylhexyl, vinyl, allyl, propynyl, and the like. Alkyl comprising unsaturations include alkenyl and alkynyl groups. Unless stated otherwise specifically in the specification, an alkyl group may be optionally substituted as described below.

“Alkylene”, “alkylenyl” or “alkylene chain” refers to a straight or branched divalent hydrocarbon chain, as described for alkyl above. Unless stated otherwise specifically in the specification, an alkylene group may be optionally substituted as described below.

“Alkoxy” refers to a radical of the formula —ORₐ. where Rₐ is an alkyl radical as defined. Unless stated otherwise specifically in the specification, an alkoxy group may be optionally substituted as described below.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group may be optionally substituted as described below.

“Alkenylene” or “alkenylenyl” refers to a straight or branched hydrocarbon chain consisting solely of carbon and hydrogen atoms, containing at least one carbon-carbon double bond, and having from two to twelve carbon atoms, as described for alkenyl above. Unless stated otherwise specifically in the specification, an alkenylene group may be optionally substituted as described below.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group may be optionally substituted as described below.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Aralkyl” refers to a radical of the formula —R^{d}-aryl where R^{d} is an alkylene chain as defined above, for example, methylene, ethylene, and the like. Unless stated otherwise specifically in the specification, the alkylene chain part of the aralkyl radical is optionally substituted as described above for an alkylene chain. Unless stated otherwise specifically in the specification, the aryl part of the aralkyl radical is optionally substituted as described above for an aryl group.

“Aralkoxy” refers to a radical bonded through an oxygen atom of the formula —O—R^{d}-aryl where R^{d} is an alkylene chain as defined above, for example, methylene, ethylene, and the like. Unless stated otherwise specifically in the specification, the alkylene chain part of the aralkyl radical is optionally substituted as described above for an alkylene chain. Unless stated otherwise specifically in the specification, the aryl part of the aralkyl radical is optionally substituted as described above for an aryl group.

“Cycloalkyl”, “carbocyclyl” or “carbocycle” refers to a stable, non-aromatic, monocyclic or polycyclic carbocyclic ring, which may include fused or bridged ring systems, which is saturated or unsaturated. Representative cycloalkyls or carbocycles include, but are not limited to, cycloalkyls having from three to fifteen carbon atoms, from three to ten carbon atoms, from three to eight carbon atoms, from three to six carbon atoms, from three to five carbon atoms, or three to four carbon atoms. Monocyclic cycloalkyls or carbocycles include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Unless otherwise stated specifically in the specification, a cycloalkyl or carbocycle group may be optionally substituted. Illustrative examples of cycloalkyl groups include, but are not limited to, the following moieties:

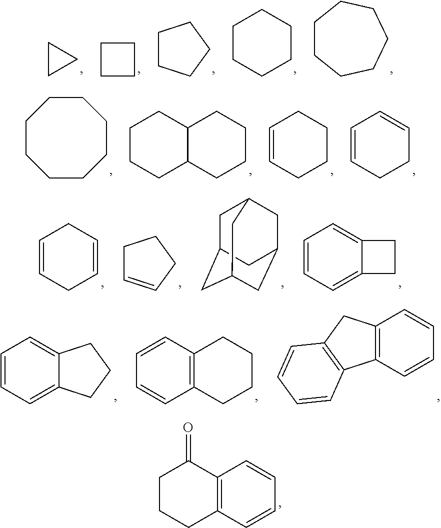

and the like.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” similarly refers to a radical of the formula —ORₐ where Rₐ is a haloalkyl radical as defined. Unless stated otherwise specifically in the specification, a haloalkoxy group may be optionally substituted as described below.

“Heterocycloalkyl” or “heterocyclyl” or “heterocyclic ring” or “heterocycle” refers to a stable 3- to 24-membered non-aromatic ring radical comprising 2 to 23 carbon atoms and from one to 8 heteroatoms selected from the group consisting of nitrogen, oxygen, phosphorous and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 12-crown-4, 15-crown-5, 18-crown-6, 21-crown-7, aza-18-crown-6, diaza-18-crown-6, aza-21-crown-7, and diaza-21-crown-7. Unless stated otherwise specifically in the specification, a heterocyclyl group may be optionally substituted. Illustrative examples of heterocycloalkyl groups, also referred to as non-aromatic heterocycles, include:

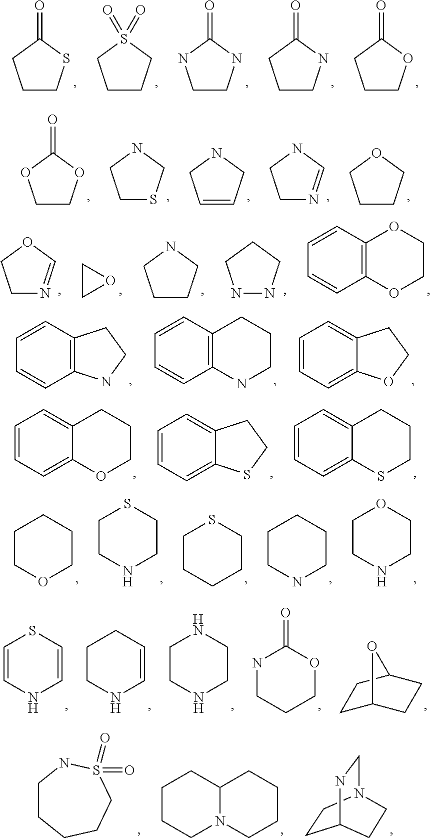

and the like. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo [b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group may be optionally substituted.

“Heteroarylalkyl” refers to a radical of the formula —R^{d}-heteroaryl, where R^{d} is an alkylene chain as defined above. If the heteroaryl is a nitrogen-containing heteroaryl, the heteroaryl is optionally attached to the alkyl radical at the nitrogen atom. Unless stated otherwise specifically in the specification, the alkylene chain of the heteroarylalkyl radical is optionally substituted as defined above for an alkylene chain. Unless stated otherwise specifically in the specification, the heteroaryl part of the heteroarylalkyl radical is optionally substituted as defined above for a heteroaryl group.

All the above groups may be either substituted or unsubstituted. The term “substituted” as used herein means any of the above groups (e.g, alkyl, alkylene, alkoxy, aryl, cycloalkyl, haloalkyl, heterocyclyl and/or heteroaryl) may be further functionalized wherein at least one hydrogen atom is replaced by a bond to a non-hydrogen atom substituent. Unless stated specifically in the specification, a substituted group may include one or more substituents selected from: oxo, amino, —CO₂H, nitrile, nitro, hydroxyl, thiooxy, alkyl, alkylene, alkoxy, aryl, cycloalkyl, heterocyclyl, heteroaryl, dialkylamines, arylamines, alkylarylamines, diarylamines, trialkylammonium (—N⁺R₃), N-oxides, imides, and enamines; a silicon atom in groups such as trialkylsilyl groups, dialkylarylsilyl groups, alkyldiarylsilyl groups, triarylsilyl groups, perfluoroalkyl or perfluoroalkoxy, for example, trifluoromethyl or trifluoromethoxy. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NH₂, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. In addition, each of the foregoing substituents may also be optionally substituted with one or more of the above substituents. Furthermore, any of the above groups may be substituted to include one or more internal oxygen, sulfur, or nitrogen atoms. For example, an alkyl group may be substituted with one or more internal oxygen atoms to form an ether or polyether group. Similarity, an alkyl group may be substituted with one or more internal sulfur atoms to form a thioether, disulfide, etc.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

“Pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable salt” refers to a salt of a compound that is pharmaceutically acceptable and that possesses the desired pharmacological activity of the parent compound.

“Pharmaceutically acceptable excipient, carrier or adjuvant” refers to an excipient, carrier or adjuvant that may be administered to a subject, together with at least one compound of the present disclosure, and which does not destroy the pharmacological activity thereof and is nontoxic when administered in doses sufficient to deliver a therapeutic amount of the compound. “Pharmaceutically acceptable vehicle” refers to a diluent, adjuvant, excipient, or carrier with which at least one compound of the present disclosure is administered.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition. In other embodiments, treatment also includes prophylactic treatment (e.g., administration of a composition described herein when an individual is suspected to be suffering from a bacterial infection).

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

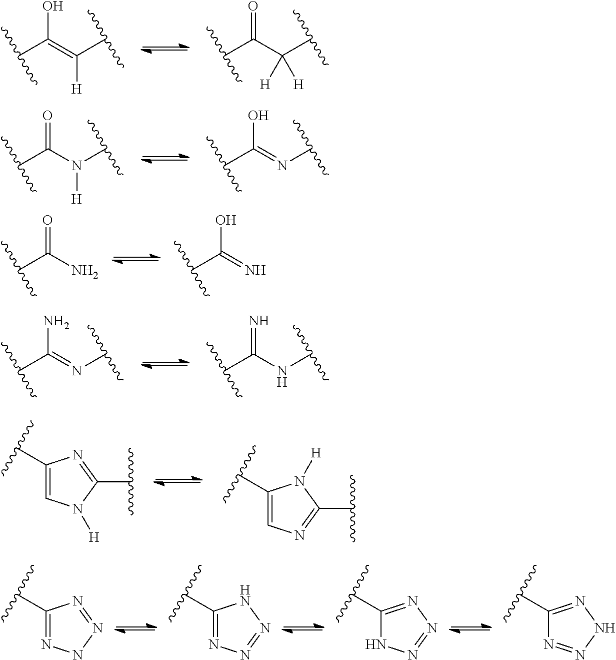

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

### Compounds

Described herein are compounds that treat drug resistant and persistent tuberculosis.

In one aspect, provided herein are compounds of Formula (I), a pharmaceutically acceptable salt, solvate, polymorph, prodrug, metabolite, N-oxide, stereoisomer, or isomer thereof:

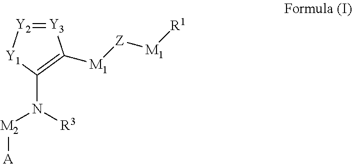

wherein:
M₁ are each independently a bond, —C(═O)— or —S(═O)₂—;
M₂ is —(CH₂)—, —C(═O)— or —S(═O)₂—;
Z is a bond or NR²;
Y₁ is S, O, or NR²;
Y₂ is CR⁴ or N;
Y₃ is CR⁵ or N;
R¹ is —O-(optionally substituted alkyl), —O-(alkenyl), —O-(alkenyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), —O-(alkyl)-(NR⁶R⁷), —NR⁶R⁷, or R⁸;
R² and R³ are each independently selected from H, optionally substituted alkyl, and optionally substituted aryl; or R¹ and R² taken together form a heterocycle;
R⁴ is H, halogen, —CN, optionally substituted alkyl, optionally substituted aryl, —R^{b}COOR^{a} or —R^{b}CH(COOR^{a})₂;
R⁵ is H, halogen, optionally substituted alkyl or cycloalkyl; or R⁴ and R⁵ taken together form a carbocycle or an optionally substituted heterocycle;
R⁶ and R⁷ are each independently selected from H and optionally substituted alkyl; or R⁶ and R⁷ taken together form an optionally substituted heterocycle with the nitrogen to which they are attached;
R⁸ is optionally substituted alkyl, optionally substituted aryl, carbocyclyl, optionally substituted aralkyl, optionally substituted heteroaryl, optionally substituted heterocyclyl, —R^{b}COOR^{a}, or —R^{b}CONR^{a}R^{a};
each R^{a} is independently selected from H and alkyl;
R^{b} is a bond or alkylenyl;
R^{c} is a bond or alkenylenyl; and
A is optionally substituted aryl, optionally substituted heterocyclyl, optionally substituted heteroaryl, optionally substituted carbocyclyl, optionally substituted aralkyl, optionally substituted heteroaralkyl, or —R^{c}-(optionally substituted heteroaryl).

In some embodiments of a compound of Formula (I), Y₃ is N. In some embodiments of a compound of Formula (I), Y₃ is CR⁵.

In some embodiments of a compound of Formula (I), Y₁ is S. In some embodiments of a compound of Formula (I), Y₁ is NR².

In some embodiments of a compound of Formula (I), Y₂ is CR⁴. In some embodiments of a compound of Formula (I), Y₂ is N.

In some embodiments of a compound of Formula (I), Y₁ is S; Y₂ is CR⁴; and Y₃ is CR⁵. In some embodiments of a compound of Formula (I), Y₁ is NR²; Y₂ is N; and Y₃ is CR⁵.

In some embodiments of a compound of Formula (I), each M₁ is a bond. In some embodiments of a compound of Formula (I), each M₁ is —C(═O)—. In some embodiments of a compound of Formula (I), each M₁ is —S(═O)₂—.

In some embodiments of a compound of Formula (I), -M₁-Z-M₁-R¹ is —C(═O)—R¹. In further embodiments of a compound of Formula (I), -M₁-Z-M₁-R¹ is —C(═O)—O-(optionally substituted alkyl). In other embodiments of a compound of Formula (I), -M₁-Z-M₁-R¹ is —C(═O)— NR⁶R⁷. In still other embodiments of a compound of Formula (I), -M₁-Z-M₁-R¹ is —C(═O)—R⁸.

In some embodiments of a compound of Formula (I), Z is a bond. In some embodiments of a compound of Formula (I), Z is NR². In some embodiments of a compound of Formula (I), Z is NR² and each M₁ is —C(═O)—.

In some embodiments of a compound of Formula (I), M₂ is —C(═O)—.

In some embodiments of a compound of Formula (I), M₁ and M₂ are both —C(═O)—. In some embodiments of a compound of Formula (I), M₁ and M₂ are both —C(═O)—; and Z is NR².

In some embodiments of a compound of Formula (I), R¹ is —O-(optionally substituted alkyl), —O-(alkenyl), —O-(alkenyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), or —O-(alkyl)-(NR⁶R⁷). In some embodiments of a compound of Formula (I), R¹ is —O-(optionally substituted alkyl). In some embodiments of a compound of Formula (I), R¹ is —O-(alkenyl). In some embodiments of a compound of Formula (I), R¹ is —O-(alkenyl). In some embodiments of a compound of Formula (I), R¹ is —O-(cycloalkyl). In some embodiments of a compound of Formula (I), R¹ is —O-(heterocyclyl). In some embodiments of a compound of Formula (I), R¹ is —O-(optionally substituted aralkyl). In some embodiments of a compound of Formula (I), R¹ is —O-(optionally substituted heteroaralkyl). In some embodiments of a compound of Formula (I), R¹ is —O-(alkyl)-(alkoxy). In some embodiments of a compound of Formula (I), R¹ is —O-(alkyl)-(aralkoxy). In some embodiments of a compound of Formula (I), R¹ is —O-(alkyl)-(heterocyclyl). In some embodiments of a compound of Formula (I), R¹ is —O-(alkyl)-(COOR^{a}). In some embodiments of a compound of Formula (I), R¹ is —O-(alkyl)-(NR⁶R⁷). In some embodiments of a compound of Formula (I), R¹ is —O-(optionally substituted alkyl); and R² and R³ are both H.

In some embodiments of a compound of Formula (I), R¹ is —NR⁶R⁷. In some embodiments of a compound of Formula (I), R⁶ and R⁷ are each independently selected from H and optionally substituted alkyl. In some embodiments of a compound of Formula (I), R⁶ and R⁷ are each independently selected from H and optionally substituted alkyl; wherein the optional substituent is halogen. In some embodiments of a compound of Formula (I), R⁶ and R⁷ are H. In some embodiments of a compound of Formula (I), R⁶ and R⁷ are each optionally substituted alkyl. In some embodiments of a compound of Formula (I), R⁶ and R⁷ are each optionally substituted alkyl; wherein the optional substituent is halogen. In some embodiments of a compound of Formula (I), R⁶ and R⁷ taken together form an optionally substituted heterocycle with the nitrogen to which they are attached. In some embodiments of a compound of Formula (I), R⁶ and R⁷ taken together form an optionally substituted heterocycle with the nitrogen to which they are attached; wherein the optional substituent is halogen.

In some embodiments of a compound of Formula (I), R¹ is R⁸. In some embodiments of a compound of Formula (I), R⁸ is optionally substituted alkyl. In some embodiments of a compound of Formula (I), R⁸ is optionally substituted aryl. In some embodiments of a compound of Formula (I), R⁸ is carbocyclyl. In some embodiments of a compound of Formula (I), R⁸ is optionally substituted aralkyl. In some embodiments of a compound of Formula (I), R⁸ is optionally substituted heteroaryl. In some embodiments of a compound of Formula (I), R⁸ is optionally substituted heterocyclyl. In some embodiments of a compound of Formula (I), R⁸ is —R^{b}COOR^{a}. In some embodiments of a compound of Formula (I), R⁸ is —R^{b}CONR^{a}R^{a}.

In some embodiments of a compound of Formula (I), R² and R³ are each independently selected from H, optionally substituted alkyl and optionally substituted aryl. In some embodiments of a compound of Formula (I), R² and R³ are H. In some embodiments of a compound of Formula (I), R² and R³ are each independently selected from H and optionally substituted alkyl. In some embodiments of a compound of Formula (I), R² and R³ are optionally substituted alkyl. In some embodiments of a compound of Formula (I), R² and R³ are optionally substituted aryl. In some embodiments of a compound of Formula (I), R² and R³ are each independently selected from optionally substituted alkyl and optionally substituted aryl. In some embodiments of a compound of Formula (I), R¹ and R² taken together form a heterocycle.

In some embodiments of a compound of Formula (I), R⁴ is H. In some embodiments of a compound of Formula (I), R⁴ is halogen. In some embodiments of a compound of Formula (I), R⁴ is —CN. In some embodiments of a compound of Formula (I), R⁴ is optionally substituted alkyl. In some embodiments of a compound of Formula (I), R⁴ is optionally substituted aryl. In some embodiments of a compound of Formula (I), R⁴ is —R^{b}COOR^{a}. In some embodiments of a compound of Formula (I), R⁴ is —R^{b}CH(COOR^{a})₂. In some embodiments of a compound of Formula (I), R⁴ is H, halogen, —CN, or optionally substituted alkyl. In some embodiments of a compound of Formula (I), R⁴ is H, halogen, —CN, optionally substituted alkyl, or optionally substituted aryl. In some embodiments of a compound of Formula (I), R⁴ is —R^{b}COOR^{a} or —R^{b}CH(COOR^{a})₂.

In some embodiments of a compound of Formula (I), R⁵ is H, halogen, optionally substituted alkyl or cycloalkyl. In some embodiments of a compound of Formula (I), R⁵ is H. In some embodiments of a compound of Formula (I), R⁵ is halogen. In some embodiments of a compound of Formula (I), R⁵ is optionally substituted alkyl. In some embodiments of a compound of Formula (I), R⁵ is cycloalkyl. In some embodiments of a compound of Formula (I), R⁵ is H, halogen, or optionally substituted alkyl. In some embodiments of a compound of Formula (I), R⁵ is H, optionally substituted alkyl, or cycloalkyl. In some embodiments of a compound of Formula (I), R⁵ is H or alkyl. In some embodiments of a compound of Formula (I), R⁵ is optionally substituted alkyl or cycloalkyl. In some embodiments of a compound of Formula (I), R⁴ and R⁵ taken together form a carbocycle or an optionally substituted heterocycle.

In one aspect, provided herein are compounds of Formula (Ia), a pharmaceutically acceptable salt, solvate, polymorph, prodrug, metabolite, N-oxide, stereoisomer, or isomer thereof:

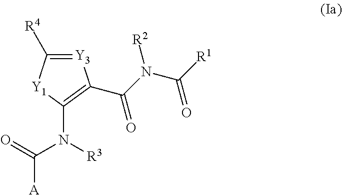

wherein:
Y₁ is S, O, or NR²;
Y₃ is CR⁵ or N;
R¹ is —O-(optionally substituted alkyl), —O-(alkenyl), —O-(alkenyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), —O-(alkyl)-(NR⁶R⁷), —NR⁶R⁷ or R⁸;
R² and R³ are each independently selected from H, optionally substituted alkyl, and optionally substituted aryl; or R¹ and R² taken together form a heterocycle;
R⁴ is H, halogen, —CN, optionally substituted alkyl, aryl, —R^{b}COOR^{a} or —R^{b}CH(COOR^{a})₂;
R⁵ is H, halogen, optionally substituted alkyl, or cycloalkyl; or R⁴ and R⁵ taken together form a carbocycle or an optionally substituted heterocycle;
R⁶ and R⁷ are each independently selected from H and optionally substituted alkyl; or R⁶ and R⁷ taken together form an optionally substituted heterocycle with the nitrogen to which they are attached;
R⁸ is optionally substituted alkyl, optionally substituted aryl, carbocyclyl, optionally substituted aralkyl, optionally substituted heteroaryl, or optionally substituted heterocyclyl;
each R^{a} is independently selected from H and alkyl;
R^{b} is a bond or alkylenyl;
R^{c} is a bond or alkenylenyl; and
A is optionally substituted aryl, optionally substituted heterocyclyl, optionally substituted heteroaryl, optionally substituted carbocyclyl, optionally substituted aralkyl, optionally substituted heteroaralkyl or —R^{c}-(optionally substituted heteroaryl).

In some embodiments of a compound of Formula (Ia), R¹ is —O-(optionally substituted alkyl), —O-(alkenyl), —O-(alkenyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), or —O-(alkyl)-(NR⁶R⁷). In some embodiments of a compound of Formula (Ia), R¹ is —O-(optionally substituted alkyl). In some embodiments of a compound of Formula (Ia), R¹ is —O-(alkenyl). In some embodiments of a compound of Formula (Ia), R¹ is —O-(alkenyl). In some embodiments of a compound of Formula (Ia), R¹ is —O-(cycloalkyl). In some embodiments of a compound of Formula (Ia), R¹ is —O-(heterocyclyl). In some embodiments of a compound of Formula (Ia), R¹ is —O-(optionally substituted aralkyl). In some embodiments of a compound of Formula (Ia), R¹ is —O-(optionally substituted heteroaralkyl). In some embodiments of a compound of Formula (Ia), R¹ is —O-(alkyl)-(alkoxy). In some embodiments of a compound of Formula (Ia), R¹ is —O-(alkyl)-(aralkoxy). In some embodiments of a compound of Formula (Ia), R¹ is —O-(alkyl)-(heterocyclyl). In some embodiments of a compound of Formula (Ia), R¹ is —O-(alkyl)-(COOR^{a}). In some embodiments of a compound of Formula (Ia), R¹ is —O-(alkyl)-(NR⁶R⁷).

In some embodiments of a compound of Formula (Ia), R¹ is —O-(optionally substituted alkyl); wherein the optionally substituted alkyl is substituted with halogen. In further embodiments of a compound of Formula (Ia), R¹ is —O-(cycloalkyl). In still further embodiments of a compound of Formula (Ia), R¹ is —O-(cyclobutyl), —O-(cyclopentyl), or —O-(cyclohexyl). In some embodiments of a compound of Formula (Ia), R¹ is —O-(heterocyclyl). In further embodiments of a compound of Formula (Ia), R¹ is —O-(optionally substituted piperidinyl), —O-(oxetanyl), or —O-(tetrahydrofuranyl), wherein the optionally substituted piperidinyl is substituted with —COCH₃. In some embodiments of a compound of Formula (Ia), R¹ is —O-(optionally substituted alkyl); and R² and R³ are both H.

In some embodiments of a compound of Formula (Ia), R¹ is —O-(optionally substituted aralkyl) or —O-(optionally substituted heteroaralkyl). In further embodiments of a compound of Formula (Ia), the optionally substituted aralkyl and the optionally substituted heteroaralkyl are substituted with a group selected from halogen, alkyl and —CF₃.

In some embodiments of a compound of Formula (Ia), R¹ is —O-(alkyl)-(heterocyclyl). In further embodiments of a compound of Formula (Ia), the heterocyclyl is morpholinyl.

In some embodiments of a compound of Formula (Ia), R¹ is —NR⁶R⁷. In some embodiments of a compound of Formula (Ia), R⁶ and R⁷ are each independently selected from H and optionally substituted alkyl. In some embodiments of a compound of Formula (Ia), R⁶ and R⁷ are each independently selected from H and optionally substituted alkyl; wherein the optional substituent is halogen. In some embodiments of a compound of Formula (Ia), R⁶ and R⁷ are H. In some embodiments of a compound of Formula (Ia), R⁶ and R⁷ are each optionally substituted alkyl. In some embodiments of a compound of Formula (Ia), R⁶ and R⁷ are each optionally substituted alkyl; wherein the optional substituent is halogen. In some embodiments of a compound of Formula (Ia), R⁶ and R⁷ taken together form an optionally substituted heterocycle with the nitrogen to which they are attached. In some embodiments of a compound of Formula (Ia), R⁶ and R⁷ taken together form an optionally substituted heterocycle with the nitrogen to which they are attached; wherein the optional substituent is halogen. In some embodiments of a compound of Formula (Ia), R⁶ and R⁷ taken together form a heterocycle with the nitrogen to which they are attached; wherein the heterocycle is selected from piperidinyl and morpholinyl.

In some embodiments of a compound of Formula (Ia), R¹ is R⁸. In some embodiments of a compound of Formula (Ia), R⁸ is optionally substituted alkyl. In some embodiments of a compound of Formula (Ia), R⁸ is optionally substituted aryl. In some embodiments of a compound of Formula (Ia), R⁸ is optionally substituted aryl; wherein the optionally substituted aryl is substituted with halogen. In some embodiments of a compound of Formula (Ia), R⁸ is carbocyclyl. In some embodiments of a compound of Formula (Ia), R⁸ is optionally substituted carbocyclylalkyl. In some embodiments of a compound of Formula (Ia), R⁸ is optionally substituted heteroaryl. In some embodiments of a compound of Formula (Ia), R⁸ is optionally substituted heteroaryl; wherein the optionally substituted heteroaryl is substituted with a group selected from alkyl, —O-(alkyl) and —NR⁶R⁷. In some embodiments of a compound of Formula (Ia), R⁸ is optionally substituted heteroarylalkyl. In some embodiments of a compound of Formula (Ia), R⁸ is optionally substituted heterocyclyl. In some embodiments of a compound of Formula (Ia), R⁸ is optionally substituted heterocyclyl; wherein the optionally substituted heterocyclyl is substituted with alkyl. In some embodiments of a compound of Formula (Ia), R² and R⁸ taken together form an optionally substituted heterocycle with the nitrogen to which they are attached. In further embodiments of a compound of Formula (Ia), the heterocycle is selected from piperidinyl, morpholinyl, thiomorpholinyl, optionally substituted diazepanyl and optionally substituted piperizanyl; wherein the optionally substituted diazepanyl and the optionally substituted piperizanyl are substituted with a group selected from alkyl, aryl, -COOtBu and —SO₂Me. In some embodiments of a compound of Formula (Ia), R⁸ is optionally substituted heterocyclylalkyl. In some embodiments of a compound of Formula (Ia), R² and R⁸ taken together form an optionally substituted heterocycle with the nitrogen to which they are attached. In some embodiments of a compound of Formula (Ia), R⁸ is optionally substituted alkyl, optionally substituted aryl, optionally substituted aralkyl, carbocyclyl, optionally substituted carbocyclylalkyl, optionally substituted heteroaryl, optionally substituted heteroarylalkyl, optionally substituted heterocyclyl, or optionally substituted heterocyclylalkyl. In some embodiments of a compound of Formula (Ia), R⁸ is optionally substituted alkyl, carbocyclyl, optionally substituted carbocyclylalkyl, optionally substituted heterocyclyl, or optionally substituted heterocyclylalkyl. In some embodiments of a compound of Formula (Ia), R⁸ is optionally substituted aryl, optionally substituted aralkyl, optionally substituted heteroaryl, or optionally substituted heteroarylalkyl. In some embodiments of a compound of Formula (Ia), R⁸ is optionally substituted alkyl, optionally substituted aralkyl, carbocyclyl, optionally substituted carbocyclylalkyl, optionally substituted heteroarylalkyl, optionally substituted heterocyclyl, or optionally substituted heterocyclylalkyl.

In some embodiments of a compound of Formula (Ia), R² and R³ are both H. In some embodiments of a compound of Formula (Ia), R² and R³ are both optionally substituted alkyl. In some embodiments of a compound of Formula (Ia), R² is hydrogen and R³ is optionally substituted alkyl. In some embodiments of a compound of Formula (Ia), R² is optionally substituted alkyl and R³ is hydrogen. In some embodiments of a compound of Formula (Ia), R² is hydrogen and R³ is optionally substituted aryl. In some embodiments of a compound of Formula (Ia), R² is optionally substituted aryl and R³ is hydrogen. In some embodiments of a compound of Formula (Ia), R² is optionally substituted aryl and R³ is optionally substituted alkyl. In some embodiments of a compound of Formula (Ia), R² is optionally substituted alkyl and R³ is optionally substituted aryl.

In some embodiments of a compound of Formula (Ia), R¹ and R² taken together form a heterocycle.

In some embodiments of a compound of Formula (Ia), R⁴ is H. In some embodiments of a compound of Formula (Ia), R⁴ is halogen. In some embodiments of a compound of Formula (Ia), R⁴ is —CN. In some embodiments of a compound of Formula (Ia), R⁴ is optionally substituted alkyl. In some embodiments of a compound of Formula (Ia), R⁴ is aryl. In some embodiments of a compound of Formula (Ia), R⁴ is —R^{b}COOR^{a}. In some embodiments of a compound of Formula (Ia), R⁴ is —R^{b}CH(COOR^{a})₂. In some embodiments of a compound of Formula (Ia), R⁴ is H, halogen, —CN, or optionally substituted alkyl. In some embodiments of a compound of Formula (Ia), R⁴ is H, halogen, —CN, optionally substituted alkyl, or aryl. In some embodiments of a compound of Formula (Ia), R⁴ is —R^{b}COOR^{a} or —R^{b}CH(COOR^{a})₂.

In some embodiments of a compound of Formula (Ia), R⁵ is H, halogen, optionally substituted alkyl or cycloalkyl. In some embodiments of a compound of Formula (Ia), R⁵ is H. In some embodiments of a compound of Formula (Ia), R⁵ is halogen. In some embodiments of a compound of Formula (Ia), R⁵ is optionally substituted alkyl. In some embodiments of a compound of Formula (Ia), R⁵ is cycloalkyl. In some embodiments of a compound of Formula (Ia), R⁵ is H, halogen, or optionally substituted alkyl. In some embodiments of a compound of Formula (Ia), R⁵ is H, optionally substituted alkyl, or cycloalkyl. In some embodiments of a compound of Formula (Ia), R⁵ is H or optionally substituted alkyl. In some embodiments of a compound of Formula (Ia), R⁵ is optionally substituted alkyl or cycloalkyl. In some embodiments of a compound of Formula (Ia), R⁴ and R⁵ taken together form a carbocycle or an optionally substituted heterocycle. In some embodiments of a compound of Formula (Ia), R⁴ and R⁵ taken together form an optionally substituted heterocycle; wherein the optionally substituted heterocycle is substituted with a group selected from alkyl, aralkyl and —SO₂Me.

In some embodiments of a compound of Formula (Ia), Y₃ is N. In some embodiments of a compound of Formula (Ia), Y₃ is CR⁵.

In some embodiments of a compound of Formula (Ia), Y₁ is NR². In some embodiments of a compound of Formula (Ia), Y₁ is O. In some embodiments of a compound of Formula (Ia), Y₁ is S. In some embodiments of a compound of Formula (Ia), Y₁ is S and Y₃ is CH.

In some embodiments of a compound of Formula (I) or Formula (Ia), A is optionally substituted aryl, optionally substituted heterocyclyl, optionally substituted heteroaryl, optionally substituted carbocyclyl, optionally substituted aralkyl, optionally substituted heteroaralkyl or —R^{c}-(optionally substituted heteroaryl); and the optionally substituted aryl, the optionally substituted heterocyclyl, the optionally substituted heteroaryl, the optionally substituted carbocyclyl, the optionally substituted aralkyl and the optionally substituted heteroaralkyl are substituted with 1-6 R¹⁰; wherein each R¹⁰ is independently selected from H, halogen, —CN, —NO₂, —CF₃, alkyl, —SR⁶, —OR⁶, —NR⁶R⁷, —NR⁶C(═O)(alkyl), —NR⁶C(═O)(cycloalkyl), —NR⁶C(═O)(heterocyclyl), —NR⁶C(═O)(aryl), —NR⁶C(═O)(heteroaryl), —C(═O)NR⁶R⁷, —C(═O)NR⁶(cycloalkyl), —C(═O)NR⁶(heterocycloalkyl), —C(═O)NR⁶(aryl), —C(═O)NR⁶(heteroaryl), —NR⁶C(═O)NR⁶R⁷, —NR⁶C(═O)NR⁷(cycloalkyl), —NR⁶C(═O)NR⁷(heterocycloalkyl), —NR⁶C(═O)NR⁷(aryl), —NR⁶C(═O)NR⁷(heteroaryl), —NR⁶C(═O)O(alkyl), —NR⁶C(═O)O(cycloalkyl), —NR⁶C(═O)O(heterocycloalkyl), —NR⁶C(═O)O(aryl), —NR⁶C(═O)O(heteroaryl), —NR⁶SO₂(alkyl), —NR⁶SO₂(cycloalkyl), —NR⁶SO₂(heterocycloalkyl), —NR⁶SO₂(aryl), —NR⁶SO₂(heteroaryl), —SO₂NR⁶R⁷, —SO₂NR⁶(cycloalkyl), —SO₂NR⁶(heterocycloalkyl), —SR⁶, —SO₂R⁶, —SO₂NR⁶(aryl), —SO₂NR⁶(heteroaryl), haloalkyl, aryl, heteroaryl, heterocyclyl and tetrazoyl.

In some embodiments of a compound of Formula (I) or Formula (Ia), A is optionally substituted aryl. In some embodiments of a compound of Formula (I) or Formula (Ia), A is optionally substituted heterocyclyl. In some embodiments of a compound of Formula (I) or Formula (Ia), A is optionally substituted carbocyclyl. In some embodiments of a compound of Formula (I) or Formula (Ia), A is optionally substituted aralkyl. In some embodiments of a compound of Formula (I) or Formula (Ia), A is optionally substituted heteroaralkyl. In some embodiments of a compound of Formula (I) or Formula (Ia), A is optionally substituted —R^{c}-(optionally substituted heteroaryl).

In some embodiments of a compound of Formula (I) or Formula (Ia), A is optionally substituted heteroaryl. In some embodiments of a compound of Formula (I) or Formula (Ia), A is selected from:

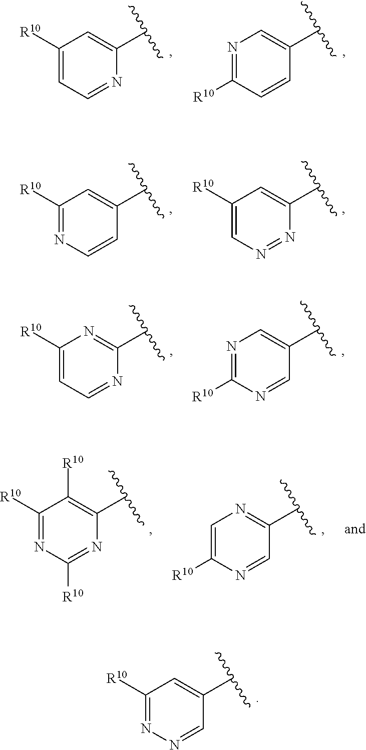

In some embodiments of a compound of Formula (I) or Formula (Ia), A is

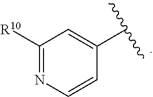

In some embodiments of a compound of Formula (I) or Formula (Ia), A is selected from

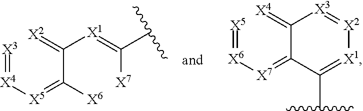

wherein X¹, X², X³, X⁴, X⁵, X⁶, and X⁷ are independently selected from N and CR¹⁰; and at least one of X¹-X⁷ is N. In some embodiments of a compound of Formula (I) or Formula (Ia), A is selected from

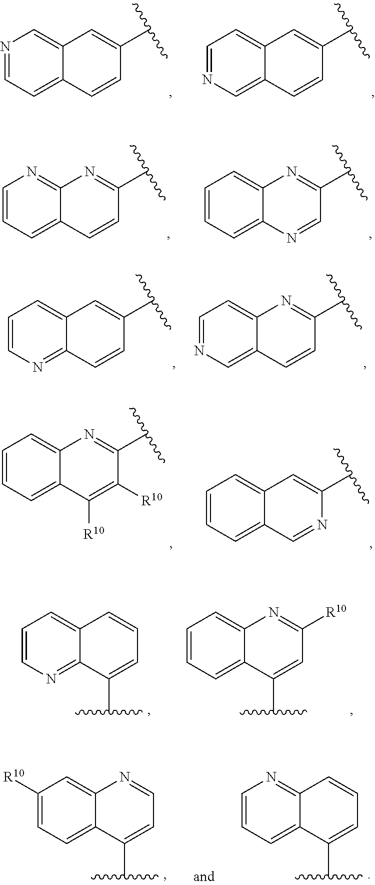

In some embodiments of a compound of Formula (I) or Formula (Ia), A is selected from

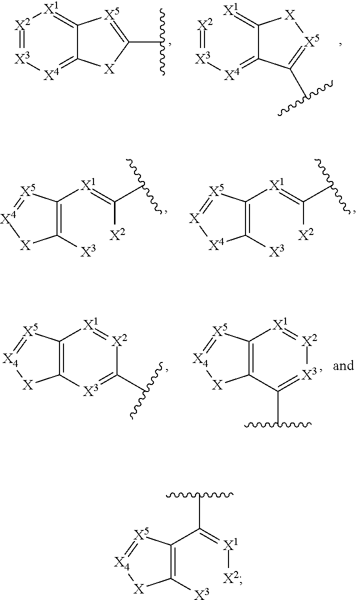

wherein X¹, X², X³, X⁴, X⁵, X⁶, and X⁷ are independently selected from N and CR¹⁰; and X is O, S, or NR². In some embodiments of a compound of Formula (I) or Formula (Ia), A is selected from

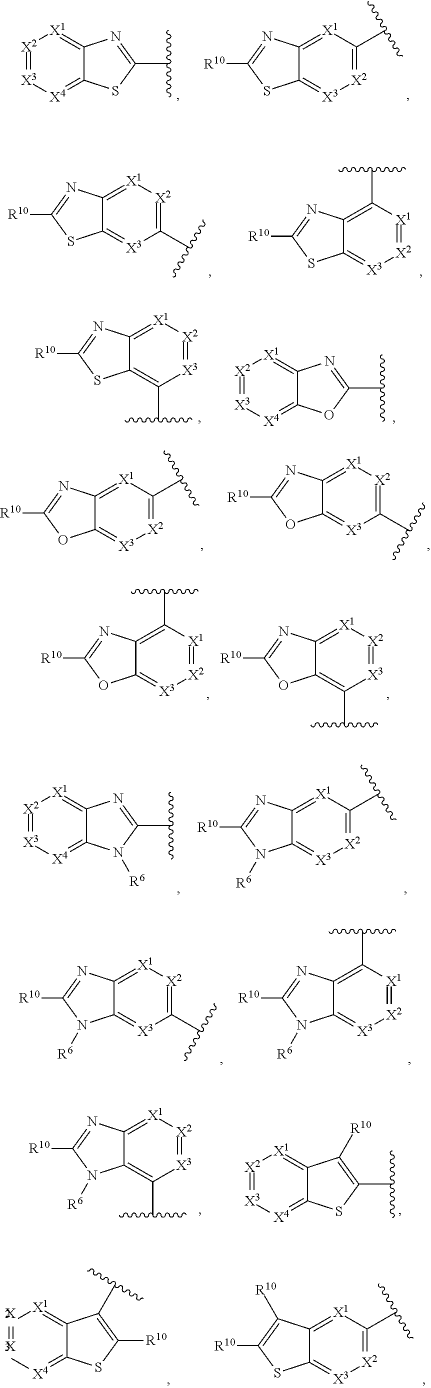

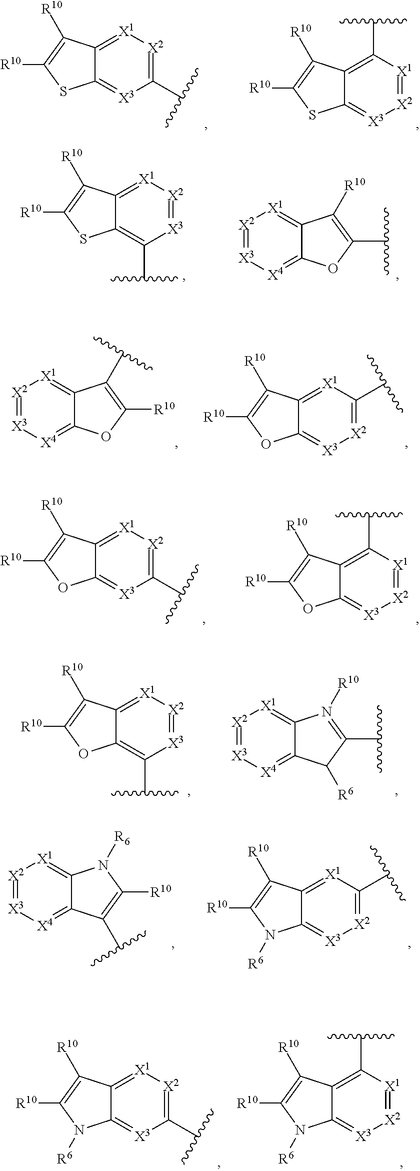

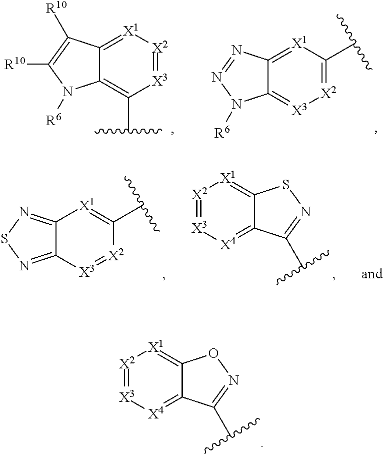

In some embodiments of a compound of Formula (I) or Formula (Ia), A is selected from

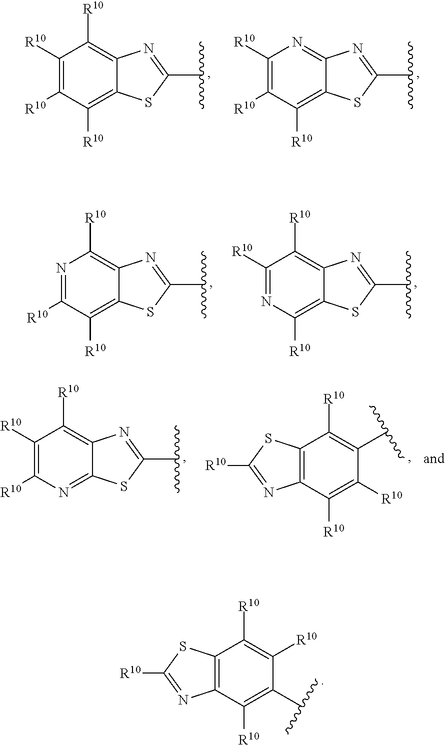

In some embodiments of a compound of Formula (I) or Formula (Ia), A is selected from

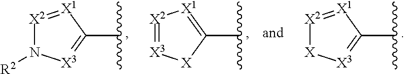

In some embodiments of a compound of Formula (I) or Formula (Ia), A is selected from

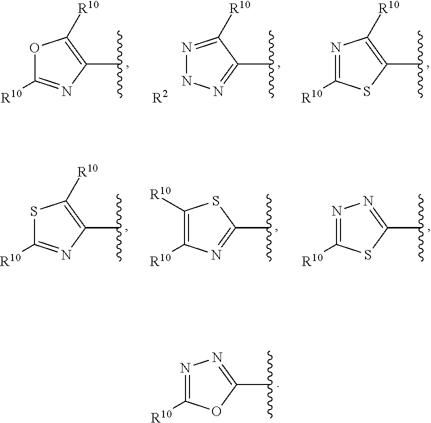

In some embodiments of a compound of Formula (I) or Formula (Ia), A is selected from

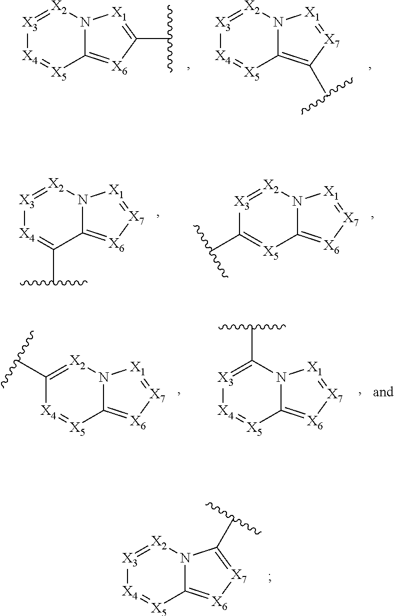

wherein X¹, X², X³, X⁴, X⁵, X⁶, and X⁷ are independently selected from N and CR¹⁰. In some embodiments of a compound of Formula (I) or Formula (Ia), A is selected from

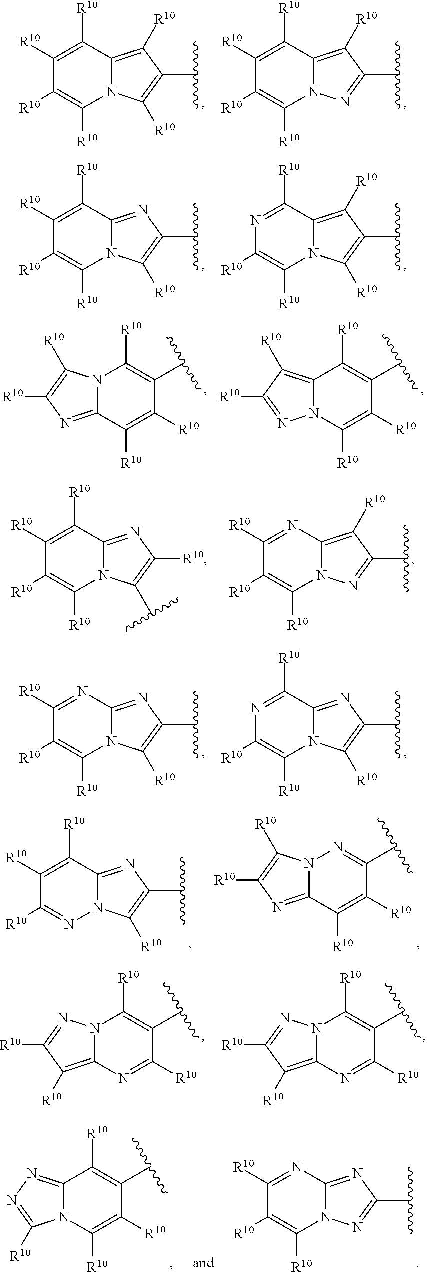

In some embodiments of a compound of Formula (I) or Formula (Ia), A is selected from

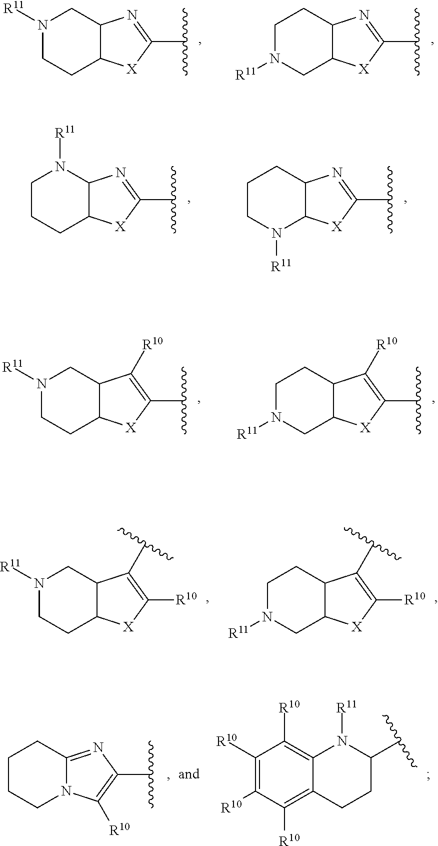

wherein X is O, S, or NR²; and R¹ is H, alkyl, aryl, heteroaryl, —SO₂-(alkyl), —SO₂-(cycloalkyl), —SO₂-(aryl), —SO₂-(heteroaryl), —SO₂-(heterocycloalkyl), —C(═O)O(alkyl), —C(═O)O(cycloalkyl), —C(═O)O(heterocycloalkyl), —C(═O)O(aryl), —C(═O)O(heteroaryl), —C(═O)NR⁶R⁷, —C(═O)NR⁶(cycloalkyl), —C(═O)NR⁶(heterocycloalkyl), —C(═O)NR⁶(aryl), —C(═O)NR⁶(heteroaryl), —C(═O)(alkyl), —C(═O)(cycloalkyl), —C(═O)(heterocycloalkyl), —C(═O)(aryl), or —C(═O)(heteroaryl). In some embodiments of a compound of Formula (I) or Formula (Ia), A is selected from

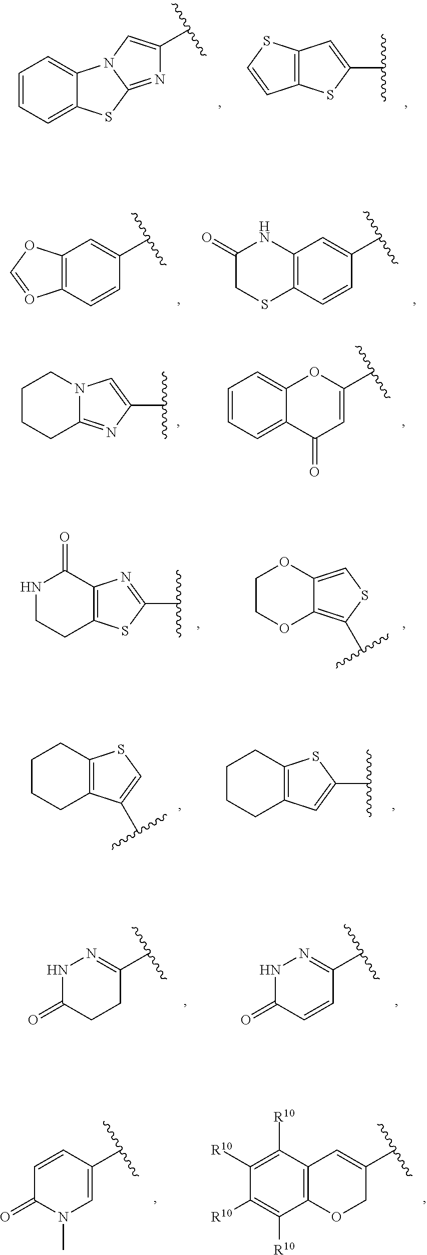

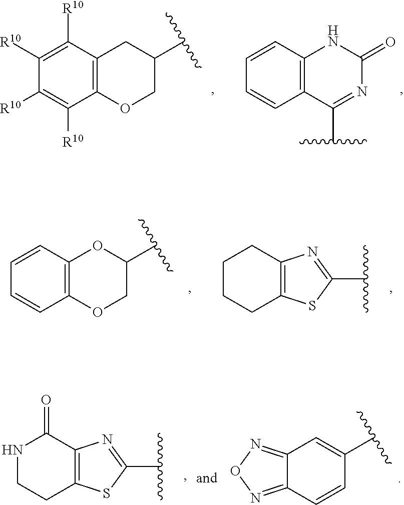

In one aspect, provided herein are compounds of Formula (Ib), a pharmaceutically acceptable salt, solvate, polymorph, prodrug, metabolite, N-oxide, stereoisomer, or isomer thereof:

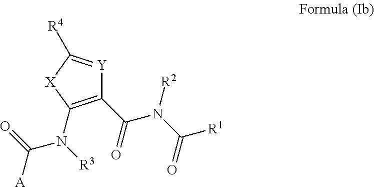

wherein:
R¹ is —O-(alkyl), —O-(haloalkyl), —O-(alkenyl), —O-(haloalkenyl), —O-(alkynyl), —O-(haloalkynyl), —O-(cycloalkyl), —O-(heterocycloalkyl), —O-(arylalkyl), —O-(alkyl)-(alkoxy), —O-(alkyl)-(NR⁶R⁷), or —NR⁶R⁷;
R² and R³ are independently selected from H, haloalkyl, and alkyl;
R⁴ is H, halogen, CN, or alkyl;
X is S or O;
Y is CR⁵ or N;
R⁵ is H, alkyl, or halogen;
each R⁶ and R⁷ is independently selected from H and alkyl; or R⁶ and R⁷ taken together form a heterocycle with the nitrogen to which they are attached; and
A is heteroaryl, aryl, or heterocyclyl.

In some embodiments of a compound of Formula (Ib), Y is N. In some embodiments of a compound of Formula (Ib), Y is CR⁵.

In some embodiments of a compound of Formula (Ib), X is S. In some embodiments of a compound of Formula (Ib), X is O.

In some embodiments of a compound of Formula (Ib), A is heterocyclyl. In some embodiments of a compound of Formula (Ib), A is aryl. In some embodiments of a compound of Formula (Ib), A is heteroaryl.

In some embodiments of a compound of Formula (Ib),
A is selected from:

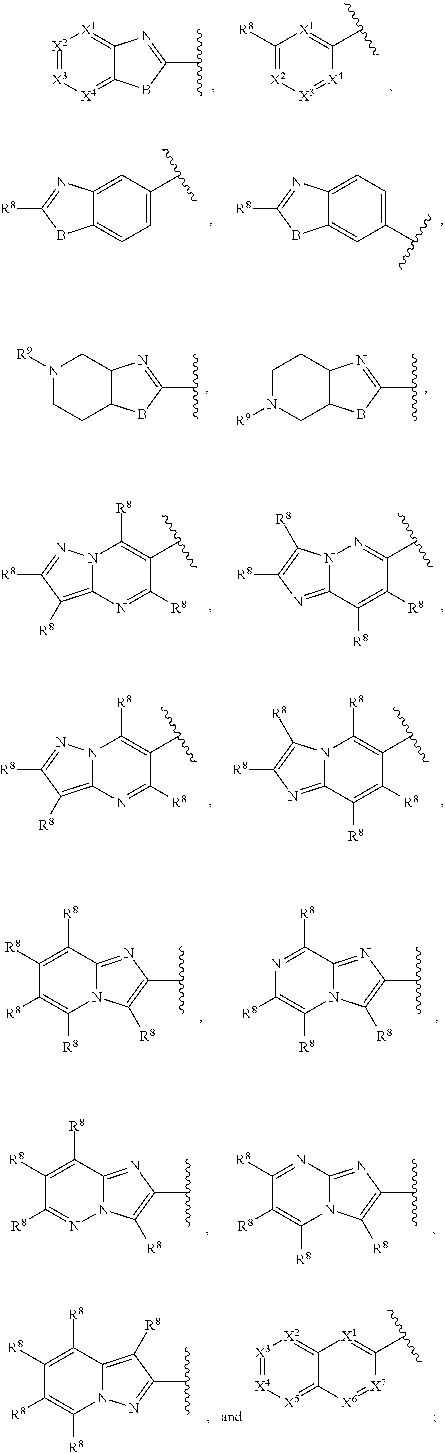

X¹, X², X³, X⁴, X⁵, X⁶, and X⁷ are independently selected from N and CR⁸;
each R⁸ is independently selected from H, halogen, CN, NO₂, alkyl, —SR⁶, —OR⁶, —NR⁶R⁷, —NR⁶C(O)(alkyl), —NR⁶C(O)(cycloalkyl), —NR⁶C(O)(heterocycloalkyl), —NR⁶C(O)(aryl), —NR⁶C(O)(heteroaryl), —C(O)NR⁶R⁷, —C(O)NR⁶(cycloalkyl), —C(O)NR⁶(heterocycloalkyl), —C(O)NR⁶(aryl), —C(O)NR⁶(heteroaryl), —NR⁶C(O)NR⁶R⁷, —NR⁶C(O)NR⁷(cycloalkyl), —NR⁶C(O)NR⁷(heterocycloalkyl), —NR⁶C(O)NR⁷(aryl), —NR⁶C(O)NR⁷(heteroaryl), —NR⁶C(O)O(alkyl), —NR⁶C(O)O(cycloalkyl), —NR⁶C(O)O(heterocycloalkyl), —NR⁶C(O)O(aryl), —NR⁶C(O)O(heteroaryl), —NR⁶SO₂ (alkyl), —NR⁶SO₂ (cycloalkyl), —NR⁶SO₂ (heterocycloalkyl), —NR⁶SO₂ (aryl), —NR⁶SO₂ (heteroaryl), —SO₂NR⁶R⁷, —SO₂NR⁶(cycloalkyl), —SO₂NR⁶(heterocycloalkyl), —SO₂NR⁶(aryl), —SO₂NR⁶(heteroaryl), haloalkyl, aryl, and heteroaryl;
R⁹ is H, alkyl, aryl, heteroaryl, —SO₂-(alkyl), —SO₂-(cycloalkyl), —SO₂-(aryl), —SO₂-(heteroaryl), —SO₂-(heterocycloalkyl), —C(O)O(alkyl), —C(O)O(cycloalkyl), —C(O)O(heterocycloalkyl), —C(O)O(aryl), —C(O)O(heteroaryl), —C(O)NR⁶R⁷, —C(O)NR⁶(cycloalkyl), —C(O)NR⁶(heterocycloalkyl), —C(O)NR⁶(aryl), —C(O)NR⁶(heteroaryl), —C(O)(alkyl), —C(O)(cycloalkyl), —C(O)(heterocycloalkyl), —C(O)(aryl), or —C(O)(heteroaryl); and
B is O, S, or NR².
   In further embodiments of a compound of Formula (Ib), X is S. In still further embodiments of a compound of Formula (Ib), X is S and Y is CH. In still further embodiments of a compound of Formula (Ib), X is S, Y is CH, and R⁴ is H.

In some embodiments of a compound of Formula (Ib), A is selected from:

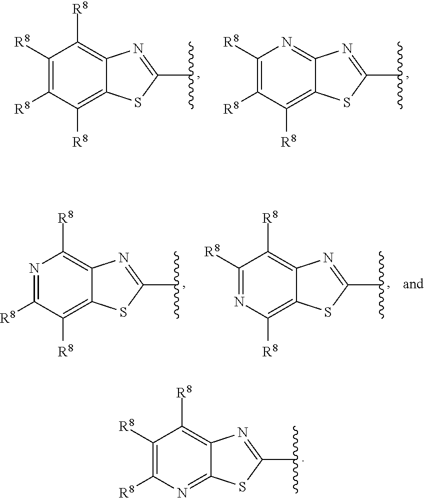

In further embodiments of a compound of Formula (Ib), X is S. In still further embodiments of a compound of Formula (Ib), X is S and Y is CH.

In some embodiments of a compound of Formula (Ib), A is selected from:

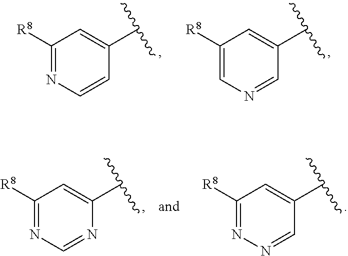

In further embodiments of a compound of Formula (Ib), X is S. In still further embodiments of a compound of Formula (Ib), X is S and Y is CH.

In some embodiments of a compound of Formula (Ib), A is selected from:

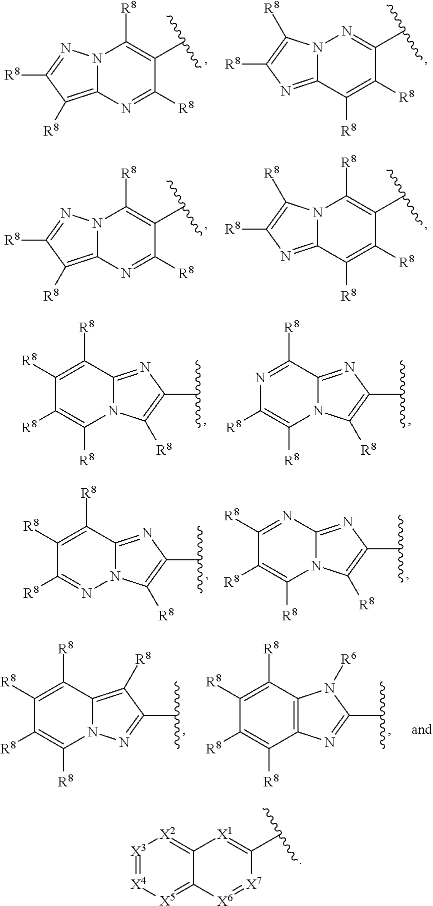

In further embodiments of a compound of Formula (Ib), X is S. In still further embodiments of a compound of Formula (Ib), X is S and Y is CH.

In some embodiments of a compound of Formula (Ib), R¹ is —O-(alkyl), —O-(haloalkyl), —O-(alkenyl), —O-(haloalkenyl), —O-(alkenyl), —O-(haloalkynyl), —O-(cycloalkyl), —O-(heterocycloalkyl), —O-(arylalkyl), —O-(alkyl)-(alkoxy), or —O-(alkyl)-(NR⁶R⁷). In some embodiments of a compound of Formula (Ib), R¹ is —O-(alkyl). In certain embodiments of a compound of Formula (Ib), R¹ is ethoxy. In some embodiments of a compound of Formula (Ib), R¹ is —O-(haloalkyl). In some embodiments of a compound of Formula (Ib), R¹ is —O-(alkenyl). In some embodiments of a compound of Formula (Ib), R¹ is —O-(haloalkenyl). In some embodiments of a compound of Formula (Ib), R¹ is —O-(alkynyl). In some embodiments of a compound of Formula (Ib), R¹ is —O-(haloalkynyl). In some embodiments of a compound of Formula (Ib), R¹ is —O-(cycloalkyl). In some embodiments of a compound of Formula (Ib), R¹ is —O-(heterocycloalkyl). In some embodiments of a compound of Formula (Ib), R¹ is —O-(arylalkyl). In some embodiments of a compound of Formula (Ib), R¹ is —O-(alkyl)-(alkoxy). In some embodiments of a compound of Formula (Ib), R¹ is —O-(alkyl)-(NR⁶R⁷). In further embodiments of a compound of Formula (Ib), each R⁶ and R⁷ is independently selected from H and optionally substituted alkyl. In other embodiments of a compound of Formula (Ib), R⁶ and R⁷ taken together form a heterocycle with the nitrogen to which they are attached.

In some embodiments of a compound of Formula (Ib), R¹ is —NR⁶R⁷. In further embodiments of a compound of Formula (Ib), each R⁶ and R⁷ is independently selected from H and alkyl. In other embodiments of a compound of Formula (Ib), R⁶ and R⁷ taken together form a heterocycle with the nitrogen to which they are attached.

In some embodiments of a compound of Formula (Ib), R² and R³ are both hydrogen. In some embodiments of a compound of Formula (Ib), R² and R³ are both alkyl. In some embodiments of a compound of Formula (Ib), R² is hydrogen and R³ is alkyl. In some embodiments of a compound of Formula (Ib), R² is alkyl and R³ is hydrogen. In some embodiments of a compound of Formula (Ib), R² is haloalkyl. In some embodiments of a compound of Formula (Ib), R³ is haloalkyl.

In one aspect, provided herein are compounds of Formula (Ic), a pharmaceutically acceptable salt, solvate, polymorph, prodrug, metabolite, N-oxide, stereoisomer, or isomer thereof:

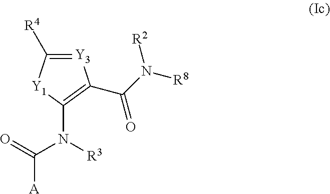

wherein:
Y₁ is S or O;
Y₃ is CR⁵ or N;
R² and R³ are each independently selected from H and optionally substituted alkyl;
R⁴ is H, halogen, —CN, optionally substituted alkyl, or optionally substituted aryl;
R⁵ is H, optionally substituted alkyl, or halogen;
R⁸ is optionally substituted alkyl, optionally substituted aryl, optionally substituted aralkyl, carbocyclyl, optionally substituted carbocyclylalkyl, optionally substituted heteroaryl, optionally substituted heteroarylalkyl, optionally substituted heterocyclyl, optionally substituted heterocyclylalkyl, —R^{b}COOR^{a} or —R^{b}CONR^{a}R^{a}; or R² and R⁸ taken together form an optionally substituted heterocycle with the nitrogen to which they are attached;
each R^{a} is independently selected from H and alkyl;
R^{b} is a bond or alkylenyl; and
A is optionally substituted heteroaryl, optionally substituted aryl or optionally substituted heterocyclyl.

In some embodiments of a compound of Formula (Ic), R⁸ is optionally substituted alkyl. In some embodiments of a compound of Formula (Ic), R⁸ is optionally substituted aryl. In some embodiments of a compound of Formula (Ic), R⁸ is optionally substituted aryl; wherein the optionally substituted aryl is substituted with halogen. In some embodiments of a compound of Formula (Ic), R⁸ is carbocyclyl. In some embodiments of a compound of Formula (Ic), R⁸ is optionally substituted carbocyclylalkyl. In some embodiments of a compound of Formula (Ic), R⁸ is optionally substituted heteroaryl. In some embodiments of a compound of Formula (Ic), R⁸ is optionally substituted heteroaryl; wherein the optionally substituted heteroaryl is substituted with a group selected from alkyl, —O-(alkyl) and —NR⁶R⁷. In some embodiments of a compound of Formula (Ic), R⁸ is optionally substituted heteroarylalkyl. In some embodiments of a compound of Formula (Ic), R⁸ is optionally substituted heterocyclyl. In some embodiments of a compound of Formula (Ic), R⁸ is optionally substituted heterocyclyl; wherein the optionally substituted heterocyclyl is substituted with alkyl. In some embodiments of a compound of Formula (Ic), R⁸ is optionally substituted heterocyclylalkyl. In some embodiments of a compound of Formula (Ic), R⁸ is —R^{b}COOR^{a}. In some embodiments of a compound of Formula (Ic), R⁸ is —R^{b}CONR^{a}R^{a}. In some embodiments of a compound of Formula (Ic), R⁸ is optionally substituted alkyl, optionally substituted aryl, optionally substituted aralkyl, carbocyclyl, optionally substituted carbocyclylalkyl, optionally substituted heteroaryl, optionally substituted heteroarylalkyl, optionally substituted heterocyclyl, or optionally substituted heterocyclylalkyl. In some embodiments of a compound of Formula (Ic), R⁸ is optionally substituted alkyl, carbocyclyl, optionally substituted carbocyclylalkyl, optionally substituted heterocyclyl, or optionally substituted heterocyclylalkyl. In some embodiments of a compound of Formula (Ic), R⁸ is optionally substituted aryl, optionally substituted aralkyl, optionally substituted heteroaryl, or optionally substituted heteroarylalkyl. In some embodiments of a compound of Formula (Ic), R⁸ is optionally substituted alkyl, optionally substituted aralkyl, carbocyclyl, optionally substituted carbocyclylalkyl, optionally substituted heteroarylalkyl, optionally substituted heterocyclyl, or optionally substituted heterocyclylalkyl.

In some embodiments of a compound of Formula (Ic), R² and R⁸ taken together form an optionally substituted heterocycle with the nitrogen to which they are attached. In further embodiments of a compound of Formula (Ic), the heterocycle is selected from piperidinyl, morpholinyl, thiomorpholinyl, optionally substituted diazepanyl and optionally substituted piperizanyl; wherein the optionally substituted diazepanyl and the optionally substituted piperizanyl are substituted with a group selected from alkyl, aryl, -COOtBu and —SO₂Me. In some embodiments of a compound of Formula (Ic), R² and R⁸ taken together form an optionally substituted heterocycle with the nitrogen to which they are attached.

In some embodiments of a compound of Formula (Ic), Y₃ is N. In some embodiments of a compound of Formula (Ic), Y₃ is CR⁵.

In some embodiments of a compound of Formula (Ic), R⁵ is H. In some embodiments of a compound of Formula (Ic), R⁵ is optionally substituted alkyl. In some embodiments of a compound of Formula (Ic), R⁵ is halogen. In some embodiments of a compound of Formula (Ic), R⁵ is H or optionally substituted alkyl.

In some embodiments of a compound of Formula (Ic), Y₁ is O. In some embodiments of a compound of Formula (Ic), Y₁ is S. In some embodiments of a compound of Formula (Ic), Y₁ is S and Y₃ is CH.

In some embodiments of a compound of Formula (Ic), R² and R³ are H. In some embodiments of a compound of Formula (Ic), R² and R³ are optionally substituted alkyl. In some embodiments of a compound of Formula (Ic), R² is optionally substituted alkyl and R³ is H. In some embodiments of a compound of Formula (Ic), R² is H and R³ is optionally substituted alkyl.

In some embodiments of a compound of Formula (Ic), R⁴ is H. In some embodiments of a compound of Formula (Ic), R⁴ is —CN. In some embodiments of a compound of Formula (Ic), R⁴ is optionally substituted alkyl. In some embodiments of a compound of Formula (Ic), R⁴ is optionally substituted aryl. In some embodiments of a compound of Formula (Ic), Y₁ is S, Y₃ is CH, and R⁴ is H. In some embodiments of a compound of Formula (Ic), Y₁ is S; Y₃ is CH; R⁴ is H; and R² and R³ are H.

In some embodiments of a compound of Formula (Ic), A is optionally substituted heteroaryl, optionally substituted aryl or optionally substituted heterocyclyl; and the optionally substituted aryl, the optionally substituted heterocyclyl, the optionally substituted heteroaryl are substituted with 1-6 R¹⁰; wherein each R¹⁰ is independently selected from H, halogen, —CN, —NO₂, —CF₃, alkyl, —SR⁶, —OR⁶, —NR⁶R⁷, —NR⁶C(═O)(alkyl), —NR⁶C(═O)(cycloalkyl), —NR⁶C(═O)(heterocyclyl), —NR⁶C(═O)(aryl), —NR⁶C(═O)(heteroaryl), —C(═O)NR⁶R⁷, —C(═O)NR⁶(cycloalkyl), —C(═O)NR⁶(heterocycloalkyl), —C(═O)NR⁶(aryl), —C(═O)NR⁶(heteroaryl), —NR⁶C(═O)NR⁶R⁷, —NR⁶C(═O)NR⁷(cycloalkyl), —NR⁶C(═O)NR⁷(heterocycloalkyl), —NR⁶C(═O)NR⁷(aryl), —NR⁶C(═O)NR⁷(heteroaryl), —NR⁶C(═O)O(alkyl), —NR⁶C(═O)O(cycloalkyl), —NR⁶C(═O)O(heterocycloalkyl), —NR⁶C(═O)O(aryl), —NR⁶C(═O)O(heteroaryl), —NR⁶SO₂(alkyl), —NR⁶SO₂(cycloalkyl), —NR⁶SO₂(heterocycloalkyl), —NR⁶SO₂(aryl), —NR⁶SO₂(heteroaryl), —SO₂NR⁶R⁷, —SO₂NR⁶(cycloalkyl), —SO₂NR⁶(heterocycloalkyl), —SR⁶, —SO₂R⁶, —SO₂NR⁶(aryl), —SO₂NR⁶(heteroaryl), haloalkyl, aryl, heteroaryl, heterocyclyl and tetrazoyl.

In some embodiments of a compound of Formula (Ic), A is selected from:

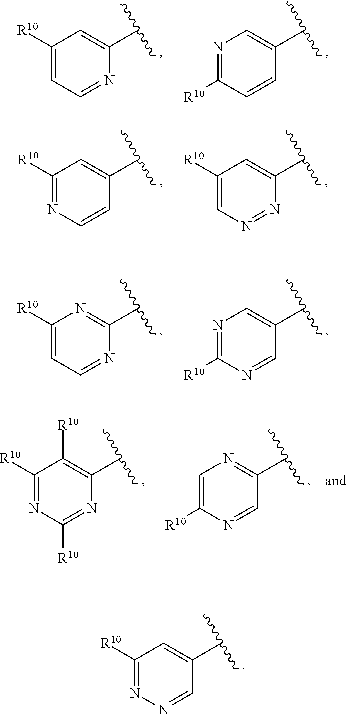

In some embodiments of a compound of Formula (Ic), A is

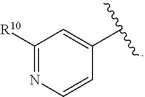

In some embodiments of a compound of Formula (Ic), A is selected from:

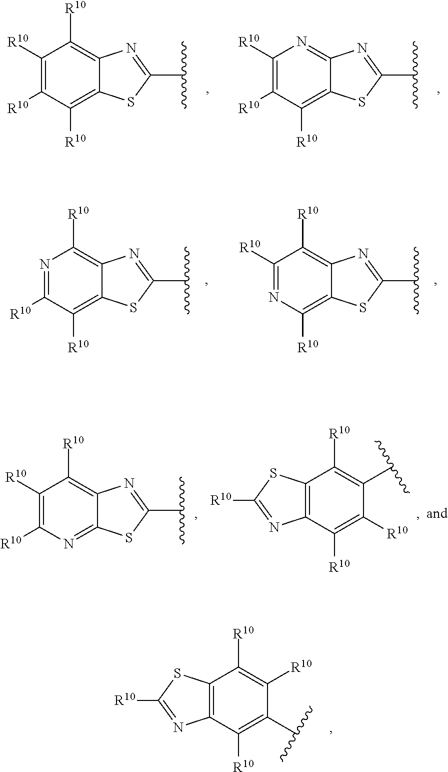

In some embodiments of a compound of Formula (Ic), A is

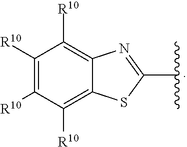

In one aspect, provided herein are compounds of Formula (Id), a pharmaceutically acceptable salt, solvate, polymorph, prodrug, metabolite, N-oxide, stereoisomer, or isomer thereof:

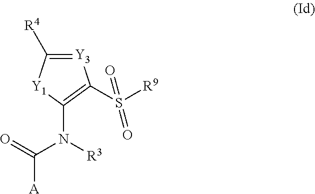

wherein:
Y₁ is S or O;
Y₃ is CR⁵ or N;
R³ is H, haloalkyl, or alkyl;
R⁴ is H, halogen, CN or optionally substituted alkyl;
R⁵ is H, optionally substituted alkyl, or halogen;
R⁹ is optionally substituted alkyl, optionally substituted heteroaryl or optionally substituted aryl; and
A is optionally substituted heteroaryl, optionally substituted aryl, or optionally substituted heterocyclyl.

In some embodiments of a compound of Formula (Id), Y₃ is N. In some embodiments of a compound of Formula (Id), Y₃ is CR⁵. In some embodiments of a compound of Formula (Id), Y₃ is CH.

In some embodiments of a compound of Formula (Id), Y₁ is O. In some embodiments of a compound of Formula (Id), Y₁ is S. In some embodiments of a compound of Formula (Id), Y₁ is S and Y₃ is CH.

In some embodiments of a compound of Formula (Id), R³ is H. In some embodiments of a compound of Formula (Id), R³ is haloalkyl. In some embodiments of a compound of Formula (Id), R³ is alkyl. In some embodiments of a compound of Formula (Id), R³ is H or alkyl. In some embodiments of a compound of Formula (Id), R³ is haloalkyl or alkyl.

In some embodiments of a compound of Formula (Id), R⁴ is H. In some embodiments of a compound of Formula (Id), R⁴ is CN. In some embodiments of a compound of Formula (Id), R⁴ is optionally substituted alkyl. In some embodiments of a compound of Formula (Id), Y₁ is S, Y₃ is CH, and R⁴ is H. In some embodiments of a compound of Formula (Id), Y₁ is S, Y₃ is CH, and R³ and R⁴ are H.

In some embodiments of a compound of Formula (Id), R⁵ is H. In some embodiments of a compound of Formula (Id), R⁵ is optionally substituted alkyl. In some embodiments of a compound of Formula (Id), R⁵ is halogen. In some embodiments of a compound of Formula (Id), R⁵ is H or optionally substituted alkyl. In some embodiments of a compound of Formula (Id), R⁵ is H or halogen. In some embodiments of a compound of Formula (Id), R⁵ is optionally substituted alkyl or halogen.

In some embodiments of a compound of Formula (Id), R⁹ is optionally substituted alkyl. In some embodiments of a compound of Formula (Id), R⁹ is optionally substituted heteroaryl. In some embodiments of a compound of Formula (Id), R⁹ is optionally substituted aryl.

In some embodiments of a compound of Formula (Id), A is optionally substituted heteroaryl. In some embodiments of a compound of Formula (Id), A is optionally substituted aryl. In some embodiments of a compound of Formula (Id), A is optionally substituted heterocyclyl.

In one aspect, provided herein are compounds of Formula (II), a pharmaceutically acceptable salt, solvate, polymorph, prodrug, metabolite, N-oxide, stereoisomer, or isomer thereof:

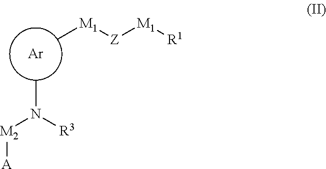

wherein:
Ar is optionally substituted aryl or optionally substituted heteroaryl;
M₁ are each independently a bond, —C(═O)— or —S(═O)₂—;
M₂ is —(CH₂)—, —C(═O)— or —S(═O)₂—;
Z is a bond or NR²;
R¹ is —O-(optionally substituted alkyl), —O-(alkenyl), —O-(alkenyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), —O-(alkyl)-(NR⁶R⁷), —NR⁶R⁷ or R⁸;
R² and R³ are each independently selected from H, optionally substituted alkyl, and optionally substituted aryl; or R¹ and R² taken together form a heterocycle;
R⁶ and R⁷ are each independently selected from H and optionally substituted alkyl; or R⁶ and R⁷ taken together form an optionally substituted heterocycle with the nitrogen to which they are attached;
R⁸ is optionally substituted alkyl, optionally substituted aryl, carbocyclyl, optionally substituted aralkyl, optionally substituted heteroaryl, optionally substituted heterocyclyl, —R^{b}COOR^{a} or —R^{b}CONR^{a}R^{a};
each R^{a} is independently selected from H and alkyl;
R^{b} is a bond or alkylenyl;
R^{c} is a bond or alkenylenyl; and
A is optionally substituted aryl, optionally substituted heterocyclyl, optionally substituted heteroaryl, optionally substituted carbocyclyl, optionally substituted aralkyl, optionally substituted heteroaralkyl or —R^{c}-(optionally substituted heteroaryl).

In some embodiments of a compound of Formula (II), Ar is optionally substituted aryl. In some embodiments of a compound of Formula (II), Ar is phenyl. In some embodiments of a compound of Formula (II), Ar is

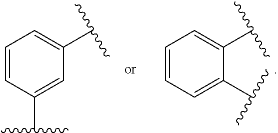 [SMILES: *c1cccc(*)c1 ; *c1ccccc1*]

In some embodiments of a compound of Formula (II), Ar is

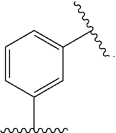 [SMILES: *c1cccc(*)c1]

In some embodiments of a compound of Formula (II), Ar is

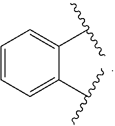 [SMILES: *c1ccccc1*]

In some embodiments of a compound of Formula (II), Ar is optionally substituted heteroaryl. In some embodiments of a compound of Formula (II), Ar is pyridinyl. In some embodiments of a compound of Formula (II), Ar is

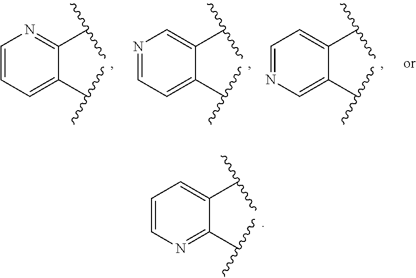 [SMILES: *c1cccnc1* ; *c1ccncc1* ; *c1ccncc1* ; *c1cccnc1*]

In some embodiments of a compound of Formula (II), Ar is

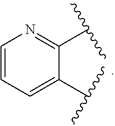 [SMILES: *c1cccnc1*]

In some embodiments of a compound of Formula (II), Ar is

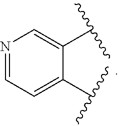 [SMILES: *c1ccncc1*]

In some embodiments of a compound of Formula (II), Ar is

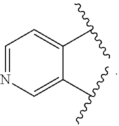 [SMILES: *c1ccncc1*]

In some embodiments of a compound of Formula (II), Ar

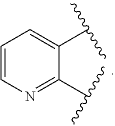 [SMILES: *c1cccnc1*]

In some embodiments of a compound of Formula (II), Ar is

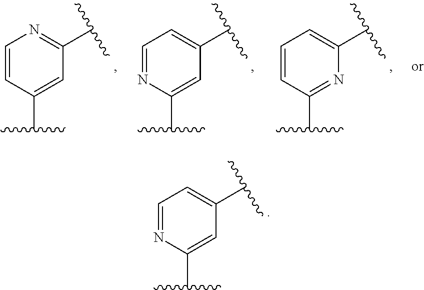 [SMILES: *c1ccnc(*)c1 ; *c1ccnc(*)c1 ; *c1cccc(*)n1 ; *c1ccnc(*)c1]

In some embodiments of a compound of Formula (II), Ar is

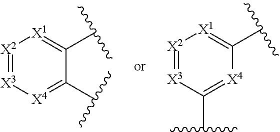

wherein X¹, X², X³, and X⁴ are independently selected from N and CR¹⁰; and each R¹⁰ is independently selected from H, halogen, —CN, —NO₂, —CF₃, alkyl, —SR⁶, —OR⁶, —NR⁶R⁷, —NR⁶C(═O)(alkyl), —NR⁶C(═O)(cycloalkyl), —NR⁶C(═O)(heterocyclyl), —NR⁶C(═O)(aryl), —NR⁶C(═O)(heteroaryl), —C(═O)NR⁶R⁷, —C(═O)NR⁶(cycloalkyl), —C(═O)NR⁶(heterocycloalkyl), —C(═O)NR⁶(aryl), —C(═O)NR⁶(heteroaryl), —NR⁶C(═O)NR⁶R⁷, —NR⁶C(═O)NR⁷(cycloalkyl), —NR⁶C(═O)NR⁷(heterocycloalkyl), —NR⁶C(═O)NR⁷(aryl), —NR⁶C(═O)NR⁷(heteroaryl), —NR⁶C(═O)O(alkyl), —NR⁶C(═O)O(cycloalkyl), —NR⁶C(═O)O(heterocycloalkyl), —NR⁶C(═O)O(aryl), —NR⁶C(═O)O(heteroaryl), —NR⁶SO₂(alkyl), —NR⁶SO₂(cycloalkyl), —NR⁶SO₂(heterocycloalkyl), —NR⁶SO₂(aryl), —NR⁶SO₂(heteroaryl), —SO₂NR⁶R⁷, —SO₂NR⁶(cycloalkyl), —SO₂NR⁶(heterocycloalkyl), —SR⁶, —SO₂R⁶, —SO₂NR⁶(aryl), —SO₂NR⁶(heteroaryl), haloalkyl, aryl, heteroaryl, heterocyclyl and tetrazoyl.

In some embodiments of a compound of Formula (II), each M₁ is a bond. In some embodiments of a compound of Formula (II), each M₁ is —C(═O)—. In some embodiments of a compound of Formula (II), each M₁ is —S(═O)₂—.

In some embodiments of a compound of Formula (II), Z is a bond. In some embodiments of a compound of Formula (II), Z is NR². In some embodiments of a compound of Formula (II), Z is NR² and each M₁ is —C(═O)—.

In some embodiments of a compound of Formula (II), M₂ is —C(═O)—.

In some embodiments of a compound of Formula (II), M₁ and M₂ are both —C(═O)—. In some embodiments of a compound of Formula (II), M₁ and M₂ are both —C(═O)—; and Z is NR².

In some embodiments of a compound of Formula (II), R¹ is —O-(optionally substituted alkyl), —O-(alkenyl), —O-(alkenyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), or —O-(alkyl)-(NR⁶R⁷). In some embodiments of a compound of Formula (II), R¹ is —O-(optionally substituted alkyl). In some embodiments of a compound of Formula (II), R¹ is —O-(alkenyl). In some embodiments of a compound of Formula (II), R¹ is —O-(alkenyl). In some embodiments of a compound of Formula (II), R¹ is —O-(cycloalkyl). In some embodiments of a compound of Formula (II), R¹ is —O-(heterocyclyl). In some embodiments of a compound of Formula (II), R¹ is —O-(optionally substituted aralkyl). In some embodiments of a compound of Formula (II), R¹ is —O-(optionally substituted heteroaralkyl). In some embodiments of a compound of Formula (II), R¹ is —O-(alkyl)-(alkoxy). In some embodiments of a compound of Formula (II), R¹ is —O-(alkyl)-(aralkoxy). In some embodiments of a compound of Formula (II), R¹ is —O-(alkyl)-(heterocyclyl). In some embodiments of a compound of Formula (II), R¹ is —O-(alkyl)-(COOR^{a}). In some embodiments of a compound of Formula (II), R¹ is —O-(alkyl)-(NR⁶R⁷). In some embodiments of a compound of Formula (II), R¹ is —O-(optionally substituted alkyl); and R² and R³ are both H.

In some embodiments of a compound of Formula (II), R¹ is —NR⁶R⁷. In some embodiments of a compound of Formula (II), R⁶ and R⁷ are each independently selected from H and optionally substituted alkyl. In some embodiments of a compound of Formula (II), R⁶ and R⁷ are each independently selected from H and optionally substituted alkyl; wherein the optional substituent is halogen. In some embodiments of a compound of Formula (II), R⁶ and R⁷ are H. In some embodiments of a compound of Formula (II), R⁶ and R⁷ are each optionally substituted alkyl. In some embodiments of a compound of Formula (II), R⁶ and R⁷ are each optionally substituted alkyl; wherein the optional substituent is halogen. In some embodiments of a compound of Formula (II), R⁶ and R⁷ taken together form an optionally substituted heterocycle with the nitrogen to which they are attached. In some embodiments of a compound of Formula (II), R⁶ and R⁷ taken together form an optionally substituted heterocycle with the nitrogen to which they are attached; wherein the optional substituent is halogen.

In some embodiments of a compound of Formula (II), R¹ is R⁸. In some embodiments of a compound of Formula (II), R⁸ is optionally substituted alkyl. In some embodiments of a compound of Formula (II), R⁸ is optionally substituted aryl. In some embodiments of a compound of Formula (II), R⁸ is carbocyclyl. In some embodiments of a compound of Formula (II), R⁸ is optionally substituted aralkyl. In some embodiments of a compound of Formula (II), R⁸ is optionally substituted heteroaryl. In some embodiments of a compound of Formula (II), R⁸ is optionally substituted heterocyclyl. In some embodiments of a compound of Formula (II), R⁸ is —R^{b}COOR^{a}. In some embodiments of a compound of Formula (II), R⁸ is —R^{b}CONR^{a}R^{a}.

In some embodiments of a compound of Formula (II), R² and R³ are each independently selected from H, optionally substituted alkyl and optionally substituted aryl. In some embodiments of a compound of Formula (II), R² and R³ are H. In some embodiments of a compound of Formula (II), R² and R³ are each independently selected from H and optionally substituted alkyl. In some embodiments of a compound of Formula (II), R² and R³ are optionally substituted alkyl. In some embodiments of a compound of Formula (II), R² and R³ are optionally substituted aryl. In some embodiments of a compound of Formula (II), R² and R³ are each independently selected from optionally substituted alkyl and optionally substituted aryl. In some embodiments of a compound of Formula (II), R¹ and R² taken together form a heterocycle.

In one aspect, provided herein are compounds of Formula (IIa), a pharmaceutically acceptable salt, solvate, polymorph, prodrug, metabolite, N-oxide, stereoisomer, or isomer thereof:

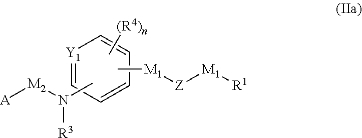

wherein:
Y₁ is N, CH, or CR⁴;
M₁ are each independently a bond, —C(═O)— or —S(═O)₂—;
M₂ is —(CH₂)—, —C(═O)— or —S(═O)₂—;
Z is a bond or NR²;
R¹ is —O-(optionally substituted alkyl), —O-(alkenyl), —O-(alkenyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), —O-(alkyl)-(NR⁶R⁷), —NR⁶R⁷ or R⁸;
R² and R³ are each independently selected from H, optionally substituted alkyl, and optionally substituted aryl; or R¹ and R² taken together form a heterocycle;
R⁴ is halogen, —CN, optionally substituted alkyl, optionally substituted alkoxy, optionally substituted aryl, —R^{b}COOR^{a} or —R^{b}CH(COOR^{a})₂;
R⁶ and R⁷ are each independently selected from H and optionally substituted alkyl; or R⁶ and R⁷ taken together form an optionally substituted heterocycle with the nitrogen to which they are attached;
R⁸ is optionally substituted alkyl, optionally substituted aryl, carbocyclyl, optionally substituted aralkyl, optionally substituted heteroaryl, optionally substituted heterocyclyl, —R^{b}COOR^{a} or —R^{b}CONR^{a}R^{a};
each R^{a} is independently selected from H and alkyl;
R^{b} is a bond or alkylenyl;
R^{c} is a bond or alkenylenyl;
n is 0, 1, 2, or 3; and
A is optionally substituted aryl, optionally substituted heterocyclyl, optionally substituted heteroaryl, optionally substituted carbocyclyl, optionally substituted aralkyl, optionally substituted heteroaralkyl or —R^{c}-(optionally substituted heteroaryl).

In some embodiments of a compound of Formula (IIa), Y₁ is N. In some embodiments of a compound of Formula (IIa), Y₁ is CR⁴. In some embodiments of a compound of Formula (IIa), Y₁ is CH.

In some embodiments of a compound of Formula (IIa), each M₁ is a bond. In some embodiments of a compound of Formula (IIa), each M₁ is —C(═O)—. In some embodiments of a compound of Formula (IIa), each M₁ is —S(═O)₂—.

In some embodiments of a compound of Formula (IIa), Z is a bond. In some embodiments of a compound of Formula (IIa), Z is NR². In some embodiments of a compound of Formula (IIa), Z is NR² and each M₁ is —C(═O)—.

In some embodiments of a compound of Formula (IIa), -M₁-Z-M₁-R¹ is —C(═O)—R¹. In further embodiments of a compound of Formula (IIa), -M₁-Z-M₁-R¹ is —C(═O)—O-(optionally substituted alkyl). In other embodiments of a compound of Formula (IIa), -M₁-Z-M₁-R¹ is —C(═O)— NR⁶R⁷. In still other embodiments of a compound of Formula (IIa), -M₁-Z-M₁-R¹ is —C(═O)—R⁸.

In some embodiments of a compound of Formula (IIa), M₂ is —C(═O)—.

In some embodiments of a compound of Formula (IIa), M₁ and M₂ are both —C(═O)—. In some embodiments of a compound of Formula (IIa), M₁ and M₂ are both —C(═O)—; and Z is NR².

In some embodiments of a compound of Formula (IIa), R¹ is —O-(optionally substituted alkyl), —O-(alkenyl), —O-(alkenyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), or —O-(alkyl)-(NR⁶R⁷). In some embodiments of a compound of Formula (IIa), R¹ is —O-(optionally substituted alkyl). In some embodiments of a compound of Formula (IIa), R¹ is —O-(alkenyl). In some embodiments of a compound of Formula (IIa), R¹ is —O-(alkenyl). In some embodiments of a compound of Formula (IIa), R¹ is —O-(cycloalkyl). In some embodiments of a compound of Formula (IIa), R¹ is —O-(heterocyclyl). In some embodiments of a compound of Formula (IIa), R¹ is —O-(optionally substituted aralkyl). In some embodiments of a compound of Formula (IIa), R¹ is —O-(optionally substituted heteroaralkyl). In some embodiments of a compound of Formula (IIa), R¹ is —O-(alkyl)-(alkoxy). In some embodiments of a compound of Formula (IIa), R¹ is —O-(alkyl)-(aralkoxy). In some embodiments of a compound of Formula (IIa), R¹ is —O-(alkyl)-(heterocyclyl). In some embodiments of a compound of Formula (IIa), R¹ is —O-(alkyl)-(COOR^{a}). In some embodiments of a compound of Formula (IIa), R¹ is —O-(alkyl)-(NR⁶R⁷). In some embodiments of a compound of Formula (IIa), R¹ is —O-(optionally substituted alkyl); and R² and R³ are both H.

In some embodiments of a compound of Formula (IIa), R¹ is —NR⁶R⁷. In some embodiments of a compound of Formula (IIa), R⁶ and R⁷ are each independently selected from H and optionally substituted alkyl. In some embodiments of a compound of Formula (IIa), R⁶ and R⁷ are each independently selected from H and optionally substituted alkyl; wherein the optional substituent is halogen. In some embodiments of a compound of Formula (IIa), R⁶ and R⁷ are H. In some embodiments of a compound of Formula (IIa), R⁶ and R⁷ are each optionally substituted alkyl. In some embodiments of a compound of Formula (IIa), R⁶ and R⁷ are each optionally substituted alkyl; wherein the optional substituent is halogen. In some embodiments of a compound of Formula (IIa), R⁶ and R⁷ taken together form an optionally substituted heterocycle with the nitrogen to which they are attached. In some embodiments of a compound of Formula (IIa), R⁶ and R⁷ taken together form an optionally substituted heterocycle with the nitrogen to which they are attached; wherein the optional substituent is halogen.

In some embodiments of a compound of Formula (IIa), R¹ is R⁸. In some embodiments of a compound of Formula (IIa), R⁸ is optionally substituted alkyl. In some embodiments of a compound of Formula (IIa), R⁸ is optionally substituted aryl. In some embodiments of a compound of Formula (IIa), R⁸ is carbocyclyl. In some embodiments of a compound of Formula (IIa), R⁸ is optionally substituted aralkyl. In some embodiments of a compound of Formula (IIa), R⁸ is optionally substituted heteroaryl. In some embodiments of a compound of Formula (IIa), R⁸ is optionally substituted heterocyclyl. In some embodiments of a compound of Formula (IIa), R⁸ is —R^{b}COOR^{a}. In some embodiments of a compound of Formula (IIa), R⁸ is —R^{b}CONR^{a}R^{a}. In some embodiments of a compound of Formula (IIa), R² and R⁸ taken together form an optionally substituted heterocycle with the nitrogen to which they are attached. In some embodiments of a compound of Formula (IIa), R⁸ is optionally substituted alkyl, optionally substituted aryl, optionally substituted aralkyl, carbocyclyl, optionally substituted carbocyclylalkyl, optionally substituted heteroaryl, optionally substituted heteroarylalkyl, optionally substituted heterocyclyl, or optionally substituted heterocyclylalkyl. In some embodiments of a compound of Formula (IIa), R⁸ is optionally substituted alkyl, carbocyclyl, optionally substituted carbocyclylalkyl, optionally substituted heterocyclyl, or optionally substituted heterocyclylalkyl. In some embodiments of a compound of Formula (IIa), R⁸ is optionally substituted aryl, optionally substituted aralkyl, optionally substituted heteroaryl, or optionally substituted heteroarylalkyl. In some embodiments of a compound of Formula (IIa), R⁸ is optionally substituted alkyl, optionally substituted aralkyl, carbocyclyl, optionally substituted carbocyclylalkyl, optionally substituted heteroarylalkyl, optionally substituted heterocyclyl, or optionally substituted heterocyclylalkyl.

In some embodiments of a compound of Formula (IIa), R² and R³ are each independently selected from H, optionally substituted alkyl, and optionally substituted aryl. In some embodiments of a compound of Formula (IIa), R² and R³ are H. In some embodiments of a compound of Formula (IIa), R² and R³ are each independently selected from H and optionally substituted alkyl. In some embodiments of a compound of Formula (IIa), R² and R³ are optionally substituted alkyl. In some embodiments of a compound of Formula (IIa), R² and R³ are optionally substituted aryl. In some embodiments of a compound of Formula (IIa), R² and R³ are each independently selected from optionally substituted alkyl and optionally substituted aryl. In some embodiments of a compound of Formula (IIa), R¹ and R² taken together form a heterocycle.

In some embodiments of a compound of Formula (IIa), R⁴ is halogen. In some embodiments of a compound of Formula (IIa), R⁴ is —CN. In some embodiments of a compound of Formula (IIa), R⁴ is optionally substituted alkyl. In some embodiments of a compound of Formula (IIa), R⁴ is optionally substituted alkoxy. In some embodiments of a compound of Formula (IIa), R⁴ is optionally substituted aryl. In some embodiments of a compound of Formula (IIa), R⁴ is —R^{b}COOR^{a}. In some embodiments of a compound of Formula (IIa), R⁴ is —R^{b}CH(COOR^{a})₂. In some embodiments of a compound of Formula (IIa), R⁴ is halogen, —CN, or optionally substituted alkyl. In some embodiments of a compound of Formula (IIa), R⁴ is halogen, —CN, optionally substituted alkyl, or optionally substituted aryl. In some embodiments of a compound of Formula (IIa), R⁴ is —R^{b}COOR^{a} or —R^{b}CH(COOR^{a})₂.

In one aspect, provided herein are compounds of Formula (IIb), a pharmaceutically acceptable salt, solvate, polymorph, prodrug, metabolite, N-oxide, stereoisomer, or isomer thereof:

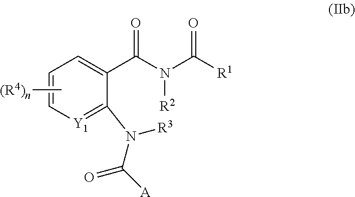

wherein:
Y₁ is N, CH, or CR⁴;
R¹ is —O-(optionally substituted alkyl), —O-(alkenyl), —O-(alkenyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), —O-(alkyl)-(NR⁶R⁷), —NR⁶R⁷ or R⁸;
R² and R³ are each independently selected from H, optionally substituted alkyl, and optionally substituted aryl; or R¹ and R² taken together form a heterocycle;
each R⁴ is independently selected from halogen, —CN, optionally substituted alkyl, optionally substituted alkoxy, optionally substituted aryl, —R^{b}COOR^{a}, and —R^{b}CH(COOR^{a})₂;
R⁶ and R⁷ are each independently selected from H and optionally substituted alkyl; or R⁶ and R⁷ taken together form an optionally substituted heterocycle with the nitrogen to which they are attached;
R⁸ is optionally substituted alkyl, optionally substituted aryl, carbocyclyl, optionally substituted aralkyl, optionally substituted heteroaryl, or optionally substituted heterocyclyl;
each R^{a} is independently selected from H and alkyl;
R^{b} is a bond or alkylenyl;
R^{c} is a bond or alkenylenyl;
n is 0, 1, 2, or 3; and
A is optionally substituted aryl, optionally substituted heterocyclyl, optionally substituted heteroaryl, optionally substituted carbocyclyl, optionally substituted aralkyl, optionally substituted heteroaralkyl or —R^{c}-(optionally substituted heteroaryl).

In some embodiments of a compound of Formula (IIb), Y₁ is N. In some embodiments of a compound of Formula (IIb), Y₁ is N and n is 0. In some embodiments of a compound of Formula (IIb), Y₁ is N and n is 1. In some embodiments of a compound of Formula (IIb), Y₁ is N and n is 2.

In some embodiments of a compound of Formula (IIb), Y₁ is CR⁴. In some embodiments of a compound of Formula (IIb), Y₁ is CH. In some embodiments of a compound of Formula (IIb), Y₁ is CH and n is 0. In some embodiments of a compound of Formula (IIb), Y₁ is CH and n is 1. In some embodiments of a compound of Formula (IIb), Y₁ is CH and n is 2.

In some embodiments of a compound of Formula (IIb), R¹ is —O-(optionally substituted alkyl), —O-(alkenyl), —O-(alkenyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), or —O-(alkyl)-(NR⁶R⁷). In some embodiments of a compound of Formula (IIb), R¹ is —O-(optionally substituted alkyl). In some embodiments of a compound of Formula (IIb), R¹ is —O-(alkenyl). In some embodiments of a compound of Formula (IIb), R¹ is —O-(alkenyl). In some embodiments of a compound of Formula (IIb), R¹ is —O-(cycloalkyl). In some embodiments of a compound of Formula (IIb), R¹ is —O-(heterocyclyl). In some embodiments of a compound of Formula (IIb), R¹ is —O-(optionally substituted aralkyl). In some embodiments of a compound of Formula (IIb), R¹ is —O-(optionally substituted heteroaralkyl). In some embodiments of a compound of Formula (IIb), R¹ is —O-(alkyl)-(alkoxy). In some embodiments of a compound of Formula (IIb), R¹ is —O-(alkyl)-(aralkoxy). In some embodiments of a compound of Formula (IIb), R¹ is —O-(alkyl)-(heterocyclyl). In some embodiments of a compound of Formula (IIb), R¹ is —O-(alkyl)-(COOR^{a}). In some embodiments of a compound of Formula (IIb), R¹ is —O-(alkyl)-(NR⁶R⁷).

In some embodiments of a compound of Formula (IIb), R¹ is —O-(optionally substituted alkyl); wherein the optionally substituted alkyl is substituted with halogen. In further embodiments of a compound of Formula (IIb), R¹ is —O-(cycloalkyl). In still further embodiments of a compound of Formula (IIb), R¹ is —O-(cyclobutyl), —O-(cyclopentyl), or —O-(cyclohexyl). In some embodiments of a compound of Formula (IIb), R¹ is —O-(heterocyclyl). In further embodiments of a compound of Formula (IIb), R¹ is —O-(optionally substituted piperidinyl), —O-(oxetanyl), or —O-(tetrahydrofuranyl), wherein the optionally substituted piperidinyl is substituted with —COCH₃. In some embodiments of a compound of Formula (IIb), R¹ is —O-(optionally substituted alkyl); and R² and R³ are both H.

In some embodiments of a compound of Formula (IIb), R¹ is —O-(optionally substituted aralkyl) or —O-(optionally substituted heteroaralkyl). In further embodiments of a compound of Formula (IIb), the optionally substituted aralkyl and the optionally substituted heteroaralkyl are substituted with a group selected from halogen, alkyl and —CF₃.

In some embodiments of a compound of Formula (IIb), R¹ is —O-(alkyl)-(heterocyclyl). In further embodiments of a compound of Formula (IIb), the heterocyclyl is morpholinyl.

In some embodiments of a compound of Formula (IIb), R¹ is —NR⁶R⁷. In some embodiments of a compound of Formula (IIb), R⁶ and R⁷ are each independently selected from H and optionally substituted alkyl. In some embodiments of a compound of Formula (IIb), R⁶ and R⁷ are each independently selected from H and optionally substituted alkyl; wherein the optional substituent is halogen. In some embodiments of a compound of Formula (IIb), R⁶ and R⁷ are H. In some embodiments of a compound of Formula (IIb), R⁶ and R⁷ are each optionally substituted alkyl. In some embodiments of a compound of Formula (IIb), R⁶ and R⁷ are each optionally substituted alkyl; wherein the optional substituent is halogen. In some embodiments of a compound of Formula (IIb), R⁶ and R⁷ taken together form an optionally substituted heterocycle with the nitrogen to which they are attached. In some embodiments of a compound of Formula (IIb), R⁶ and R⁷ taken together form an optionally substituted heterocycle with the nitrogen to which they are attached; wherein the optional substituent is halogen. In some embodiments of a compound of Formula (IIb), R⁶ and R⁷ taken together form a heterocycle with the nitrogen to which they are attached; wherein the heterocycle is selected from piperidinyl and morpholinyl.

In some embodiments of a compound of Formula (IIb), R¹ is R⁸. In some embodiments of a compound of Formula (IIb), R⁸ is optionally substituted alkyl. In some embodiments of a compound of Formula (IIb), R⁸ is optionally substituted aryl. In some embodiments of a compound of Formula (IIb), R⁸ is optionally substituted aryl; wherein the optionally substituted aryl is substituted with halogen. In some embodiments of a compound of Formula (IIb), R⁸ is carbocyclyl. In some embodiments of a compound of Formula (IIb), R⁸ is optionally substituted carbocyclylalkyl. In some embodiments of a compound of Formula (IIb), R⁸ is optionally substituted heteroaryl. In some embodiments of a compound of Formula (IIb), R⁸ is optionally substituted heteroaryl; wherein the optionally substituted heteroaryl is substituted with a group selected from alkyl, —O-(alkyl) and —NR⁶R⁷. In some embodiments of a compound of Formula (IIb), R⁸ is optionally substituted heteroarylalkyl. In some embodiments of a compound of Formula (IIb), R⁸ is optionally substituted heterocyclyl. In some embodiments of a compound of Formula (IIb), R⁸ is optionally substituted heterocyclyl; wherein the optionally substituted heterocyclyl is substituted with alkyl. In some embodiments of a compound of Formula (IIb), R² and R⁸ taken together form an optionally substituted heterocycle with the nitrogen to which they are attached. In further embodiments of a compound of Formula (IIb), the heterocycle is selected from piperidinyl, morpholinyl, thiomorpholinyl, optionally substituted diazepanyl and optionally substituted piperizanyl; wherein the optionally substituted diazepanyl and the optionally substituted piperizanyl are substituted with a group selected from alkyl, aryl, -COOtBu and —SO₂Me. In some embodiments of a compound of Formula (IIb), R⁸ is optionally substituted heterocyclylalkyl. In some embodiments of a compound of Formula (IIb), R² and R⁸ taken together form an optionally substituted heterocycle with the nitrogen to which they are attached. In some embodiments of a compound of Formula (IIb), R⁸ is optionally substituted alkyl, optionally substituted aryl, optionally substituted aralkyl, carbocyclyl, optionally substituted carbocyclylalkyl, optionally substituted heteroaryl, optionally substituted heteroarylalkyl, optionally substituted heterocyclyl, or optionally substituted heterocyclylalkyl. In some embodiments of a compound of Formula (IIb), R⁸ is optionally substituted alkyl, carbocyclyl, optionally substituted carbocyclylalkyl, optionally substituted heterocyclyl, or optionally substituted heterocyclylalkyl. In some embodiments of a compound of Formula (IIb), R⁸ is optionally substituted aryl, optionally substituted aralkyl, optionally substituted heteroaryl, or optionally substituted heteroarylalkyl. In some embodiments of a compound of Formula (IIb), R⁸ is optionally substituted alkyl, optionally substituted aralkyl, carbocyclyl, optionally substituted carbocyclylalkyl, optionally substituted heteroarylalkyl, optionally substituted heterocyclyl, or optionally substituted heterocyclylalkyl.

In some embodiments of a compound of Formula (IIb), R⁴ is halogen. In some embodiments of a compound of Formula (IIb), R⁴ is —CN. In some embodiments of a compound of Formula (IIb), R⁴ is optionally substituted alkyl. In some embodiments of a compound of Formula (IIb), R⁴ is optionally substituted alkoxy. In some embodiments of a compound of Formula (IIb), R⁴ is optionally substituted aryl. In some embodiments of a compound of Formula (IIb), R⁴ is —R^{b}COOR^{a}. In some embodiments of a compound of Formula (IIb), R⁴ is —R^{b}CH(COOR^{a})₂. In some embodiments of a compound of Formula (IIb), R⁴ is halogen, —CN, or optionally substituted alkyl. In some embodiments of a compound of Formula (IIb), R⁴ is halogen, —CN, optionally substituted alkyl, or optionally substituted aryl. In some embodiments of a compound of Formula (IIb), R⁴ is —R^{b}COOR^{a} or —R^{b}CH(COOR^{a})₂.

In some embodiments of a compound of Formula (IIb), Y₁ is N; n is 1; and R⁴ is halogen, —CN, or optionally substituted alkyl. In some embodiments of a compound of Formula (IIb), Y₁ is CH; n is 1; and R⁴ is halogen, —CN, or optionally substituted alkyl.

In some embodiments of a compound of Formulas (II), (IIa), or (IIb), A is optionally substituted aryl, optionally substituted heterocyclyl, optionally substituted heteroaryl, optionally substituted carbocyclyl, optionally substituted aralkyl, optionally substituted heteroaralkyl or —R^{c}-(optionally substituted heteroaryl); and the optionally substituted aryl, the optionally substituted heterocyclyl, the optionally substituted heteroaryl, the optionally substituted carbocyclyl, the optionally substituted aralkyl and the optionally substituted heteroaralkyl are substituted with 1-6 R¹⁰; wherein each R¹⁰ is independently selected from H, halogen, —CN, —NO₂, —CF₃, alkyl, —SR⁶, —OR⁶, —NR⁶R⁷, —NR⁶C(═O)(alkyl), —NR⁶C(═O)(cycloalkyl), —NR⁶C(═O)(heterocyclyl), —NR⁶C(═O)(aryl), —NR⁶C(═O)(heteroaryl), —C(═O)NR⁶R⁷, —C(═O)NR⁶(cycloalkyl), —C(═O)NR⁶(heterocycloalkyl), —C(═O)NR⁶(aryl), —C(═O)NR⁶(heteroaryl), —NR⁶C(═O)NR⁶R⁷, —NR⁶C(═O)NR⁷(cycloalkyl), —NR⁶C(═O)NR⁷(heterocycloalkyl), —NR⁶C(═O)NR⁷(aryl), —NR⁶C(═O)NR⁷(heteroaryl), —NR⁶C(═O)O(alkyl), —NR⁶C(═O)O(cycloalkyl), —NR⁶C(═O)O(heterocycloalkyl), —NR⁶C(═O)O(aryl), —NR⁶C(═O)O(heteroaryl), —NR⁶SO₂(alkyl), —NR⁶SO₂(cycloalkyl), —NR⁶SO₂(heterocycloalkyl), —NR⁶SO₂(aryl), —NR⁶SO₂(heteroaryl), —SO₂NR⁶R⁷, —SO₂NR⁶(cycloalkyl), —SO₂NR⁶(heterocycloalkyl), —SR⁶, —SO₂R⁶, —SO₂NR⁶(aryl), —SO₂NR⁶(heteroaryl), haloalkyl, aryl, heteroaryl, heterocyclyl and tetrazoyl.

In some embodiments of a compound of Formulas (II), (IIa), or (IIb), A is optionally substituted aryl. In some embodiments of a compound of Formulas (II), (IIa), or (IIb), A is optionally substituted heterocyclyl. In some embodiments of a compound of Formulas (II), (IIa), or (IIb), A is optionally substituted carbocyclyl. In some embodiments of a compound of Formulas (II), (IIa), or (IIb), A is optionally substituted aralkyl. In some embodiments of a compound of Formulas (II), (IIa), or (IIb), A is optionally substituted heteroaralkyl. In some embodiments of a compound of Formulas (II), (IIa), or (IIb), A is optionally substituted —R^{c}-(optionally substituted heteroaryl).

In some embodiments of a compound of Formulas (II), (IIa), or (IIb), A is optionally substituted heteroaryl. In some embodiments of a compound of Formulas (II), (IIa), or (IIb), A is selected from:

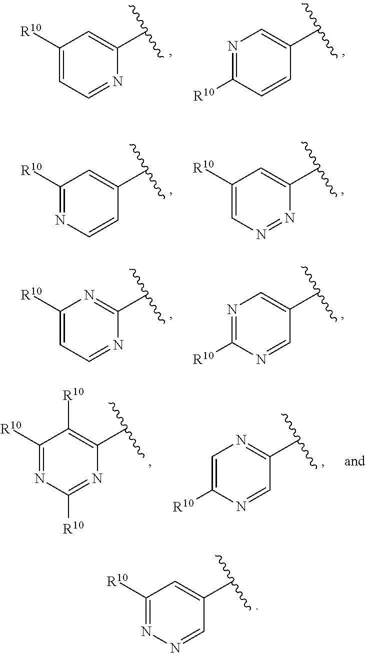

In some embodiments of a compound of Formulas (II), (IIa), or (IIb), A is

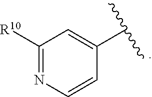

In some embodiments of a compound of Formulas (II), (IIa), or (IIb), A is selected from

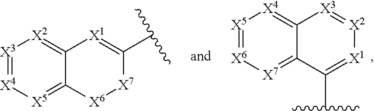

wherein X¹, X², X³, X⁴, X⁵, X⁶, and X⁷ are independently selected from N and CR¹⁰; and at least one of X¹-X⁷ is N. In some embodiments of a compound of Formulas (II), (IIa), or (IIb), A is selected from

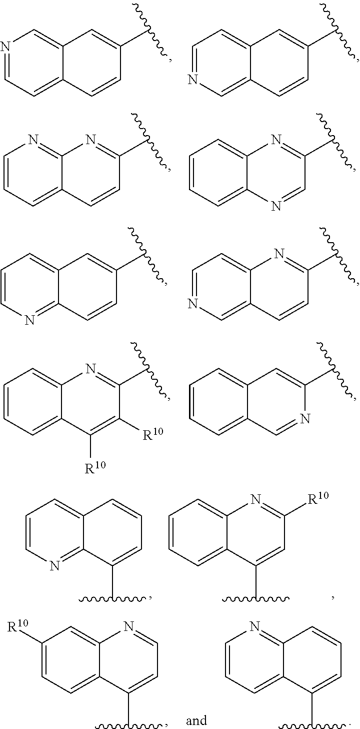

In some embodiments of a compound of Formulas (II), (IIa), or (IIb), A is selected from

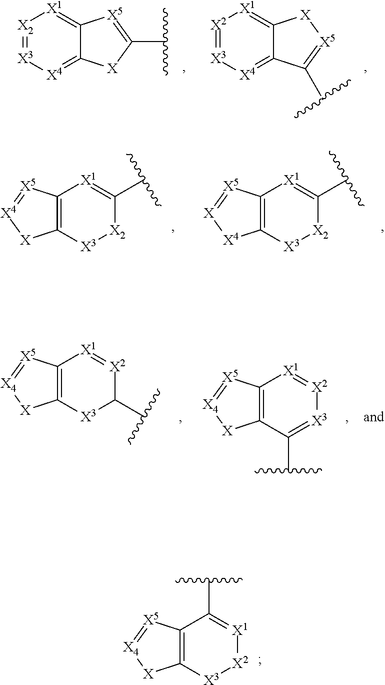

wherein X¹, X², X³, X⁴, X⁵, X⁶, and X⁷ are independently selected from N and CR¹⁰; and X is O, S, or NR². In some embodiments of a compound of Formulas (II), (IIa), or (IIb), A is selected from

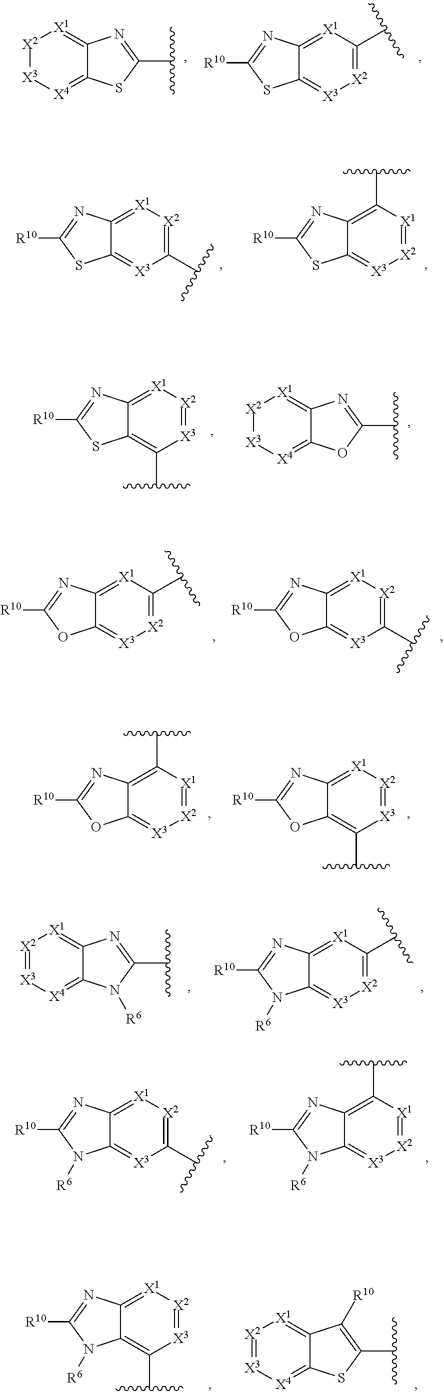

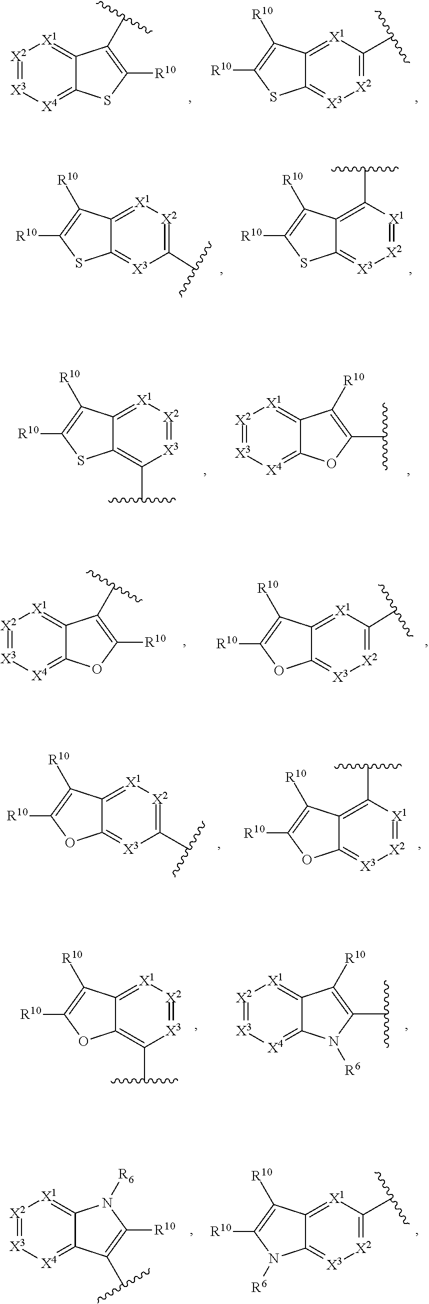

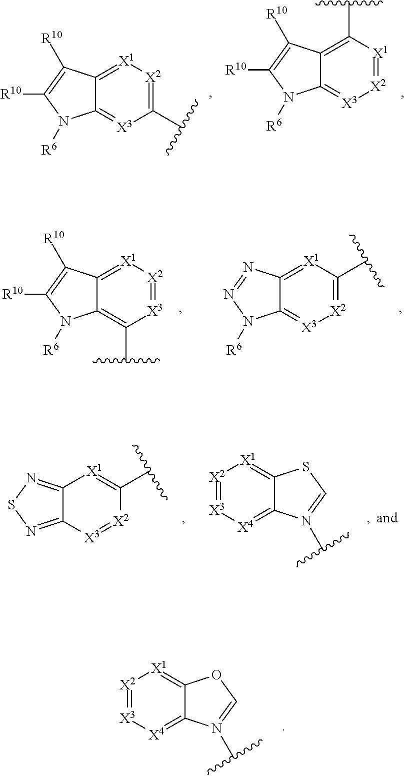

In some embodiments of a compound of Formulas (II), (IIa), or (IIb), A is selected from

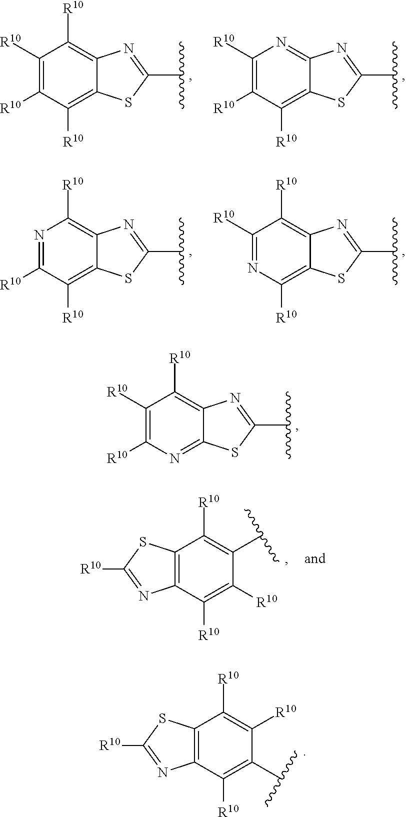

In some embodiments of a compound of Formulas (II), (IIa), or (IIb), A is selected from

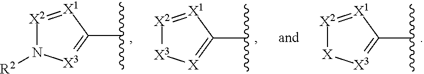

In some embodiments of a compound of Formulas (II), (IIa), or (IIb), A is selected from

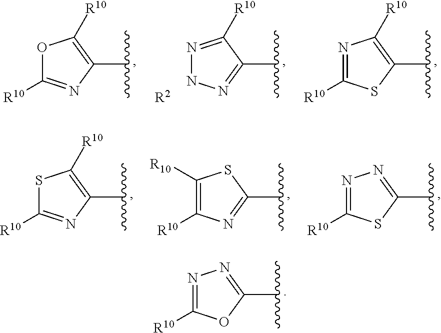

In some embodiments of a compound of Formulas (II), (IIa), or (IIb), A is selected from

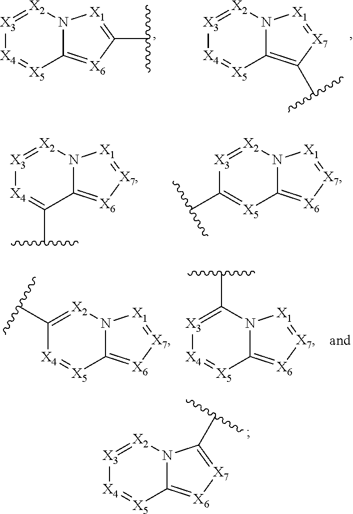

wherein X¹, X², X³, X⁴, X⁵, X⁶, and X⁷ are independently selected from N and CR¹⁰. In some embodiments of a compound of Formulas (II), (IIa), or (IIb), A is selected from

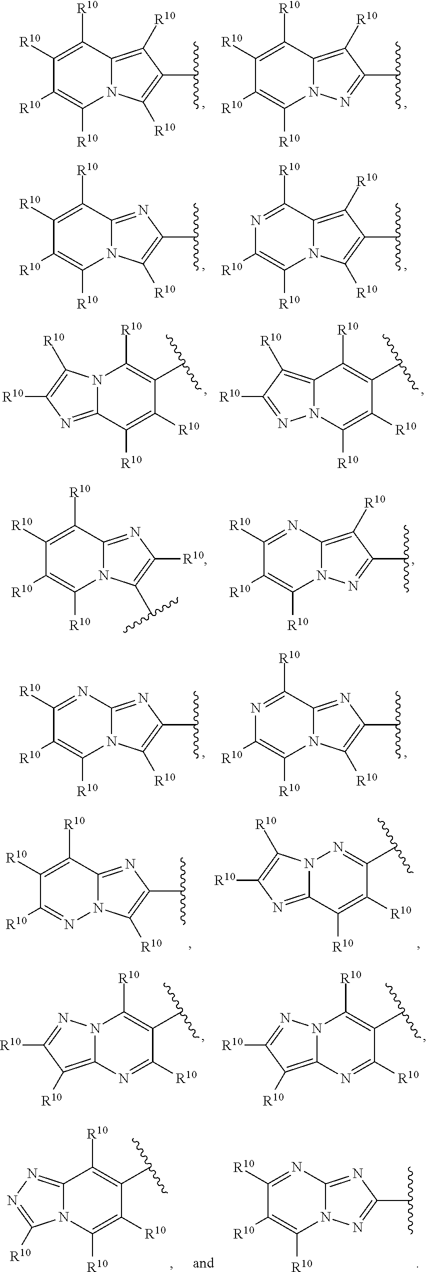

In some embodiments of a compound of Formulas (II), (IIa), or (IIb), A is selected from

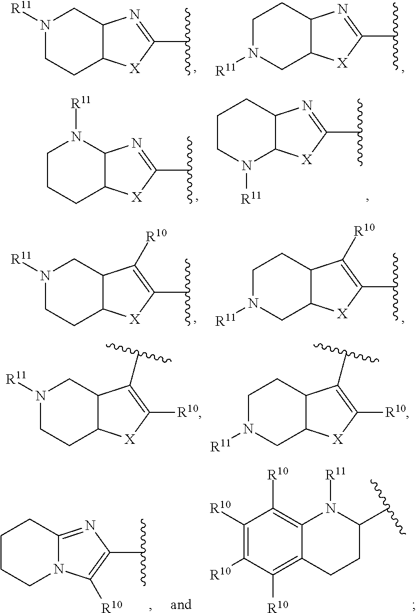

wherein X is O, S, or NR²; and R¹ is H, alkyl, aryl, heteroaryl, —SO₂-(alkyl), —SO₂-(cycloalkyl), —SO₂-(aryl), —SO₂-(heteroaryl), —SO₂-(heterocycloalkyl), —C(═O)O(alkyl), —C(═O)O(cycloalkyl), —C(═O)O(heterocycloalkyl), —C(═O)O(aryl), —C(═O)O(heteroaryl), —C(═O)NR⁶R⁷, —C(═O)NR⁶(cycloalkyl), —C(═O)NR⁶(heterocycloalkyl), —C(═O)NR⁶(aryl), —C(═O)NR⁶(heteroaryl), —C(═O)(alkyl), —C(═O)(cycloalkyl), —C(═O)(heterocycloalkyl), —C(═O)(aryl), or —C(═O)(heteroaryl). In some embodiments of a compound of Formulas (II), (IIa), or (IIb), A is selected from

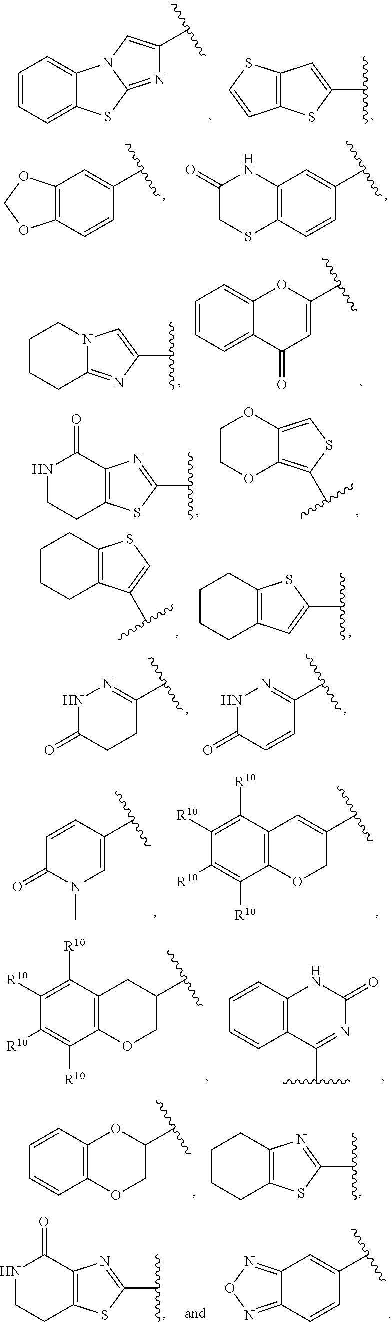

In one aspect, provided herein are compounds of Formula (IIc), a pharmaceutically acceptable salt, solvate, polymorph, prodrug, metabolite, N-oxide, stereoisomer, or isomer thereof:

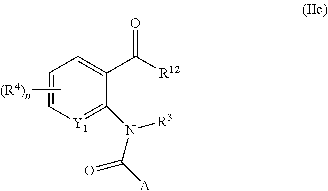

wherein:
Y₁ is N, CH, or CR⁴;
R¹² is —NR²R⁸ or —OR²;
R² and R³ are each independently selected from H and optionally substituted alkyl;
each R⁴ is independently selected from halogen, —CN, optionally substituted alkyl, optionally substituted alkoxy, and optionally substituted aryl;
R⁸ is optionally substituted alkyl, optionally substituted aryl, optionally substituted aralkyl, carbocyclyl, optionally substituted carbocyclylalkyl, optionally substituted heteroaryl, optionally substituted heteroarylalkyl, optionally substituted heterocyclyl, optionally substituted heterocyclylalkyl, —R^{b}COOR^{a} or —R^{b}CONR^{a}R^{a}; or R² and R⁸ taken together form an optionally substituted heterocycle with the nitrogen to which they are attached;
each R^{a} is independently selected from H and alkyl;
R^{b} is a bond or alkylenyl;
n is 0, 1, 2, or 3; and
A is optionally substituted heteroaryl, optionally substituted aryl or optionally substituted heterocyclyl.

In some embodiments of a compound of Formula (IIc), Y₁ is N. In some embodiments of a compound of Formula (IIc), Y₁ is N and n is 0. In some embodiments of a compound of Formula (IIc), Y₁ is N and n is 1. In some embodiments of a compound of Formula (IIc), Y₁ is N and n is 2.

In some embodiments of a compound of Formula (IIc), Y₁ is CR⁴. In some embodiments of a compound of Formula (IIc), Y₁ is CH. In some embodiments of a compound of Formula (IIc), Y₁ is CH and n is 0. In some embodiments of a compound of Formula (IIc), Y₁ is CH and n is 1. In some embodiments of a compound of Formula (IIc), Y₁ is CH and n is 2.

In some embodiments of a compound of Formula (IIc), R² and R³ are H. In some embodiments of a compound of Formula (IIc), R² and R³ are each independently optionally substituted alkyl. In some embodiments of a compound of Formula (IIc), R² and R³ are the same and optionally substituted alkyl. In some embodiments of a compound of Formula (IIc), R² is H and R³ is optionally substituted alkyl. In some embodiments of a compound of Formula (IIc), R² is optionally substituted alkyl and R³ is H.

In some embodiments of a compound of Formula (IIc), R¹² is —NR²R⁸. In some embodiments of a compound of Formula (IIc), R¹² is —OR². In some embodiments of a compound of Formula (IIc), R¹² is —OR² and R² is optionally substituted alkyl.

In some embodiments of a compound of Formula (IIc), R⁸ is optionally substituted alkyl. In some embodiments of a compound of Formula (IIc), R⁸ is optionally substituted aryl. In some embodiments of a compound of Formula (IIc), R⁸ is optionally substituted aryl; wherein the optionally substituted aryl is substituted with halogen. In some embodiments of a compound of Formula (IIc), R⁸ is carbocyclyl. In some embodiments of a compound of Formula (IIc), R⁸ is optionally substituted carbocyclylalkyl. In some embodiments of a compound of Formula (IIc), R⁸ is optionally substituted heteroaryl. In some embodiments of a compound of Formula (IIc), R⁸ is optionally substituted heteroaryl; wherein the optionally substituted heteroaryl is substituted with a group selected from alkyl, —O-(alkyl) and —NR⁶R⁷. In some embodiments of a compound of Formula (IIc), R⁸ is optionally substituted heteroarylalkyl. In some embodiments of a compound of Formula (IIc), R⁸ is optionally substituted heterocyclyl. In some embodiments of a compound of Formula (IIc), R⁸ is optionally substituted heterocyclyl; wherein the optionally substituted heterocyclyl is substituted with alkyl. In some embodiments of a compound of Formula (IIc), R² and R⁸ taken together form an optionally substituted heterocycle with the nitrogen to which they are attached. In further embodiments of a compound of Formula (IIc), the heterocycle is selected from piperidinyl, morpholinyl, thiomorpholinyl, optionally substituted diazepanyl and optionally substituted piperizanyl; wherein the optionally substituted diazepanyl and the optionally substituted piperizanyl are substituted with a group selected from alkyl, aryl, -COOtBu and —SO₂Me. In some embodiments of a compound of Formula (IIc), R⁸ is optionally substituted heterocyclylalkyl. In some embodiments of a compound of Formula (IIc), R⁸ is —R^{b}COOR^{a}. In some embodiments of a compound of Formula (IIc), R⁸ is —R^{b}CONR^{a}R^{a}. In some embodiments of a compound of Formula (IIc), R² and R⁸ taken together form an optionally substituted heterocycle with the nitrogen to which they are attached. In some embodiments of a compound of Formula (IIc), R⁸ is optionally substituted alkyl, optionally substituted aryl, optionally substituted aralkyl, carbocyclyl, optionally substituted carbocyclylalkyl, optionally substituted heteroaryl, optionally substituted heteroarylalkyl, optionally substituted heterocyclyl, or optionally substituted heterocyclylalkyl. In some embodiments of a compound of Formula (IIc), R⁸ is optionally substituted alkyl, carbocyclyl, optionally substituted carbocyclylalkyl, optionally substituted heterocyclyl, or optionally substituted heterocyclylalkyl. In some embodiments of a compound of Formula (IIc), R⁸ is optionally substituted aryl, optionally substituted aralkyl, optionally substituted heteroaryl, or optionally substituted heteroarylalkyl. In some embodiments of a compound of Formula (IIc), R⁸ is optionally substituted alkyl, optionally substituted aralkyl, carbocyclyl, optionally substituted carbocyclylalkyl, optionally substituted heteroarylalkyl, optionally substituted heterocyclyl, or optionally substituted heterocyclylalkyl.

In some embodiments of a compound of Formula (IIc), A is optionally substituted heteroaryl. In some embodiments of a compound of Formula (IIc), A is optionally substituted aryl. In some embodiments of a compound of Formula (IIc), A is optionally substituted heterocyclyl. In some embodiments of a compound of Formula (IIc), A is optionally substituted heteroaryl, optionally substituted aryl or optionally substituted heterocyclyl; and the optionally substituted aryl, the optionally substituted heterocyclyl, the optionally substituted heteroaryl are substituted with 1-6 R¹⁰; wherein each R¹⁰ is independently selected from H, halogen, —CN, —NO₂, —CF₃, alkyl, —SR⁶, —OR⁶, —NR⁶R⁷, —NR⁶C(═O)(alkyl), —NR⁶C(═O)(cycloalkyl), —NR⁶C(═O)(heterocyclyl), —NR⁶C(═O)(aryl), —NR⁶C(═O)(heteroaryl), —C(═O)NR⁶R⁷, —C(═O)NR⁶(cycloalkyl), —C(═O)NR⁶(heterocycloalkyl), —C(═O)NR⁶(aryl), —C(═O)NR⁶(heteroaryl), —NR⁶C(═O)NR⁶R⁷, —NR⁶C(═O)NR⁷(cycloalkyl), —NR⁶C(═O)NR⁷(heterocycloalkyl), —NR⁶C(═O)NR⁷(aryl), —NR⁶C(═O)NR⁷(heteroaryl), —NR⁶C(═O)O(alkyl), —NR⁶C(═O)O(cycloalkyl), —NR⁶C(═O)O(heterocycloalkyl), —NR⁶C(═O)O(aryl), —NR⁶C(═O)O(heteroaryl), —NR⁶SO₂(alkyl), —NR⁶SO₂(cycloalkyl), —NR⁶SO₂(heterocycloalkyl), —NR⁶SO₂(aryl), —NR⁶SO₂(heteroaryl), —SO₂NR⁶R⁷, —SO₂NR⁶(cycloalkyl), —SO₂NR⁶(heterocycloalkyl), —SR⁶, —SO₂R⁶, —SO₂NR⁶(aryl), —SO₂NR⁶(heteroaryl), haloalkyl, aryl, heteroaryl, heterocyclyl and tetrazoyl.

In some embodiments of a compound of Formula (IIc), A is selected from:

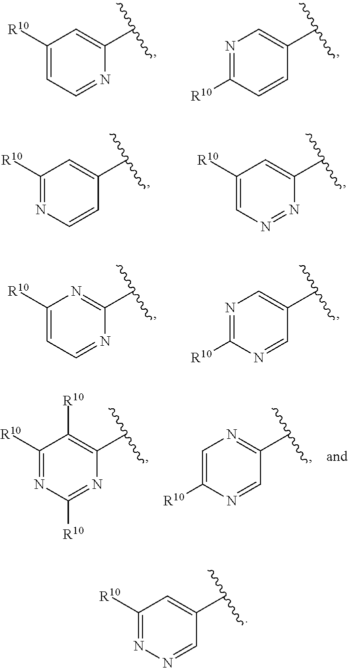

In some embodiments of a compound of Formula (IIc), A is

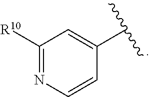

In some embodiments of a compound of Formula (IIc), A is selected from:

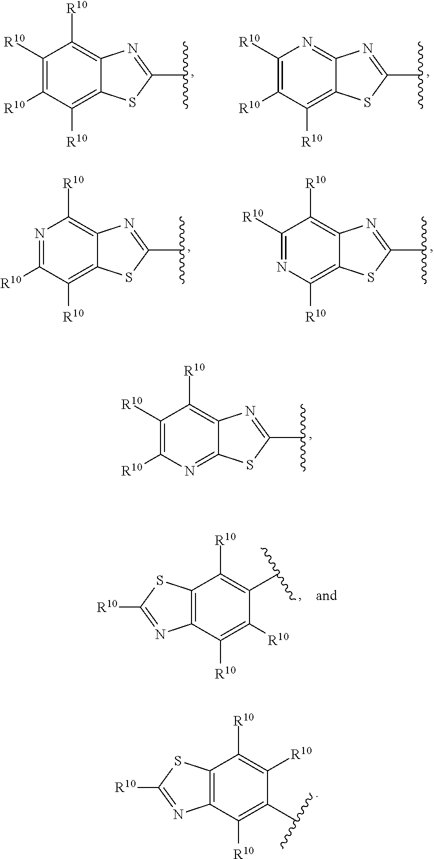

In some embodiments of a compound of Formula (IIc), A is

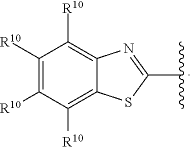

In some embodiments of a compound of Formula (IIc), R⁴ is halogen. In some embodiments of a compound of Formula (IIc), R⁴ is —CN. In some embodiments of a compound of Formula (IIc), R⁴ is optionally substituted alkyl. In some embodiments of a compound of Formula (IIc), R⁴ is optionally substituted alkoxy. In some embodiments of a compound of Formula (IIc), R⁴ is optionally substituted aryl. In some embodiments of a compound of Formula (IIc), R⁴ is halogen, —CN, or optionally substituted alkyl. In some embodiments of a compound of Formula (IIc), R⁴ is halogen, —CN, alkyl, or aryl.

In one aspect, provided herein are compounds, or pharmaceutically acceptable salts, solvates, polymorphs, prodrugs, metabolites, N-oxides, stereoisomers, or isomers thereof, selected from:

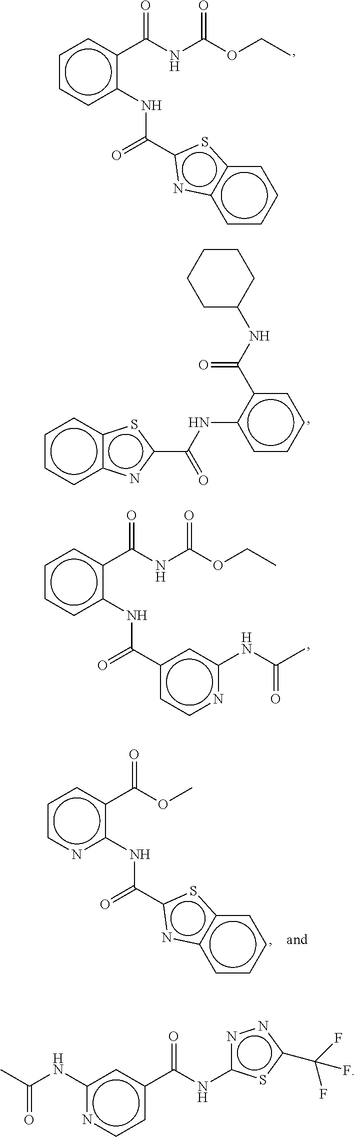

### Preparation of Compounds

Described herein are compounds of Formulas (I), (Ia), (Ib), (Ic), (Id), (II), (IIa), (IIb), and (IIc) that treat drug resistant and persistent tuberculosis, and processes for their preparation. Also described herein are pharmaceutically acceptable salts, pharmaceutically acceptable solvates, pharmaceutically active metabolites, and pharmaceutically acceptable prodrugs of such compounds. Pharmaceutical compositions comprising at least one such compound or a pharmaceutically acceptable salt, pharmaceutically acceptable solvate, pharmaceutically active metabolite or pharmaceutically acceptable prodrug of such compound, and a pharmaceutically acceptable excipient are also provided.

Compounds of Formulas (I), (Ia), (Ib), (Ic), (Id), (II), (IIa), (IIb), and (IIc) may be synthesized using standard synthetic reactions known to those of skill in the art or using methods known in the art. The reactions can be employed in a linear sequence to provide the compounds or they may be used to synthesize fragments which are subsequently joined by the methods known in the art.

The starting material used for the synthesis of the compounds described herein may be synthesized or can be obtained from commercial sources, such as, but not limited to, Aldrich Chemical Co. (Milwaukee, Wis.), Bachem (Torrance, Calif.), or Sigma Chemical Co. (St. Louis, Mo.). The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001); Green and Wuts, PROTECTIVE GROUPS IN ORGANIC SYNTHESIS 3^{rd} Ed., (Wiley 1999); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); and Larock's Comprehensive Organic Transformations (VCH Publishers Inc., 1989). (all of which are incorporated by reference in their entirety). Other methods for the synthesis of compounds described herein may be found in International Patent Publication No. WO 01/01982901, Arnold et al. Bioorganic & Medicinal Chemistry Letters 10 (2000) 2167-2170; Burchat et al. Bioorganic & Medicinal Chemistry Letters 12 (2002) 1687-1690. General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein.

The products of the reactions may be isolated and purified, if desired, using conventional techniques, including, but not limited to, filtration, distillation, crystallization, chromatography and the like. Such materials may be characterized using conventional means, including physical constants and spectral data.

Compounds described herein may be prepared as a single isomer or a mixture of isomers.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the metabolites, pharmaceutically acceptable salts, esters, prodrugs, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds of the invention and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Polymorphs

In some embodiments, the compounds described herein exist as polymorphs. The invention provides for methods of treating diseases by administering such polymorphs. The invention further provides for methods of treating diseases by administering such polymorphs as pharmaceutical compositions.

Thus, the compounds described herein include all their crystalline forms, known as polymorphs. Polymorphs include the different crystal packing arrangements of the same elemental composition of a compound. In certain instances, polymorphs have different X-ray diffraction patterns, infrared spectra, melting points, density, hardness, crystal shape, optical and electrical properties, stability, and solubility. In certain instances, various factors such as the recrystallization solvent, rate of crystallization, and storage temperature cause a single crystal form to dominate.

### Prodrugs

In some embodiments, the compounds described herein exist in prodrug form. The invention provides for methods of treating diseases by administering such prodrugs. The invention further provides for methods of treating diseases by administering such prodrugs as pharmaceutical compositions.

Prodrugs are generally drug precursors that, following administration to an individual and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they are easier to administer than the parent drug. They are, for instance, bioavailable by oral administration whereas the parent is not. In certain instances, the prodrug also has improved solubility in pharmaceutical compositions over the parent drug. An example, without limitation, of a prodrug would be a compound as described herein which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyamino acid) bonded to an acid group where the peptide is metabolized to reveal the active moiety. (See for example Bundgaard, “Design and Application of Prodrugs” in A Textbook of Drug Design and Development, Krosgaard-Larsen and Bundgaard, Ed., 1991, Chapter 5, 113-191, which is incorporated herein by reference).

In some embodiments, prodrugs are designed as reversible drug derivatives, for use as modifiers to enhance drug transport to site-specific tissues. The design of prodrugs to date has been to increase the effective water solubility of the therapeutic compound for targeting to regions where water is the principal solvent.

Additionally, prodrug derivatives of compounds described herein can be prepared by methods described herein are otherwise known in the art (for further details see Saulnier et al., Bioorganic and Medicinal Chemistry Letters, 1994, 4, 1985). By way of example only, appropriate prodrugs can be prepared by reacting a non-derivatized compound with a suitable carbamylating agent, such as, but not limited to, 1,1-acyloxyalkylcarbanochloridate, para-nitrophenyl carbonate, or the like. Prodrug forms of the herein described compounds, wherein the prodrug is metabolized in vivo to produce a derivative as set forth herein are included within the scope of the claims. Indeed, some of the herein-described compounds are prodrugs for another derivative or active compound.

In some embodiments, prodrugs include compounds wherein an amino acid residue, or a polypeptide chain of two or more (e. g., two, three or four) amino acid residues is covalently joined through an amide or ester bond to a free amino, hydroxy or carboxylic acid group of compounds of the present invention. The amino acid residues include but are not limited to the 20 naturally occurring amino acids and also includes 4-hydroxyproline, hydroxylysine, demosine, isodemosine, 3-methylhistidine, norvaline, beta-alanine, gamma-aminobutyric acid, cirtulline, homocysteine, homoserine, ornithine and methionine sulfone. In other embodiments, prodrugs include compounds wherein a nucleic acid residue, or an oligonucleotide of two or more (e. g., two, three or four) nucleic acid residues is covalently joined to a compound of the present invention.

Pharmaceutically acceptable prodrugs of the compounds described herein also include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters. Compounds having free amino, amido, hydroxy or carboxylic groups can be converted into prodrugs. For instance, free carboxyl groups can be derivatized as amides or alkyl esters. In certain instances, all of these prodrug moieties incorporate groups including but not limited to ether, amine and carboxylic acid functionalities.

Hydroxy prodrugs include esters, such as though not limited to, acyloxyalkyl (e.g. acyloxymethyl, acyloxyethyl) esters, alkoxycarbonyloxyalkyl esters, alkyl esters, aryl esters, phosphate esters, sulfonate esters, sulfate esters and disulfide containing esters; ethers, amides, carbamates, hemisuccinates, dimethylaminoacetates and phosphoryloxymethyloxycarbonyls, as outlined in Advanced Drug Delivery Reviews 1996, 19, 115.

Amine derived prodrugs include, but are not limited to the following groups and combinations of groups:

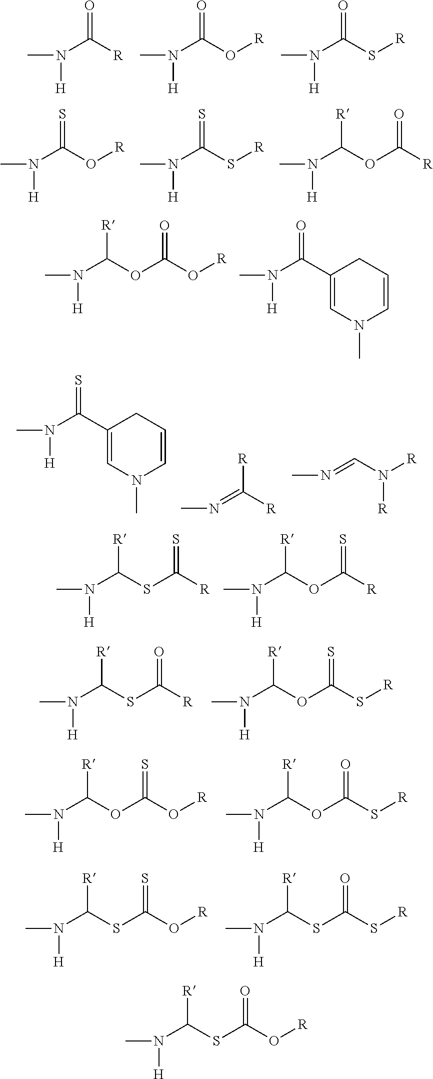

as well as sulfonamides and phosphonamides.

In certain instances, sites on any aromatic ring portions are susceptible to various metabolic reactions, therefore incorporation of appropriate substituents on the aromatic ring structures, can reduce, minimize or eliminate this metabolic pathway.

### Metabolites

In some embodiments, compounds of Formulas (I), (Ia), (Ib), (Ic), (Id), (II), (IIa), (IIb), and (IIc) are susceptible to various metabolic reactions. Therefore, in some embodiments, incorporation of appropriate substituents into the structure will reduce, minimize, or eliminate a metabolic pathway. In specific embodiments, the appropriate substituent to decrease or eliminate the susceptibility of an aromatic ring to metabolic reactions is, by way of example only, a halogen, or an alkyl group.

In additional or further embodiments, the compounds of Formulas (I), (Ia), (Ib), (Ic), (Id), (II), (IIa), (IIb), and (IIc) described herein are metabolized upon administration to an organism in need to produce a metabolite that is then used to produce a desired effect, including a desired therapeutic effect.

### Pharmaceutical Compositions/Formulations

In another aspect, provided herein are pharmaceutical composition comprising a compound of Formulas (I), (Ia), (Ib), (Ic), (Id), (II), (IIa), (IIb), or (IIc) as described herein, or a pharmaceutically acceptable salt, polymorph, solvate, prodrug, N-oxide, stereoisomer, or isomer thereof, and a pharmaceutically acceptable excipient.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Provided herein are pharmaceutical compositions that include a compound of Formulas (I), (Ia), (Ib), (Ic), (Id), (II), (IIa), (IIb), or (IIc) and at least one pharmaceutically acceptable inactive ingredient. In some embodiments, the compounds described herein are administered as pharmaceutical compositions in which a compound of Formulas (I), (Ia), (Ib), (Ic), (Id), (II), (IIa), (IIb), or (IIc) is mixed with other active ingredients, as in combination therapy. In other embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In yet other embodiments, the pharmaceutical compositions include other therapeutically valuable substances.

A pharmaceutical composition, as used herein, refers to a mixture of a compound of Formulas (I), (Ia), (Ib), (Ic), (Id), (II), (IIa), (IIb), or (IIc) with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including a compound of Formulas (I), (Ia), (Ib), (Ic), (Id), (II), (IIa), (IIb), or (IIc) are manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound of Formulas (I), (Ia), (Ib), (Ic), (Id), (II), (IIa), (IIb), or (IIc) as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, compounds described herein exist in unsolvated form or in solvated forms with pharmaceutically acceptable solvents such as water, ethanol, and the like. The solvated forms of the compounds presented herein are also considered to be disclosed herein.

Pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical preparations that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

### Combination Treatment

The compounds according to Formulas (I), (Ia), (Ib), (Ic), (Id), (II), (IIa), (IIb), and (IIc) may be used in combination with one or more additional antibiotic agents. The antibiotic agent may be selected from an aminoglycoside, ansamycin, carbacephem, carbapenem, cephalosporin, glycopeptide, lincosamide, lipopeptide, macrolide, monobactam, nitrofurans, penicillin, polypeptide, quinolone, sulfonamide, or tetracycline antibiotic. Examples of antibiotic agents include, but are not limited to, Aminoglycoside derivatives like amikacin, gentamicin, kanamycin, neomycin, netilmicin, tobramicin, paromomycin; Ansamycin derivatives like geldanamycin, herbimycin; Carbacephem derivatives like loracarbef, Carbapenem derivatives like ertapenem, doripenem, imipenem, meropenem; Cephalosporin derivatives like cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole; Glycopeptide derivatives like teicoplanin, vancomycin, telavancin; Lincosamides like clindamycin, lincomycin; Lipopeptide derivatives like daptomycin; Macrolide derivatives like azithromycin, clarithromycin, dirithromycin, erythromycin, roxithromycin, troleandomycin; telithreomycin, spectinomycin; Monobactam derivatives like aztreonam; Nitrofuran derivatives like furazolidone, nitrofurantoin; Penicillin derivatives like amoxicillin, ampicillin, azlocillin, carbinicillin, cloxacillin, dicloxacillin, flucloxacillin, mezlocillin, methicillin, nafcillin, oxacillin, penicillin G, penicillin V, piperacillin, temocillin, ticarcillin; Penicillin combinations like amoxicillin/clavulanate, ampicillin/sulbactam, piperacillin/tazobactam, ticarcillin/clavulanate; Polypeptide derivatives like bacitracin, colistin, polymyxin B; Quinolone derivatives like ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin; Sulfonamide derivatives like mafenide, sulfonamidochrysoidine, sulfacetamide, sulfadiazine, silver sulfadiazine, sulfamethoxazole, sulfanilimide, sulfasalazine, sulfisoxazole, trimethoprim, trimethoprim/sulfamethoxazole; Tetracyclin derivatives like demeclocycline, doxycycline, minocycline, oxytetracycline, tetracycline; Derivatives against mycobacteria like clofazimine, dapsone, capreomycin, cycloserine, ethambutol, ethioamide, isoniazid, pyrazinamide, rifampin, refampicin, rifabutin, rifapentine, streptomycin; or other antibiotic agents like arsphenamine, chloramphenicol, fosfomycin, fusidic acid, linezolid, metronidazole, mupirocin, platensimycin, quinupristin/dalfopristin, rifaximin, thiampheniol, tigecycline, tinidazole. In preferred embodiments, the antibiotic agent is useful in the treatment of tuberculosis and/or infection with Mycobacterium tuberculosis and may be selected from with rifampicin, TMC207, or isoniazid.

### Administration of Pharmaceutical Composition

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In some embodiments, compounds of Formulas (I), (Ia), (Ib), (Ic), (Id), (II), (IIa), (IIb), and (IIc) and compositions thereof are administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained.

### Methods

Further provided herein is a method to treat drug resistant and persistent tuberculosis in a mammal, the method comprising administering to the mammal a compound of Formulas (I), (Ia), (Ib), (Ic), (Id), (II), (IIa), (IIb), or (IIc) or as described above and below. In some embodiments, the method further comprises administering an additional antibiotic agent.

### EXAMPLES

### List of Abbreviations

As used above, and throughout the description of the invention, the following abbreviations, unless otherwise indicated, shall be understood to have the following meanings
ACN acetonitrile
Bn benzyl
BOC or Boc tert-butyl carbamate
BOP benzotriazol-1-yl-oxytris (dimethylamino) phosphonium
t-Bu tert-butyl
Cbz benzyl carbamate
Cy Cyclohexyl
DBU 1,8-Diazabicyclo[5.4.0]undec-7-ene
DCC dicyclohexylcarbodiimide
DCM dichloromethane (CH₂Cl₂)
DIC 1,3-diisopropylcarbodiimide
DEAD diethyl azodicarboxylate
DIAD diisopropyl azodicarboxylate
DIEA diisopropylethylamine
DMAP 4-(N,N-dimethylamino)pyridine
DMP reagent Dess-Martin Periodinane reagent
DMF dimethylformamide
DMA N,N-Dimethylacetamide
DME 1,2-Dimethoxy-ethane
DMSO dimethylsulfoxide
Dppf 1,1′-Bis(diphenylphosphino)ferrocene
EDCI 1-ethyl-3-(3-dimethylaminopropyl) carbodiimide HCl
eq equivalent(s)
Et ethyl
Et₂O diethyl ether
EtOH ethanol
EtOAc ethyl acetate
HOAt 1-hydroxy-7-azabenzotriazole
HOBT 1-hydroxybenztriazole
HOSu N-hydroxysuccinamide
HPLC high performance liquid chromatography
LAH lithium aluminum anhydride
Me methyl
MeI methyliodide
MeOH methanol
MOMCl methoxymethylchloride
MOM methoxymethyl
MS mass spectroscopy
NMP N-methyl-pyrrolidin-2-one
NMR nuclear magnetic resonance
PyBOP benzotriazole-1-yl-oxytris-pyrrolidino-phosphonium Hexafluorophosphate
SPHOS 2-Dicyclohexylphosphino-2′,6′-dimethoxybiphenyl
TBD 1,5,7-triazabicyclo[4.4.0]-dec-5-ene
RP-HPLC reverse phase-high pressure liquid chromatography
TBS tert-butyldimethylsilyl
TBSCl tert-butyldimethylsilyl chloride
TBTU O-(Benzotriazol-1-yl)-N,N,N′,N′-tetramethyluronium
TEOC 2-Trimethylsilylethyl Carbamate
TFA trifluoroacetic acid
Tf₂O triflate anhydride
TMG 1,1,3,3-Tetramethylguanidine
THF tetrahydrofuran
THP tetrahydropyran
TLC thin layer chromatography
XPHOS 2-Dicyclohexylphosphino-2′,4′,6′-triisopropylbiphenyl

### General Examples for the Preparation of Compounds of the Invention

The starting materials and intermediates for the compounds of this invention may be prepared by the application or adaptation of the methods described below, their obvious chemical equivalents, or, for example, as described in literature such as The Science of Synthesis, Volumes 1-8. Editors E. M. Carreira et al. Thieme publishers (2001-2008). Details of reagent and reaction options are also available by structure and reaction searches using commercial computer search engines such as Scifinder (www.cas.org) or Reaxys (www.reaxys.com).

### Part A: Thiophen-2-Amine Starting Materials:

### Starting Material (2):

The following final compounds utilized (1) in the final stage of synthesis: 1, 43, 54-61, 63-97.

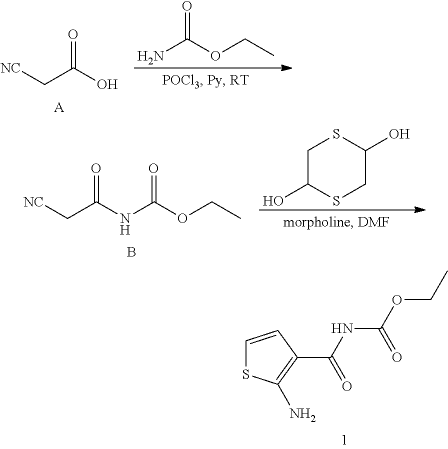

To a solution of A (120 g, 1410 mmol), ethyl carbamate (108 g, 706 mmol) in dry toluene (500 mL) at 0° C. under nitrogen was slowly added DMF (29 ml), then POCl₃ (66 ml, 730 mmol). Reaction mixture was heated at 70° C. for 1.5 h, monitored by TLC. After cooling to room temperature, solvent and POCl₃ were removed, and reaction quenched with ice water (100 mL). Precipitate was filtered to give B (210 g, 95%) as white solid.

To a solution of B (210 g, 1345 mmol), 1,4-dithiane-2,5-diol (108 g, 706 mmol) in DMF (1300 mL) at 0° C. under nitrogen was added morpholine (117 g, 1345 mmol). Reaction mixture was heated at 65° C. for 3 h, monitored by TLC. Solvent was removed in vacuo, and the mixture poured into ice water (500 ml), extracted with EtOAc, washed with water and brine and concentrated. The residue was recrystallized from EtOAc and hexane to give (1) as brown solid.

### Starting Material (2):

The following compounds utilized (2) in the final stage of synthesis: 41.

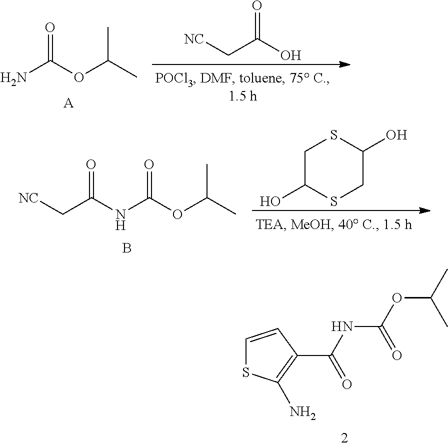

To a solution of A (4.2 g, 48 mmol), isopropyl carbamate (A) (5.0 g, 48 mmol) in dry toluene (18 mL) at 0° C. under nitrogen was added DMF (1.2 ml), then POCl₃ (2.4 ml, 26 mmol). Reaction mixture was heated at 75° C. for 1.5 h, monitored by TLC. After cooling to room temperature, solvent and POCl₃ were removed, and reaction quenched with ice water (50 mL). White solid was filtered to give B (3.5 g, 42%).

To a solution of B (3.5 g, 21 mmol), 1,4-dithiane-2,5-diol (1.7 g, 11 mmol) in MeOH (100 mL) at 0° C. under nitrogen was added EtN₃ (2.3 g, 23 mmol). Reaction mixture was heated at 40° C. for 1.5 h, monitored by TLC. Solvent was removed in vacuo, and the reaction dissolved in a mixture of solvent (i-PrOH:DCM=1:4) and washed with Sat. aq. NH₄Cl. Organic layer was concentrated. Crude material was purified by column chromatography (silica gel, EtOAc:PE=1:2-1:1) to give (2) (3.5 g, 75%) as a yellow solid.

### Starting Material (3):

The following compounds utilized (3) in the final stage of synthesis: 37.

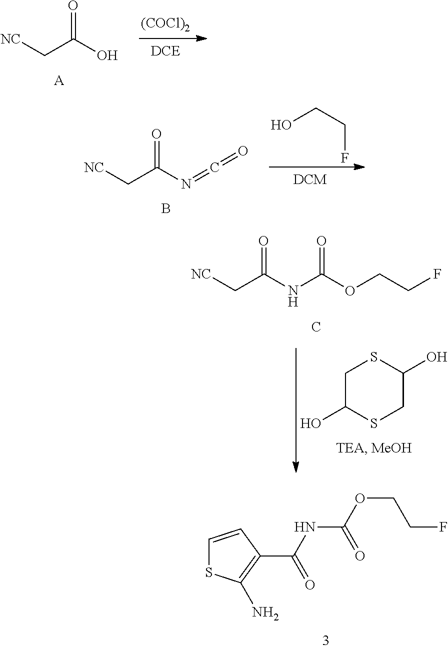

To a solution of A (1.0 g, 11.9 mmol) in dry DCE (10 mL) at 0° C. under nitrogen was added oxalyl chloride (1.5 ml, 17.3 mmol). Reaction mixture was heated at 75° C. for 1.5 h to give B, monitored by TLC. After cooling to 0° C., 2-fluoroethanol (4.7 ml, 55.6 mmol) was added, and the resulting dark brown suspension stirred at room temperature for 30 min. Afterwards, reaction was quenched with water, dissolved in solvent mixture (i-PrOH:DCM=1:4), and filtered. Filtration cake was washed with a mixture of solvent (i-PrOH:DCM=1:4), and the filtrate washed with water and brine, then dried and concentrated to give crude C. To a solution of C and 1,4-dithiane-2,5-diol (0.24 g, 1.61 mmol) in MeOH (15 mL) at 0° C. under nitrogen was added EtN₃ (0.4 ml, 3.2 mmol). Reaction mixture was heated at 40° C. for 1.5 h, monitored by TLC. Solvent was removed in vacuo, and the reaction dissolved in a mixture of solvent (i-PrOH:DCM=1:4) and washed with Sat. aq. NH₄Cl. Organic layer was concentrated. Crude material was purified by column chromatography (silica gel, EtOAc:PE=1:2) to give 3 (210 mg, 8%, over three steps).

### Starting Material (4):

Starting material (4) synthesized in an analogous manner to (3), substituting 2-fluoroethanol with methoxyethanol:

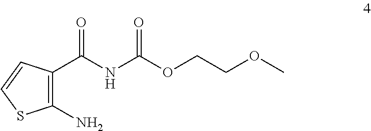 [SMILES: COCCOC(=O)NC(=O)c1ccsc1N]

### Starting Material (5):

The following compounds utilized (5) in the final stage of synthesis: 62.

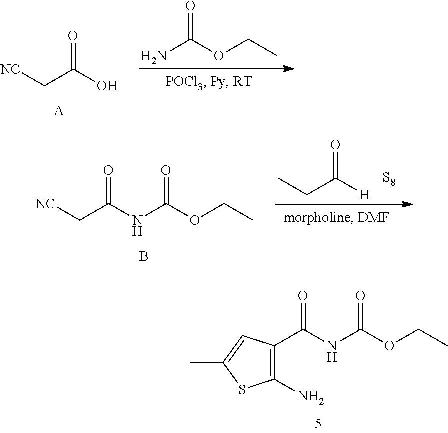

To a solution of A (120 g, 1410 mmol), ethyl carbamate (108 g, 706 mmol) in dry toluene (500 mL) at 0° C. under nitrogen was added DMF (29 ml), then POCl₃ (66 ml, 730 mmol). Reaction mixture was heated at 70° C. for 1.5 h, monitored by TLC. After cooling to room temperature, solvent and POCl₃ were removed, and reaction quenched with ice water (100 mL). White solid was collected to give B (210 g, 95%).

To a solution of B (25 g, 160 mmol), propionaldehyde (9.3 g, 160 mmol) and S₈ (5.1 g, 160 mmol) in DMF (30 mL) at 0° C. under nitrogen was added morpholine (7.9 g, 90 mmol). Reaction mixture was heated at 50° C. for 30 min, monitored by TLC. Solvent was removed in vacuo, and the mixture poured into ice water (50 ml), extracted with EtOAc, washed with water and brine and concentrated. Crude material was purified by column chromatography (silica gel, EtOAc:PE=1:1) to give 5 (6.8 g, 18%) as a yellow solid.

### Part B: Carboxylic Acid Starting Materials:

A majority of the carboxylic acid starting materials were purchased by Bioduro. The reaction schemes of starting materials synthesized in Bioduro laboratories are as follows:

### Starting Material (1):

The following final compounds utilized (1) in the final stage of synthesis: 1, 18, 37, 41.

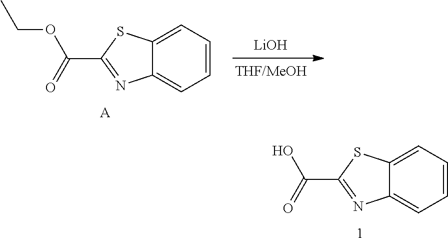

### Method A

To a solution of A (10 g, 48 mmol) in a mixture of solvent (THF:MeOH=1:1, v/v) (50 mL) at 0° C. under nitrogen was added aq.LiOH (72 ml, 2M, 145 mmol). Reaction mixture was stirred at room temperature for 2-12 h, monitored by TLC. Mixture was acidified with aq.HCl (6M) to pH=3-4, and precipitate filtered to give 1 (5 g, 58%) as a white solid.

### Method B

To a solution of A (10 g, 48 mmol) in THF (50 mL) at 0° C. under nitrogen was added aq.NaOH (72 ml, 2M, 145 mmol). Reaction mixture was stirred at room temperature for 30-120 min, monitored by TLC. Mixture was acidified with aq.HCl (6M) to pH=3-4, and precipitate filtered to give 1 (7 g, 81%) as a white solid.

### Starting Material (2):

The following final compounds utilized (2) in the final stage of synthesis: 83.

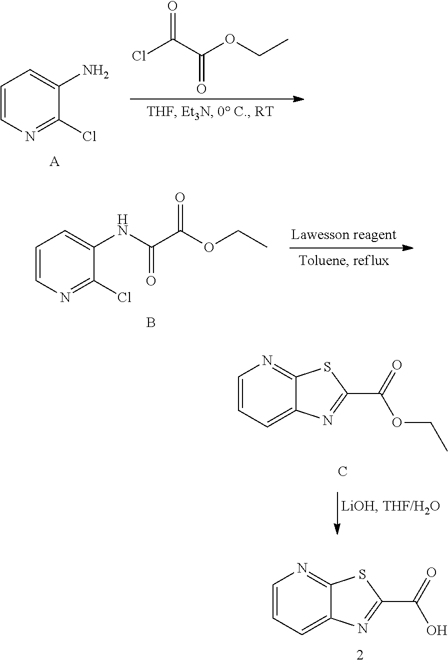

To a solution of A (30 g, 234 mmol) and Et₃N (52 g, 516 mmol) in dry THF (400 mL) at 0° C. under nitrogen was added ethyl 2-chloro-2-oxoacetate (31 ml, 281 mmol) dropwise. Reaction was stirred at room temperature for 4 h, monitored by TLC. Solvent was removed in vacuo, and the mixture poured into ice water (50 ml), extracted with EtOAc, washed with water and brine and concentrated. The crude material was purified by column chromatography (silica gel, EtOAc:PE=1:1) to give B (40 g, 75%) as a white solid.

A mixture of B (5 g, 22 mmol) and Lawesson's reagent (7.1 g, 18 mmol) in toluene (40 ml) was heated at 110° C. under nitrogen for 2 h, monitored by TLC. The mixture was dissolved in EtOAc, and filtered. Filtration cake was washed with EtOAc, and the filtrate extracted with EtOAc, washed with water and brine, dried and concentrated. The crude material was purified by column chromatography (silica gel, EtOAc:PE=1:4-1:2-1:1). Product was recrystallized with EtOAc and hexane to give pure C (1.2 g, 26%) as a yellow solid and a crude target product (3.1 g, 60% of purity) as a yellow solid.

To a solution of C (1.2 g, 5.7 mmol) in a mixture of solvent (THF:MeOH=1:1, v/v) (50 mL) at 0° C. under nitrogen was added aq.LiOH (7.2 ml, 2M, 14.2 mmol). Reaction was stirred at room temperature for 2-12 h, monitored by TLC. The mixture was acidified with aq.HCl (6M) to pH=3-4, and precipitate collected to give 2 (0.6 g, 58%) as a white solid.

### Additional Synthetic Information:

The following compounds were prepared using the identical reaction with the following starting materials:

| Target compound number | Starting material (A) structure |
|---|---|
| 82 | 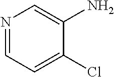 |

### Starting Material (3):

The following final compounds utilized (3) in the final stage of synthesis: 85

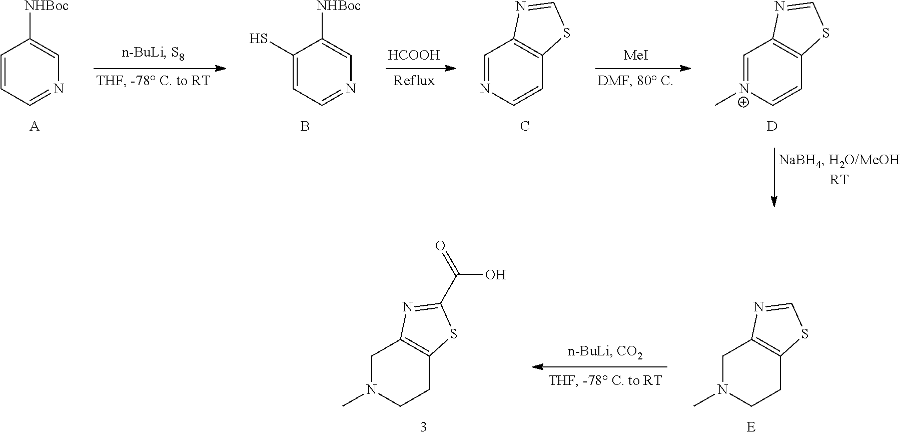

To a stirred solution of A (10 g, 51 mmol) in anhydrous THF (50 ml) was added n-BuLi (2.5 M in hexane, 52 ml, 130 mmol) dropwise at −78° C. under nitrogen. Reaction mixture was warmed to 0° C., and stirred at 0° C. for 2 h, then recooled to −78° C. To the mixture was added S₈ in one portion. Reaction warmed to room temperature and stirred for 1 h, monitored by TLC. Water was added and reaction acidified with aq.HCl (2M) to pH=3-4, and extracted with DCM, washed with water and brine, then dried and concentrated. Crude material was purified by column chromatography (silica gel, EtOAPE=1:3-1:2-1:1) to give a B (5.5 g, 47%) as a yellow solid.

A solution of B (5.5 g, 24 mmol) in HCOOH (45 ml) was heated to 100° C. under nitrogen for 4 h, monitored by TLC. The mixture was concentrated and to the residue was added aq. NaOH (5M) at 0° C. to basify to pH=7-8. Mixture was extracted with EtOAc, washed with water and brine, then dried and concentrated. The crude material was purified by column chromatography (silica gel, EtOAc:PE=1:1) to give a C (2.5 g, 76%) as a yellow solid.

To a solution of C (2.5 g, 18 mmol) in DMF (6.0 ml) was added methyl iodide (2.4 ml, 36 mmol) under nitrogen. Reaction was heated to 80° C. in a sealed tube for 1 h, monitored by TLC. The mixture was concentrated under reduced pressure and the white solid collected to give D (5.4 g, 100% crude).

To a solution of D (5.4 g, 18 mmol) in MeOH (20 ml) was added sodium borohydride (2.4 g, 64 mmol) at 0° C. Mixture was stirred at room temperature for 1 h, monitored by TLC. Reaction was concentrated under reduced pressure. Sat. aq. NaHCO₃ and EtOAc were added and organic layer extracted with EtOAc, washed with water and brine, then dried and concentrated. Crude material was purified by column chromatography (silica gel, EtOAc:PE=1:2→EtOAc:MeOH=30:1) to give E (2.4 g, 85%, over two steps) as a yellow oil.

To a stirred solution of E (0.73 g, 4.7 mmol) in anhydrous THF (15 ml) was added n-BuLi (2.5 M in hexane, 2.3 ml, 5.7 mmol) dropwise at −78° C. under nitrogen. Reaction mixture was stirred at −78° C. for 40 min, monitored by TLC (quenched with DMF before TLC). After release of CO₂ gas for 20 min (temperature was quickly increased to −50° C. and recooled to −78° C.), mixture was warmed to room temperature and stirred for 20 min, and concentrated in vacuo. Residue washed with hexane to give 3 (0.6 g, 64%) as a yellow solid.

### Starting Material (4):

The following final compounds utilized (4) in the final stage of synthesis: 57.

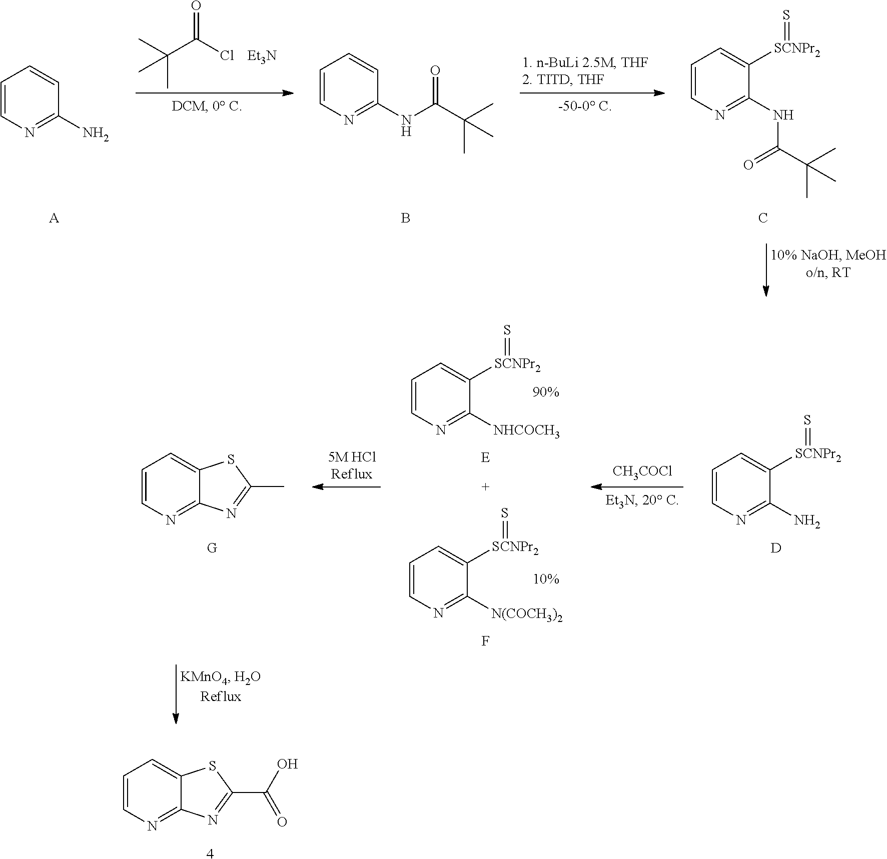

To a solution of A (11 g, 116 mmol) in dry DCM (100 ml) was added Et₃N (14.7 g, 145 mmol) and a solution of pivaloyl chloride (15.3 g, 128 mmol) in dry DCM (20 ml) at 0° C. Reaction mixture was stirred at 0° C. for 1 h, then warmed to room temperature and stirred for 12 h under nitrogen, monitored by TLC. Cold water (50 ml) was added, and the mixture extracted with DCM, washed with Sat. aq. NaHCO₃, water and brine, then dried and concentrated. The crude material was purified by column chromatography (silica gel, EtOAc:PE=1:2) to give B (12 g, 84%) as a white solid.

To a stirred solution B (2 g, 11 mmol) in anhydrous THF (10 ml) was added n-BuLi (2.5 M in hexane, 9.6 ml, 24 mmol) dropwise at −50° C. under nitrogen. Reaction mixture was stirred at 0° C. for 4 h and recooled to −50° C. A solution of TITD (4.2 g, 12 mmol) in dry THF (5 ml) was added slowly at −50° C. Mixture was warmed to room temperature and stirred for 45 min. Ether (10 ml) was added and the solution washed with water and brine, then concentrated in vacuo. Residue washed with EtOAc to give C (1.3 g, 33%) as a white solid.

A mixture of C (1.3 g, 3.68 mmol) and NaOH/MeOH (10% wt, 40 ml) was stirred at room temperature for 12 h under nitrogen, monitored by TLC. Filtration of resulting suspension gave D (0.7 g, 71%).

To a stirred solution of D (0.7 g, 2.6 mmol) in anhydrous DCM (15 ml) was added Et₃N (1.5 ml) dropwise at 0° C. under nitrogen, followed by appropriate acid chloride (8.6 mmol). Reaction mixture was stirred at room temperature for 12 h. Mixture was poured into cold water (20 ml) and extracted with DCM, washed with Sat. aq. NaHCO₃, water and brine, then dried and concentrated. The crude material was purified by column chromatography (silica gel, EtOAc:PE=1:2) to give a mixture of E and F (0.8 g, 90%) as a yellow oil.

A mixture of E and F (0.8 g) and aq. HCl (5M, 30 ml) was heated to 100° C. for 5 h under nitrogen, monitored by TLC. The cooled solution was washed with ether (80 ml), and basified with NaOH (2M) to pH=7-8 at 0° C. Mixture was poured into cold water (20 ml) and extracted with DCM, washed with Sat. aq. NaHCO₃, water and brine, then dried and concentrated to give G (310 mg, 80%).

To a stirred solution of G (310 mg, 2.07 mmol) in water (20 ml) was added KMnO₄ (490 mg, 3.1 mmol). Reaction mixture was stirred at 100° C. for 17 h. The precipitate was removed and filtrate mixed with water and DCM. Aqueous layer was acidified with aq. HCl (6M) to pH=5-6, and concentrated in vacuo. The residue was washed with water and ether to give 4 (98 mg, 26%) as a white solid.

### Building Block for Starting Materials (5) and (6):

The following starting material (F) is common to the reagent syntheses (5) and (6) for compounds 58 and 59 respectively.

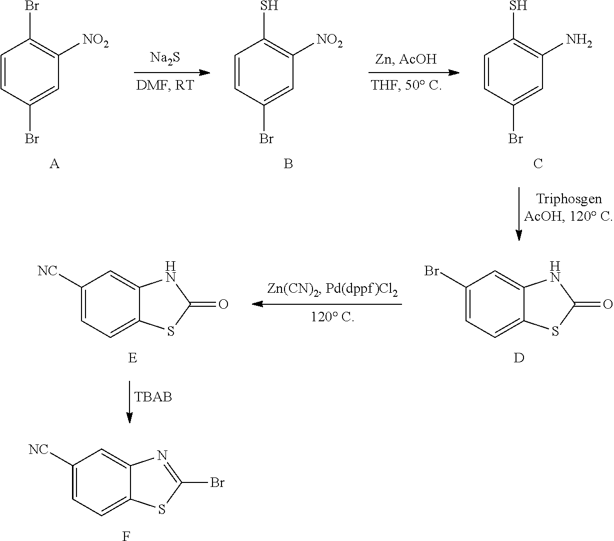

To a solution of A (20 g, 71 mmol) in DMF (200 ml) was added Na₂S (20 g, 320 mmol). Reaction mixture was stirred at room temperature for 16 h under nitrogen, monitored by TLC. Solution was mixed with water (200 ml), acidified with aq. HCl (3 M) to pH=3-4. Yellow precipitate was collected to give B (14 g, 84%).

Zn (70 g, 1077 mmol) was added to a solution of B (28 g, 120 mmol) in AcOH (500 ml). Reaction mixture was stirred at 65° C. for 15 h under nitrogen to give C, monitored by TLC. The mixture was cooled to room temperature, and triphosgene (25 g, 84 mmol) added slowly at 0° C. Mixture was refluxed for 18 h, monitored by TLC. Solution was then concentrated and hydrolyzed with water. Yellow solid was collected to give D (20 g, 73%, over two steps).

To a solution of D (5 g, 22 mmol) in DMF (15 ml) was added Zn(CN)₂ (5 g, 43 mmol), Pd(dppf)Cl₂ (1.5 g, 2.2 mmol) and Pd₂(dba)₃ (0.6 g, 1.1 mmol). Reaction mixture was stirred at 120° C. for 4 h under nitrogen, monitored by TLC. The mixture was extracted with EtOAc, washed with water and brine, then dried and concentrated. Crude material was purified by column chromatography (silica gel, EtOAc:PE=1:4) to give E (1.0 g, 26%) as a yellow solid.

A mixture of E (0.7 g, 4.0 mmol), Bu₄NBr (TBAB, 1.54 g, 4.8 mmol) and P₂O₅ (1.36 g, 9.5 mmol) in dry toluene (15 ml) was heated at 100° C. for 3 h under nitrogen, monitored by TLC. Mixture was extracted with EtOAc, washed with water and brine and then dried and concentrated. The crude material was purified by column chromatography (silica gel, EtOAc:PE=1:5) to give F (0.4 g, 42%) as a yellow solid.

### Starting Material (5):

The following final compounds utilized (5) in the final stage of synthesis: 58.

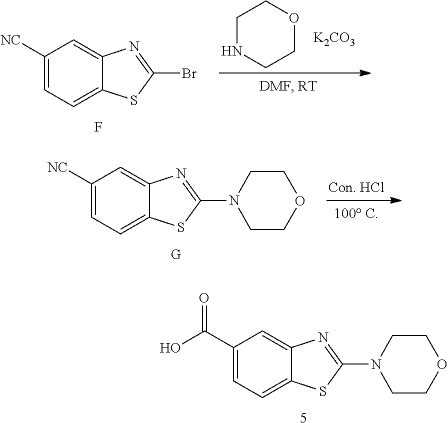

To a solution of F (0.2 g, 0.84 mmol) in dry DMF (10 ml) was added morpholine (0.1 g, 1.0 mmol) and K₂CO₃ (0.3 g, 2.1 mmol). Reaction mixture was stirred at room temperature for 12 h under nitrogen, monitored by TLC. Mixture was extracted with EtOAc, washed with water and brine, then dried and concentrated. The crude material was purified by column chromatography (silica gel, EtOAc:PE=1:4) to give G (0.11 g, 53%) as a yellow solid.

A mixture of G (0.11 g, 0.45 mmol) and Con. HCl (4 ml) was heated at 100° C. for 3 h under nitrogen, monitored by TLC. The mixture was concentrated under reduced pressure to give 5 (0.11 g, 100%).

### Starting Material (6):

The following final compounds utilized (6) in the final stage of synthesis: 59.

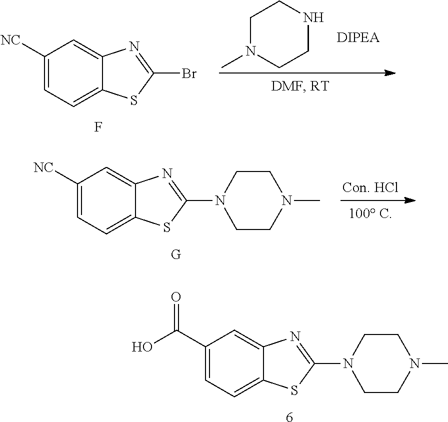

To a solution of F (0.2 g, 0.84 mmol) in dry DMF (10 ml) was added 1-methylpiperazine (0.1 g, 1.0 mmol) and DIPEA (0.4 g, 3.0 mmol). Reaction mixture was stirred at room temperature for 12 h under nitrogen, monitored by TLC. Mixture was extracted with EtOAc, washed with water and brine then dried and concentrated. The crude material was purified by column chromatography (silica gel, EtOAc:PE=1:4) to give G (0.15 g, 69%) as a yellow solid.

A mixture of G (0.15 g, 0.58 mmol) and Con. HCl (5 ml) was heated at 100° C. for 3 h under nitrogen, monitored by TLC. Mixture was concentrated under reduced pressure to give 6 (0.15 g, 100%).

### Part C: Condensation of Carboxylic Acid and Thiphen-2-Amine:

### Method A:

The following compounds were synthesized via Method A: 1, 18, 37, 41, 54-61, 63, 65, 66, 69-74, 77-79, 80, 82-92, 94-97.

### Example Scheme: Compound 82

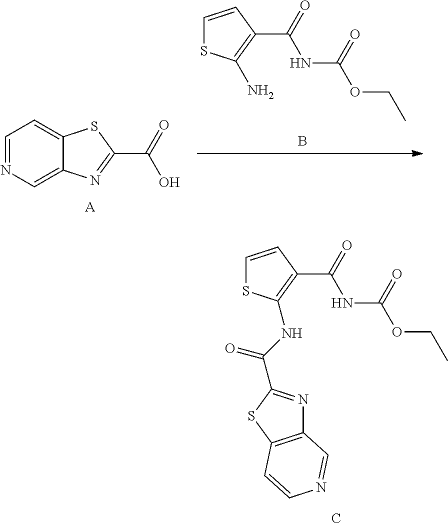

To a solution of carboxylic acid (50 mg, 0.277 mmol) in DCM (0.5 mL) at 0° C. under nitrogen was added ethyl 2-aminothiophene-3-carbonylcarbamate (59 mg, 0.227 mmol), Et₃N (84 mg, 0.831 mmol) and T3P (50% wt in EA, 0.3 ml, 0.554 mmol). Reaction mixture was stirred at 25° C. for 2-12 h, monitored by TLC. The mixture was poured into cold water (10 ml) and extracted with DCM (20 ml), washed with water and brine and then dried and concentrated. The crude material was purified by column chromatography (silica gel, EtOAc:PE=1:2) to give C (10 mg, 10%) as a yellow solid.

### Additional Synthetic Information:

The following compounds were prepared using the identical reaction with the following starting materials:

| Target compound | Thiophen starting material | Carboxylic acid starting |
|---|---|---|
| number | structure | material |
| 1 | 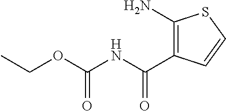 | 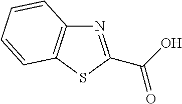 [SMILES: O=C(O)c1nc2ccccc2s1] |
| 18 | 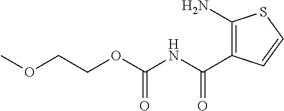 [SMILES: COCCOC(=O)NC(=O)c1ccsc1N] | 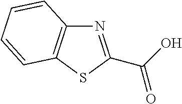 [SMILES: O=C(O)c1nc2ccccc2s1] |
| 37 | 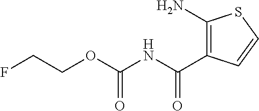 [SMILES: Nc1sccc1C(=O)NC(=O)OCCF] | 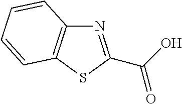 [SMILES: O=C(O)c1nc2ccccc2s1] |
| 41 | 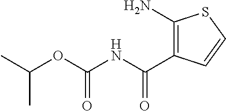 | 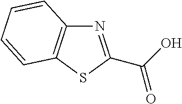 [SMILES: O=C(O)c1nc2ccccc2s1] |
| 54 | 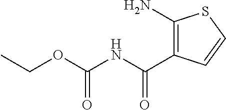 | 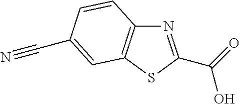 [SMILES: N#Cc1ccc2nc(C(=O)O)sc2c1] |
| 55 | 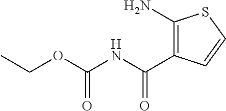 | 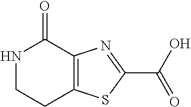 [SMILES: O=C(O)c1nc2c(s1)CCNC2=O] |
| 56 | 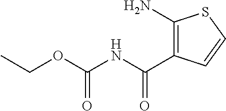 | 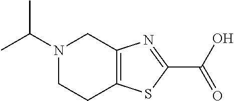 |
| 57 | 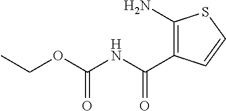 | 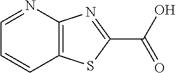 [SMILES: O=C(O)c1nc2ncccc2s1] |
| 58 | 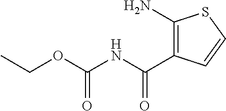 | 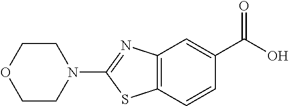 [SMILES: O=C(O)c1ccc2sc(N3CCOCC3)nc2c1] |
| 59 | 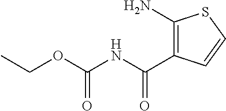 | 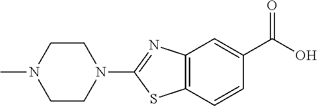 [SMILES: CN1CCN(c2nc3cc(C(=O)O)ccc3s2)CC1] |
| 60 | 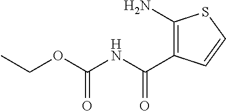 | 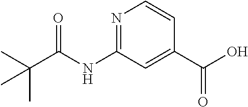 [SMILES: CC(C)(C)C(=O)Nc1cc(C(=O)O)ccn1] |
| 61 | 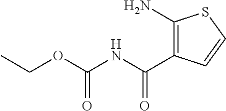 | 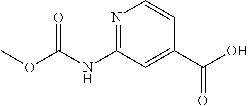 |
| 63 | 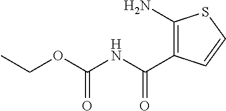 | 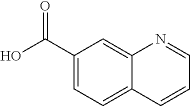 [SMILES: O=C(O)c1ccc2cccnc2c1] |
| 65 | 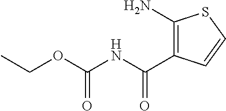 | 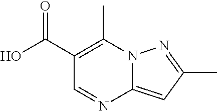 |
| 66 | 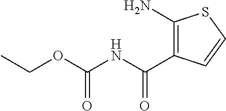 | 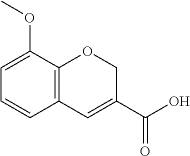 |
| 69 | 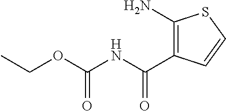 | 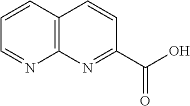 [SMILES: O=C(O)c1ccc2cccnc2n1] |
| 70 | 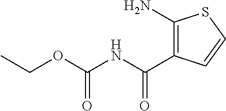 | 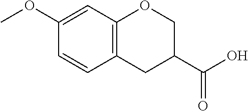 |
| 71 | 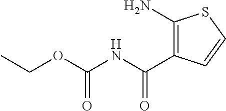 | 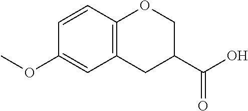 |
| 72 | 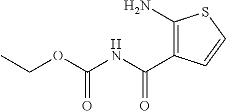 | 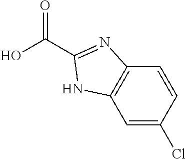 [SMILES: O=C(O)c1nc2ccc(Cl)cc2[nH]1] |
| 73 | 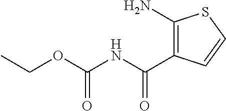 | 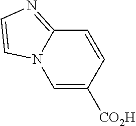 [SMILES: O=C(O)c1ccc2nccn2c1] |
| 74 | 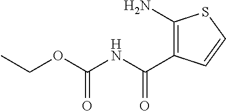 | 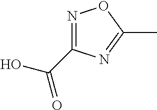 |
| 77 | 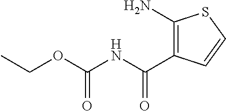 | 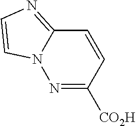 [SMILES: O=C(O)c1ccc2nccn2n1] |
| 78 | 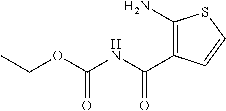 | 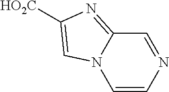 [SMILES: O=C(O)c1cn2ccncc2n1] |
| 79 | 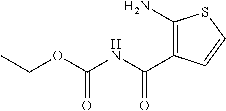 | 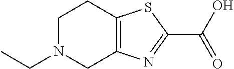 |
| 80 | 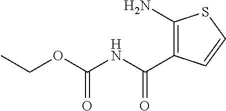 | 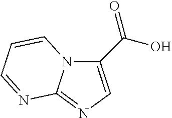 [SMILES: O=C(O)c1cnc2ncccn12] |
| 83 | 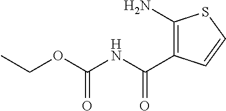 | 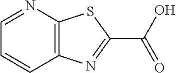 [SMILES: O=C(O)c1nc2cccnc2s1] |
| 84 | 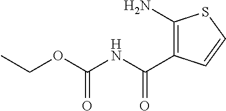 | 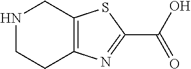 [SMILES: O=C(O)c1nc2c(s1)CNCC2] |
| 85 | 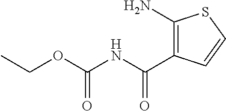 | 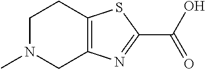 |
| 86 | 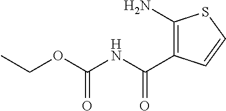 | 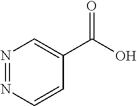 |
| 87 | 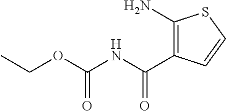 | 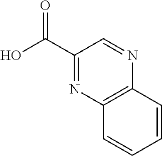 [SMILES: O=C(O)c1cnc2ccccc2n1] |
| 88 | 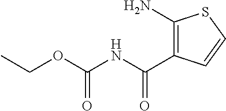 | 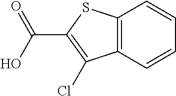 [SMILES: O=C(O)c1sc2ccccc2c1Cl] |
| 89 | 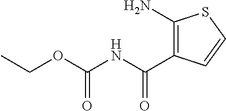 | 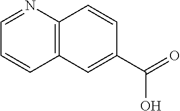 [SMILES: O=C(O)c1ccc2ncccc2c1] |
| 90 | 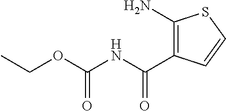 | 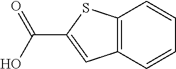 [SMILES: O=C(O)c1cc2ccccc2s1] |
| 91 | 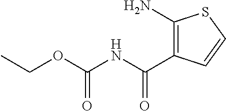 | 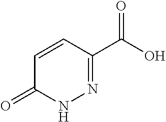 |
| 92 | 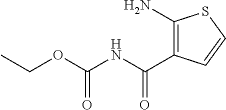 | 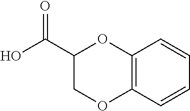 [SMILES: O=C(O)C1COc2ccccc2O1] |
| 94 | 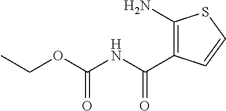 | 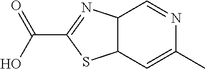 |
| 95 | 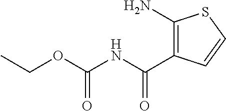 | 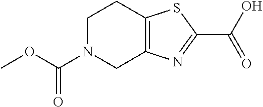 [SMILES: COC(=O)N1CCc2sc(C(=O)O)nc2C1] |
| 96 | 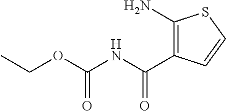 | 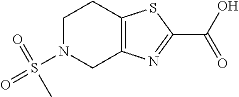 [SMILES: CS(=O)(=O)N1CCc2sc(C(=O)O)nc2C1] |
| 97 | 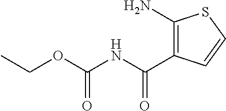 | 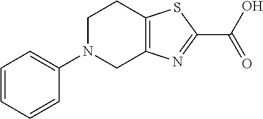 [SMILES: O=C(O)c1nc2c(s1)CCN(c1ccccc1)C2] |

### Method B:

The following compounds were synthesized via Method B: 43, 58, 64, 67, 68, 81, 93.

Example Scheme: Compound 58

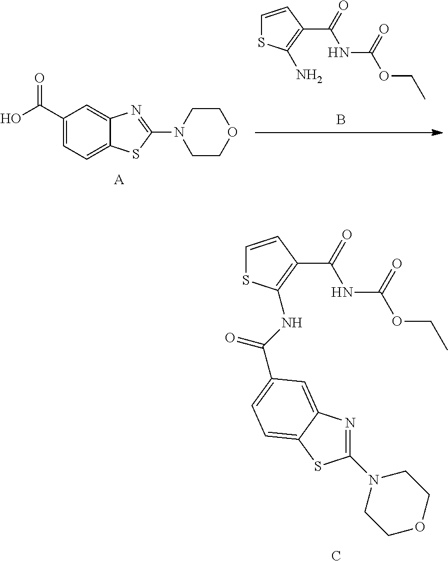

To a solution of carboxylic acid (0.11 g, 0.448 mmol) and B (0.1 g, 0.448 mmol) in pyridine (2 mL) at −30° C.-0° C. under nitrogen was added POCl₃ (0.3 ml). Reaction was stirred at −30° C.-0° C.-RT for 20-60 min, monitored by TLC. Mixture was poured into cold water (10 ml) and extracted with DCM (20 ml), washed with Sat. aq. NaHCO₃, water and brine, then dried and concentrated. Crude material was purified by column chromatography (silica gel, EtOAc:PE=1:4) to give C (0.1 g, 48%) as a yellow solid.

### Additional Synthetic Information:

The following compounds were prepared using the identical reaction with the following starting materials

| Target compound | Thiophene starting material | |
|---|---|---|
| number | structure | Carboxylic acid starting material |
| 43 | 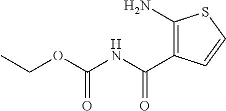 | 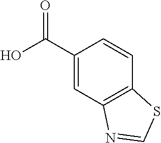 [SMILES: O=C(O)c1ccc2scnc2c1] |
| 64 | 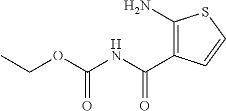 | 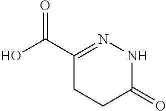 |
| 67 | 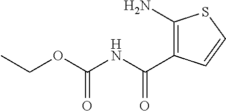 | 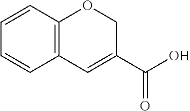 [SMILES: O=C(O)C1=Cc2ccccc2OC1] |
| 68 | 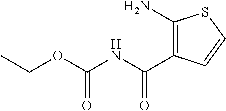 | 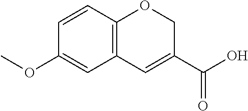 |
| 75 | 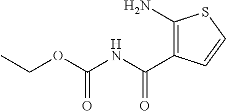 | 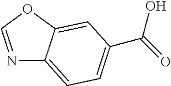 [SMILES: O=C(O)c1ccc2ncoc2c1] |
| 81 | 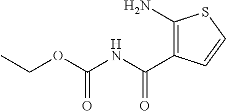 | 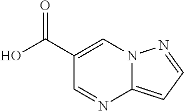 [SMILES: O=C(O)c1cnc2ccnn2c1] |
| 93 | 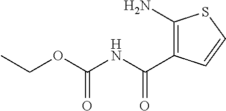 | 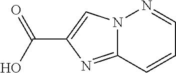 [SMILES: O=C(O)c1cn2ncccc2n1] |

### Method C:

The following compounds were synthesized via Method C: 62, 76.

Generalized Example Scheme: Compound 76

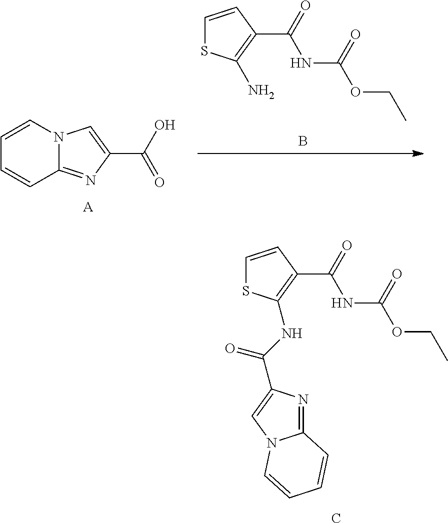

To a solution of carboxylic acid (1.1 g, 7 mmol) in dry DCM (10 mL) at 0° C. under nitrogen was added B (1.5 mg, 7 mmol), Et3N (2.1 g, 21 mmol), EDCI (4.7 g, 23.8 mmol), HOBt (2.8 g, 21 mmol) and DMAP (150 mg, catalytic). Reaction mixture was stirred at 25° C. for 2-12 h, monitored by TLC. Mixture was poured into cold water (10 ml) and extracted with DCM (20 ml), washed with water and brine, then dried and concentrated. Crude material was purified by column chromatography (silica gel, EtOAc:PE=1:2) to give C (751 mg, 30%) as a yellow solid.

### Additional Synthetic Information:

The following compounds were prepared using the identical reaction with the following starting materials

| Target compound | Thiophene starting material | Carboxylic acid starting |
|---|---|---|
| number | structure | material |
| 62 | 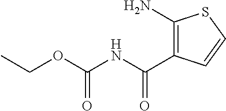 | 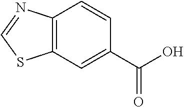 [SMILES: O=C(O)c1ccc2ncsc2c1] |

Additional compounds were prepared using similar protocols as described above. These compounds are listed in Table A.

### BIOLOGICAL EXAMPLES

### Example I

### High Throughput Screen for Inhibitors of Biofilm Formation Inhibition

A diverse chemical library (˜70,000 compounds) was used for the primary screen. This in-house compound library was created based on a chemoinformatic analysis of scaffold chemical diversity, historical proprietary screen hit rates (>300 Mio data points from the HTS database) and commercial availability. 105/ml M. smegmatis cells were plated in 384 well plates in biofilm formation medium (M63 salts minimal medium supplemented with 2% glucose, 0.5% Casamino Acids, 1 mM MgSO₄, and 0.7 mM CaCl₂). RIF and TMC207 were used as positive controls, and DMSO (0.1%) as a negative control. Cells were treated with 10 μM compound, incubated for 3 days, and the OD of each well was determined with an EnVision Multilabel Reader. The average Z′ and coefficient values are 0.512 and 8.7%, respectively. A high stringency cutoff (3-fold inhibition) was used to pick hits that are most likely growth inhibitors (hit rate 0.03%), and a low stringency cutoff (2-fold inhibition) to include hits that inhibit biofilm formation without significant growth inhibition (hit rate 0.17%).

### Example II

### In Vitro Activity Assays

For kinetic killing assays, exponentially growing cultures of mycobacteria were diluted in fresh media to an OD600 of 0.1-0.2. Various drugs were added to the culture at the indicated concentrations. The number of colony forming units (CFU) at the start of the experiment was estimated by plating appropriate dilutions of the culture onto 7H10 agar plates. The effect of drug was monitored by plating for CFU at the indicated time points. All experiments were carried out in triplicate. MICs were determined by a turbidity assay. Threefold serial dilutions in DMSO were prepared for each compound. Mtb cultures (OD=0.04) were incubated with compounds at 37° C. for 5 days and OD600 was determined with an Envision plate reader. All experiments were carried out in duplicate. For assays under starvation conditions, a log-phase growing Mtb culture was centrifuged and the cell pellet was washed twice with PBS, resuspended in PBS with Tyloxapol (0.05%) (OD=0.3), and incubated with DMSO, TCA1 (7.5 μg/mL), and RIF (2 μg/mL). All experiments were carried out in triplicate. For intracellular macrophage assays, J744.1 murine macrophage cells were infected with Mtb at a MOI of 1:3 and incubated for 2 hours at 37° C. After washing the cell monolayer three times, 20 μM amikacin was added and the culture was incubated for an additional 2 hours to kill remaining extracellular bacteria. Infected cells were then incubated in the presence of serial dilutions of compounds for 5 days. Cells were washed three times and lysed in each well; the lysate was transferred to a 96 well plate for serial dilution, and then plated on 7H11 agar medium for CFU assays. All experiments were carried out in triplicate.

### Example III

### In Vivo Efficacy Experiments

Six- to eight-week-old-female BALB/c mice (US National Cancer Institute) were infected via aerosol with a low dose (approximately 50 bacilli) of Mtb H37Rv. Infection dose was verified by plating the inoculum and the whole lung homogenates onto 7H10 plates at 24 hrs post-infection. Treatment of BALB/c mice began at either 2 weeks or 4 weeks post-infection with RIF (10 mg/kg) and INH (25 mg/kg) administered ad libitum in drinking water (changed once every two days). TCA1 was administered by oral gavage, once daily for 5 days per week at a dosage of either 40 mg/kg or 100 mg/kg for the indicated durations. At predetermined time points, or at humane end-points, animals were heavily sedated, euthanized and tissues collected for culture and pathology. Treatment efficacy was assessed on the basis of CFU in the lungs and spleen of treated mice compared to untreated controls and bacterial burden in these organs prior to treatment start. Organs were homogenized in PBS containing Tween-80 (0.05%) and various dilutions were placed on 7H10 plates. Plates were incubated at 37° C. for three weeks and CFU on the various plates recorded. All animal experimental protocols were approved by the Animal Care and Usage Committee of AECOM.

### Example IV

### Genome-Wide Transcriptional Analysis

Triplicate 10 ml cultures of mycobacteria were grown to log phase for transcriptional profiling of planktonic cells or for three weeks in pellicle media for transcriptional profiling of pellicle cells. For TCA1 treatment, log phase cultures were treated with 3.75 μg/ml TCA1 or DMSO vehicle for 12 hours. Cells were harvested, washed, and re-suspended in 1 ml RNA Protect reagent (Qiagen) and incubated 4 h at room temperature (21° C.). All transcriptional profiling procedures, including RNA extraction, DNase treatment, cDNA synthesis, labeling, microarray hybridization, washing, scanning, and data analysis were performed as previously described [Vilcheze C, Weinrick B, Wong K W, Chen B, & Jacobs W R, Jr. (2010) NAD+ auxotrophy is bacteriocidal for the tubercle bacilli. Mol Microbiol 76:365-377]. Microarray data have been deposited in the US National Center for Biotechnology Information Gene Expression Omnibus (GEO series accession number GSE37392). For qPCR experiments, diluted cDNA was used as a template at 50 ng per reaction for real time PCR reactions containing primer sets designed by Primer 3 and SYBR Green PCR Master Mix (Applied Biosystems) in accordance with the manufacturers' instructions. These reactions were carried out on an ABI 9700HT real-time PCR cycler (Applied Biosystems).

### Example V

### DprE1 Competition Assay

DprE1 was incubated with serial dilutions of TCA1 for 15 min. BTZ-BODIPY was added and the sample incubated for 1 h at 37° C. BTZ-BODIPY is a fluorescent BTZ derivative which reacts in the presence of farnesylphosphoribose (FPR) with DprE1 forming a covalent bond. Samples are then analyzed by SDS-PAGE (fluorescence and Coomassie staining).

### Example VI

### Crystallization and Structure Determination

Mycobacterium tuberculosis DprE1 (Rv3790) was prepared for crystallization [Batt S M, et al. (2012) Structural basis of inhibition of Mycobacterium tuberculosis DprE1 by benzothiazinone inhibitors. Proc Natl Acad Sci USA 109:11354-11359]. Prior to setting up crystallization experiments, the TCA1 inhibitor (in DMSO) was incubated with concentrated protein (˜35 mg/ml) for 30 min at a molar ratio of 3 TCA1:1 DprE1. Crystals were grown by sitting drop vapor diffusion and appeared over a reservoir consisting of 40-43% (w/v) polypropylene glycol 400 and 0.1 M imidazole, pH 7.0. Crystals were mounted into nylon loops directly from the drop and frozen in liquid nitrogen. X-ray diffraction data to 2.6 Å resolution were recorded on beamline 102 of the Diamond Light Source. The crystals were in space group P21, with two molecules of the complex in the crystallographic asymmetric unit. Initial phases were obtained by molecular replacement (PHASER) [McCoy A J, et al. (2007) Phaser crystallographic software. J Appl Crystallogr 40:658-674], using the apo structure of DprE1 (PDB entry 4FDP) [Batt S M, et al. (2012) Structural basis of inhibition of Mycobacterium tuberculosis DprE1 by benzothiazinone inhibitors. Proc Natl Acad Sci USA 109:11354-11359] as a search model. Following refinement of the molecular replacement solution, density for TCA1 was clearly visible in the active sites of the two crystallographically distinct copies of DprE1. Density shape and stereochemical constraints allowed us to unequivocally place the inhibitor in the active site of DprE1. Model rebuilding and structure refinement (COOT) [Emsley P, Lohkamp B, Scott W G, & Cowtan K (2010) Features and development of Coot. Acta Crystallogr D Biol Crystallogr 66:486-501], REFMAC5 [Murshudov G N, et al. (2011) REFMAC5 for the refinement of macromolecular crystal structures. Acta Crystallogr D Biol Crystallogr 67:355-367], PHENIX.REFINE [Adams P D, et al. (2010) PHENIX: a comprehensive Python-based system for macromolecular structure solution. Acta Crystallogr D Biol Crystallogr 66:213-221] led to final R-factors of 23.7% and 17.6% for the test and working sets, respectively.

### Example VII

### Affinity-Based Proteomics and Photo-Affinity Labeling

H37Ra cells were lysed with homogenization buffer [60 mM β-glycerophosphate, 15 mM p-nitrophenyl phosphate, 25 mM Mops (pH 7.2), 15 mM MgCl2, 1 mM DTT, protease inhibitors, and 0.5% Nonidet P-40]. Cell lysates were centrifuged at 16,000×g for 20 min at 4° C. and the supernatant was collected. Total protein concentration in the supernatant was determined by a BCA protein assay kit (Pierce). The lysates (1 mg) were then added to the affinity resin (30 μl) and the loading buffer [50 mM Tris.HCl (pH 7.4), 5 mM NaF, 250 mM NaCl, 5 mM EDTA, 5 mM EGTA, protease inhibitors, 0.1% Nonidet P-40] was added to a final volume of 1 ml (for the competition experiment, TCA1 was added to a final concentration of 50 μM). After rotating at 4° C. for 1 h, the mixture was centrifuged at 16,000×g for 1 min at 4° C., and the supernatant was removed. The affinity resin was then washed for 5 times with cold loading buffer and eluted by boiling with Laemmli sample buffer (Invitrogen) at 95° C. for 3 min. Samples were loaded and separated on a 4-20% Tris-glycine gel (Invitrogen). The gel band was extracted and analyzed by proteomics. For the photo-affinity experiments, E. coli cells overexpressing MoeW and native cells were lysed and the photo-affinity probe was added to cell lysates (1 mg) in 50 μL PBS and incubated for 2 h at room temperature followed by UV irradiation with a UV lamp for 20 min. The reaction mixtures were then subjected to click-chemistry with rhodamine-azide (100 μM) and incubated for 2 h at room temperature with gentle mixing. The reactions were terminated by the addition of pre-chilled acetone (0.5 mL), placed at −20° C. for 30 min and centrifuged at 16,000×g for 10 min at 4° C. to precipitate proteins. The pellet was washed two times with 200 μL of pre-chilled methanol, resuspended in 25 μL 1×standard reducing SDS-loading buffer and heated for 10 min at 95° C.; samples were loaded for separation by SDS-PAGE, then visualized by in-gel fluorescent scanning.

### Example VIII

### In Vitro Assays in Nitrate-Only Media

An Mtb culture was re-suspended under nitrogen-limiting conditions [Malm S, et al. (2009) The roles of the nitrate reductase NarGHJI, the nitrite reductase NirBD and the response regulator GlnR in nitrate assimilation of Mycobacterium tuberculosis. Microbiology 155:1332-1339] (a basal medium [1 L of the basal medium contains 1 g KH₂PO₄, 2.5 g Na₂HPO₄, 2 g K₂SO₄ and 2 ml of trace elements; 1 L of trace elements contained 40 mg ZnCl₂, 200 mg FeCl₃.6H₂O, 10 mg CuCl₂.4H₂O, 10 mg MnCl₂.4H₂O, 10 mg Na₂B₄O₇.10H₂O and 10 mg (NH₄)₆Mo₇O₂₄.4H₂O)] supplemented with NaNO₃ as sole source of nitrogen, 0.5 mM MgCl₂, 0.5 mM CaCl₂, 10% ADS, 0.2% glycerol and 0.05% Tween 80) and incubated for 24 hours. 7.5 μg/ml of TCA1 was then added to the culture and incubated for 30 days. CFU assay was used to determine the bacterial viability at each time point.

All experiments were carried out in triplicate.

### Example IX

### MoCo Inhibition Assay

The synthesis of MoCo form “A” dephospho was carried out according to the procedures described. The 1H-NMR spectrum matches what has been reported in the literature [(a) Taylor E C, Ray P S, & Darwish I S (1989) Studies on the Molybdenum Cofactor. Determination of the Structure and Absolute Configuration of Form A. J Am Chem Soc 111:7664-7665; (b) Mohr D, Kazimierczuk Z, & Pfleiderer W (1992) Pteridines. Part XCVII. Synthesis and properties of 6-thioxanthopterin and 7-thioisoxanthopterin. Helv. Chim. Acta. 75:2317-2326]. Conversion of all sources of molybdopterin to Form “A” dephospho was performed by following the methods previously reported with slight modifications [(a) Williams M J, Kana B D, & Mizrahi V (2011) Functional analysis of molybdopterin biosynthesis in mycobacteria identifies a fused molybdopterin synthase in Mycobacterium tuberculosis. J Bacteriol 193:98-106; (b) Johnson M E & Rajagopalan K V (1987) Involvement of chlA, E, M, and N loci in Escherichia coli molybdopterin biosynthesis. J Bacteriol 169:117-125]. 100 ml of Mtb culture was harvested and the pellet was re-suspended in extraction solution (2 mL, 10 mM sodium ascorbate). The cells were lysed and centrifuged at 16,000×g; the supernatant was collected and treated with acidic iodine solution at 95° C. for 25 min and the excess iodine was removed by adding sodium ascorbate. After centrifugation, the solution was neutralized with ammonium hydroxide, then concentrated and dephosphorylated using calf intestinal phosphatase (NEB) at 37° C. for 3 h. HPLC analysis was performed using Agilent C18 column (150×4.6 mm, 10-μm of particle size) with gradient elution by buffer A (50 mM ammonium acetate) and buffer B (MeOH) (97% A to 93% A in 14 min and 97% B wash from 15 min to 22 min). Fluorescence detection was at 370/450 nm.

### Example X

### Cell-Free Assay for DPA Biosynthesis

### Preparation of the Mycobacterial Membrane and Cell Envelope Enzymatic Fraction.

About 2 g cell pellet of Mycobacterium smegmatis mc²155 grown in the LB medium (Invitrogen) supplemented with 0.05% Tween 80, was washed with Buffer A [50 mM MOPS (pH 7.9), 5 mM 2-mercaptoethanol and 10 mM MgCl₂] and suspended in 10 ml of the same buffer. The cells were disintegrated by probe sonication performed in 30 s pulses with 90 s cooling pauses, repeated 20 times. The sonicate was centrifuged at 15,600×g for 20 min at 4° C. and the resulting pellet was used for preparation of the cell envelope enzymatic fraction, as described [Mikusova K, et al. (2005) Decaprenylphosphoryl arabinofuranose, the donor of the D-arabinofuranosyl residues of mycobacterial arabinan, is formed via a two-step epimerization of decaprenylphosphoryl ribose. J Bacteriol 187:8020-8025] with minor modifications. Briefly, the pellet was homogenized with Buffer A to the volume 4 ml of the final suspension, to which 6 ml of Percoll (GE Healthcare) was added and the mixture was centrifuged at 15,600×g for 60 min at 4° C. White upper band was collected and Percoll was removed from the sample by repeated washings with Buffer A and centrifugations at 15,600×g for 20 min at 4° C. The final pellet was resuspended in 400 μl of Buffer A resulting in the sample with the protein concentration of 6.8 mg/ml, which was used as the source of the cell envelope enzyme in the cell free reactions. Membrane fraction with protein concentration of 49 mg/ml was prepared by centrifugation of 15,600×g supernatant of the sonicate at 100,000×g, as described [Mikusova K, et al. (2005) Decaprenylphosphoryl arabinofuranose, the donor of the D-arabinofuranosyl residues of mycobacterial arabinan, is formed via a two-step epimerization of decaprenylphosphoryl ribose. J Bacteriol 187:8020-8025].

### Composition and Analysis of the Cell Free Reactions

The reaction mixtures contained 75,000 dpm of phospho-[¹⁴C]-ribose diphosphate [Scherman M S, et al. (1996) Polyprenylphosphate-pentoses in mycobacteria are synthesized from 5-phosphoribose pyrophosphate. J Biol Chem 271:29652-29658], 0.1 mM NADH, 3.125% DMSO, 500 μg of membrane protein or 200 μg of the cell envelope protein and Buffer A in the final volume of 80 μl. TCA1 and BTZ043 dissolved in DMSO were added to the reaction mixtures in the final concentration of 25 μg/ml. For dose-dependence experiment TCA1 was added in the final concentrations 1, 3, 6, 12 and 25 μg/ml in the reaction mixtures. After 1 h incubation at 37° C., the reactions were stopped by the addition of 1.5 ml of CHCl₃/CH₃OH (2:1). After 20 min extraction of the reaction products at RT, 170 μl of Buffer A was added, the tubes were thoroughly mixed and then briefly centrifuged at 3,000×g to achieve separation of two phases of the mixture [Folch J, Lees M, & Sloane Stanley G H (1957) A simple method for the isolation and purification of total lipides from animal tissues. J Biol Chem 226:497-509]. Upper, aqueous phase containing unreacted radiolabelled substrate was discarded; bottom, organic phase was transferred to the new tube and dried under the stream of N₂ at RT. The organic extract was dissolved in 40 μl of CHCl₃/CH₃OH/H₂O/conc. NH₄OH (65:25:3.6:0.5) and analysed by TLC on aluminium-backed silica gel plates (F₂₅₄, Merck) in CHCl₃/CH₃OH/1M CH₃COONH₄/conc. NH₄OH/H₂O (180:140:9:9:23). Radiolabeled compounds were visualized by autoradiography [BioMax MR film (Kodak)].

### Example XI

### Biological Assay and Analytical Data

Table A shows the biological data for compounds 1-460 as well as analytical data (NMR and/or MS) for selected compounds.

The MIC of compounds of Formulas (I), (Ia), (Ib), (Ic), (Id), (II), (IIa), (IIb), and (IIc) was determined in a microplate alamar blue assay (MABA; listed as “I” in Table A) or turbidity (listed as “II” in Table A) assay. The MIC of compounds of Formulas (I), (Ia), (Ib), (Ic), (Id), (II), (IIa), (IIb), and (IIc) in the assay was graded as A: <1 μg/mL; B: 1-3 μg/mL; and C: >3 μg/mL.

The compounds of Formulas (I), (Ia), (Ib), (Ic), (Id), (II), (IIa), (IIb), and (IIc) were also evaluated under starvation conditions in a low-oxygen-recovery assay (LORA; listed as “III” in Table A). The MIC of compounds of Formulas (I), (Ia), (Ib), (Ic), (Id), (II), (IIa), (IIb), and (IIc) in the assay was graded as A: <5 μg/mL; B: 5-25 μg/mL; and C: >25 μg/mL.

The compounds of Formulas (I), (Ia), (Ib), (Ic), (Id), (II), (IIa), (IIb), and (IIc) were further evaluated in cytotoxicity assays using either Vero cells (listed as “IV” in Table A) or Huh7 cells (listed as “V” in Table A). The LC50 was graded as A: >50 μg/mL; B: 50-25 μg/mL; and C: <25 μg/mL.

TABLE A

| Cpd | | | | | | | LCMS and/or |
|---|---|---|---|---|---|---|---|
| ID | Structure | I | II | III | IV | V | NMR Data |
| 1 | 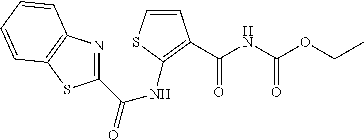 [SMILES: CCOC(=O)NC(=O)c1ccsc1NC(=O)c1nc2ccccc2s1] | B | B | C | A | A | ES MS M/Z = 376 (M + 1) ¹H NMR (400 MHz, CD₃OD) 8.24 (d, J = 8.2 Hz, 1H), 8.16 (d, J = 8.0 Hz, 1H), 7.68 (t, J = 7.6 Hz, 1H), 7.62 (t, J = 7.6 Hz, 1H), 7.56 (d, J = 5.9 Hz, 1H), 7.10 (d, J = 5.9 Hz, 1H), 4.33 (q, J = 7.1 Hz, 2H), 1.39 (t, J = 7.1 Hz, 3H). |
| 2 | 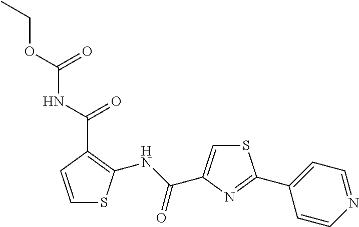 [SMILES: CCOC(=O)NC(=O)c1ccsc1NC(=O)c1csc(-c2ccncc2)n1] | | B | | | | |
| 3 | 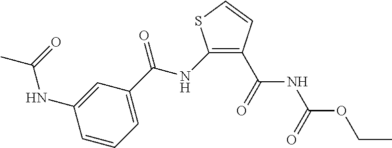 [SMILES: CCOC(=O)NC(=O)c1ccsc1NC(=O)c1cccc(NC(C)=O)c1] | | B | | | A | |
| 4 | 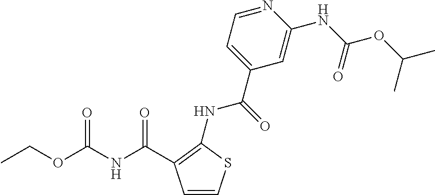 [SMILES: CCOC(=O)NC(=O)c1ccsc1NC(=O)c1ccnc(NC(=O)OC(C)C)c1] | | B | | | A | |
| 5 | 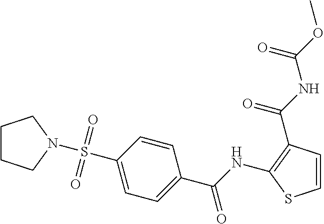 [SMILES: COC(=O)NC(=O)c1ccsc1NC(=O)c1ccc(S(=O)(=O)N2CCCC2)cc1] | | A | | | | |
| 6 | 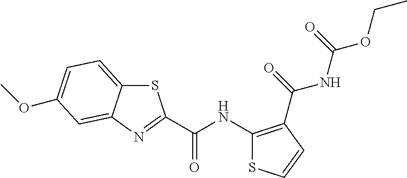 [SMILES: CCOC(=O)NC(=O)c1ccsc1NC(=O)c1nc2cc(OC)ccc2s1] | | A | | | | |
| 7 | 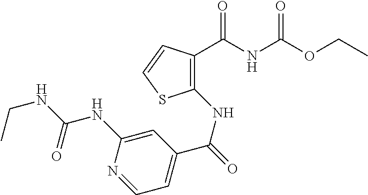 [SMILES: CCNC(=O)Nc1cc(C(=O)Nc2sccc2C(=O)NC(=O)OCC)ccn1] | | A | | | | |
| 8 | 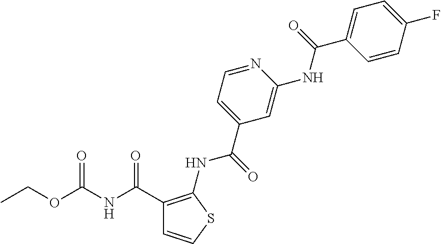 [SMILES: CCOC(=O)NC(=O)c1ccsc1NC(=O)c1ccnc(NC(=O)c2ccc(F)cc2)c1] | | B | | | | |
| 9 | 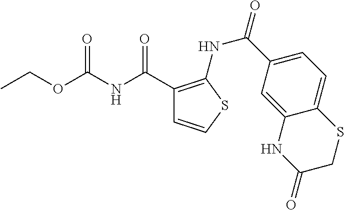 [SMILES: CCOC(=O)NC(=O)c1ccsc1NC(=O)c1ccc2c(c1)NC(=O)CS2] | | B | | | | |
| 10 | 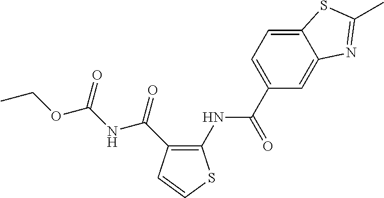 [SMILES: CCOC(=O)NC(=O)c1ccsc1NC(=O)c1ccc2sc(C)nc2c1] | A | A | | B | A | |
| 11 | 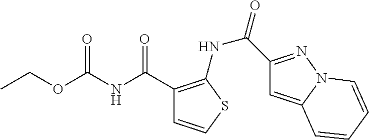 [SMILES: CCOC(=O)NC(=O)c1ccsc1NC(=O)c1cc2ccccn2n1] | | B | | | A | |
| 12 | 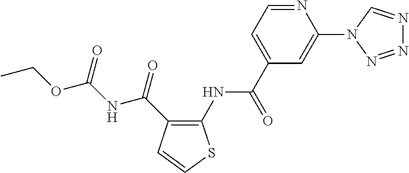 [SMILES: CCOC(=O)NC(=O)c1ccsc1NC(=O)c1ccnc(-n2cnnn2)c1] | | B | | | A | |
| 13 | 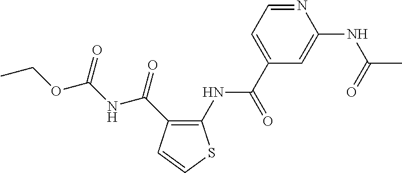 [SMILES: CCOC(=O)NC(=O)c1ccsc1NC(=O)c1ccnc(NC(C)=O)c1] | | A | | | A | |
| 14 | 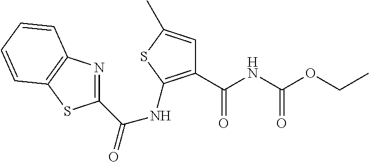 [SMILES: CCOC(=O)NC(=O)c1cc(C)sc1NC(=O)c1nc2ccccc2s1] | A | | | | | ES MS M/Z = 390 (M + 1) ¹H NMR (400 MHz, CDCl₃) 8.08 (d, J = 8.2 Hz, 1H), 8.02 (d, J = 8.2 Hz, 1H), 7.54 (t, J = 8.2 Hz, 1H), 7.48 (t, J = 8.2 Hz, 1H), 7.06 (s, 1H), 4.18 (q, J = 7.1 Hz, 2H), 2.35 (s, 3H), 1.25 (t, J = 7.2 Hz, 3H). |
| 15 | 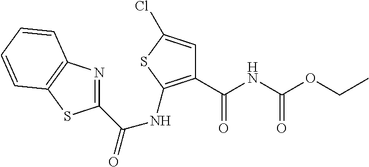 [SMILES: CCOC(=O)NC(=O)c1cc(Cl)sc1NC(=O)c1nc2ccccc2s1] | A | | | | | ¹H NMR (400 MHz, CDCl₃) 13.088 (br, 1H), 10.837 (br, 1H), 8.326 (d, J = 10 Hz, 1H), 7.880 (s, 1H), 7.691 (m, 2H), 4.241 (m, 2H), 2.285 (s, 3H), 1.301 (t, J = 7.2 Hz, 3H). |
| 16 | 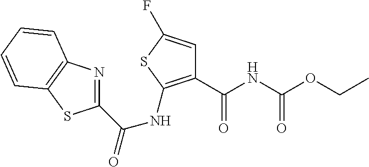 [SMILES: CCOC(=O)NC(=O)c1cc(F)sc1NC(=O)c1nc2ccccc2s1] | A | | | B | | ¹H NMR (400 MHz, DMSO-d₆) 10.762 (s, 1H), 8.304 (d, J = 8 Hz, 2H), 7.693- 7.646 (m, 2H), 7.495 (s, 1H), 4.217 (q, J = 7.0 Hz, 2H), 1.285 (t, J = 7.1 Hz, 3H); ¹⁹F NMR (376 MHz, DMSO-d₆) δ −139.886 (s) |
| 17 | 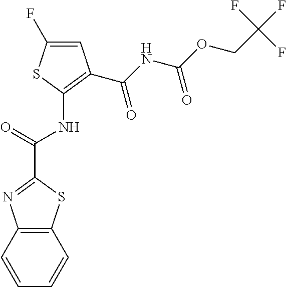 [SMILES: O=C(NC(=O)c1cc(F)sc1NC(=O)c1nc2ccccc2s1)OCC(F)(F)F] | A | | | C | | ¹H NMR (400 MHz, CD₃OD) 8.224 (d, J = 7.9 Hz, 1H), 8.148 (d, J = 7.4 Hz, 1H), 7.667-7.607 (m, 2H), 7.166 (d, J = 3.6 Hz, 1H), 4.777 (q, J = 8.7 Hz, 2H); ¹⁹F NMR (376 MHz, CD₃OD) −74.961 (s), −140.976 (s) |
| 18 | 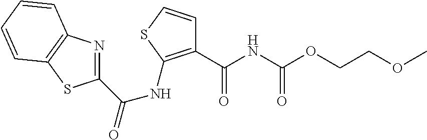 [SMILES: COCCOC(=O)NC(=O)c1ccsc1NC(=O)c1nc2ccccc2s1] | A | | C | A | | ES-MS M/Z = 406.1 (M + 1) ¹H NMR (400 MHz, DMSO-d₆) 8.303 (d, J = 9.3 Hz, 2H), 7.784 (d, J = 6.0 Hz, 1H), 7.770 (m, 2H), 7.188 (d, J = 5.9 Hz, 2H), 4.295 (t, J = 4.5 Hz, 2H), 3.602 (t, J = 4.5 Hz, 2H), 3.290 (s, 3H). |
| 19 | 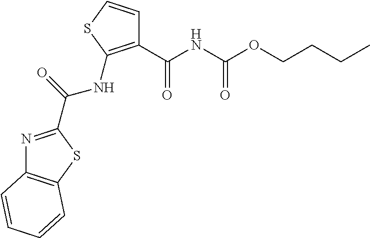 [SMILES: CCCCOC(=O)NC(=O)c1ccsc1NC(=O)c1nc2ccccc2s1] | A | | | | | ¹H NMR (500 MHz, CDCl₃) 8.354 (d, J = 8.5 Hz, 1H), 8.079 (d, J = 8.0 Hz, 1H), 7.9 (s, 1H), 7.170 (d, J = 6.0 Hz, 1H), 7.044 (d, J = 5.5 Hz, 1H), 4.377 (d, J = 6.5 Hz, 2H), 1.822-1.792 (m, 1H), 1.557-1.527 (m, 1H), 1.1 (t, J = 6.8 Hz, 3H). |
| 20 | 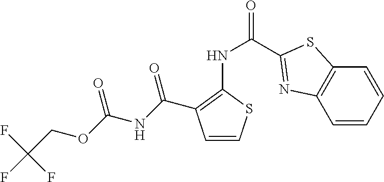 [SMILES: O=C(NC(=O)c1ccsc1NC(=O)c1nc2ccccc2s1)OCC(F)(F)F] | A | | | C | | ES-MS M/Z = 394 (M + 1) ¹H NMR (301 MHz, CDCl₃) 12.99 (s, 1H), 8.23 (d, J = 8.1 Hz, 1H), 8.15 (s, 1H), 7.58 (m, 2H), 7.16 (d, J = 6.0 Hz, 1H), 6.97 (d, J = 6.0 Hz, 1H), 4.77 (m, 1H), 4.58 (m, 2H), 4.47 (m, 1H). |
| 21 | 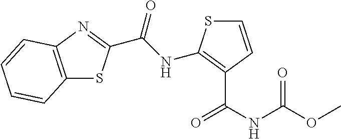 [SMILES: COC(=O)NC(=O)c1ccsc1NC(=O)c1nc2ccccc2s1] | A | | | | | |
| 22 | 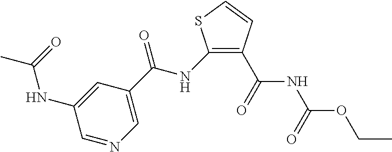 [SMILES: CCOC(=O)NC(=O)c1ccsc1NC(=O)c1cncc(NC(C)=O)c1] | A | | | | | ¹H NMR (500 MHz, DMSO-d₆) 10.89 (s, 1H), 10.57 (s, 1H), 9.04 (s, 1H), 8.84 (s, 1H), 8.63 (s, 1H), 7.79 (d, J = 10 Hz, 1H), 7.18 (d, J = 5 Hz, 1H), 4.28 (q, J = 10 Hz, 2H), 2.20 (s, 3H), 1.35 (t, J = 10 Hz, 3H). |
| 23 | 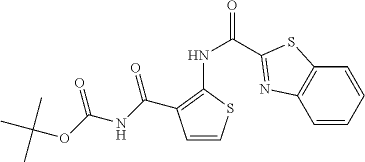 [SMILES: CC(C)(C)OC(=O)NC(=O)c1ccsc1NC(=O)c1nc2ccccc2s1] | A | | | | | ¹H NMR (400 MHz, DMSO-d₆) 10.572 (s, 1H), 8.312-8.282 (m, 2H), 7.741 (d, J = 6.0 Hz, 1H), 7.696 (m, 2H), 7.177 (d, J = 5.9 Hz, 1H), 1.507 (s, 9H). |
| 24 | 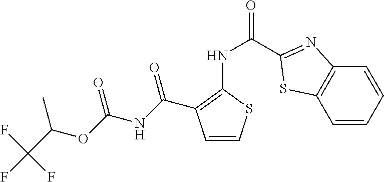 [SMILES: CC(OC(=O)NC(=O)c1ccsc1NC(=O)c1nc2ccccc2s1)C(F)(F)F] | A | | | C | | ¹H NMR (400 MHz, DMSO-d₆) 8.301 (d, J = 8.7 Hz, 2H), 7.769 (d, J = 6.0 Hz, 1H), 7.696 (m, 2H), 7.204 (d, J = 6.0 Hz, 1H), 5.557-5.523 (m, 1H), 1.460 (d, J = 6.6 Hz, 3H). |
| 25 | 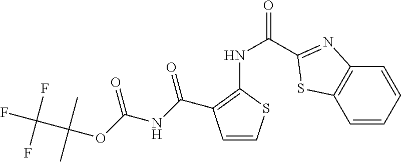 [SMILES: CC(C)(OC(=O)NC(=O)c1ccsc1NC(=O)c1nc2ccccc2s1)C(F)(F)F] | A | | | B | | ¹H NMR (400 MHz, DMSO-d₆) 8.301 (d, J = 8.7 Hz, 2H), 7.769 (d, J = 6.0 Hz, 1H), 7.696 (m, 2H), 7.204 (d, J = 6.0 Hz, 1H), 5.557-5.523 (m, 1H), 1.460 (d, J = 6.6 Hz, 3H). |
| 26 | 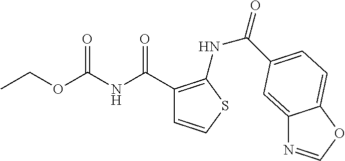 [SMILES: CCOC(=O)NC(=O)c1ccsc1NC(=O)c1ccc2ocnc2c1] | A | | | A | | ¹H NMR (400 MHz, DMSO-d₆) 10.819 (s, 1H), 8.940 (s, 1H), 8.344 (t, J = 1.2 Hz, 1H), 8.034 (6, J = 1.2 Hz, 1H), 7.718 (d, J = 6.0 Hz, 1H), 7.087 (d, J = 5.9 Hz, 1H), 4.207 (q, J = 7.1 Hz, 1H), 1.275 (t, J = 7.1 Hz, 1H). |
| 27 | 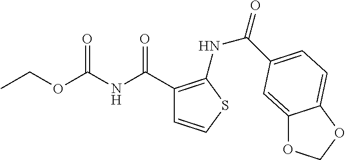 [SMILES: CCOC(=O)NC(=O)c1ccsc1NC(=O)c1ccc2c(c1)OCO2] | A | | | C | | ¹H NMR (500 MHz, DMSO-d₆) 10.84 (s, 1H), 7.78 (d, J = 5 Hz, 1H), 7.58 (d, J = 10 Hz, 1H), 7.48 (s, 1H), 7.23 (d, J = 10 Hz, 1H), 7.22 (d, J = 5 Hz, 1H), 6.25 (s, 2H), 4.28 (q, J = 5 Hz, 1H), 1.35 (t, J = 5 Hz, 1H). |
| 29 | 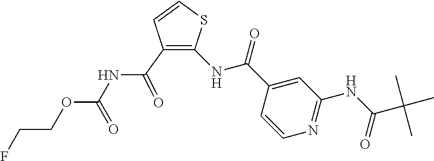 [SMILES: CC(C)(C)C(=O)Nc1cc(C(=O)Nc2sccc2C(=O)NC(=O)OCCF)ccn1] | A | | C | B | | ¹H NMR (500 MHz, DMSO-d₆) 10.20 (s, 1H), 8.63 (s, 1H), 8.62 (s, 1H), 7.78 (d, J = 5.0 Hz, 1H), 7.56-7.54 (m, 1H), 7.16 (d, J = 5.0 Hz, 1H), 4.78-4.65 (m, 2H), 4.49-4.40 (m, 2H), 1.28 (s, 9H). |
| 30 | 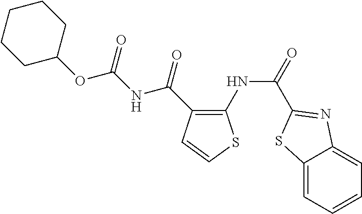 [SMILES: O=C(NC(=O)c1ccsc1NC(=O)c1nc2ccccc2s1)OC1CCCCC1] | A | | | A | | ¹H NMR (400 MHz, DMSO-d₆) 10.784 (s, 1H), 8.306-8.284 (m, 2H), 7.761 (d, J = 6.0 Hz, 1H), 7.695 (m, 7H), 7.186 (d, J = 5.8 Hz, 1H), 4.740-4.707 (m, 1H), 1.908-1.091 (m, 10H). |
| 31 | 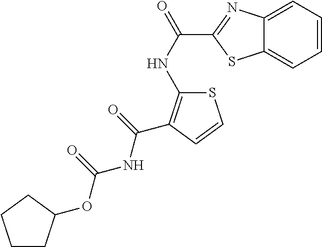 [SMILES: O=C(NC(=O)c1ccsc1NC(=O)c1nc2ccccc2s1)OC1CCCC1] | A | | | A | | ¹H NMR (400 MHz, DMSO-d₆) 10.758 (s, 1H), 8.296 (d, J = 7.8 Hz, 1H), 7.751 (d, J = 6.0 Hz, 1H), 7.694 (m, 7H), 7.183 (d, J = 5.8 Hz, 1H), 7.740-7.704 (m, 1H), 5.145-5.130 (m, 1H), 1.969- 1.584 (m, 8H). |
| 32 | 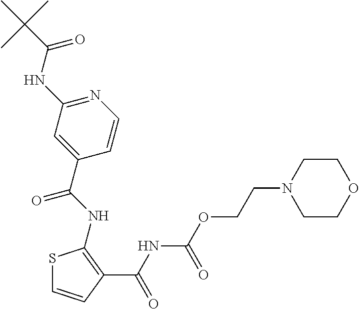 [SMILES: CC(C)(C)C(=O)Nc1cc(C(=O)Nc2sccc2C(=O)NC(=O)OCCN2CCOCC2)ccn1] | C | | C | A | | ¹H NMR (400 MHz, DMSO-d₆) 10.159 (br, 1H), 8.581 (br, 2H), 7.721 (br, 1H), 7.519 (br, 1H), 7.127 (br, 1H), 4.28 (m, 2H), 3.565 (m, 4H), 2.493-2.475 (m, 6H buried inside the DMSO peak), 1.250 (s, 9H). |
| 33 | 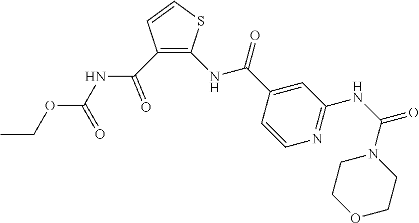 [SMILES: CCOC(=O)NC(=O)c1ccsc1NC(=O)c1ccnc(NC(=O)N2CCOCC2)c1] | A | | C | A | | ¹H NMR (500 MHz, DMSO-d₆) 10.89 (s, 1H), 9.65 (s, 1H), 8.58 (d, J = 5 Hz, 1H), 8.39 (s, 1H), 7.80 (d, J = 10 Hz, 1H), 7.46 (t, J = 5 Hz, 1H), 7.20 (d, J = 5 Hz, 1H), 4.28 (q, J = 5 Hz, 1H), 3.68-3.66 (m, 4H), 3.57-3.55 (m, 4H), 1.35 (t, J = 5 Hz, 1H). |
| 34 | 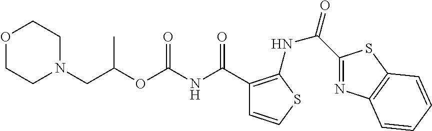 [SMILES: CC(CN1CCOCC1)OC(=O)NC(=O)c1ccsc1NC(=O)c1nc2ccccc2s1] | C | | B | C | | ¹H NMR (500 MHz, CD₃OD) 8.30-8.28 (m, 2H), 7.74-7.61 (m, 3H), 7.17 (m, 1H), 5.29 (m, 1H), 3.77 (m, 4H), 2.73- 2.57 (m, 6H), 1.44 (d, J = 5.0 Hz, 3H). |
| 35 | 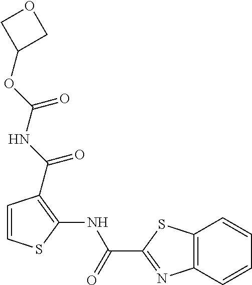 [SMILES: O=C(NC(=O)c1ccsc1NC(=O)c1nc2ccccc2s1)OC1COC1] | A | | C | C | | ¹H NMR (500 MHz, CD₃OD) 8.30 (d, J = 10.0 Hz, 1H), 8.23 (d, J = 10.0 Hz, 1H), 7.75-7.63 (m, 3H), 7.18 (d, J = 5.0 Hz, 1H), 5.66-5.57 (m, 1H), 5.04 (m, 2H), 4.84-4.82 (m, 2H). |
| 36 | 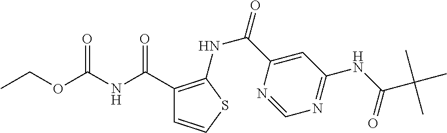 [SMILES: CCOC(=O)NC(=O)c1ccsc1NC(=O)c1cc(NC(=O)C(C)(C)C)ncn1] | B | | C | A | | ¹H NMR(400 MHz, CD₃OD) 8.976 (d, J = 1.2 Hz, 1H), 8.876 (d, J = 1.2 Hz, 1H), 7.506 (d, J = 5.9 Hz, 1H), 7.043 (d, J = 5.9 Hz, 1H), 4.283 (q, J = 7.2 Hz, 1H), 1.335 (t, J = 7.2 Hz, 1H). |
| 37 | 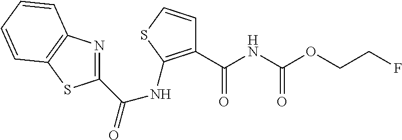 [SMILES: O=C(NC(=O)c1ccsc1NC(=O)c1nc2ccccc2s1)OCCF] | A | | C | A | | ES-MS M/Z = 394.0 (M + 1) ¹H NMR(400 MHz, CD₃OD) 8.218 (d, J = 8.2 Hz, 1H), 8.143 (d, J = 7.8 Hz, 1H), 7.599 (m, 2H), 7.562 (d, J = 5.9 Hz, 1H), 7.080 (d, J = 6.0 Hz, 1H), 4.765- 4.626 (m, 1H), 4.534-4.441 (m, 2H). |
| 38 | 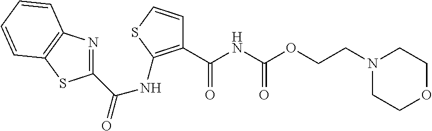 [SMILES: O=C(NC(=O)c1ccsc1NC(=O)c1nc2ccccc2s1)OCCN1CCOCC1] | A | | | A | | ES-MS M/Z = 461 (M + 1) ¹H NMR (400 MHz, CD₃OD) 8.185 (d, J = 8.2 Hz, 1H), 8.117 (d, J = 7.6 Hz, 1H), 7.64 (m, 2H), 7.508 (d, J = 5.9 Hz, 1H), 7.066 (d, J = 6.4 Hz, 1H), 4.404 (t, J = 5.6 Hz, 2H), 3.731-3.719 (m, 4H), 2.777 (t, J = 5.6 Hz, 2H), 2.619 (m, 4H). |
| 39 | 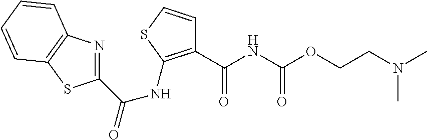 [SMILES: CN(C)CCOC(=O)NC(=O)c1ccsc1NC(=O)c1nc2ccccc2s1] | C | | | A | | ¹H NMR (500 MHz, CD₃OD) 8.243- 8.181 (m, 2H), 7.710-7.644 (m, 2H), 7.566 (m, 1H), 7.108 (m, 1H), 4.530 (m, 2H), 3.034 (m, 2H), 2.608 (s, 6H). |
| 40 | 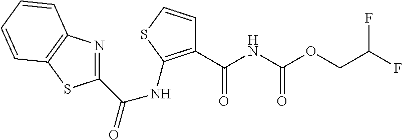 [SMILES: O=C(NC(=O)c1ccsc1NC(=O)c1nc2ccccc2s1)OCC(F)F] | A | | | C | | ¹H NMR (400 MHz, CD₃OD) 8.218 (d, J = 7.9 Hz, 1H), 8.149 (d, J = 7.2 Hz, 1H), 7.660 (m, 2H), 7.559 (d, J = 5.9 Hz, 1H), 7.080 (d, J = 5.9 Hz, 1H), 6.170 (tt, J = 54.7 and 3.6 Hz, 1H), 4.470 (td, J = 14.2 and 3.6 Hz, 1H); ¹⁹F NMR (376 MHz, CD₃OD) −127.492 (s). |
| 41 | 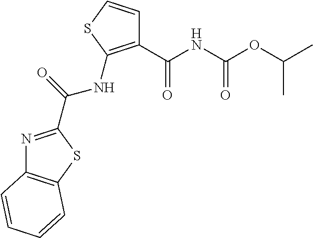 [SMILES: CC(C)OC(=O)NC(=O)c1ccsc1NC(=O)c1nc2ccccc2s1] | A | | C | C | | ES-MS M/Z = 390.1 (M + 1) ¹H NMR (500 MHz, DMSO-d₆) 10.863 (s, 1H), 8.381 (d, J = 8.4 Hz, 2H), 7.844 (d, J = 5.9 Hz, 1H), 7.771-7.725 (m, 2H), 7.262 (d, J = 5.7 Hz, 1H), 5.084- 5.033 (m, 1H), 1.380 (d, J = 6.2 Hz, 6H). |
| 42 | 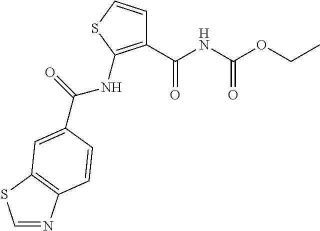 [SMILES: CCOC(=O)NC(=O)c1ccsc1NC(=O)c1ccc2ncsc2c1] | A | | | C | | ¹H NMR (500 MHz, DMSO-d₆) 10.839 (s, 1H), 9.632 (s,1H), 8.855 (s, 1H), 8.326 (d, J = 8.6 Hz, 1H), 8.070 (d, J = 8.7 Hz, 1H), 7.742 (d, J = 5.9 Hz, 1H), 7.111 (d, J = 5.9 Hz, 1H), 4.225 (q, J = 7.1 Hz, 1H), 1.292 (t, J = 7.1 Hz, 1H). |
| 43 | 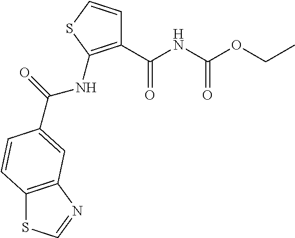 [SMILES: CCOC(=O)NC(=O)c1ccsc1NC(=O)c1ccc2scnc2c1] | A | | B | A | | ES-MS M/Z = 376.0 (M + 1) ¹H NMR (400 MHz, DMSO-d₆) 10.825 (s, 1H), 9.572 (s, 1H), 8.604 (d, J = 1.4 Hz, 1H), 8.438 (d, J = 8.5 Hz, 1H), 8.026 (dd, J = 8.4 and 1.6 Hz, 1H), 7.729 (d, J = 6.0 Hz, 1H), 7.097 (d, J = 5.9 Hz, 1H), 4.213 (q, J = 7.1 Hz, 1H), 1.279 (t, J = 7.1 Hz, 1H). |
| 44 | 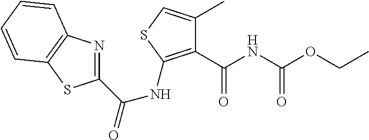 [SMILES: CCOC(=O)NC(=O)c1c(C)csc1NC(=O)c1nc2ccccc2s1] | B | | | A | | |
| 45 | 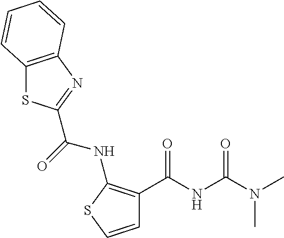 [SMILES: CN(C)C(=O)NC(=O)c1ccsc1NC(=O)c1nc2ccccc2s1] | C | | | A | | ¹H NMR (400 MHz, DMSO-d₆) 10.132 (s, 1H), 8.304-8.267 (m, 2H), 7.686- 7.633 (m, 2H), 7.595 (d, J = 5.9 Hz, 1H), 7.216 (d, J = 5.8 Hz, 1H), 2.974 (s, 6H). |
| 46 | 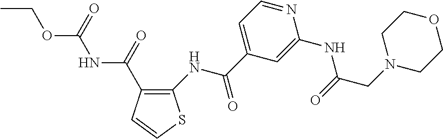 [SMILES: CCOC(=O)NC(=O)c1ccsc1NC(=O)c1ccnc(NC(=O)CN2CCOCC2)c1] | A | | C | A | | ¹H NMR (400 MHz, CDCl₃) 9.785 (br, 1H), 8.829 (s, 1H), 8.479 (d, J = 5.1 Hz, 1H), 7.945 (br, 1H), 7.580 (d, J = 5.3 Hz, 1H), 7.085 (d, J = 6.0 Hz, 1H), 4.359-4.306 (m, 2H), 3.85 (br, 4H), 3.288 (br, 2H), 2.717 (br, 4H), 1.368 (t, J = 7.1 Hz, 3H). |
| 47 | 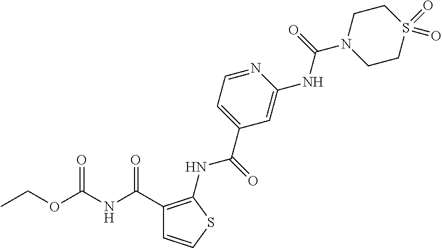 [SMILES: CCOC(=O)NC(=O)c1ccsc1NC(=O)c1ccnc(NC(=O)N2CCS(=O)(=O)CC2)c1] | A | | C | A | | ¹H NMR (500 MHz, Acetone-d₆) 9.692 (br, 1H), 8.801 (br, 1H), 8.590-8.517 (m, 2H), 7.697 (d, J = 6.0 Hz, 1H), 7.480 (m, 1H), 7.088 (d, J = 5.5 Hz, 1H), 4.254-4.226 (m, 2H), 4.146 (m, 4H), 3.250-3.241 (m, 4H), 1.292 (t, J = 7.0 Hz, 3H). |
| 48 | 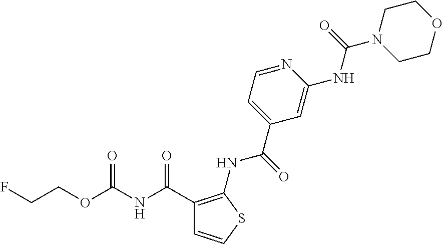 [SMILES: O=C(NC(=O)c1ccsc1NC(=O)c1ccnc(NC(=O)N2CCOCC2)c1)OCCF] | A | | C | A | | ¹H NMR (400 MHz, CDCl₃) 8.524 (s, 1H), 8.366 (t, J = 4.5 Hz, 1H), 8.258 (br, 1H), 7.483 (dd, J = 5.2 and 1.6 Hz, 1H), 7.474 (br, 1H), 7.480 (m, 1H), 7.122 (d, J = 5.9 Hz, 1H), 6.871 (d, J = 6.0 Hz, 1H), 4.756-4.473 (m, 4H), 3.785-3.761 (m, 4H), 3.566-3.554 (m, 4H). |
| 49 | 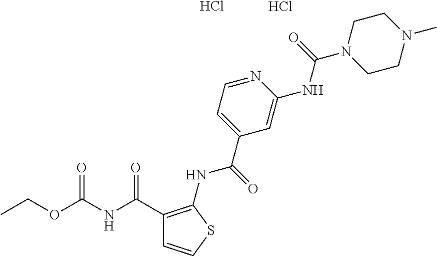 [SMILES: Cl ; Cl ; CCOC(=O)NC(=O)c1ccsc1NC(=O)c1ccnc(NC(=O)N2CCN(C)CC2)c1] | B | | C | A | | ¹H NMR (400 MHz, CD₃OD) 8.50 (br, 1H), 8.34 (br, 1H), 7.57 (br, 1H), 7.54 (d, J = 4 Hz, 1H), 7.06 (d, J = 4 Hz, 1H), 4.2 (m, 2H), 4.29 (q, J = 8 Hz, 2H), 3.58-3.30 (m, 6H buried inside the solvent peak), 2.98 (s, 3H), 1.36 (t, J = 8 Hz, 3H). |
| 50 | 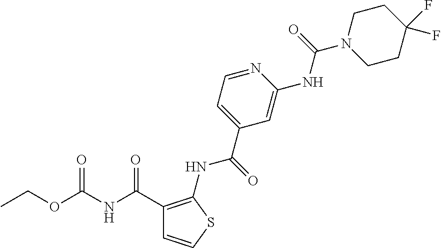 [SMILES: CCOC(=O)NC(=O)c1ccsc1NC(=O)c1ccnc(NC(=O)N2CCC(F)(F)CC2)c1] | A | | C | B | | ¹H NMR (400 MHz, CDCl₃) 8.55 (s, 1H), 8.38 (t, J = 4 Hz, 1H), 7.89 (br, 1H), 7.50 (dd, J = 8 and 4 Hz, 1H), 7.36 (br, 1H), 7.06 (d, J = 4 Hz, 1H), 6.92 (d, J = 4 Hz, 1H), 4.33 (q, J = 8 and 4 Hz, 2H), 3.68 (m, 4H), 2.11-2.04 (m, 4H), 1.37 (t, J = 8 Hz, 3H). |
| 51 | 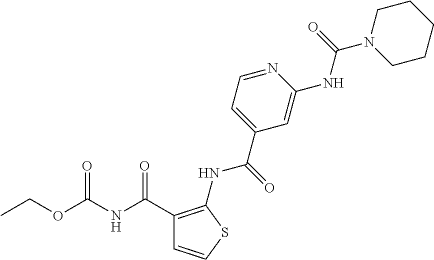 [SMILES: CCOC(=O)NC(=O)c1ccsc1NC(=O)c1ccnc(NC(=O)N2CCCCC2)c1] | A | | C | B | | ¹H NMR (400 MHz, CDCl₃) 8.60 (s, 1H), 8.34 (t, J = 4 Hz, 1H), 7.96 (br, 1H), 7.46-7.45 (m, 2H), 7.06 (d, J = 4 Hz, 1H), 6.88 (d, J = 4 Hz, 1H), 4.33 (q, J = 8 Hz, 2H), 3.53-3.51 (m, 4H), 1.66-1.64 (m, 6H), 1.37 (t, J = 8 Hz, 3H). |
| 52 | 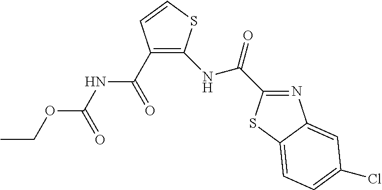 [SMILES: CCOC(=O)NC(=O)c1ccsc1NC(=O)c1nc2cc(Cl)ccc2s1] | | A | | | A | |
| 53 | 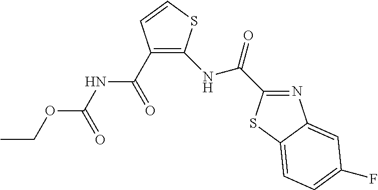 [SMILES: CCOC(=O)NC(=O)c1ccsc1NC(=O)c1nc2cc(F)ccc2s1] | A | A | | C | B | |
| 54 | 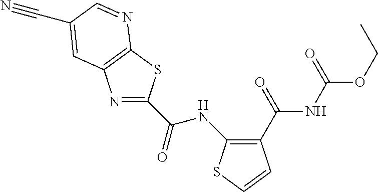 [SMILES: CCOC(=O)NC(=O)c1ccsc1NC(=O)c1nc2cc(C#N)cnc2s1] | A | | C | | | ES-MS M/Z = 402 (M + 1) ¹H NMR (301 MHz, CDCl₃) 13.11 (s, 1H), 8.94 (s, 1H), 8.77 (s, 1H), 7.93 (s, 1H), 7.12 (d, J = 6.0 Hz, 1H), 7.01 (d, J = 6.0 Hz, 1H), 4.36 (q, J = 7.1 Hz, 2H), 1.38 (t, J = 7.1 Hz, 3H). |
| 55 | 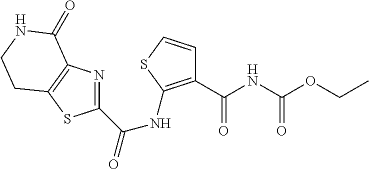 [SMILES: CCOC(=O)NC(=O)c1ccsc1NC(=O)c1nc2c(s1)CCNC2=O] | C | | C | | | ES-MS M/Z = 395 (M + 1) ¹H NMR (301 MHz, DMSO-d₆) 12.92 (s, 1H), 10.85 (s, 1H), 8.23 (s, 1H), 7.73 (d, J = 5.8 Hz, 1H), 7.19 (d, J = 5.8 Hz, 1H), 4.21 (q, J = 7.1 Hz, 2H), 3.56 (dd, J = 6.7, 2.5 Hz, 2H), 3.15 (t, J = 7.0 Hz, 2H), 1.28 (t, J = 7.1 Hz, 3H). |
| 56 | 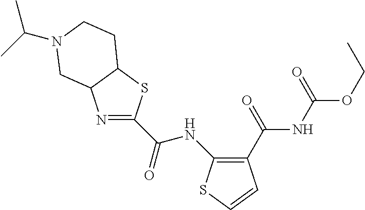 [SMILES: CCOC(=O)NC(=O)c1ccsc1NC(=O)C1=NC2CN(C(C)C)CCC2S1] | A | | C | | | ES-MS M/Z = 423 (M + 1) ¹H NMR (301 MHz, CD₃OD) 7.49 (d, J = 6.3 Hz, 1H), 7.01 (d, J = 6.3 Hz, 1H), 4.28 (q, J = 7.1 Hz, 2H), 3.88 (s, 2H), 3.04 (s, 3H), 2.99 (s, 2H), 1.36 (m, 3H), 1.22 (d, J = 6.5 Hz, 6H). |
| 57 | 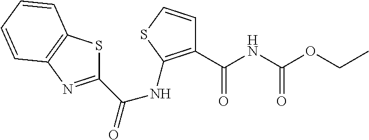 [SMILES: CCOC(=O)NC(=O)c1ccsc1NC(=O)c1nc2ccccc2s1] | A | | C | | | ES-MS M/Z = 377 (M + 1) ¹H NMR (301 MHz, DMSO-d₆) 13.18 (s, 1H), 10.88 (s, 1H), 8.90 (dd, J = 4.5, 1.5 Hz, 1H), 8.81 (dd, J = 8.1,1.5 Hz, 1H), 7.79 (d, J = 5.7 Hz, 1H), 7.67 (dd, J = 8.1, 4.5 Hz, 1H), 7.22 (d, J = 5.7 Hz, 1H), 4.26 (q, J = 6.9 Hz, 2H), 1.30 (t, J = 7.2 Hz, 3H) |
| 58 | 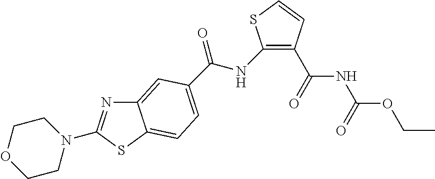 [SMILES: CCOC(=O)NC(=O)c1ccsc1NC(=O)c1ccc2sc(N3CCOCC3)nc2c1] | A | | B | A | | ES-MS M/Z = 461 (M + 1) ¹H NMR (400 MHz, CDCl₃) 12.58 (s, 1H), 8.28 (s, 1H), 7.92 (s, 1H), 7.78 (d, J = 22.6 Hz, 2H), 7.08 (s, 1H), 6.90 (s, 1H), 4.36 (d, J = 6.7 Hz, 2H), 3.88 (s, 4H), 3.71 (s, 4H), 1.28 (s, 3H). |
| 59 | 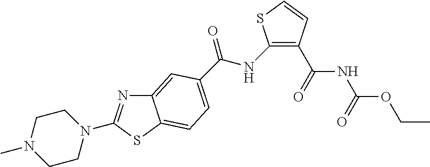 [SMILES: CCOC(=O)NC(=O)c1ccsc1NC(=O)c1ccc2sc(N3CCN(C)CC3)nc2c1] | A | | B | A | | ES-MS M/Z = 474 (M + 1) ¹H NMR (301 MHz, CDCl₃) 12.54 (s, 1H), 8.21 (s, 1H), 7.89 (s, 1H), 7.73 (dd, J = 22.2, 8.1 Hz, 2H), 7.03 (d, J = 6.0 Hz, 1H), 6.85 (d, J = 5.9 Hz, 1H), 4.32 (q, J = 7.1 Hz, 2H), 3.83-3.69 (m, 4H), 2.75-2.62 (m, 4H), 2.42 (s, 3H), 1.36 (t, J = 7.1 Hz, 3H). |
| 60 | 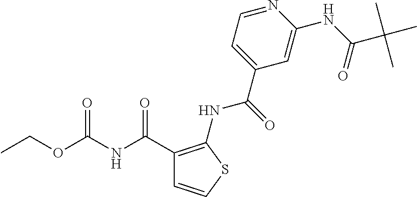 [SMILES: CCOC(=O)NC(=O)c1ccsc1NC(=O)c1ccnc(NC(=O)C(C)(C)C)c1] | A | A | C | A | A | ES-MS M/Z = 419 (M + 1) ¹H NMR (301 MHz, DMSO-d₆) 12.57 (s, 1H), 10.83 (s, 1H), 10.16 (s, 1H), 8.60 (d, J = 4.9 Hz, 2H), 7.73 (d, J = 6.0 Hz, 1H), 7.53 (s, 1H), 7.13 (d, J = 5.9 Hz, 1H), 4.21 (d, J = 7.1 Hz, 2H), 1.33- 1.20 (m, 12H). |
| 61 | 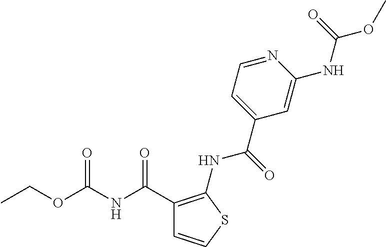 [SMILES: CCOC(=O)NC(=O)c1ccsc1NC(=O)c1ccnc(NC(=O)OC)c1] | B | A | B | A | B | ES-MS M/Z = 393 (M + 1) ¹H NMR (301 MHz, DMSO-d₆) 12.31 (s, 1H), 10.82 (s, 1H), 8.75 (d, J = 4.8 Hz, 2H), 7.97 (d, J = 5.3 Hz, 2H), 7.72 (d, J = 6.0 Hz, 1H), 7.12 (d, J = 5.7 Hz, 1H), 4.21 (dd, J = 14.1, 7.0 Hz, 2H), 2.86 (s, 3H), 1.29 (t, J = 7.1 Hz, 3H). |
| 62 | 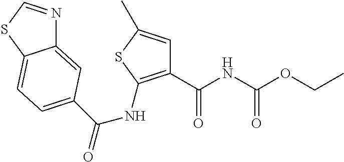 [SMILES: CCOC(=O)NC(=O)c1cc(C)sc1NC(=O)c1ccc2scnc2c1] | B | | C | B | | ES-MS M/Z = 390 (M + 1) ¹H NMR (301 MHz, CDCl₃) 12.60 (s, 1H), 9.10 (s, 1H), 8.84 (s, 1H), 8.09 (s, 1H), 7.78 (s, 1H), 6.68 (s, 1H), 4.32 (q, J = 7.1 Hz, 2H), 2.45 (s, 3H), 1.37 (t, J = 7.1 Hz, 3H). |
| 63 | 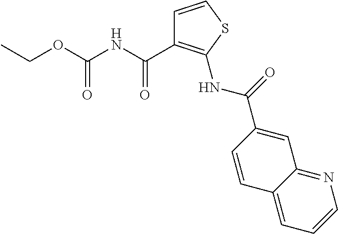 [SMILES: CCOC(=O)NC(=O)c1ccsc1NC(=O)c1ccc2cccnc2c1] | A | | B | A | | ES-MS M/Z = 370 (M + 1) ¹H NMR (301 MHz, DMSO-d₆) 12.85 (s, 1H), 10.85 (s, 1H), 9.06 (dd, J = 4.1, 1.7 Hz, 1H), 8.59 (s, 1H), 8.52 (d, J = 8.7 Hz, 1H), 8.24 (d, J = 8.5 Hz, 1H), 8.12 (d, J = 1.7 Hz, 1H),7.75 (d, 7 = 6.0 Hz, 1H), 7.70 (dd, J = 8.3, 4.2 Hz, 1H), 7.13 (d, J = 5.9 Hz, 1H), 4.23 (q, J = 7.1 Hz, 2H), 1.30 (t, J = 7.1 Hz, 3H). |
| 64 | 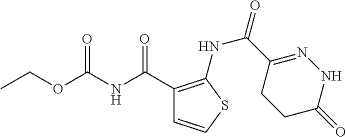 [SMILES: CCOC(=O)NC(=O)c1ccsc1NC(=O)C1=NNC(=O)CC1] | A | | A | A | | ES-MS M/Z = 339 (M + 1) ¹H NMR (301 MHz, DMSO-d₆) 12.59 (s, 1H), 11.48 (s, 1H), 10.70 (s, 1H), 7.68 (d, J = 6.0 Hz, 1H), 7.04 (d, J = 6.0 Hz, 1H), 4.20 (q, J = 6.9 Hz, 2H), 2.86 (t, J = 8.4 Hz, 2H), 2.49 (t, J = 8.4 Hz, 2H), 1.27 (t, J = 7.1 Hz, 3H). |
| 65 | 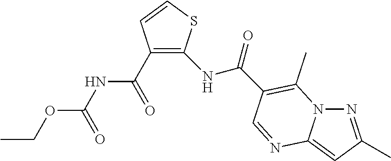 [SMILES: CCOC(=O)NC(=O)c1ccsc1NC(=O)c1cnc2cc(C)nn2c1C] | A | | A | A | | ES-MS M/Z = 388 (M + 1) ¹H NMR (301 MHz, CDCl₃) 12.37 (s, 1H), 8.76 (s, 1H), 7.86 (s, 1H), 7.07 (d, J = 6.0 Hz, 1H), 6.93 (d, J = 6.0 Hz, 1H), 6.60 (s, 1H), 4.33 (q, J = 7.2 Hz, 2H), 3.17 (s, 3H), 2.58 (s, 3H), 1.37 (t, J = 7.1 Hz, 3H). |
| 66 | 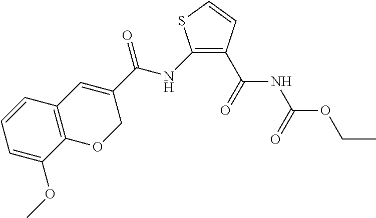 [SMILES: CCOC(=O)NC(=O)c1ccsc1NC(=O)C1=Cc2cccc(OC)c2OC1] | A | | A | A | | ES-MS M/Z = 403 (M + 1) ¹H NMR (301 MHz, CDCl₃) 12.26 (s, 1H), 7.92 (s, 1H), 7.36 (s, 1H), 7.26 (s, 1H), 7.02 (d, J = 6.0 Hz, 1H), 6.90 (s, 2H), 6.84 (d, J = 6.0 Hz, 1H), 5.18 (s, 1H), 4.33 (q, J = 7.1 Hz, 2H), 3.89 (s, 3H), 1.37 (t, J = 7.1 Hz, 3H). |
| 67 | 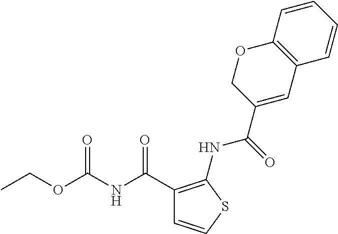 [SMILES: CCOC(=O)NC(=O)c1ccsc1NC(=O)C1=Cc2ccccc2OC1] | A | | A | A | | ES-MS M/Z = 373 (M + 1) ¹H NMR(301 MHz, DMSO-d₆) 12.19 (s, 1H), 10.77 (s, 1H), 7.69 (d, J = 5.7 Hz, 1H), 7.41 (t, J = 6.0 Hz, 2H), 7.31 (t, J = 6.0 Hz, 1H), 7.04 (m, 2H), 6.90 (d, J = 5.7 Hz, 1H), 5.07 (s, 1H), 4.21 (q, J = 6.9 Hz, 2H), 1.29 (t, J = 7.1 Hz, 3H). |
| 68 | 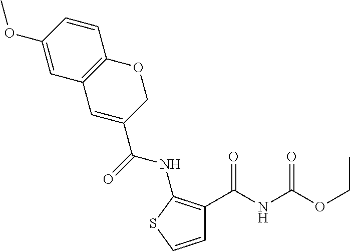 [SMILES: CCOC(=O)NC(=O)c1ccsc1NC(=O)C1=Cc2cc(OC)ccc2OC1] | A | | A | A | | ES-MS M/Z = 403 (M + 1) ¹H NMR (301 MHz, CDCl₃) 12.28 (s, 1H), 7.96 (s, 1H), 7.34 (d, J = 6.0 Hz, 1H), 7.04 (d, J = 6.0 Hz, 1H), 6.81 (m, 4H), 5.05 (s, 2H), 4.32 (q, J = 7.1 Hz, 2H), 3.79 (s, 3H), 1.38 (t, J = 7.1 Hz, 3H). |
| 69 | 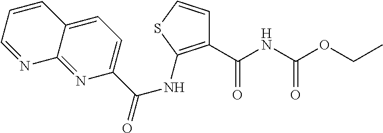 [SMILES: CCOC(=O)NC(=O)c1ccsc1NC(=O)c1ccc2cccnc2n1] | A | | A | C | | ES-MS M/Z = 371 (M + 1) ¹H NMR (301 MHz, DMSO-d₆) 10.77 (s, 1H), 9.26 (q, J = 1.8 Hz, 1H), 8.80 (d, J = 8.4 Hz, 1H), 8.65 d, J = 6.0 Hz, 1H), 8.41 (d, J = 8.4 Hz, 1H), 7.80 (m, 2H), 7.16 (d, J = 6.0 Hz, 1H), 4.26 (q, J = 6.9 Hz, 2H), 1.31 (t, J = 7.1 Hz, 3H). |
| 70 | 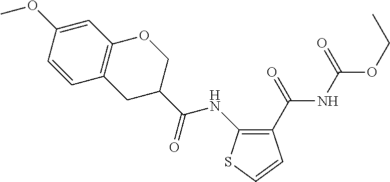 [SMILES: CCOC(=O)NC(=O)c1ccsc1NC(=O)C1COc2cc(OC)ccc2C1] | C | | C | A | | ES-MS M/Z = 405 (M + 1) ¹H NMR (301 MHz, CDCl₃) 11.91 (s, 1H), 7.84 (s, 1H), 7.00 (m, 1H), 6.85 (d, J = 6.0 Hz, 1H), 6.51 (d, J = 6.0 Hz, 1H), 6.45 (m, 1H), 4.52 (s, 1H), 4.35 (q, J = 7.1 Hz, 2H), 4.31 (m, 1H), 3.77 (s, 3H), 3.11 (m, 3H), 1.38 (t, J = 7.1 Hz, 3H). |
| 71 | 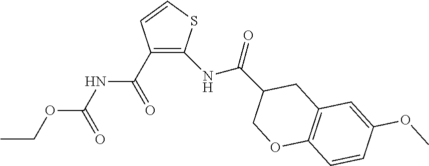 [SMILES: CCOC(=O)NC(=O)c1ccsc1NC(=O)C1COc2ccc(OC)cc2C1] | B | | A | A | | ES-MS M/Z = 405 (M + 1) ¹H NMR (301 MHz, DMSO-d₆) δ 11.81 (s, 1H), 10.70 (s, 1H), 7.60 (d, J = 6.0 Hz, 1H), 6.99 (d, J = 6.0 Hz, 1H), 6.70 (m, 3H), 4.38 (q, J = 3.0 Hz, 1H), 4.22 (q, J = 7.1 Hz, 2H), 4.16 (m, 1H), 3.68 (s, 3H), 3.05 (m, 2H), 1.37 (t, J = 7.1 Hz, 3H). |
| 72 | 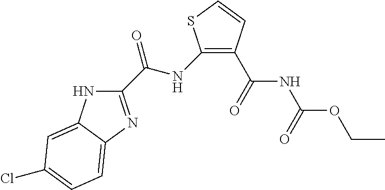 [SMILES: CCOC(=O)NC(=O)c1ccsc1NC(=O)c1nc2ccc(Cl)cc2[nH]1] | B | C | B | A | A | ES-MS M/Z = 393 (M + 1) ¹H NMR (301 MHz, DMSO-d₆) 13.03 (s, 1H), 10.80 (s, 1H), 7.92 (m, 1H), 7.74 (d, J = 6.0 Hz, 1H), 7.59 (d, J = 6.0 Hz, 1H), 7.39 (m, 1H), 7.14 (d, J = 6.0 Hz, 1H), 4.22 (q, J = 7.1 Hz, 2H), 1.30 (t, J = 7.1 Hz, 3H). |
| 73 | 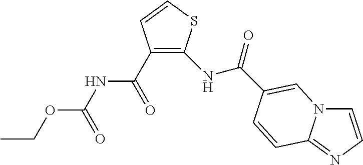 [SMILES: CCOC(=O)NC(=O)c1ccsc1NC(=O)c1ccc2nccn2c1] | B | | C | A | | ES-MS M/Z=359 (M + 1) ¹H NMR (301 MHz, CD₃OD) 9.54 (s, 1H), 8.41 (m, 2H), 8.14 (m, 2H), 7.54 (d, J = 6.3Hz, 1H), 7.05 (d, J = 6.3Hz, 1H), 4.30 (q, J = 7.1 Hz, 2H), 1.37 (t, J = 7.1 Hz, 3H). |
| 74 | 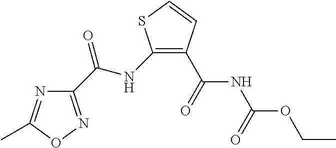 [SMILES: CCOC(=O)NC(=O)c1ccsc1NC(=O)c1noc(C)n1] | C | | C | B | | ES-MS M/Z = 325 (M + 1) ¹H NMR (301 MHz, DMSO-d₆) 12.82 (s, 1H), 10.84 (s, 1H), 7.75 (d, J = 5.9 Hz, 1H), 7.19 (d, J = 5.2 Hz, 1H), 4.21 (q, J = 7.1Hz, 2H), 2.75 (s, 3H), 1.28 (t, J = 7.1 Hz, 3H). |
| 75 | 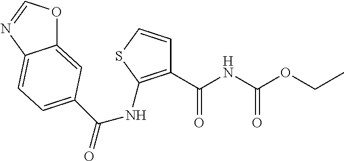 [SMILES: CCOC(=O)NC(=O)c1ccsc1NC(=O)c1ccc2ncoc2c1] | C | | C | A | | ES-MS M/Z = 360 (M + 1) ¹H NMR (301 MHz, CDCl₃) 12.68 (s, 1H), 8.24 (m, 4H), 7.07 (d, J = 6.0 Hz, 1H), 6.90 (d, J = 6.0 Hz, 1H), 4.33 (q, J = 7.1 Hz, 2H), 1.37 (t, J = 7.1 Hz, 3H). |
| 76 | 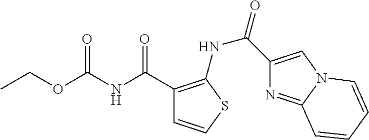 [SMILES: CCOC(=O)NC(=O)c1ccsc1NC(=O)c1cn2ccccc2n1] | B | C | A | A | A | ES-MS M/Z = 359 (M + 1) ¹H NMR (301 MHz, DMSO-d₆) 12.89 (s, 1H), 10.74 (s, 1H), 8.64 (m, 2H), 7.77 (m, 2H), 7.43 (m, 1H), 7.07 (m, 2H), 4.23 (d, J = 6.9 Hz, 2H), 1.29 (t, J = 7.1 Hz, 3H). |
| 77 | 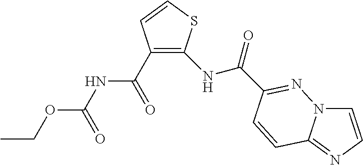 [SMILES: CCOC(=O)NC(=O)c1ccsc1NC(=O)c1ccc2nccn2n1] | B | | C | A | | ES-MS M/Z = 360 (M + 1) ¹H NMR (400 MHz, DMSO-d₆) 13.06 (s, 1H), 10.84 (s, 1H), 8.66-8.45 (m, 1H), 8.40 (dd, J = 9.5, 0.8 Hz, 1H), 8.04 (d, J = 1.3 Hz, 1H), 7.88 (d, J = 9.5 Hz, 1H), 7.76 (d, J = 5.9 Hz, 1H), 7.17 (dd, J = 5.9, 0.8 Hz, 1H), 4.23 (q, J = 7.1 Hz, 2H), 1.31 (t, J = 7.1 Hz, 3H). |
| 78 | 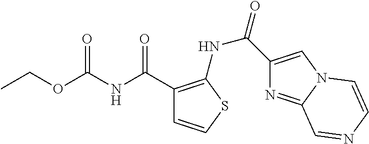 [SMILES: CCOC(=O)NC(=O)c1ccsc1NC(=O)c1cn2ccncc2n1] | C | | C | | | ES-MS M/Z = 361 (M + 1) ¹H NMR (301 MHz, DMSO-d₆) 12.96 (s, 1H), 10.78 (s, 1H), 9.34 (s, 1H), 8.79 (s, 1H), 8.72-8.62 (m, 1H), 8.03 (d, J = 4.6 Hz, 1H), 7.73 (d, J = 5.7 Hz, 1H), 7.10 (d, J = 5.4 Hz, 1H), 4.22 (q, J = 7.1 Hz, 2H), 1.30 (t, J = 7.1 Hz, 3H). |
| 79 | 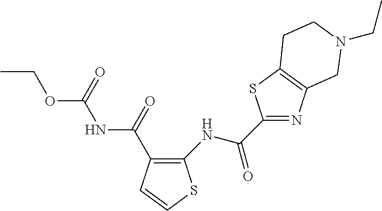 [SMILES: CCOC(=O)NC(=O)c1ccsc1NC(=O)c1nc2c(s1)CCN(CC)C2] | B | | C | | | ES-MS M/Z = 409 (M + 1) ¹H NMR (301 MHz, CDCl₃) 12.71 (s, 1H), 7.82 (s, 1H), 7.03 (d, J = 5.3 Hz, 1H), 6.90 (d, J = 5.9 Hz, 1H), 4.33 (d, J = 6.6 Hz, 2H), 3.77 (s, 2H), 3.02 (s, 2H), 2.84 (s, 2H), 2.69 (d, J = 6.9 Hz, 2H), 1.37 (t, J = 6.9 Hz, 3H), 1.22 (t, J = 7.0 Hz, 3H). |
| 80 | 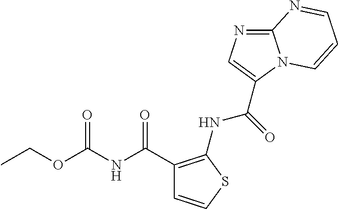 [SMILES: CCOC(=O)NC(=O)c1ccsc1NC(=O)c1cnc2ncccn12] | B | | C | | | ES-MS M/Z = 360 (M + 1) ¹H NMR (301 MHz, DMSO-d₆) 12.40 (s, 1H), 10.80 (s, 1H), 9.72 (s, 1H), 8.80 (s, 1H), 7.70 (s, 1H), 7.35 (d, J = 22.1 Hz, 2H), 7.19-6.91 (m, 2H), 4.21 (d, J = 6.9 Hz, 2H), 1.34-1.24 (m, 6H). |
| 81 | 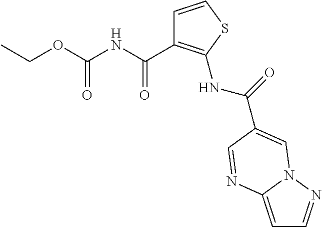 [SMILES: CCOC(=O)NC(=O)c1ccsc1NC(=O)c1cnc2ccnn2c1] | A | | C | | | ES-MS M/Z = 360 (M + 1) ¹H NMR (301 MHz, CD₃OD) 9.55 (s, 1H), 9.09 (s, 1H), 8.38 (m, 1H), 7.49 (m, 1H), 6.97(m, 1H), 6.82 (m, 1H), 4.36 (q, J = 7.1 Hz, 2H), 1.39 (t, J = 7.1 Hz, 3H). |
| 82 | 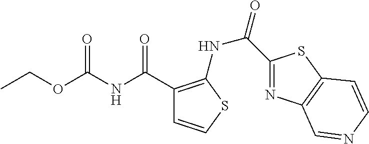 [SMILES: CCOC(=O)NC(=O)c1ccsc1NC(=O)c1nc2cnccc2s1] | A | | A | A | | ES-MS M/Z = 377 (M + 1) ¹H NMR (301 MHz, CDCl₃) 13.06 (s, 1H), 9.55 (s, 1H), 8.63 (d, J = 5.6 Hz, 1H), 7.92 (d, J = 5.5 Hz, 1H), 7.08 (d, J = 5.8 Hz, 1H), 6.97 (d, J = 5.9 Hz, 1H), 4.33 (q, J = 7.1 Hz, 2H), 1.36 (t, J = 7.1 Hz, 3H). |
| 83 | 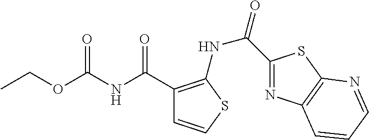 [SMILES: CCOC(=O)NC(=O)c1ccsc1NC(=O)c1nc2cccnc2s1] | A | | A | A | | ES-MS M/Z=377 (M + 1) ¹H NMR (301 MHz, CDCl₃) 13.02 (s, 1H), 8.70 (d, J = 4.0 Hz, 1H), 8.47 (d, J = 8.3 Hz, 1H), 7.96 (s, 1H), 7.53 (dd, J = 8.0, 4.6 Hz, 1H), 7.11 (d, J = 6.0 Hz, 1H), 6.95 (d, J = 5.9 Hz, 1H), 4.31 (dd, J = 14.1, 7.0 Hz, 2H), 1.34 (t, J = 7.0 Hz, 3H). |
| 84 | 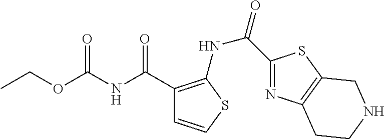 [SMILES: CCOC(=O)NC(=O)c1ccsc1NC(=O)c1nc2c(s1)CNCC2] | B | | C | A | | ES-MS M/Z = 381 (M + 1) ¹H NMR (301 MHz, DMSO-d₆) 12.84 (s, 1H), 10.83 (s, 1H), 9.26 (s, 1H), 7.74 (d, J = 6.0 Hz, 1H), 7.16 (d, J = 6.0 Hz, 1H), 4.56 (s, 2H), 4.20 (q, J = 7.1 Hz, 2H), 3.55 (s, 2H), 3.17 (s, 2H), 1.28 (t, J = 7.2 Hz, 3H) |
| 85 | 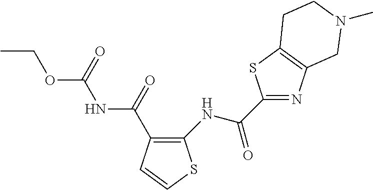 [SMILES: CCOC(=O)NC(=O)c1ccsc1NC(=O)c1nc2c(s1)CCN(C)C2] | B | | A | A | | ES-MS M/Z = 395 (M + 1) ¹H NMR (301 MHz, CD₃OD) 8.21 (s, 2H), 7.50 (d, J = 5.9 Hz, 1H), 7.03 (d, J = 5.9 Hz, 1H), 4.29 (q, J = 7.1 Hz, 2H), 4.06 (s, 2H), 3.19 (s, 4H), 2.78 (s, 3H), 1.36 (t, J = 7.1 Hz, 3H). |
| 86 | 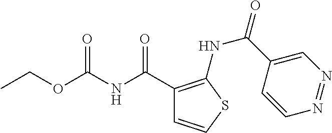 [SMILES: CCOC(=O)NC(=O)c1ccsc1NC(=O)c1ccnnc1] | B | | B | A | | ES-MS M/Z = 321 (M + 1) ¹H NMR (301 MHz, DMSO-d₆) 12.61 (s, 1H), 10.86 (s, 1H), 9.60 (s, 1H), 9.55 (d, J = 4.7 Hz, 1H), 8.07 (dd, J = 5.4, 2.3 Hz, 1H), 7.71 (d, J = 6.0 Hz, 1H), 7.17 (d, J = 5.8 Hz, 1H), 4.21 (q, J = 7.1 Hz, 2H), 1.28 (t, J = 7.0 Hz, 3H). |
| 87 | 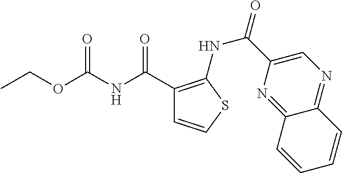 [SMILES: CCOC(=O)NC(=O)c1ccsc1NC(=O)c1cnc2ccccc2n1] | A | | A | A | | ES-MS M/Z = 371 (M + 1) ¹H NMR (301 MHz, DMSO-d₆) 13.36 (s, 1H), 10.81 (s, 1H), 9.60 (s, 1H), 8.24 (d, J = 7.3 Hz, 2H), 8.13-7.97 (m, 2H), 7.76 (d, J = 5.9 Hz, 1H), 7.17 (d, J = 5.8 Hz, 1H), 4.23 (dd, J = 14.1, 7.1 Hz, 2H), 1.31 (t, J = 7.1 Hz, 3H). |
| 88 | 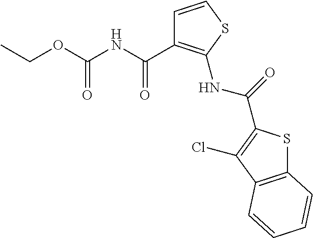 [SMILES: CCOC(=O)NC(=O)c1ccsc1NC(=O)c1sc2ccccc2c1Cl] | A | | B | A | | ES-MS M/Z = 409 (M + 1) ¹H NMR (301 MHz, DMSO-d₆) 12.89 (s, 1H), 10.82 (s, 1H), 8.18 (d, J = 6.9 Hz, 1H), 8.01 (d, J = 9.1 Hz, 1H), 7.74 (d, J = 6.0 Hz, 1H), 7.65 (t, J = 2.9 Hz, 2H), 7.16 (d, J = 6.0 Hz, 1H), 4.21 (dd, J = 14.1, 7.1 Hz, 2H), 1.29 (t, J = 7.1 Hz, 3H). |
| 89 | 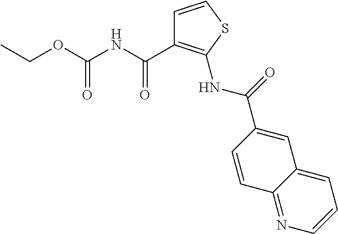 [SMILES: CCOC(=O)NC(=O)c1ccsc1NC(=O)c1ccc2ncccc2c1] | B | | B | A | | ES-MS M/Z = 370 (M + 1) ¹H NMR (301 MHz, DMSO-d₆) 12.73 (s, 1H), 10.84 (s, 1H), 9.05 (d, J = 2.6 Hz, 1H), 8.65 (s, 1H), 8.22 (d, J = 3.5 Hz, 2H), 7.74 (d, J = 6.0 Hz, 1H), 7.67 (dd, J = 8.3, 4.2 Hz, 1H), 7.11 (d, J = 5.8 Hz, 1H), 4.22 (q, J = 7.1 Hz, 2H), 1.29 (t, J = 7.1 Hz, 3H). |
| 90 | 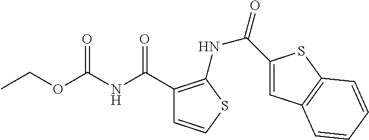 [SMILES: CCOC(=O)NC(=O)c1ccsc1NC(=O)c1cc2ccccc2s1] | A | | A | B | | ES-MS M/Z = 375 (M + 1) ¹H NMR (301 MHz, DMSO-d₆) 12.62 (s, 1H), 10.83 (s, 1H), 8.19 (s, 1H), 8.13 (t, J = 7.7 Hz, 2H), 7.73 (d, J = 6.0 Hz, 1H), 7.52 (dd, J = 9.3, 7.3 Hz, 2H), 7.11 (d, J = 6.0 Hz, 1H), 4.22 (q, J = 7.1 Hz, 2H), 1.29 (t, J = 7.1 Hz, 3H). |
| 91 | 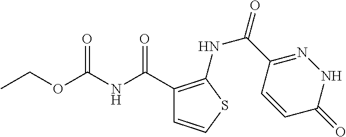 [SMILES: CCOC(=O)NC(=O)c1ccsc1NC(=O)c1ccc(=O)[nH]n1] | B | | A | A | | ES-MS M/Z = 337 (M + 1) ¹H NMR (301 MHz, DMSO-d₆) 13.77 (s, 1H), 12.82 (s, 1H), 10.76 (s, 1H), 7.94 (d, J = 9.9 Hz, 1H), 7.71 (d, J = 5.7 Hz, 1H), 7.09 (d, J = 6.0 Hz, 1H), 7.00 (d, J = 9.9 Hz, 1H), 4.20 (q, J = 6.9 Hz, 2H), 1.27 (t, J = 7.1 Hz, 3H). |
| 92 | 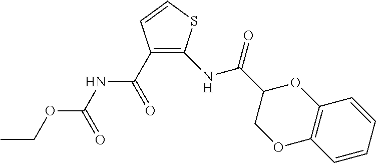 [SMILES: CCOC(=O)NC(=O)c1ccsc1NC(=O)C1COc2ccccc2O1] | C | | B | B | | ES-MS M/Z = 377 (M + 1) ¹H NMR (301 MHz, CD₃OD) 7.44 (d, J = 5.9 Hz, 1H), 7.27 (d, J = 6.4 Hz, 1H), 6.97 (d, J = 5.9 Hz, 1H), 6.93-6.83 (m, 3H), 4.46 (dd, J = 10.8, 4.3 Hz, 2H), 4.28 (q, J = 7.1 Hz, 2H), 1.35 (t, J = 7.1 Hz, 3H). |
| 93 | 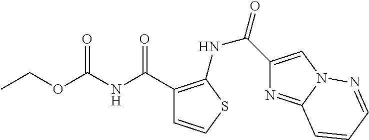 [SMILES: CCOC(=O)NC(=O)c1ccsc1NC(=O)c1cn2ncccc2n1] | A | | A | A | | ES-MS M/Z = 360 (M + 1) ¹H NMR (301 MHz, DMSO-d₆) 12.92 (s, 1H), 10.74 (s, 1H), 8.96 (s, 1H), 8.67 (d, J = 2.9 Hz, 1H), 8.39 (d, J = 9.4 Hz, 1H), 7.72 (d, J = 5.9 Hz, 1H), 7.41 (dd, J = 9.4, 4.4 Hz, 1H), 7.08 (d, J = 5.8 Hz, 1H), 4.22 (dd, J = 14.1, 7.0 Hz, 2H), 1.30 (t, J = 7.1 Hz, 3H). |
| 94 | 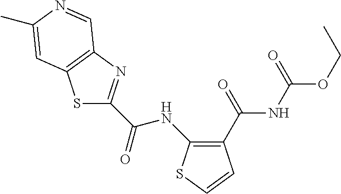 [SMILES: CCOC(=O)NC(=O)c1ccsc1NC(=O)c1nc2cnc(C)cc2s1] | A | | C | A | | ES-MS M/Z = 391 (M + 1) ¹H NMR (301 MHz, DMSO-d₆) 13.07 (s, 1H), 10.87 (s, 1H), 9.45 (s, 1H), 8.17 (s, 1H), 7.77 (d, J = 6.0 Hz, 1H), 7.20 (d, J = 5.9 Hz, 1H), 4.23 (q, J = 7.1 Hz, 2H), 1.38 (t, J = 7.1 Hz, 3H). |
| 95 | 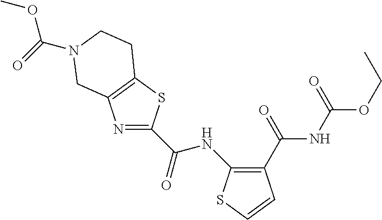 [SMILES: CCOC(=O)NC(=O)c1ccsc1NC(=O)c1nc2c(s1)CCN(C(=O)OC)C2] | A | | C | | | ES-MS M/Z = 439 (M + 1) ¹H NMR (301 MHz, CDCl₃) 12.75 (s, 1H), 7.81 (s, 1H), 7.04 (d, J = 5.9 Hz, 1H), 6.91 (d, J = 5.9 Hz, 1H), 4.77 (s, 2H), 4.34 (q, J = 6.9 Hz, 2H), 3.83 (t, J = 6.6 Hz, 2H), 3.77 (s, 3H), 2.99 (d, J = 6.9 Hz, 2H), 1.38 (t, J = 6.9 Hz, 3H). |
| 96 | 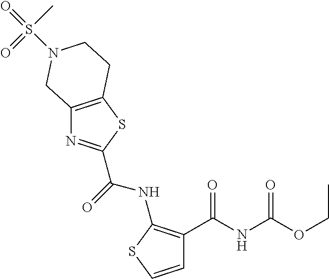 [SMILES: CCOC(=O)NC(=O)c1ccsc1NC(=O)c1nc2c(s1)CCN(S(C)(=O)=O)C2] | A | | C | | | ES-MS M/Z = 459 (M + 1) ¹H NMR (301 MHz, DMSO-d₆) 12.85 (s, 1H), 10.80 (s, 1H), 7.74 (d, J = 5.9 Hz, 1H), 7.15 (d, J = 5.9 Hz, 1H), 4.55 (s, 2H), 4.22 (q, J = 6.9 Hz, 2H), 3.58 (s, 2H), 3.31 (s, 2H), 3.05 (s, 3H), 1.28 (t, J = 6.9 Hz, 3H). |
| 97 | 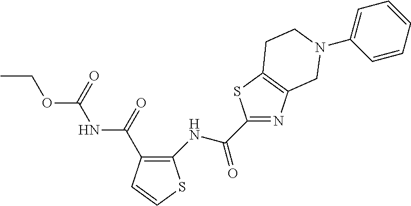 [SMILES: CCOC(=O)NC(=O)c1ccsc1NC(=O)c1nc2c(s1)CCN(c1ccccc1)C2] | A | | C | A | | ES-MS M/Z = 457 (M + 1) ¹H NMR (301 MHz, CD₃OD) 7.52 (s, 1H), 7.49 (m, 2H), 7.07 (m, 3H), 6.87 (m, 1H), 4.47 (s, 2H), 4.29 (q, J = 6.9 Hz, 2H), 3.69 (s, 2H), 3.11 (s, 2H), 1.37 (t, J = 6.9 Hz, 3H). |
| 98 | 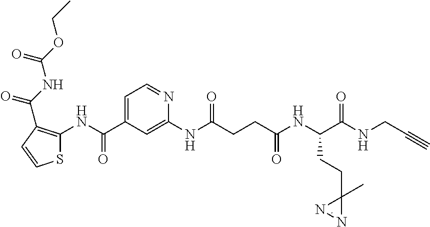 [SMILES: C#CCNC(=O)[C@H](CCC1(C)N=N1)NC(=O)CCC(=O)Nc1cc(C(=O)Nc2sccc2C(=O)NC(=O)OCC)ccn1] | | B | | | | |
| 99 | 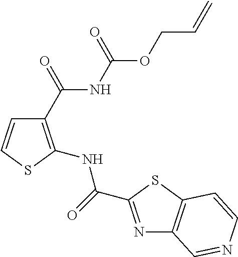 [SMILES: C=CCOC(=O)NC(=O)c1ccsc1NC(=O)c1nc2cnccc2s1] | A | | A | B | | ES-MS M/Z = 389.0 (M + 1) ¹H NMR: 13.1 (s, 1H), 10.98 (s, 1H), 9.7 (s, 1H), 8.7 (d, J = 5.6 Hz), 8.47 (d, 6.8 Hz, 1H), 7.8 (d, J = 6.0 Hz, 1H), 7.2 (d, 6.0 Hz, 1H), 6.0 (m, 1H), 5.4 (m, 1H), 5.30 (m, 1H), 4.7 (m, 2H) |
| 100 | 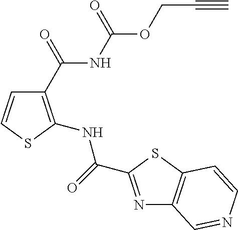 [SMILES: C#CCOC(=O)NC(=O)c1ccsc1NC(=O)c1nc2cnccc2s1] | A | | C | B | | ES-MSM/Z = 386.9 (M + 1) ¹H NMR: 13.08 (s, 1H), 11.10 (s, 1H), 9.62 (s, 1H), 8.71 (d, J = 5.6 Hz, 1H), 8.39 (dd, J = 0.8 Hz, 1H), 7.80 (d, J = 6 Hz, 1H), 7.25 (d, J = 6 Hz, 1H), 4.88 (d, J = 2.4 Hz, 2H), 3.68 (t, J = 2.4 Hz, 1H) |
| 101 | 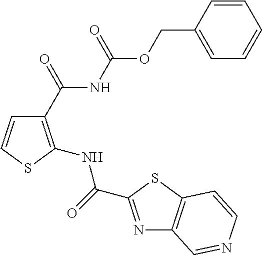 [SMILES: O=C(NC(=O)c1ccsc1NC(=O)c1nc2cnccc2s1)OCc1ccccc1] | A | | B | A | | ES-MS M/Z = 439.1 (M + 1) ¹H NMR: 13.11 (s, 1H), 11.0 (s, 1H), 9.62 (s, 1H), 8.7 (d, J = 8 Hz, 1H), 8.40 (d, J = 4 Hz, 1H), 7.79 (d, 8 Hz, 1H), 7.45 (m, 5H), 7.2 (d, J = 4 Hz, 1H), 5.27 (s, 2H) |
| 102 | 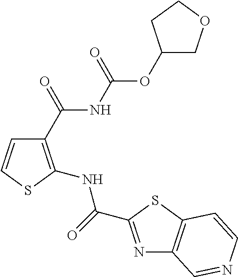 [SMILES: O=C(NC(=O)c1ccsc1NC(=O)c1nc2cnccc2s1)OC1CCOC1] | A | | A | A | | ES-MS M/Z = 418.9 (M + 1) ¹H NMR: 13.3 (s, 1H), 10.19 (s, 1H), 9.99 (m, 1H), 9.11 (m, 2H), 7.76 (d, J = 4Hz, 1H), 7.27 (d, J = 8 Hz, 1H), 5.43 (m, 1H), 3.89 (m, 4H), 1.5 (m, 2H) |
| 103 | 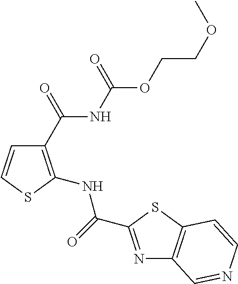 [SMILES: COCCOC(=O)NC(=O)c1ccsc1NC(=O)c1nc2cnccc2s1] | A | | C | A | | ES-MS M/Z = 406.9 (M + 1) ¹H NMR (400 MHz, DMSO-d₆) 13.13 (s, 1H), 11.02 (s, 1H), 9.74 (d, J = 0.9 Hz, 1H), 8.74 (d, J = 5.7 Hz, 1H), 8.51 (dd, J = 5.6, 0.9 Hz, 1H), 7.82 (d, J = 5.9 Hz, 1H), 7.24 (d, J = 5.9 Hz, 1H), 4.37- 4.23 (m, 2H), 3.71-3.50 (m, 2H), 3.30 (s, 3H). |
| 104 | 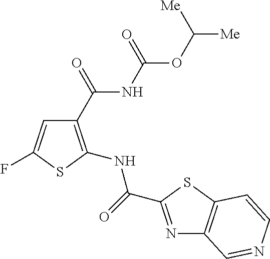 [SMILES: CC(C)OC(=O)NC(=O)c1cc(F)sc1NC(=O)c1nc2cnccc2s1] | A | | A | C | | ES-MS M/Z = 409 (M + 1) ¹H NMR (400 MHz, CD₃OD) 9.51 (s, 1H), 8.66 (s, 1H), 8.32 (d, J = 5.7 Hz, 1H), 7.19 (d, J = 3.5 Hz, 1H), 5.10 (m, 1H), 1.36 (dd, J = 18.2, 6.3 Hz, 6H). |
| 105 | 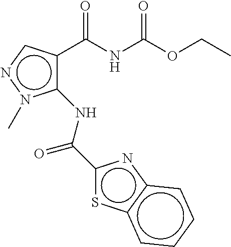 | C | | | | | ES-MS M/Z = 374.1 (M + 1) ¹H NMR (400 MHz, DMSO-d₆) 8.216- 8.142 (m, 2H), 7.660-7.590 (m, 3H), 4.091-4.078 (m, 2H), 3.601 (s, 3H), 1.064-1.030 (t, J = 6.8 Hz, 3H). |
| 106 | 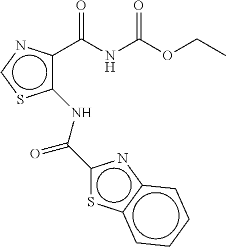 | C | | | | | ¹HNMR(400 MHz, DMSO-d₆) 10.251 (s, 1H), 8.846 (s, 1H), 8.369-8.335 (m, 2 H), 7.756-7.672 (m, 2H), 4.238 (q, J = 6.8 Hz, 2H), 1.292 (t, J = 7.2 Hz, 3H). |
| 107 | 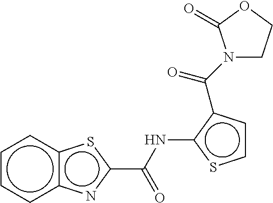 | C | | | | | ES-MS M/Z = 374.0 (M + 1) ¹H NMR (400 MHz, DMSO-d₆) 8.313- 8.252 (m, 2H), 7.705-7.627 (m, 2H), 7.290 (d, J = 5.8 Hz, 1H), 7.130 (d, J = 5.8 Hz, 1H), 4.456 (t, J = 7.7 Hz, 2H), 4.114 (t, J = 7.8 Hz, 2H). |
| 108 | 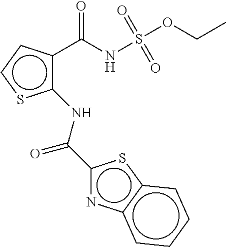 | C | | | | | ¹H NMR (400 MHz, CD₃OD) 8.264- 8.242 (m, 1H), 8.128-8.105 (m, 1H), 7.635-7.615 (m, 2H), 7.412 (d, J = 5.7 Hz, 1H), 6.907 (d, J = 5.7 Hz, 1H), 4.290 (q, J = 7.1 Hz, 2H), 1.354 (t, J = 7.1 Hz, 2H). |
| 109 | 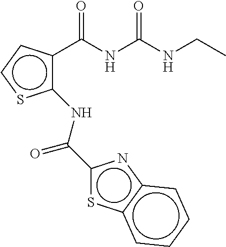 | C | | | | | ES-MS M/Z = 375.1 (M + 1) ¹H NMR (400 MHz, DMSO-d₆) 10.472 (s, 1H), 8.538 (m, 1H), 8.314-8.263 (m, 2H), 7.858 (d, J = 5.9 Hz, 1H), 7.698 (m, 2H), 7.207 (d, J = 6.0 Hz, 1H), 3.282 (2H buried inside the H₂O peak), 1.162 (t, J = 7.2 Hz, 3H). |
| 110 | 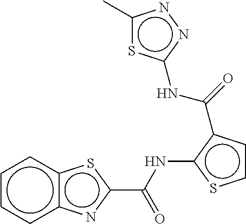 | C | | | | | ES-MS M/Z = 402 (M + 1) ¹H NMR (500 MHz, DMSO-d₆) 8.398- 8.352 (m, 2H), 8.1 (br, 1H), 7.788- 7.734 (m, 2H), 7.350 (br, 1H), 2.755 (s, 3H). |
| 111 | 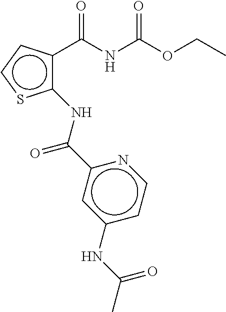 | C | | | | | ES-MS M/Z = 377.1 (M + 1) ¹H NMR (400 MHz, DMSO-d₆) 10.724 (s, 1H), 10.663 (s, 1H), 8.623 (d, J = 5.5 Hz, 1H), 8.392 (d, J = 2.0 Hz, 1H), 7.819 (d, J = 2.2 Hz, 1H), 7.700 (d, J = 5.9 Hz, 1H), 7.091, (d, J = 5.9 Hz, 1H), 4.195 (q, J = 7.1 Hz, 2H), 2.119 (s, 3H), 1.267 (t, J = 7.1 Hz, 3H). |
| 112 | 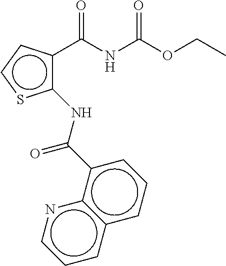 | C | | C | A | | ES-MS M/Z = 370 (M + 1) ¹H NMR (301 MHz, DMSO-d₆) 15.79 (s, 1H), 10.64 (s, 1H), 9.15 (d, J = 2.5 Hz, 1H), 8.70 (dd, J = 28.3, 7.9 Hz, 2H), 8.34 (d, J = 8.0 Hz, 1H), 7.95-7.74 (m, 2H), 7.63 (d, J = 5.9 Hz, 1H), 7.07 (d, J = 6.0 Hz, 1H), 4.21 (q, J = 7.0 Hz, 2H), 1.30 (t, J = 7.1 Hz, 3H). |
| 113 | 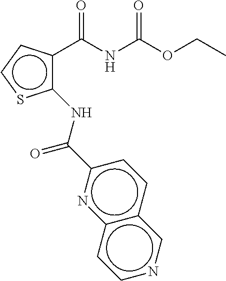 | C | | C | A | | ES-MS M/Z = 371 (M + 1) ¹H NMR (301 MHz, DMSO-d₆ 13.49 (s, 1H), 10.80 (s, 1H), 9.59 (s, 1H), 8.99- 8.83 (m, 2H), 8.44 (d, J = 8.5 Hz, 1H), 8.03 (d, J = 6.0 Hz, 1H), 7.75 (d, J = 6.0 Hz, 1H), 7.17 (d, J = 5.9 Hz, 1H), 4.23 (d, J = 7.1 Hz, 2H), 1.29 (dd, J = 17.8, 10.7 Hz, 3H). |
| 114 | 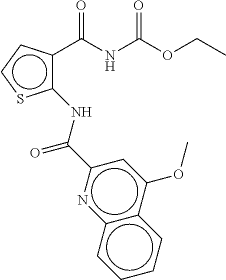 | C | | C | A | | ES-MS M/Z = 400 (M + 1) ¹H NMR (301 MHz, CD₃OD) 13.49 (s, 1H), 10.77 (s, 1H), 8.25 (d, J = 8.2 Hz, 1H), 8.11 (d, J = 8.1 Hz, 1H), 7.91 (d, J = 7.0 Hz, 1H), 7.74 (dd, J = 6.8, 3.5 Hz, 2H), 7.14 (d, J = 5.9 Hz, 1H), 4.24 (dd, J = 15.3, 8.2 Hz, 5H), 1.32 (t, J = 7.1 Hz, 3H). |
| 115 | 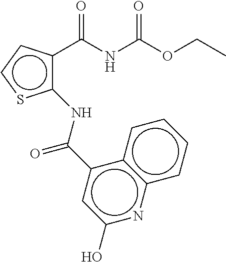 | C | | C | A | | ES-MS M/Z = 386 (M + 1) ¹H NMR (301 MHz, DMSO-d₆) 12.13 (d, J = 11.5 Hz, 1H), 10.80 (s, 1H), 7.82 (d, J = 7.4 Hz, 1H), 7.68 (d, J = 5.9 Hz, 1H), 7.62-7.50 (m, 1H), 7.39 (d, J = 8.0 Hz, 1H), 7.27-7.12 (m, 1H), 7.01 (d, J = 8.6 Hz, 1H), 6.90 (d, J = 1.7 Hz, 1H), 4.18 (q, J = 7.1 Hz, 2H), 1.25 (dd, J = 11.8, 4.7 Hz, 3H). |
| 116 | 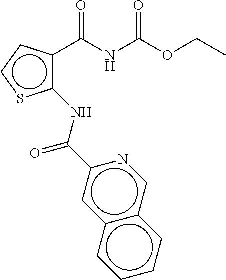 | C | | C | A | | ES-MS M/Z = 370 (M + 1) ¹H NMR (301 MHz, DMSO-d₆) 13.48 (s, 1H), 10.74 (s, 1H), 9.52-7.10 (m, 8H), 4.23 (dd, J = 14.1, 7.1 Hz, 2H), 1.29 (t, J = 7.1 Hz, 3H). |
| 117 | 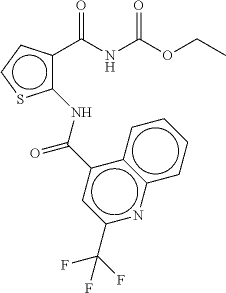 | C | | C | C | | ES-MS M/Z = 438 (M + 1) ¹H NMR (400 MHz, DMSO-d₆) 12.32 (s, 1H), 10.86 (s, 1H), 8.36 (m, 3H), 8.01 (m, 2H), 7.71 (d, J = 6.0 Hz, 1H), 7.25 (d, J = 6.0 Hz, 1H), 4.20 (q, J = 7.1 Hz, 2H), 1.28 (t, J = 7.1 Hz, 3H). |
| 118 | 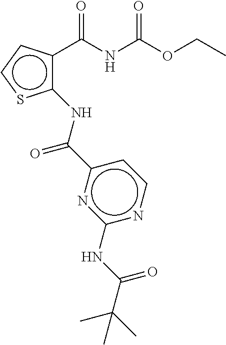 | C | | B | A | | ¹H NMR (500 MHz, CD₃OD) 9.025 (d, J = 4.8 Hz, 1H), 7.973 (d, J = 4.9 Hz, 1H), 7.617 (d, J = 5.9 Hz, 1H), 7.153 (d, J = 6.0 Hz, 1H), 4.391 (q, J = 7.1 Hz, 1H), 1.451 (t, J = 7.2 Hz, 1H). |
| 119 | 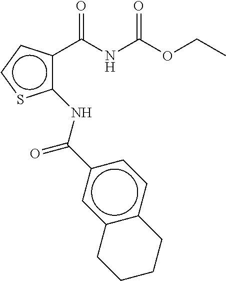 | C | | C | A | | ES-MSM/Z = 373 (M + 1) ¹H NMR (301 MHz, CDCl₃) 12.43 (s, 1H), 7.96 (s, 1H), 7.76 (m, 2H), 7.07 (m, 1H), 7.06 (m, 1H), 6.85 (m, 1H), 4.33 (q, J = 7.1 Hz, 2H), 2.84 (m, 3H), 1.83 (m, 5H), 1.37 (t, J = 7.1 Hz, 3H). |
| 120 | 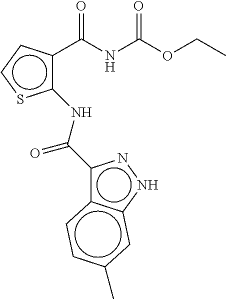 | C | | C | A | | ES-MS M/Z = 373 (M + 1) ¹H NMR (301 MHz, CD₃OD + CDCl₃) 8.13 (m, 1H), 7.61 (s, 1H), 7.42 (m, 2H), 7.13 (d, J = 5.9 Hz, 1H), 6.86 (d, J = 5.2 Hz, 1H), 4.28 (q, J = 7.1 Hz, 2H), 2.49 (s, 3H), 1.35 (t, J = 7.1 Hz, 3H). |
| 121 | 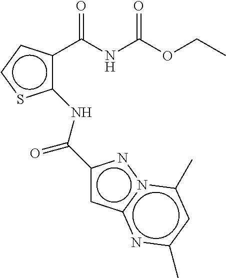 | C | | C | A | | ES-MS M/Z = 388 (M + 1) ¹H NMR (301 MHz, DMSO-d₆) 12.87 (s, 1H), 10.74 (s, 1H), 7.71 (d, J = 6.0 Hz, 1H), 7.13 (m, 3H), 4.21 (q, J = 7.2 Hz, 2H), 2.80 (s, 3H), 2.49 (s, 3H), 1.29 (t, J = 7.2 Hz, 3H). |
| 122 | 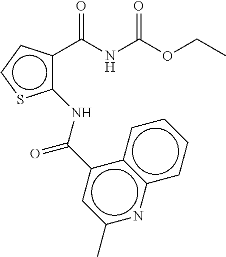 | C | | C | A | | ES-MS M/Z = 384 (M + 1) ¹H NMR (301 MHz, CDCl₃) 12.25 (s, 1H), 8.36 (m, 1H), 8.07 (m, 2H), 7.74 (m, 1H), 7.58 (m, 2H), 7.11 (m, 1H), 6.93 (m, 1H), 4.29 (q, J = 7.1 Hz, 2H), 2.81 (s, 3H), 1.31 (t, J = 7.1 Hz, 3H). |
| 123 | 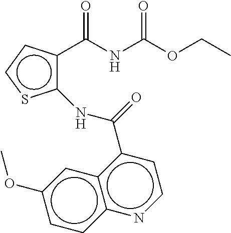 | C | | B | A | | ES-MS M/Z = 400 (M + 1) ¹H NMR (301 MHz, DMSO-d₆) 12.38 (s, 1H), 10.82 (s, 1H), 8.95 (d, J = 4.5 Hz, 1H), 8.09-7.71 (m, 5H), 7.17 (d, J = 5.7 Hz, 1H), 4.18 (dd, J = 14.1, 7.1 Hz, 2H), 3.90 (s, 3H), 1.26 (t, J = 7.1 Hz, 3H). |
| 124 | 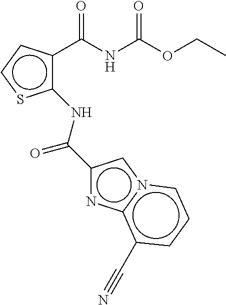 | C | | A | A | | ES-MS M/Z = 384 (M + 1) ¹H NMR (301 MHz, DMSO-d₆) 12.81 (s, 1H), 10.75 (s, 1H), 8.92 (d, J = 7.0 Hz, 1H), 8.83 (s, 1H), 8.17 (t, J = 9.0 Hz, 1H), 7.71 (d, J = 6.0 Hz, 1H), 7.23 (t, J = 7.0 Hz, 1H), 7.10 (d, J = 5.7 Hz, 1H), 4.22 (q, J = 7.1 Hz, 2H), 1.30 (t, J = 7.1 Hz, 3H). |
| 125 | 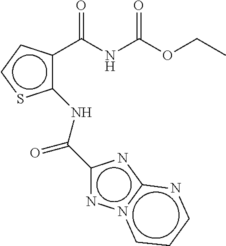 | C | | B | A | | ES-MS M/Z = 361 (M + 1) ¹H NMR (301 MHz, DMSO-d₆) 13.11 (s, 1H), 10.80 (s, 1H), 9.58 (m, 1H), 9.06 (m, 1H), 7.76 (m, 1H), 7.55 (m, 1H), 7.16 (m, 1H), 4.22 (q, J = 7.1 Hz, 2H), 1.30 (t, J = 7.1 Hz, 3H). |
| 126 | 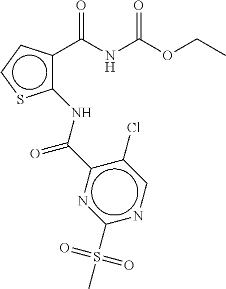 | C | | C | C | | ES-MS M/Z = 433 (M + 1) ¹H NMR (301 MHz, DMSO-d₆) 13.37 (s, 1H), 10.84 (s, 1H), 9.46 (s, 1H), 7.77 (d, J = 6.0 Hz, 1H), 7.21 (d, J = 5.7 Hz, 1H), 4.21 (q, J = 7.2 Hz, 2H), 3.65 (s, 3H), 1.29 (t, 7 = 7.2 Hz, 3H). |
| 127 | 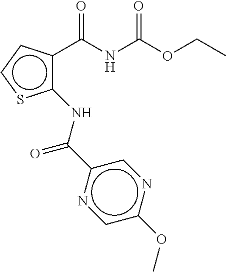 | C | | C | A | | ES-MS M/Z = 351 (M + 1) ¹H NMR (301 MHz, CDCl₃) 13.08 (s, 1H), 8.98 (s, 1H), 8.25 (d, J = 6.0 Hz, 1H), 7.91 (d, J = 6.0 Hz, 1H), 7.07 (d, J = 6.0 Hz, 1H), 6.90 (d, J = 6.0 Hz, 1H), 4.33 (q, J = 7.1 Hz, 2H), 4.07 (s, 3H), 1.37 (t, J = 7.1 Hz, 3H). |
| 128 | 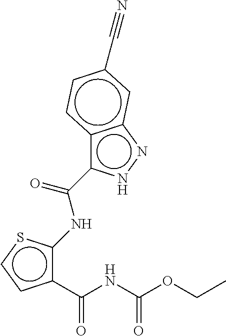 | C | | C | A | | ES-MS M/Z = 384 (M + 1) ¹H NMR (301 MHz, DMSO-d₆) 12.92 (s, 1H), 10.75 (s, 1H), 8.36 (m, 2H), 7.70 (m, 2H), 7.07 (m, 1H), 4.22 (q, J = 7.1 Hz, 2H), 1.29 (t, J = 7.1 Hz, 3H). |
| 129 | 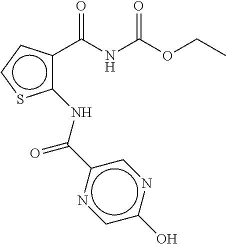 | C | | C | A | | ES-MS M/Z = 337 (M + 1) ¹H NMR (301 MHz, DMSO-d₆) δ 13.03 (s, 1H), 10.80 (s, 1H), 8.00-7.14 (m, 5H), 4.22 (q, J = 6.9 Hz, 2H), 1.29 (t, J = 7.1 Hz, 3H). |
| 130 | 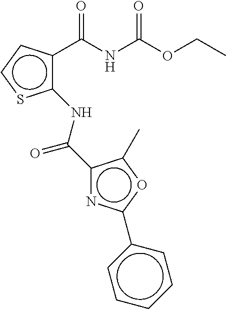 | C | | C | A | | ES-MS M/Z = 400 (M + 1) ¹H NMR (301 MHz, DMSO-d₆) 12.65 (s, 1H), 10.74 (s, 1H), 8.01 (m, 2H), 7.70 (d, J = 6.0 Hz, 1H), 7.61 (m, 3H), 7.07 (d, J = 6.0 Hz, 1H), 4.22 (q, J = 7.0 Hz, 2H), 2.75 (s, 3H), 1.31 (t, J = 7.1 Hz, 3H). |
| 131 | 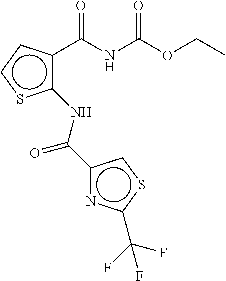 | C | | B | C | | ES-MS M/Z = 394 (M + 1) ¹H NMR (301 MHz, DMSO-d₆) 12.87 (s, 1H), 10.83 (s, 1H), 8.95 (s, 1H), 7.75 (d, J = 6.0 Hz, 1H), 7.19 (d, J = 6.0 Hz, 1H), 4.21 (q, J = 7.1 Hz, 2H), 1.29 (t, J = 7.1 Hz, 3H). |
| 132 | 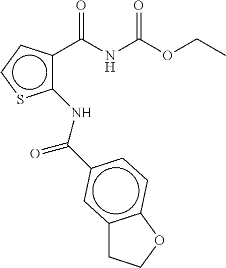 | C | | B | A | | ES-MS M/Z = 361 (M + 1) ¹H NMR (301 MHz, DMSO-d₆) 7.83 (m, 2H), 7.47 (m, 1H), 6.90 (m, 2H), 4.87 (m, 2H), 4.67 (m, 2H), 4.29 (q, J = 7.1 Hz, 2H), 1.36 (t, J = 7.1 Hz, 3H). |
| 133 | 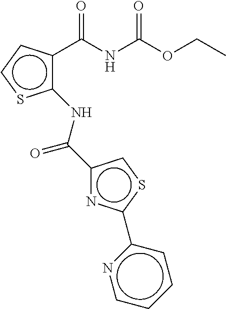 | C | | C | A | | ES-MS M/Z = 403 (M + 1) ¹H NMR (301 MHz, DMSO-d₆) 8.70 (m, 2H), 8.14 (m, 2H), 7.72 (d, J = 5.7 Hz, 1H), 7.60 (m, 1H), 7.10 (d, J = 5.7 Hz, 1H), 4.23 (q, J = 7.1 Hz, 2H), 1.31 (t, J = 7.1 Hz, 3H). |
| 134 | 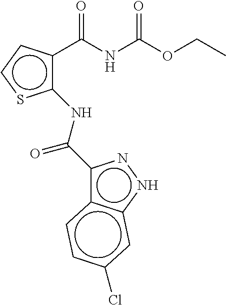 | C | | C | A | | ES-MS M/Z = 393 (M + 1) ¹H NMR (301 MHz, CD₃OD) 8.21 (m, 1H), 7.58 (m, 1H), 7.41 (d, J = 6.0 Hz, 1H), 7.23 (m, 1H), 6.85 (d, J = 6.0 Hz, 1H), 4.29 (q, J = 7.1 Hz, 2H), 1.35 (t, J = 7.1 Hz, 3H). |
| 135 | 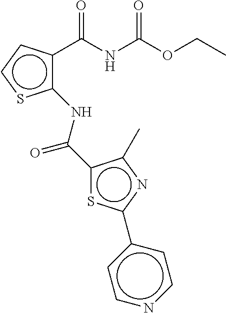 | C | | C | A | | ES-MS M/Z = 417 (M + 1) ¹H NMR (301 MHz, DMSO-d₆) 12.32 (s, 1H), 10.82 (s, 1H), 8.75 (m, 2H), 7.96 (m, 2H), 7.72 (m, 1H), 7.11 (m, 1H), 4.21 (q, J = 7.1 Hz, 2H), 2.86 (s, 3H), 1.28 (t, J = 7.1 Hz, 3H). |
| 136 | 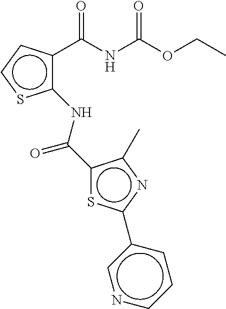 | C | | C | A | | ES-MS M/Z = 417 (M + 1) ¹H NMR (301 MHz, DMSO-d₆) 12.32 (s, 1H), 10.83 (s, 1H), 9.21 (m, 1H), 8.75 (m, 1H), 8.41 (m, 1H), 7.73 (m, 1H), 7.59 (m, 1H), 7.12 (m, 1H), 4.22 (q, J = 7.1 Hz, 2H), 2.86 (s, 3H), 1.28 (t, J = 7.1 Hz, 3H). |
| 137 | 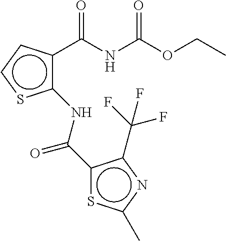 | C | | C | A | | ES-MS M/Z = 408 (M + 1) ¹H NMR (301 MHz, CDCl₃) 12.21 (s, 1H), 7.89 (s, 1H), 6.94 (m, 2H), 4.31 (q, J = 7.1 Hz, 2H), 2.80 (s, 3H), 1.35 (t, J = 7.1 Hz, 3H). |
| 138 | 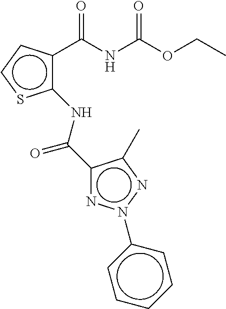 | C | | C | A | | ES-MS M/Z = 400 (M + 1) ¹H NMR (301 MHz, DMSO-d₆) 12.75 (s, 1H), 10.80 (s, 1H), 8.05 (d, J = 6.0 Hz, 2H), 7.74-7.63 (m, 4H), 7.12 (m, 1H), 4.21 (m, 2H), 2.51 (s, 3H), 1.29 (m, 3H). |
| 139 | 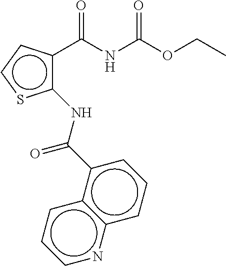 | C | | C | A | | ES-MS M/Z = 370 (M + 1) ¹H NMR (301 MHz, CDCl₃) 12.42 (s, 1H), 9.22-7.83 (m, 9H), 4.30 (dd, J = 14.1, 7.1 Hz, 2H), 1.26 (t, J = 7.1 Hz, 3H). |
| 140 | 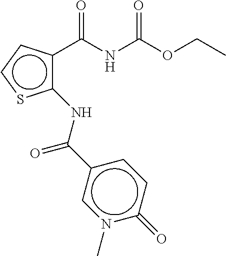 | C | | B | A | | ES-MS M/Z = 350 (M + 1) ¹H NMR (301 MHz, DMSO-d₆) 14.13 (s, 1H), 8.33 (m, 1H), 8.16 (m, 1H), 7.08 (m, 1H), 6.46 (d, J = 5.7 Hz, 1H), 6.32 (d, J = 5.7 Hz, 1H), 4.17 (dd, J = 14.1, 7.1 Hz, 2H), 3.51 (s, 3H), 1.29 (t, J = 7.1 Hz, 3H). |
| 141 | 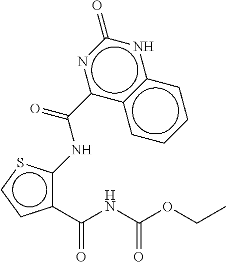 | C | | C | A | | ES-MS M/Z = 387 (M + 1) ¹H NMR (301 MHz, CD₃OD) 7.47 (d, J = 6.0 Hz, 1H), 7.35-7.19 (m, 2H), 7.04- 6.93 (m, 3H), 4.27 (q, J = 7.2 Hz, 2H), 1.34 (t, J = 7.2 Hz, 3H). |
| 142 | 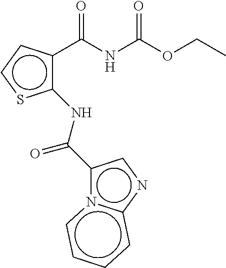 | C | | C | B | | ES-MS M/Z = 359 (M + 1) ¹H NMR (301 MHz, DMSO-d₆) 12.80 (s, 1H), 10.74 (s, 1H), 8.64 (m, 2H), 7.81- 7.70 (m, 2H), 7.43 (m, 1H), 7.06 (m, 2H), 4.23 (d, J = 6.9 Hz, 2H), 1.29 (m, 3H). |
| 143 | 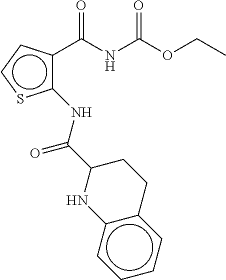 | C | | B | A | | ES-MS M/Z = 374 (M + 1) ¹H NMR (301 MHz, DMSO-d₆) 12.29 (s, 1H), 10.63 (s, 1H), 7.61 (d, J = 5.7 Hz, 1H), 7.01-6.52 (m, 6H), 4.15 (dd, J = 14.1, 7.1 Hz, 2H), 2.27 (m, 1H), 2.24- 1.35 (m, 4H), 1.29 (t, J = 7.1 Hz, 3H). |
| 144 | 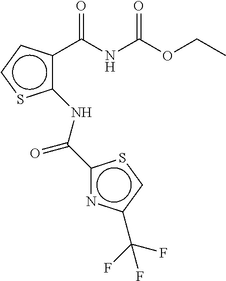 | C | | C | A | | ES-MS M/Z = 394 (M + 1) ¹H NMR (301 MHz, CDCl₃) 12.74 (s, 1H), 8.49 (s, 1H), 7.97 (m, 1H), 7.09 (m, 1H), 6.92 (d, J = 5.7 Hz, 1H), 4.32 (q, J = 7.1 Hz, 2H), 1.29 (t, J = 7.1 Hz, 3H). |
| 145 | 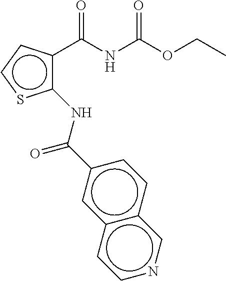 | C | | C | A | | ES-MS M/Z = 370 (M + 1) ¹H NMR (301 MHz, DMSO-d₆) 12.73 (s, 1H), 10.85 (s, 1H), 9.47 (s, 1H), 8.66- 8.08 (m, 5H), 7.74 (m, 1H), 7.14 (m, 1H), 4.22 (q, J = 7.1 Hz, 2H), 1.29 (t, J = 7.1 Hz, 3H). |
| 146 | 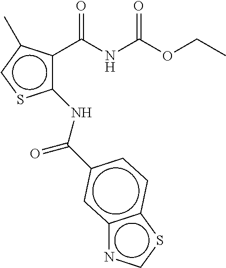 | C | | C | A | | ES-MS M/Z = 390 (M + 1) ¹H NMR (301 MHz, DMSO-d₆) 11.47 (s, 1H), 10.68 (s, 1H), 9.54 (s, 1H), 8.64 (d, J = 4.8 Hz, 1H), 8.36 (d, J = 4.5 Hz, 1H), 8.01 (t, J = 4.5 Hz, 1H), 6.77 (d, J = 5.7 Hz, 1H), 4.17 (dd, J = 14.1, 7.0 Hz, 2H), 2.24 (s, 3H), 1.26 (t, J = 7.1 Hz, 3H). |
| 147 | 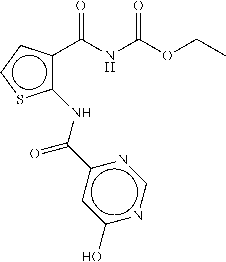 | C | | C | A | | ES-MS M/Z = 337 (M + 1) ¹H NMR (301 MHz, DMSO-d₆) 13.12 (s, 1H), 10.77 (s, 1H), 8.50 (m, 1H), 7.74 (m, 1H), 7.16 (m, 1H), 6.96 (m, 1H), 4.20 (dd, J = 14.1, 7.1 Hz, 2H), 1.28 (t, J = 7.1 Hz, 3H). |
| 148 | 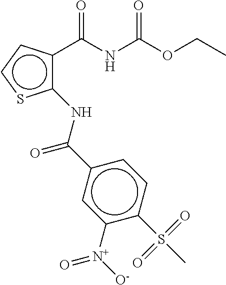 | | C | | | | |
| 149 | 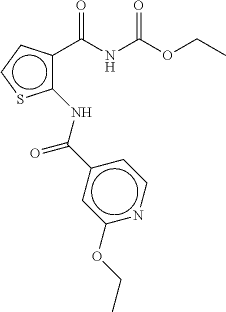 | | C | | | | |
| 150 | 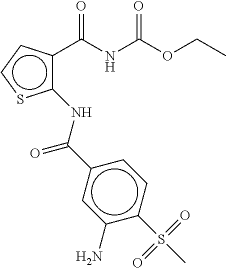 | | C | | | | |
| 151 | 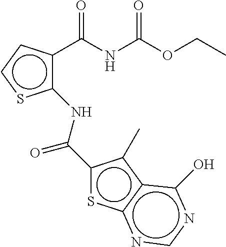 | | C | | | | |
| 152 | 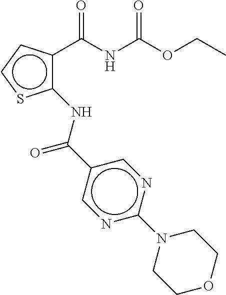 | | C | | | | |
| 153 | 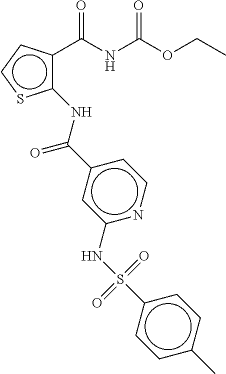 | | C | | | | |
| 154 | 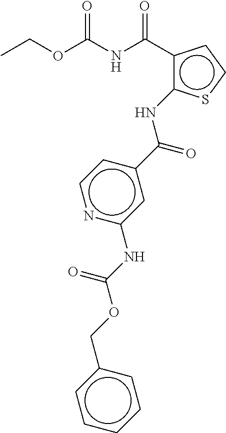 | | C | | | | |
| 155 | 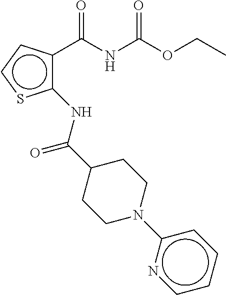 | | C | | | | |
| 156 | 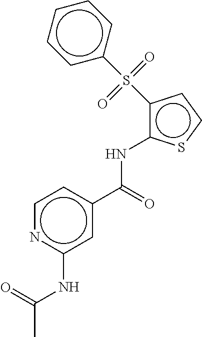 | | C | | | | |
| 157 | 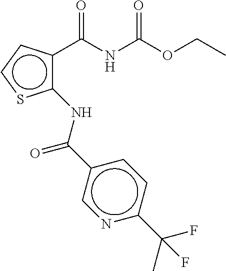 | | C | | | | |
| 158 | 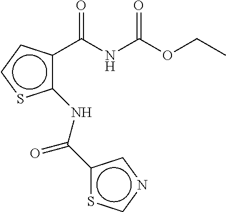 | | C | | | | |
| 159 | 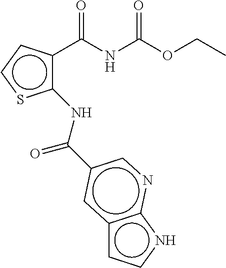 | | C | | | | |
| 160 | 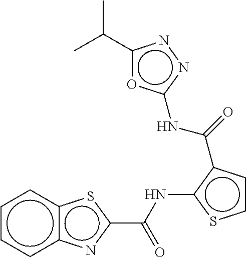 | | C | | | | |
| 161 | 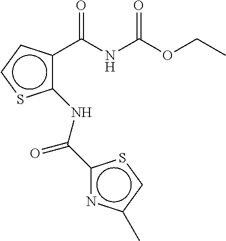 | | C | | | | |
| 162 | 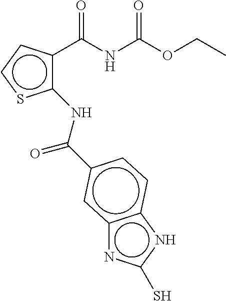 | | C | | | | |
| 163 | 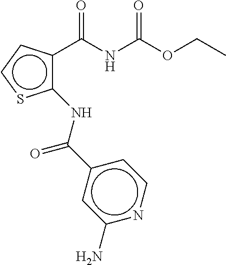 | | C | | | | |
| 164 | 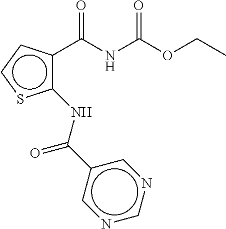 | | C | | | | |
| 165 | 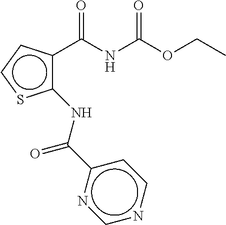 | | C | | | | |
| 166 | 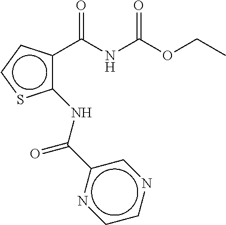 | | C | | | | |
| 167 | 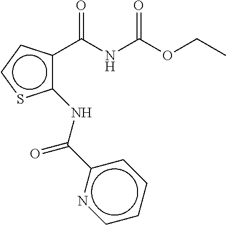 | | C | | | | |
| 168 | 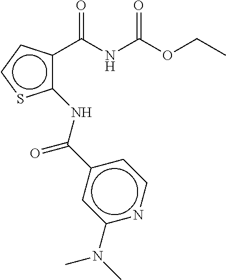 | | C | | | | |
| 169 | 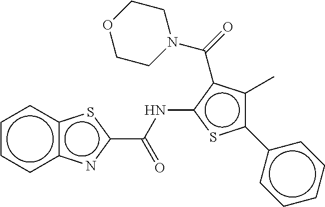 | | C | | | | |
| 170 | 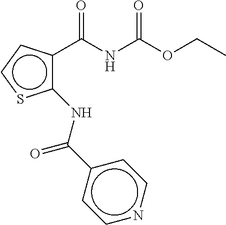 | | C | | | | |
| 171 | 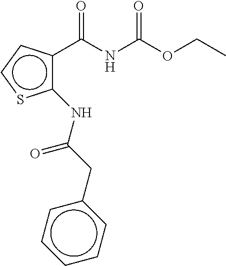 | | C | | | | |
| 172 | 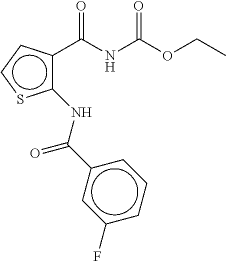 | | C | | | | |
| 173 | 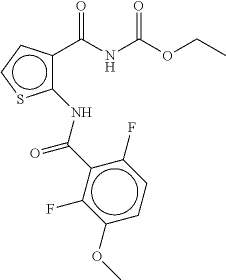 | | C | | | | |
| 174 | 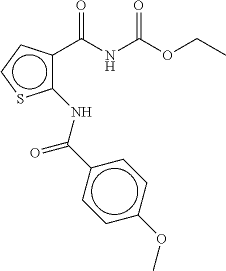 | | C | | | | |
| 175 | 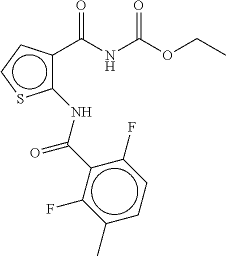 | | C | | | | |
| 176 | 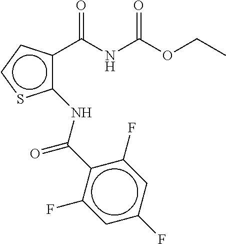 | | C | | | | |
| 177 | 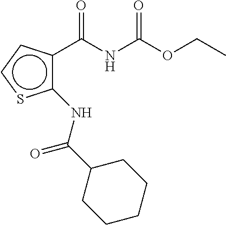 | | C | | | | |
| 178 | 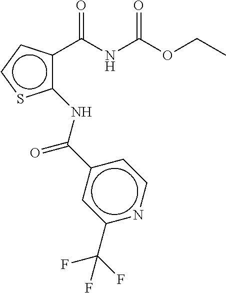 | | C | | | | |
| 179 | 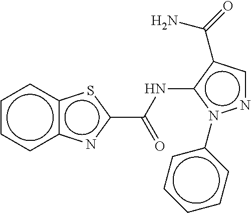 | | C | | | | |
| 180 | 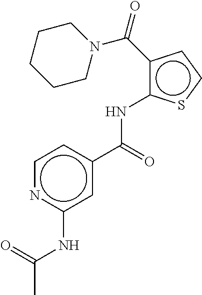 | | C | | | | |
| 181 | 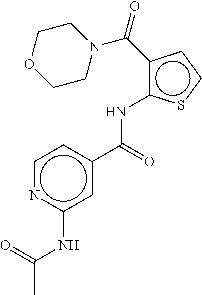 | | C | | | | |
| 182 | 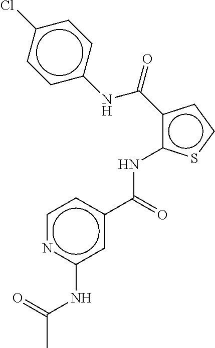 | | C | | | | |
| 183 | 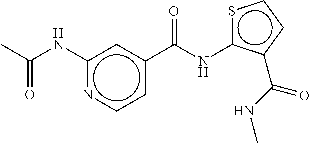 | | C | | | | |
| 184 | 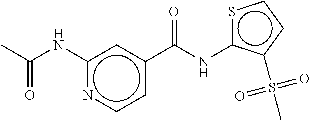 | | C | | | | |
| 185 | 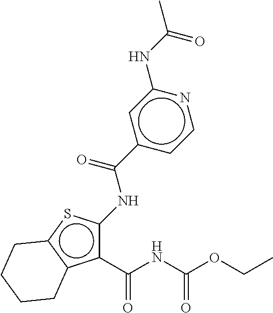 | | C | | | | |
| 186 | 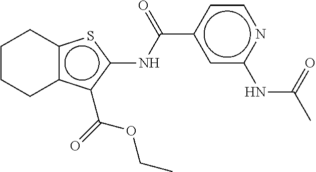 | | C | | | | |
| 187 | 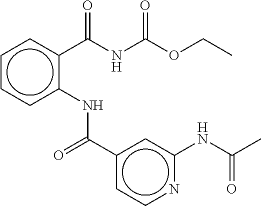 | | C | | | | |
| 188 | 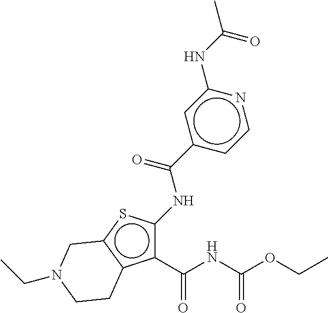 | | C | | | | |
| 189 | 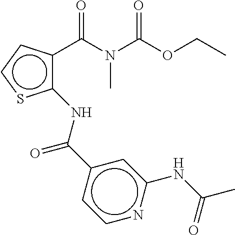 | | C | | | | |
| 190 | 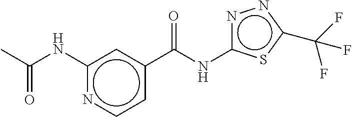 | | C | | | | |
| 191 | 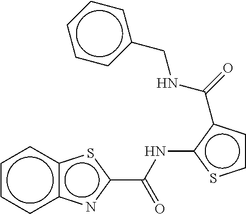 | | C | | | | |
| 192 | 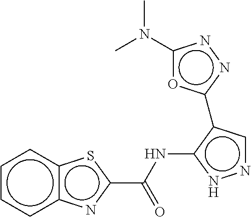 | | C | | | | |
| 193 | 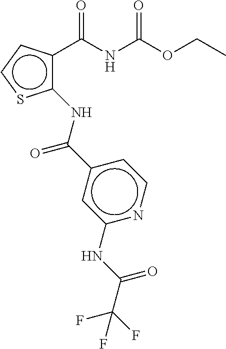 | | C | | | | |
| 194 | 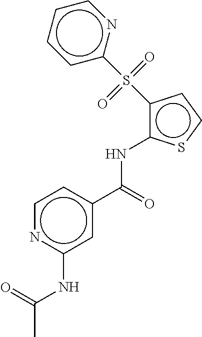 | | C | | | | |
| 195 | 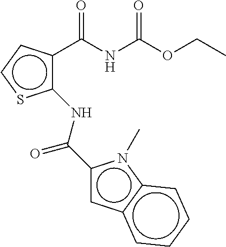 | | C | | | | |
| 196 | 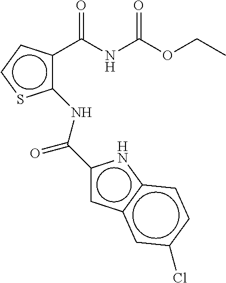 | | C | | | | |
| 197 | 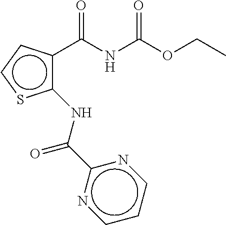 | | C | | | | |
| 198 | 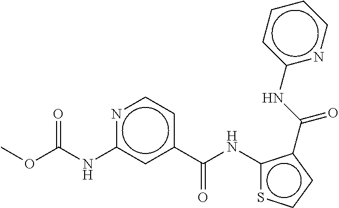 | | C | | | | |
| 199 | 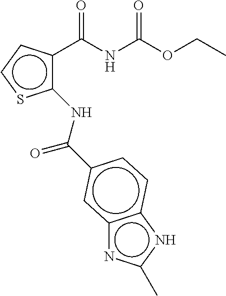 | | C | | | | |
| 200 | 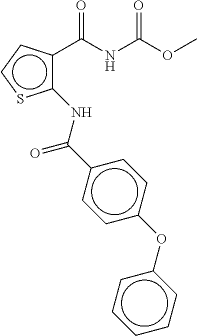 | | C | | | | |
| 201 | 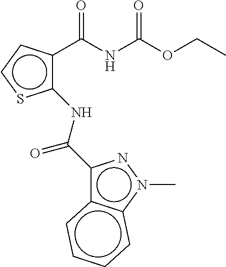 | | C | | | | |
| 202 | 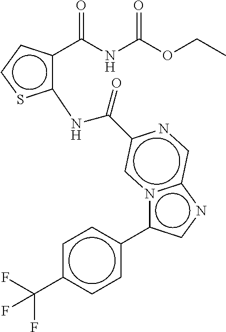 | | C | | | | |
| 203 | 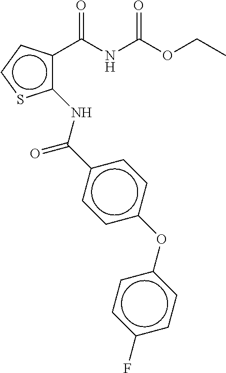 | | C | | | | |
| 204 | 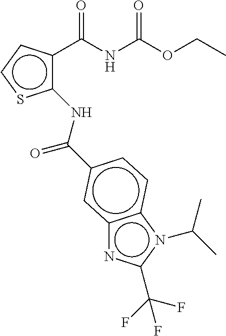 | | C | | | | |
| 205 | 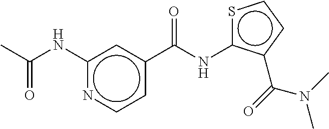 | | C | | | | |
| 206 | 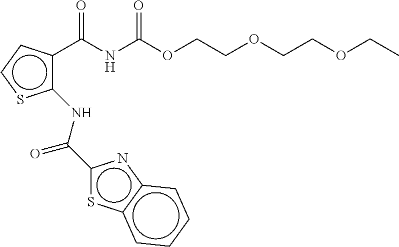 | | C | | | | |
| 207 | 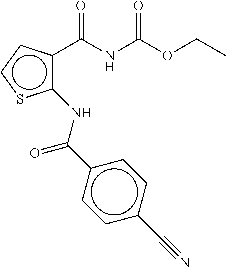 | | C | | | | |
| 208 | 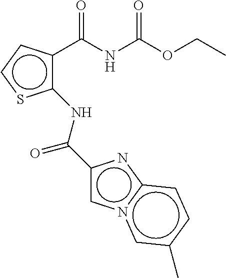 | C | | C | A | | ES-MS M/Z = 372.9 (M + 1) ¹H NMR (400 MHz, CD₃OD) 1.36 (s, 3H) 2.51 (s, 3H) 4.22-4.37 (m, 2H) 7.03-7.14 (m, 1H) 7.50-7.60 (m, 1H) 7.81-7.89 (m, 1H) 7.91-8.02 (m, 1H) 8.62-8.71 (m, 1H) 8.83-8.91 (m, 1H) |
| 209 | 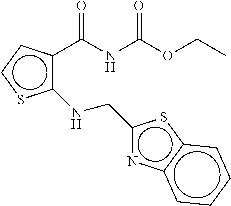 | | | | A | | ES-MS M/Z = 361.9 (M + 1) ¹H NMR (400 MHz, CD₃OD) 1.33-1.37 (t, 3H, J = 6.8 Hz), 4.24-4.29 (m, 2H), 4.96 (m, 2H), 6.37-6.38 (m, 1H), 7.24-7.25 (m, 1H), 7.41-7.45 (m, 1H), 7.51-7.55 (m, 1H), 7.96-7.98 (m, 2H) |
| 210 | 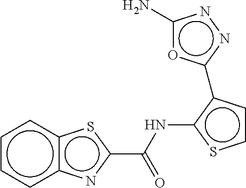 | | | | A | | ES-MS M/Z = 343.9 (M + 1) ¹H NMR (400 MHz, DMSO-d₆) 7.20- 7.22 (m, 1H), 7.34-7.37 (m, 3H),7.63- 7.70 (t, 2H), 8.25-8.31 (t, 2H), 12.26- 12.29 (m, 1H). |
| 211 | 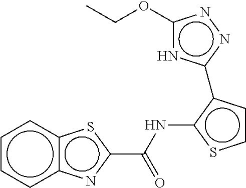 | | | | A | | ES-MS M/Z = 371.9 (M + 1) ¹H NMR (400 MHz, CDCl₃) 1.60-1.64 (t, 3H, J = 6.8 Hz), 4.87-4.93 (m, 2H), 7.00-7.01 (m, 1H), 7.40-7.41 (m, 2H), 7.54-7.60 (m, 2H), 7.99-8.04 (m, 2H) |
| 212 | 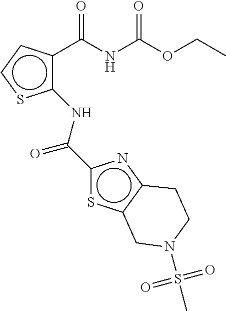 | | | | | | ES-MS M/Z = 459 (M + 1) ¹H NMR (400 MHz, DMSO-d₆) 1.29 (t, J = 7.03 Hz, 3H) 3.00 (s, 3H) 3.08 (br. s., 2H) 3.62-3.65 (m, 2H) 4.18-4.25 (m, 2H) 4.64-4.70 (m, 2H) 7.12- 7.18 (m, 1H) 7.72-7.77 (m, 1H) 10.81 (s, 1H) 12.80-12.87 (m, 1H) |
| 213 | 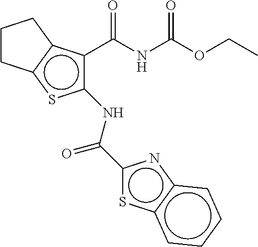 | | | | A | | ES-MS M/Z = 521.1 (M + 1) ¹H NMR (400 MHz, CD₃OD) 1.33 (br. s., 3H) 4.25-4.32 (m, 2H) 4.38-4.45 (m, 2H) 4.51-4.58 (m, 2 H) 4.81- 4.85 (m, 2H) 4.93-4.96 (m, 2H) 7.55 (d, J = 2.21 Hz, 7H) 8.13 (d, J = 7.28 Hz, 2H) |
| 214 | 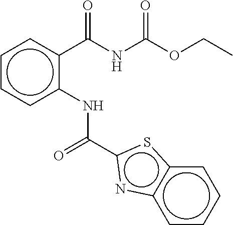 | | | | A | | ES-MS M/Z = 369.9 (M + 1) ¹H NMR (400 MHz, CD₃OD) 1.38-1.34 (t, 3H, 7.2 Hz), 4.34- 4.29 (q, 2H, J = 7.2 Hz), 7.35- 7.3 (t, 1H, J = 6.8 Hz), 7.61- 7.60 (t, 1H, J = 7.2Hz), 7.68-7.66 (m, 2H), 7.86-7.83 (d, 1H, J = 9.6 Hz), 8.15-8.13 (d, 1H, J = 7.6 Hz), 8.27- 8.25 (d, 1H, J = 8.0 Hz) 8.65-8.63 (d, 1H, J = 8.4 Hz) |
| 215 | 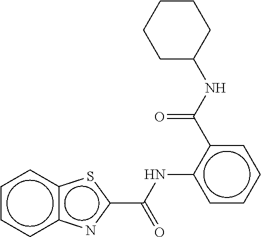 | | | | A | | ES-MS M/Z = 280.1 (M + 1) ¹H NMR (400 MHz, DMSO-d₆) 7.453- 7.759 (m, 5H), 7.881-7.900 (m, 1H), 8.205-8.281 (m, 2H), 11.331 (s, 1H) |
| 216 | 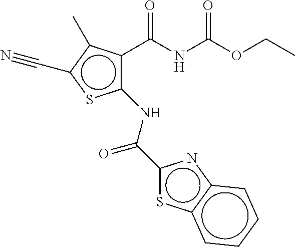 | | | | A | | ES-MS M/Z = 325.9 de-carbamate ¹H NMR (400 MHz, CDCl₃) 12.82- 13.04 (m, 1H), 8.16-8.21 (m, 1H), 7.93-7.99 (m, 1H), 7.47-7.59 (m, 2H), 4.42-4.49 (m, 2H), 2.57 (s, 3H), 1.47 (d, J = 7.09 Hz, 3H) |
| 217 | 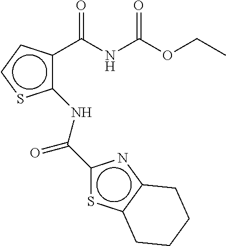 | A | | C | A | | ES-MS M/Z = 380 (M + 1) ¹H NMR (400 MHz, CDCl₃) 1.35 (t, J = 7.09 Hz, 3H) 1.88 (br. s., 4H) 2.87 (br. s., 4H) 4.24-4.40 (m, 1H) 6.82- 6.92 (m, 1H) 6.96-7.09 (m, 1H) 7.74 - 7.90 (m, 1H) 12.60-12.75 (m, 1H). |
| 218 | 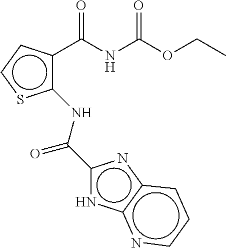 | | | | | | ES-MS M/Z = 360.0 (M + 1) |
| 219 | 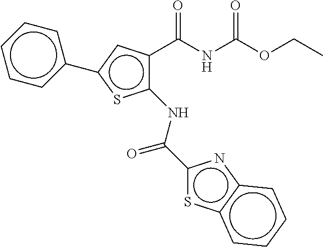 | | | | | | ES-MS M/Z = 452.2 (M + 1) |
| 220 | 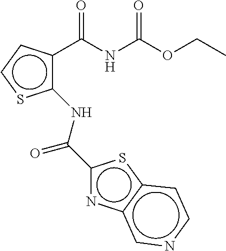 | | | | | | ES-MS M/Z = 391.1 (M + 1) |
| 221 | 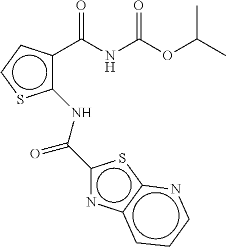 | | | | | | ES-MS M/Z = 391.1(M + 1) |
| 222 | 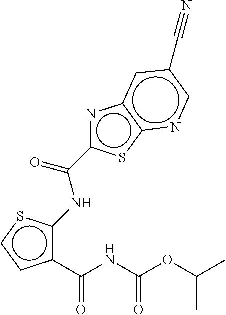 | | | | | | ES-MS M/Z = 416.1 (M + l) |
| 223 | 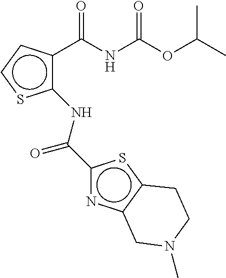 | | | | | | ES-MS M/Z = 409.1 (M + 1) |
| 224 | 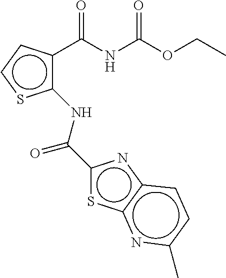 | | | | A | | ES-MS M/Z = 391 (M + 1) ¹H NMR (400 MHz, DMSO-d₆) 1.28- 1.31 (t, 3H, J = 7.2 Hz), 2.68 (s, 3H), 4.20-4.25 (m, 2H), 7.19-7.21 (d, 1H, J = 6.0 Hz), 7.62-7.64 (d, 1H, J = 8.4 Hz), 7.76-7.78 (d, 1H, J = 5.6 Hz), 8.61-8.63 (d, 1H, J = 8.8 Hz), 8.60 (s, 1H), 10.86 (s, 1H), 13.05 (s, 1H) |
| 225 | 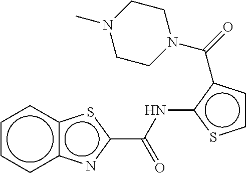 | | | | B | | ES-MS M/Z = 387.0 (M + 1) ¹H NMR (400 MHz, DMSO-d₆) 2.70- 2.84 (m, 3H) 3.09-3.21 (m, 2H) 3.43- 3.49 (m, 2H) 3.52-3.57 (m, 2H) 4.23-4.48 (m, 2H) 7.17-7.22 (m, 1H) 7.26-7.31 (m, 1H) 7.59-7.75 (m, 2H) 8.21-8.37 (m, 2H) 12.00-12.08 (m, 1H) |
| 226 | 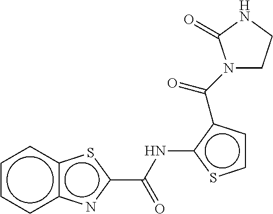 | | | | A | | ES-MS M/Z= 372.9 (M + 1) ¹H NMR (400 MHz, DMSO-d₆) 8.17- 8.33 (m, 2H), 7.59-7.68 (m, 3H), 7.28- 7.45 (m, 3H), 6.49-6.61 (m, 1H), 3.58- 3.75 (m, 2H), 3.15-3.25 (m, 2H) |
| 227 | 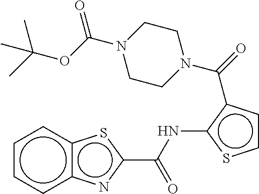 | | | | A | | ES-MS M/Z= 473.1 (M + 1) ¹H NMR (400 MHz, DMSO-d₆) 1.34- 1.44 (m, 9H) 3.40-3.45 (m, 4H) 3.57- 3.63 (m, 4H) 7.11-7.15 (m, 1H) 7.20-7.25 (m, 1H) 7.60-7.68 (m, 2H) 8.20-8.28 (m, 2H) |
| 228 | 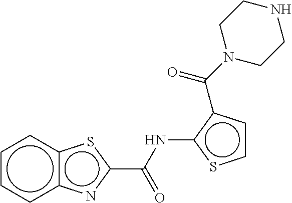 | | | | C | | ES-MS M/Z = 372.9 (M + 1) ¹H NMR (400 MHz, DMSO-d₆) 3.19- 3.25 (m, 4H) 3.83-3.89 (m, 4H) 7.19- 7.22 (m, 1H) 7.26-7.29 (m, 1H) 7.62-7.72 (m, 2H) 8.24-8.33 (m, 2H) 9.51-9.61 (m, 2H) 11.97-12.01 (m, 1H) |
| 229 | 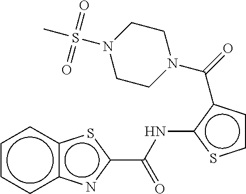 | | | | A | | ES-MS M/Z = 473 (M + 1) ¹H NMR (400 MHz, DMSO-d₆) 2.87 (s, 3H) 3.18-3.25 (m, 4H) 3.68-3.76 (m, 4H) 7.11-7.16 (m, 1H) 7.23-7.28 (m, 1H) 7.59-7.69 (m, 2H) 8.21-8.30 (m, 2H) 12.01-12.05 (m, 1H) |
| 230 | 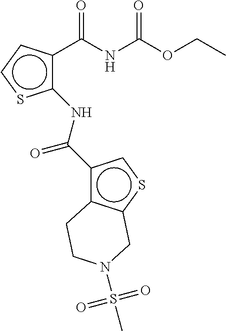 | | | | A | | ES-MS M/Z = 480 (M + 23) ¹H NMR (400 MHz, CDCl₃) 1.38- 1.35 (t, 3H, J = 7.2 Hz), 3.25-3.22 (m, 2H), 2.87 (s, 3H), 3.65-3.62 (m, 2H), 4.36-4.30 (q, 2H, J = 7.2 Hz), 4.56 (s, 2H), 6.87-6.85 (d, 1H, J = 5.6 Hz), 7.04-7.03 (d, 1H, 6.0 Hz),7.87 (s, 1H), 7.26 (s, 1H), 12.31 (s, 1H), 8.0 (s, 1H), 12.31 (s, 1H) |
| 231 | 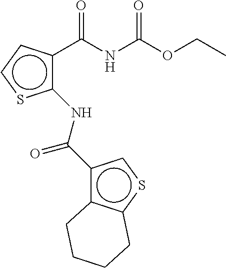 | | | | C | | ES-MS M/Z = 378.9 (M + 1) ¹H NMR (400 MHz, CD₃OD) 1.37- 1.33 (t, 3H, J = 6.8 Hz), 1.86- 1.85 (m, 4H), 2.93 (m, 2H), 2.81 (m, 2H), 4.28-4.27 (q, 2H, J = 6.8 Hz), 6.95- 6.93 (d, 1H, 6.0 Hz),7.48- 7.46 (d, 1H,J = 6.0Hz),7.96 (s, 1H). |
| 232 | 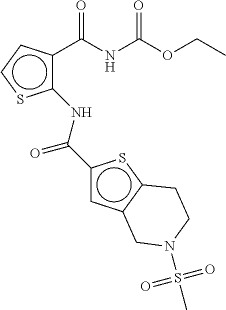 | | | | A | | ES-MS M/Z = 458.1 (M + 1) ¹H NMR (400 MHz, DMSO-d₆) 1.27- 1.24 (t, 3H, J = 7.6 Hz), 3.0 (m, 2H), 2.94 (s, 3H),4.20-4.17 (q, 2H, J = 7.6 Hz), 4.37 (s, 2H), 7.05- 7.03 (d, 1H, J = 6.0 Hz), 7.63 (s, 1H), 7.69-7.67 (d, 1H, J = 6.0 Hz) 10.77 (s, 1H), 12.38 (s, 1H). |
| 233 | 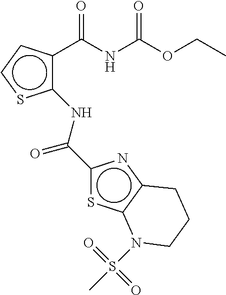 | | | | A | | ES-MS M/Z = 459 (M + 1) ¹H NMR (400 MHz, DMSO-d₆) 1.23- 1.29 (m, 3H) 1.99-2.07 (m, 2H) 2.87- 2.92 (m, 2H) 3.21 (s, 3H) 3.75-3.80 (m, 2H) 4.15-4.22 (m, 2H) 7.08-7.11 (m, 1H) 7.68-7.71 (m, 1H) 10.74-10.77 (m, 1H) 12.70-12.73 (m, 1H) |
| 234 | 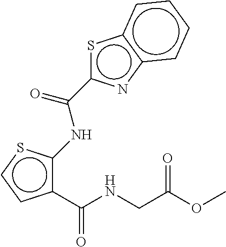 | | | | A | | ES-MS M/Z = 375.9 (M + 1) ¹H NMR (400 MHz, DMSO-d₆) 3.67 (s, 3H) 4.06-4.11 (m, 2H) 7.19-7.23 (m, 1H) 7.53-7.56 (m, 1H) 7.59-7.69 (m, 2H) 8.23-8.30 (m, 2H) 8.98-9.05 (m, 1H) 13.36-13.40 (m, 1H) |
| 235 | 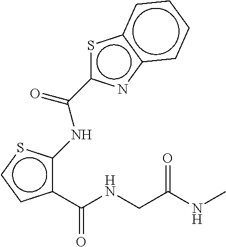 | | | | A | | ES-MS M/Z = 397 (M + 23) ¹H NMR (400 MHz, DMSO-d₆) 2.61- 2.65 (m, 3H), 3.87-3.92 (m, 2 H) 7.19- 7.25 (m, 1H) 7.57-7.61 (m, 1H) 7.62-7.71 (m, 2H) 7.92-7.98 (m, 1H) 8.26-8.33 (m, 2H) 8.84-8.91 (m, 1H) 13.51-13.55 (m, 1H) |
| 236 | 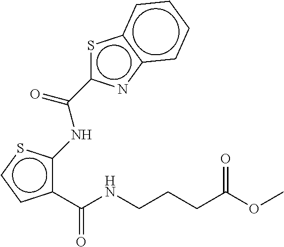 | | | | A | | ES-MS M/Z = 404.0 (M + 1) ¹H NMR (400 MHz, DMSO-d₆) 1.20 (s, 3H) 2.60-2.66 (m, 2H) 3.50-3.61 (m, 2H) 4.04-4.14 (m, 2H) 7.16-7.22 (m, 1H) 7.48-7.55 (m, 1H) 7.61-7.73 (m, 2H) 8.24-8.35 (m, 2H) 8.61- 8.68 (m, 1H) 13.53-13.64 (m, 1H) |
| 237 | 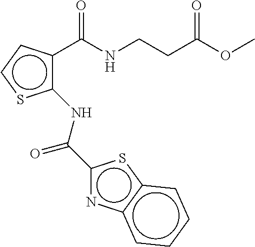 | | | | B | | ES-MS M/Z = 403.9 (M + 23) ¹H NMR (400 MHz, DMSO-d₆) 1.75- 1.85 (m, 2H) 2.35-2.41 (m, 2H) 3.33- 3.40 (m, 2H) 3.57 (s, 3H) 7.14-7.19 (m, 1H) 7.50-7.55 (m, 1H) 7.58-7.69 (m, 2H) 8.26 (s, 2H) 8.51-8.58 (m, 1H) 13.56-13.62 (m, 1H) |
| 238 | 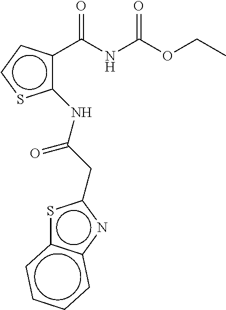 | | | | B | | ES-MS M/Z = 406 (M + 17) ¹H NMR (400 MHz, DMSO-d₆) 1.3- 1.27 (t, 3H, J = 6.8 Hz), 4.23- 4.18 (q, 2H, J = 6.8 Hz), 5.16 (s, 1H), 7.03-6.98 (m, 3H), 7.22-7.20 (m, 1H), 7.36-7.34 (d, 2H, J = 7.2 Hz), 7.62- 7.61 (d, 1H, J = 6.0 Hz), 11.0 (s, 1H), 10.74 (s, 1H), 12.12 (s, 1H). |
| 239 | 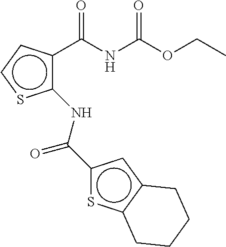 | | | | A | | ES-MS M/Z = 379 (M + 1) ¹H NMR (400 MHz, CDCl₃) 12.275 (s, 1H), 7.856 (s, 1H), 7.511 (s, 1H), 7.009-6.994 (d, 1H), 6.827-6.812 (d, 1H), 4.348-4.295 (m, 2H), 2.822-2.794 (m, 2H), 2.655-2.627 (m, 2H), 1.863- 1.806 (m, 4H), 1.373-1.337 (m, 3H) |
| 240 | 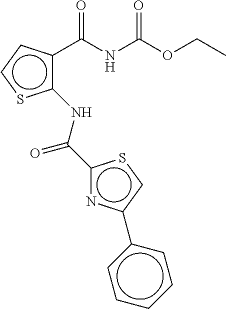 | | | | A | | ES-MS M/Z= 402 M + 1) ¹H NMR (400 MHz, DMSO-d₆) 1.29- 1.26 (t, 3H, J = 7.2 Hz), 4.21- 4.19 (q, 2H, J = 7.2 Hz), 7.15- 7.13 (d, 1H, J = 6.0 Hz),7.42 (m, 1H), 7.53-7.50 (m, 2H), 7.73-7.72 (d, 2H, J = 6.4 Hz), 8.06-8.05 (d, 1H, J = 7.2 Hz), 8.60 (s, 1H), 10.80 (s, 1H), 13.01 (s, 1H). |
| 241 | 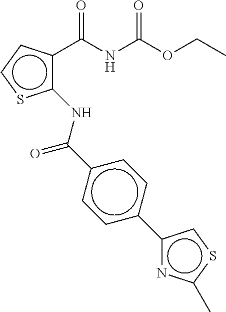 | | | | A | | ES-MS M/Z = 416 (M + 1) ¹H NMR (400 MHz, DMSO-d₆) 1.31- 1.27 (t, 3H, J = 7.2 Hz), 2.75 (s, 3H), 4.25-4.20 (q, 2H, J = 7.2 Hz), 7.10-7.08 (d, 1H, J = 6.0 Hz), 7.74-7.73 (d, 1H, J = 6.0 Hz), 8.01-8.00 (d, 2H, J = 8.4 Hz), 8.19-8.16 (m, 3H), 10.81 (s, 1H), 12.64 (s, 1H). |
| 242 | 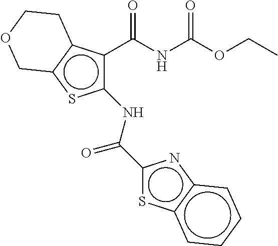 | | | C | A | | ES-MS M/Z = 454 (M + 23) |
| 243 | 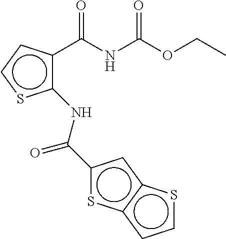 | | | | | | ES-MS M/Z = 381.0 (M + 1) |
| 244 | 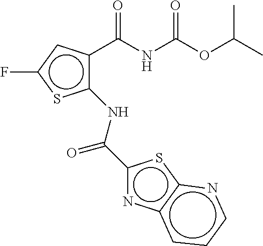 | C | | C | | | ES-MS M/Z = 383.0 (M + 1) |
| 245 | 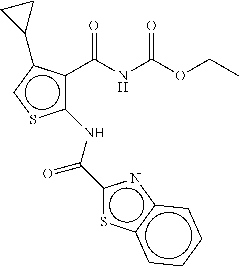 | C | | C | | | |
| 246 | 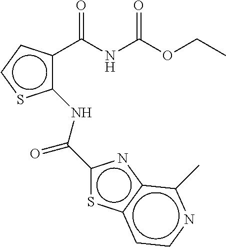 | | | | | | ES-MS M/Z = 462 (M + 1) ¹H NMR (400 MHz, DMSO-d₆) 1.21- 1.37 (m, 3H) 3.86-3.97 (m, 4H) 4.02- 4.11 (m, 4H) 4.16-4.26 (m, 2H) 6.90-7.06 (m, 1H) 7.10-7.20 (m, 1H) 7.69-7.87 (m, 1H) 8.25-8.40 (m, 1H) 10.70-10.86 (m, 1H) 13.24-13.41 (m, 1H) |
| 247 | 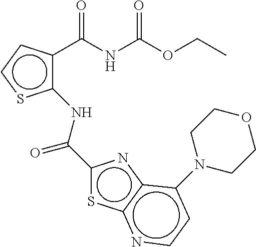 | A | | B | B | | ES-MS M/Z = 462 (M + 1) ¹H NMR (400 MHz, DMSO-d₆) 1.21- 1.37 (m, 3H) 3.86-3.97 (m, 4H) 4.02- 4.11 (m, 4H) 4.16-4.26 (m, 2H) 6.90-7.06 (m, 1H) 7.10-7.20 (m, 1H) 7.69-7.87 (m, 1H) 8.25-8.40 (m, 1H) 10.70-10.86 (m, 1H) 13.24-13.41 (m, 1H). |
| 248 | 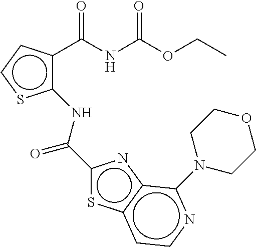 | A | | C | A | | ES-MS M/Z = 462 (M + 1) ¹H NMR (400 MHz, DMSO-d₆) 1.252- 1.287 (m, 3H), 3.748 (s, 0.674H), 3.851 (m, 4H), 4.061 (s, 0.720H), 4.171-4.224 (m, 6H), 7.116-7.131 (d, J = 6.0 Hz, 1H), 7.297-7.309 (d, J = 4.8 Hz, 1H), 7.740-7.753 (d, J = 5.2 Hz, 1H), 8.094-8.107 (d, J = 5.2 Hz, 1H), 10.757 (s, 1H), 13.348 (S, 1H). |
| 249 | 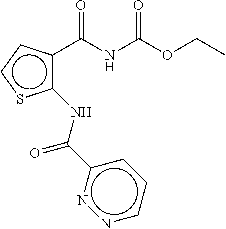 | C | | C | | | ES-MS M/Z = 321 (M + 1) ¹H NMR (400 MHz, DMSO-d₆) 13.55 (s, 1H), 10.82 (s, 1H), 9.54 (dd, J = 5.1, 1.7 Hz, 1H), 8.40 (dd, J = 8.5, 1.7 Hz, 1H), 8.04 (dd, J = 8.5, 5.0 Hz, 1H), 7.77 (d, J = 5.9 Hz, 1H), 7.18 (d, J = 5.9 Hz, 1H), 4.23 (q, J = 7.1 Hz, 2H), 1.30 (t, J = 7.1 Hz, 3H). |
| 250 | 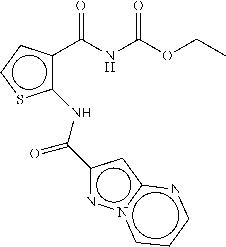 | A | | B | | | ES-MS M/Z = 360 (M + 1) ¹H NMR (400 MHz, DMSO-d₆) 12.88 (s, 1H), 10.81 (s, 1H), 9.40 (dt, J = 6.9, 1.3 Hz, 1H), 8.73 (dd, J = 4.0, 1.7 Hz, 1H), 7.75 (d, J = 5.9 Hz, 1H), 7.48-7.23 (m, 2H), 7.23-7.08 (m, 1H), 4.23 (q, J = 7.1 Hz, 2H), 1.30 (t, J = 7.1 Hz, 3H). |
| 251 | 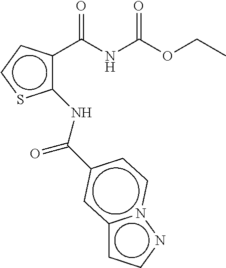 | A | | B | | | ES-MS M/Z = 359 (M + 1) ¹H NMR (400 MHz, DMSO-d₆) 12.66 (s, 1H), 10.86 (s, 1H), 8.92 (d, J = 7.3 Hz, 1H), 8.37 (d, J = 1.8 Hz, 1H), 8.19 (d, J = 2.4 Hz, 1H), 7.75 (d, J = 5.9 Hz, 1H), 7.28 (dd, J = 7.3, 2.1 Hz, 1H), 7.13 (d, J = 5.9 Hz, 1H), 7.03 (d, J = 2.1 Hz, 1H), 4.23 (q, J = 7.1 Hz, 2H), 1.30 (t, J = 7.1 Hz, 3H). |
| 252 | 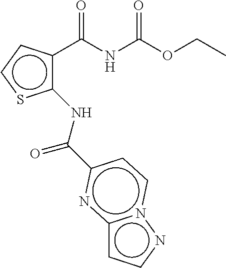 | C | | C | | | ES-MS M/Z = 360 (M + 1) ¹H NMR (400 MHz, DMSO-d₆) 13.24 (s, 1H), 10.83 (s, 1H), 9.44-9.37 (m, 1H), 8.48 (d, J = 2.5 Hz, 1H), 7.80-7.67 (m, 2H), 7.17 (d, J = 6.0 Hz, 1H), 7.12 (dd, J = 2.4, 1.0 Hz, 1H), 4.23 (q, J = 7.1 Hz, 2H), 1.31 (t, J = 7.1 Hz, 3H). |
| 253 | 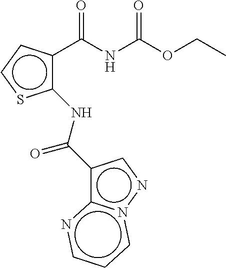 | C | | C | | | ES-MS M/Z = 359 (M + 1) ¹H NMR (400 MHz, DMSO-d₆) 12.86 (s, 1H), 10.66 (s, 1H), 9.41 (dd, J = 7.0, 1.6 Hz, 1H), 8.93 (dd, J = 4.2, 1.7 Hz, 1H), 8.79 (s, 1H), 7.64 (d, J = 5.9 Hz, 1H), 7.38 (dd, J = 7.0, 4.2 Hz, 1H), 7.03 (dd, J = 5.9, 0.8 Hz, 1H), 4.22 (q, J = 7.1 Hz, 2H), 1.30 (t, J = 7.1 Hz, 3H). |
| 254 | 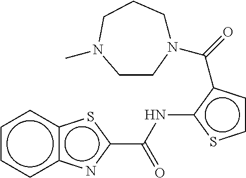 | C | | C | A | | ES-MS M/Z = 401.1 (M + 1) ¹H NMR (400 MHz, CD₃OD) 2.24-2.35 (m, 2H) 2.98 (s, 3H) 3.42 (d, J = 6.36 Hz, 1H) 3.50-3.61 (m, 2H) 3.67- 3.78 (m, 2 H) 3.83-3.92 (m, 1H) 3.94- 4.01 (m, 1H) 4.24-4.33 (m, 1H) 7.10-7.15 (m, 1H) 7.16-7.22 (m, 1H) 7.53-7.65 (m, 2 H) 8.08-8.18 (m, 2H) |
| 255 | 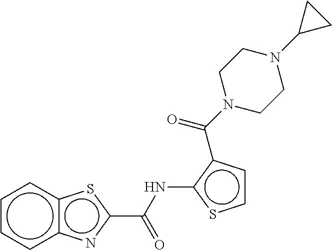 | C | | C | A | | ES-MS M/Z = 401 (M + 1) ¹H NMR (400 MHz, DMSO-d₆) 1.79- 2.10 (m, 4H) 2.72 (d, J = 3.91 Hz, 2H) 3.00-3.16 (m, 2H) 3.20-3.40 (m, 3H) 4.07- 4.20 (m, 1H) 7.14-7.22 (m, 1H) 7.51-7.75 (m, 3H) 8.18-8.30 (m, 2H) 8.53-8.62 (m, 1H) 10.30- 10.43 (m, 1H) 13.56 (s, 1H) |
| 256 | 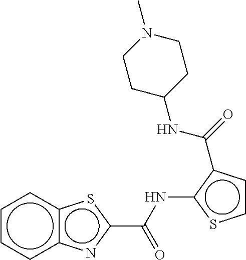 | C | | B | C | | ES-MS M/Z = 413.1 (M + 1) ¹H NMR (400 MHz, DMSO-d₆) 0.75- 0.83 (m, 2H) 1.02-1.13 (m, 2H) 2.69- 2.93 (m, 2H) 3.42-3.58 (m, 4H) 4.25-4.43 (m, 2H) 7.15-7.21 (m, 1H) 7.24-7.30 (m, 1H) 7.58-7.70 (m, 2H) 8.19- 8.30 (m, 2H) 12.03 (s, 1H) |
| 257 | 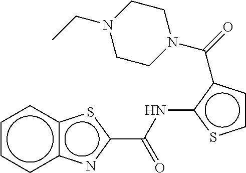 | C | | B | B | | ES-MS M/Z = 401 (M + 1) ¹H NMR (400 MHz, DMSO-d₆) 1.18- 1.27 (m, 1H) 3.04-3.18 (m, 1H) 3.47- 3.54 (m, 1H) 4.27-4.41 (m, 1H) 7.14-7.21 (m, 1H) 7.24-7.29 (m, 1H) 7.59-7.70 (m, 1H) 8.19-8.25 (m, 1H) 8.25-8.30 (m, 1H) 10.17-10.31 (m, 1H) 12.02 (s, 1H) |
| 258 | 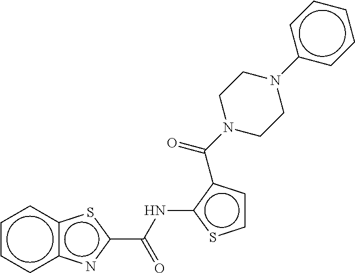 | C | | C | A | | ES-MS M/Z = 449.2 (M + 1) ¹H NMR (400 MHz, DMSO-d₆) 3.21- 3.29 (m, 4H) 3.71-3.85 (m, 4H) 6.77- 6.84 (m, 1H) 6.94-7.01 (m, 2H) 7.13-7.30 (m, 4H) 7.58-7.70 (m, 2H) 8.19-8.30 (m, 2 H) 12.04-12.11 (m, 1H) |
| 259 | 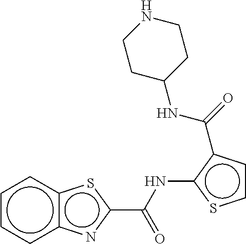 | C | | C | A | | ES-MS M/Z = 387 (M − 101) ¹H NMR (400 MHz, DMSO-d₆) 1.39 (s, 9H) 1.75-1.82 (m, 2H) 2.78-2.90 (m, 2H) 3.92-4.02 (m, 2 H) 4.02-4.20 (m, 2H) 7.14-7.19 (m, 1H) 7.56- 7.69 (m, 3H) 8.21-8.31 (m, 3H) 13.59-13.63 (m, 1H). |
| 260 | 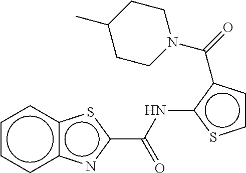 | C | | C | B | | ES-MS M/Z = 386.1 (M + 1) ¹H NMR (400 MHz, DMSO-d₆) 0.90 (d, J = 6.11 Hz, 3H) 1.09-1.21 (m, 2H) 1.59-1.72 (m, 3H) 2.89-3.02 (m, 2H) 4.14- 4.26 (m, 2H) 7.09 (s, 1H) 7.20-7.24 (m, 1H) 7.59-7.69 (m, 2H) 8.20-8.30 (m, 2H) 12.04 (s, 1H) |
| 261 | 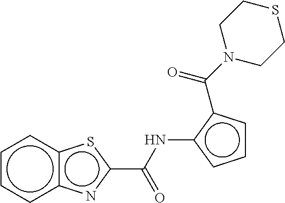 | C | | C | A | | ES-MS M/Z = 390 (M + 1) ¹H NMR (400 MHz, DMSO-d₆) 2.67- 2.76 (m, 4 H) 3.78-3.88 (m, 4H) 7.07- 7.12 (m, 1H) 7.21-7.26 (m, 1H) 7.58-7.70 (m, 2H) 8.20-8.30 (m, 2H) 11.93 (s, 1H) |
| 262 | 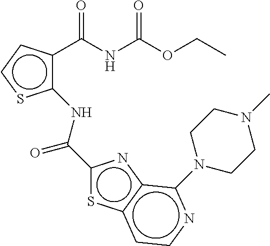 | B | | B | B | | ES-MS M/Z = 475.2 (M + 1) ¹H NMR (400 MHz, DMSO-d₆) 1.29 (t, J = 6.97 Hz, 3H) 2.43-2.46 (m, 7 H) 3.39-3.45 (m, 4H) 4.16 (d, J = 6.85 Hz, 2H) 6.60-6.69 (m, 1H) 7.16 (d, J = 5.14 Hz, 1H) 7.33 (d, J = 3.67 Hz, 1H) 7.98- 8.08 (m, 1H) 12.96 (br. s., 1H) |
| 263 | 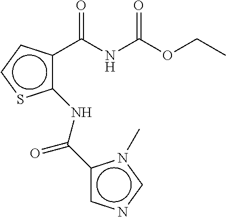 | C | | C | A | | ES-MS M/Z = 323 (M + 1) ¹H NMR (400 MHz, DMSO-d₆) 1.25 (t, J = 7.09 Hz, 3H) 3.88 (s, 3H) 4.18 (d, J = 7.09 Hz, 2H) 6.98-7.05 (m, 1H) 7.64 (br. s., 2H) 7.95 (s, 1H) 10.74 (s, 1H) 12.18-12.25 (m, 1H) |
| 264 | 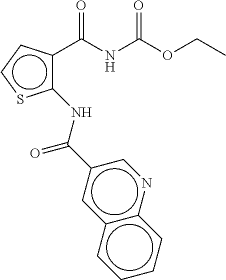 | A | | B | C | | ES-MS M/Z = 370 (M + 1) ¹H NMR (400 MHz, DMSO-d₆) 12.72 (s, 1H), 10.88 (s, 1H), 9.36 (d, J = 2.3 Hz, 1H), 8.98 (d, J = 2.3 Hz, 1H), 8.27 (dd, J = 8.2, 1.4 Hz, 1H), 8.16 (dd, J = 8.3, 1.1 Hz, 1H), 7.96 (ddd, J = 8.4, 6.9, 1.5 Hz, 1H), 7.84-7.71 (m, 2H), 7.15 (d, J = 5.9 Hz, 1H), 4.23 (q, J = 7.1 Hz, 2H), 1.30 (t, J = 7.1 Hz, 3H). |
| 265 | 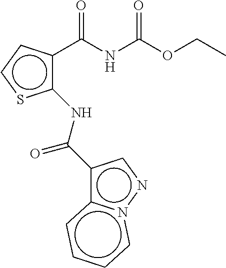 | C | | C | B | | ES-MS M/Z = 359 (M + 1) ¹H NMR (400 MHz, DMSO-d₆) 12.27 (s, 1H), 10.78 (s, 1H), 9.04-8.85 (m, 1H), 8.52 (s, 1H), 8.36-8.22 (m, 1H), 7.76- 7.65 (m, 1H), 7.32-7.17 (m, 1H), 7.03 (d, J = 5.9 Hz, 1H), 6.12 (s, 1H), 4.23 (q, J = 7.1 Hz, 2H), 1.30 (t, J = 7.1 Hz, 3H). |
| 266 | 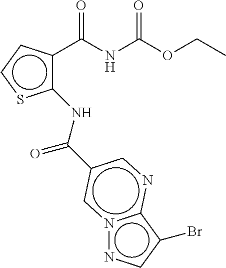 | A | | C | B | | ES-MS M/Z = 437 (M + 1) ¹H NMR (400 MHz, DMSO-d₆) 12.46 (s, 1H), 10.87 (s, 1H), 9.68 (d, J = 2.1 Hz, 1H), 8.99 (d, J = 2.1 Hz, 1H), 8.64 (s, 1H), 7.70 (d, J = 5.9 Hz, 1H), 7.15 (d, J = 5.9 Hz, 1H), 4.23 (q, J = 7.1 Hz, 2H), 1.29 (t, J = 7.1 Hz, 3H). |
| 267 | 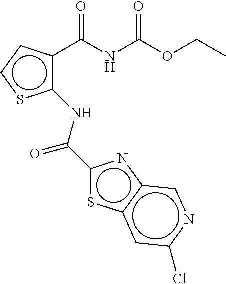 | A | | C | A | | ES-MS M/Z = 411 (M + 1) ¹H NMR (400 MHz, DMSO-d₆) 1.28- 1.31 (t, 3H, J = 7.2 Hz), 4.20- 4.25 (q, 2H, J = 7.2 Hz), 7.22-7.24 (d, 1H, J = 6.0 Hz), 7.78-7.79 (d, 1H, J = 5.6 Hz), 8.52 (s, 1H), 9.45 (s, 1H), 10.89 (s, 1H), 13.10 (s, 1H) |
| 268 | 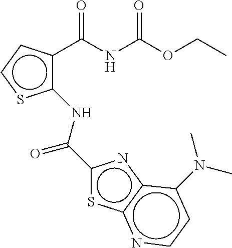 | A | | C | A | | ES-MS M/Z = 420 (M + 1) ¹H NMR (400 MHz, DMSO-d₆) 1.27 (t, J = 7.06 Hz, 3H) 3.59 (s, 6H) 4.13-4.27 (m, 2H) 6.88 (d, J = 6.62 Hz, 1H) 7.16 (d, J = 5.95 Hz, 1H) 7.75 (d, J = 5.95 Hz, 1H) 8.32 (d, J = 6.62 Hz, 1H) 10.80 (s, 1H) 13.20 (s, 1H) |
| 269 | 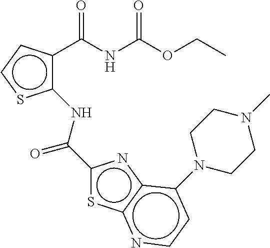 | A | | B | A | | ES-MS M/Z = 475.1 (M + 1) ¹H NMR (400 MHz, CD₃OD) 1.35 (s, 3H) 3.08 (s, 3H) 3.47-3.61 (m, 2H) 3.71-3.87 (m, 2H) 4.04-4.18 (m, 2H) 4.22-4.38 (m, 2H) 5.27-5.51 (m, 2H) 7.00-7.13 (m, 1H) 7.16-7.28 (m, 1H) 7.47-7.64 (m, 1H) 8.35-8.49 (m, 1H). |
| 270 | 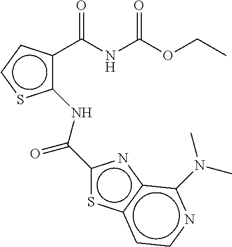 | A | | C | A | | ES-MS M/Z = 420 (M + 1) ¹H NMR (400 MHz, CDCl₃) 1.31 (t, j = 7.21 Hz, 3H) 3.58 (s, 6H) 4.26 (d, J = 7.09 Hz, 2H) 6.84-6.89 (m, 1H) 7.02 (dd, J = 9.29, 5.62 Hz, 2 H) 7.77- 7.85 (m, 1H) 8.01-8.07 (m, 1H) 12.97-13.04 (m, 1H) |
| 271 | 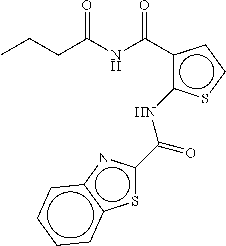 | | | | | | ES-MS M/Z = 373.9 (M + 1) ¹H NMR (400 MHz, DMSO-d₆) 0.945- 0.983 (m, 3H), 1.610-1.665 (m, 2H), 2.772 (s, 2H), 7.201-7.213 (m, 1H), 7.501-7.630 (m, 2H), 7.793-7.810 (d, 1H), 8.401-8.491 (m, 2H), 10.822 (s, 1H), 13.052 (s, 1H) |
| 272 | 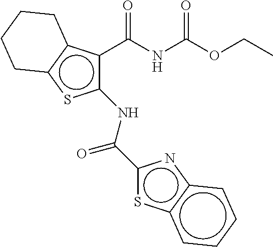 | | | | | | ES-MS M/Z = 430 (M + 1) ¹H NMR (400 MHz, DMSO-d₆) 11.93- 12.05 (m, 1H), 10.37-10.46 (m, 1H), 8.19-8.35 (m, 2H), 7.58-7.75 (m, 2H), 4.17 (s, 2H), 2.70 (br. s, 5H), 1.67- 1.84 (m, 3H), 1.24 (s, 3H) |
| 273 | 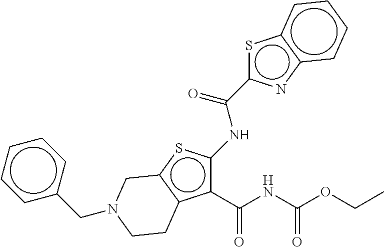 | | | | | | ES-MS M/Z = 521.1 (M + 1) ¹H NMR (400 MHz, DMSO-d₆) 1.26 (br. s., 3H) 3.00-3.08 (m, 1H) 3.16-3.23 (m, 1H) 3.56-3.76 (m, 2H) 4.19 (d, J = 5.52 Hz, 2H) 4.29-4.52 (m, 4H) 7.52 (d, J = 3.51 Hz, 3H) 7.56-7.61 (m, 2H) 7.63-7.75 (m, 2H) 8.19-8.35 (m, 2H) 10.67-10.77 (m, 1H) 12.08 (br. s., 1H) |
| 274 | 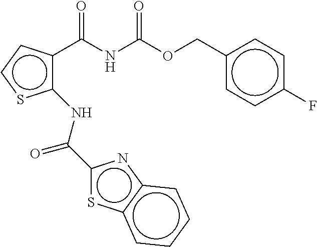 | B | | C | C | | ES-MS M/Z = 477.9 (M + 23) |
| 275 | 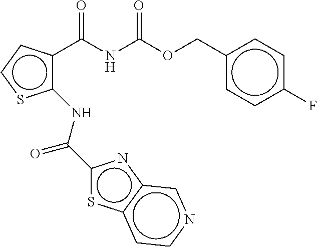 | C | | C | B | | ES-MS M/Z = 456.9 (M + 1) |
| 276 | 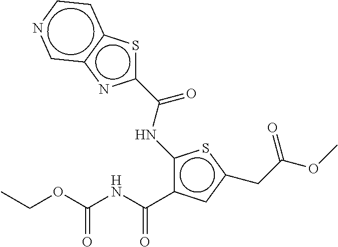 | C | | C | B | | ES-MS M/Z = 449 (M + 1) ¹H NMR (400 MHz, CDCl₃) 10.84 (s, 1H), 9.61 (s, 1H), 8.70 (d, J = 5.5 Hz, 1H), 8.39 (dd, J = 5.5, 1.0 Hz, 1H), 7.64 (s, 1H), 4.22 (q, J = 7.1 Hz, 2H), 3.92 (s, 2H), 3.68 (s, 3H), 1.29 (t, J = 7.1 Hz, 3H). |
| 277 | 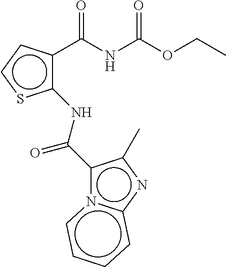 | | | | | | ES-MS M/Z = 373 (M + 1) ¹H NMR (400 MHz, DMSO-d₆) 1.27- 1.24 (t, 3H, J = 6.8 Hz), 2.84 (s, 3H), 4.21- 4.15 (q, 2H, J = 6.8Hz), 7.04-7.03 (d, 1H, J = 6.0 Hz), 7.18-7.15 (t, 1H, J = 6.8), 7.6- 7.5 (m, 1H), 7.70-7.67 (m, 2H), 9.45- 9.43 (d, 1H, J = 6.8 Hz), 10.73 (s, 1H), 12.04 (s, 1H). |
| 278 | 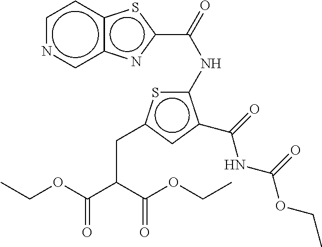 | C | | C | A | | ES-MS M/Z = 549 (M + 1) ¹H NMR (400 MHz, DMSO-d₆) 13.04 (s, 1H), 10.80 (s, 1H), 9.66 (s, 1H), 8.72 (d, J = 5.6 Hz, 1H), 8.43 (d, J = 5.6 Hz, 1H), 7.62 (s, 1H), 4.22 (q, J = 7.1 Hz, 2H), 4.18-4.05 (m, 4H), 3.84 (t, J = 7.5 Hz, 1H), 3.26 (d, J = 7.5 Hz, 2H), 1.29 (t, J = 7.1 Hz, 3H), 1.16 (t, J = 7.1 Hz, 6H). |
| 279 | 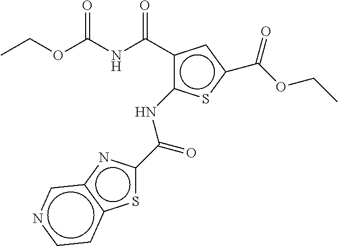 | C | | C | B | | ES-MS M/Z = 449 (M + 1) ¹H NMR (400 MHz, DMSO-d₆) 13.28 (s, 1H), 11.14 (s, 1H), 9.65 (s, 1H), 8.72 (d, J = 5.6 Hz, 1H), 8.65 (s, 1H), 8.43 (d, J = 5.6 Hz, 1H), 4.32 (q, J = 7.1 Hz, 2H), 4.23 (q, J = 7.1 Hz, 2H), 1.33 (t, J = 6.9 Hz, 3H), 1.29 (t, J = 7.1 Hz, 3H). |
| 280 | 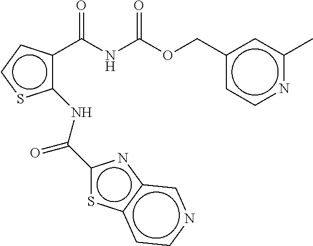 | C | | C | B | | ES-MS M/Z = 454 (M + 1) |
| 281 | 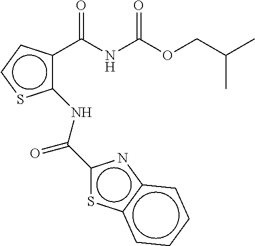 | A | | C | B | | ES-MS M/Z = 404 (M + 1) 1H NMR: 7.40 (d, J = 8.8 Hz, 1H), 7.34 (d, J = 8 Hz, 1H), 6.8 (m, 1H), 6.28 (d, J = 6 Hz, 1H), 3.23 (d, J = 6.8 Hz, 2H), 1.2 (m, 1H), 0.2 (d, J = 6.8 Hz, 6H) |
| 282 | 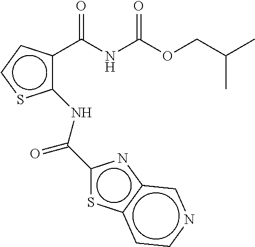 | C | | C | B | | ES-MS M/Z = 404.7 (M + 1) |
| 283 | 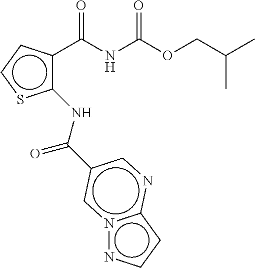 | B | | C | B | | ES-MS M/Z = 388.0 (M + 1) |
| 284 | 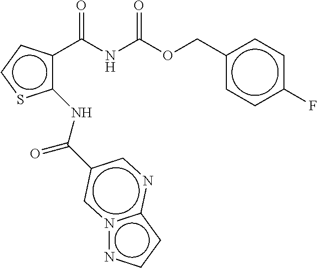 | C | | C | B | | ES-MS M/Z = 439.8 (M + 1) |
| 285 | 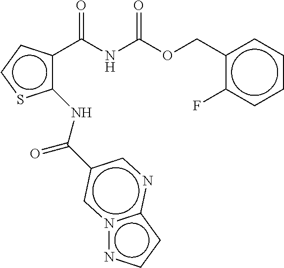 | C | | C | C | | ES-MS M/Z = 440.0 (M + 1) |
| 286 | 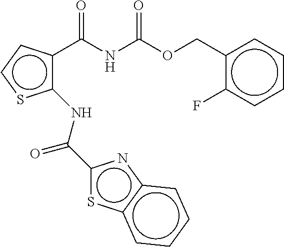 | B | | B | C | | ES-MS M/Z = 445.9 (M + 1) |
| 287 | 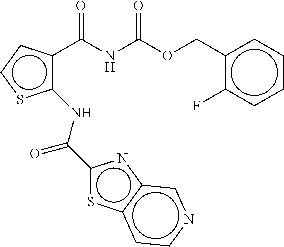 | B | | B | B | | ES-MS M/Z = 457.0 (M + 1) |
| 288 | 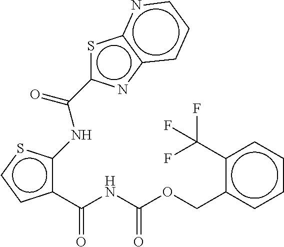 | C | | B | C | | ES-MS M/Z = 506.9 (M + 1) |
| 289 | 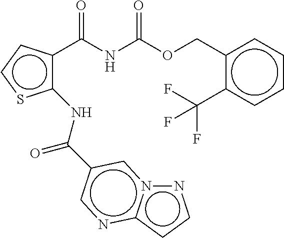 | C | | C | C | | ES-MS M/Z = 490.0 (M + 1) |
| 290 | 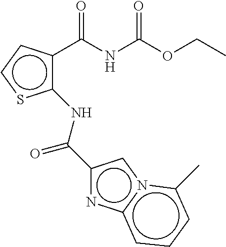 | C | | C | A | | ES-MS M/Z = 373 (M + 1) ¹H NMR (400 MHz, CD₃OD) 1.36 (t, J = 7.06 Hz, 3H) 2.93 (s, 3H) 4.31 (d, J = 7.28 Hz, 2H) 7.07-7.16 (m, 1H) 7.39-7.48 (m, 1H) 7.53-7.61 (m, 1H) 7.79-7.88 (m, 1H) 7.98-8.09 (m, 1H) 8.85-8.95 (m, 1H) |
| 291 | 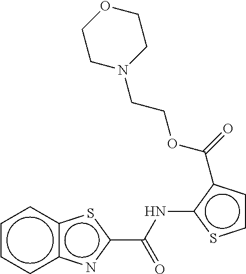 | C | | C | A | | ES-MS M/Z = 418 (M + 1) ¹H NMR (400 MHz, DMSO-d₆) 3.34- 3.40 (m, 2H) 3.66-3.77 (m, 4H) 3.79- 3.90 (m, 2H) 4.08-4.16 (m, 2H) 4.78-4.85 (m, 2H) 7.06-7.14 (m, 1H) 7.40-7.48 (m, 1H) 7.58-7.73 (m, 2H) 8.11-8.26 (m, 2 H) |
| 292 | 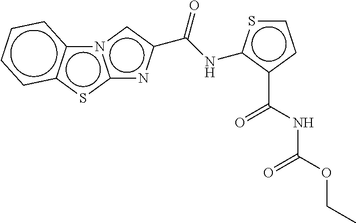 | C | | C | A | | ES-MS M/Z = 414.9 (M + 1) ¹H NMR (400 MHz, DMSO-d₆) 1.32- 1.28 (t, 3H, J = 6.8 Hz), 4.25- 4.20 (q, 2H, J = 6.8 Hz), 7.07- 7.06 (d, 1H, J = 5.6), 7.53- 7.51 (m, 1H), 7.61-7.59 (m, 1H), 7.72- 7.71 (d, 1H, J = 6.0 Hz), 8.13- 8.11 (d, 1H, J = 8.0 Hz), 8.24- 8.22 (d, 1H J = 8.0 Hz), 9.17 (s, 1H), 10.75 (s, 1H), 12.78 (s, 1H). |
| 293 | 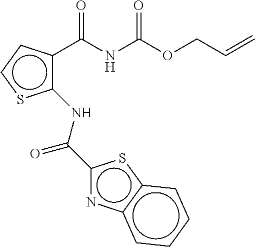 | A | | A | C | | ES-MS M/Z = 388.0 (M + 1) ¹H NMR: 13.07 (s, 1H), 10.95 (s, 1H), 8.32 (dd, J = 9.2 Hz, 2H), 7.79 (d, J = 8.0 Hz, 1H), 7.69 (m, 2H), 7.22 (d, J = 6Hz, 1H), 6.03 (ddt, J = 17.2, 5.2, 4.8 Hz, 1H), 5.45 (dq, J = 17.2, 1.6 Hz, 1H), 5.31 (dq, J = 17.2, 1.6, 1H), 4.72 (dt, J = 5.6, 1.2 Hz, 2H) |
| 294 | 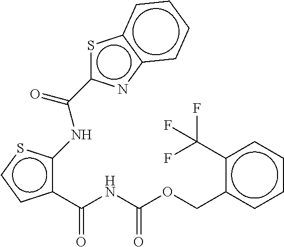 | C | | C | B | | ES-MS M/Z = 505.7 (M + 1) |
| 295 | 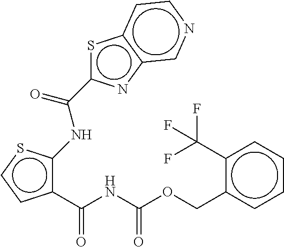 | C | | B | C | | ES-MS M/Z = 506.6 (M + 1) |
| 296 | 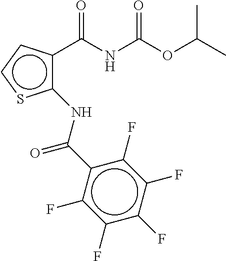 | C | | B | C | | ES-MS M/Z = 422.7 (M + 1) |
| 297 | 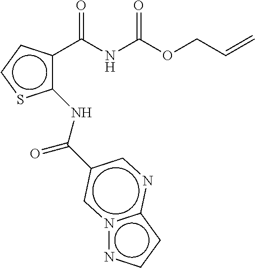 | A | | A | C | | ES-MS M/Z = 394 (M + 24) |
| 298 | 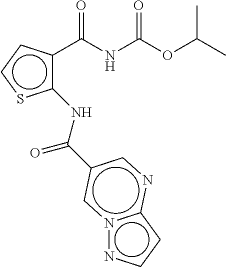 | A | | A | C | | ES-MS M/Z = 374 (M + 1) ¹H NMR (400 MHz, DMSO-d₆) 12.49 (s, 1H), 10.81 (s, 1H), 9.64 (d, J = 2.1 Hz, 1H), 8.93 (d, J = 2.2 Hz, 1H), 8.48 (d, J = 2.3 Hz, 1H), 7.70 (d, J = 5.9 Hz, 1H), 7.14 (d, J = 5.9 Hz, 1H), 6.94 (d, J = 2.3 Hz, 1H), 4.99 (m, 1H), 1.31 (d, J = 6.2 Hz, 6H). |
| 299 | 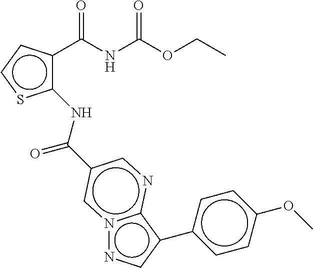 | A | | C | C | | ES-MS M/Z = 466 (M + 1) ¹H NMR (400 MHz, CDCl₃) 12.78 (s, 1H), 9.43 (s, 1H), 9.12 (s, 1H), 8.60 (s, 1H), 8.17-7.80 (m, 3H), 7.23-6.83 (m, 4H), 4.39 (m, 2H), 3.89 (s, 3H), 1.41 (t, J = 7.0 Hz, 3H). |
| 300 | 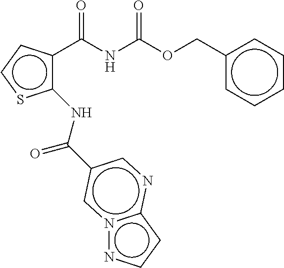 | C | | C | B | | ES-MS M/Z = 443.9 (M + 1) |
| 301 | 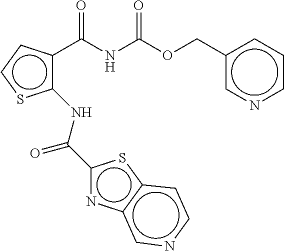 | C | | C | B | | ES-MS M/Z = 439.9 (M + 1) |
| 302 | 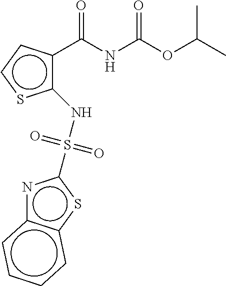 | C | | C | C | | ES-MS M/Z = 447.69 (M + 1) |
| 303 | 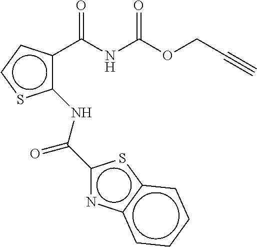 | A | | C | C | | ES-MS M/Z = 407.9 (M + 1) ¹H NMR: 13.05 (s, 1H), 11.08 (s, 1H), 8.33 (dt, J = 8 Hz, 4 Hz, 2H), 7.71 (m, 3H), 7.22 (d, J = 4 Hz, 1H), 4.88 (d, J = 2.4 Hz, 2H), 3.68 (t, J = 4 Hz, 1H) |
| 304 | 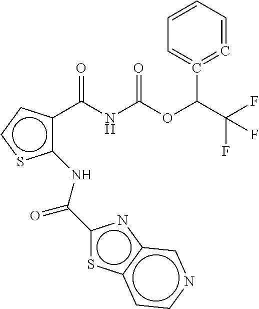 | B | | C | B | | ES-MS M/Z = 506.9 (M + 1) |
| 305 | 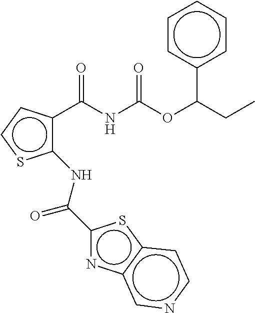 | B | | C | C | | ES-MS M/Z = 467 (M + 1) ¹H NMR (400 MHz, DMSO-d₆) 13.09 (s, 1H), 10.98 (s, 1H), 9.57 (s, 1H), 8.70 (d, J = 5.5 Hz, 1H), 8.38 (d, J = 5.5 Hz, 1H), 7.81 (d, J = 5.9 Hz, 1H), 7.49- 7.38 (m, 5H), 7.37-7.30 (m, 1H), 7.25 (d, J = 6.0 Hz, 1H), 5.69 (t, J = 6.8 Hz, 1H), 2.04-1.81 (m, 2H), 0.93 (t, J = 7.3 Hz, 3H). |
| 306 | 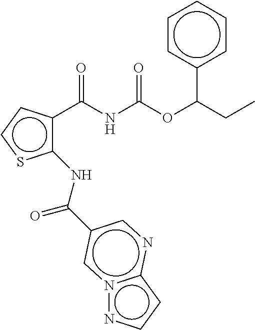 | C | | C | C | | ES-MS M/Z = 472 (M + 1) ¹H NMR (400 MHz, DMSO-d₆) 12.41 (s, 1H), 10.94 (s, 1H), 9.63 (d, J = 2.2 Hz, 1H), 8.92 (d, J = 2.2 Hz, 1H), 8.47 (d, J = 2.3 Hz, 1H), 7.70 (d, J = 5.9 Hz, 1H), 7.48-7.36 (m, 4H), 7.36-7.27 (m, 1H), 7.16 (d, J = 5.9 Hz, 1H), 6.93 (d, J = 2.3 Hz, 1H), 5.69 (t, J = 6.8 Hz, 1H), 1.89 (ddq, J = 28.0, 13.9, 6.9 Hz, 2H), 0.91 (t, J = 7.3 Hz, 3H). |
| 307 | 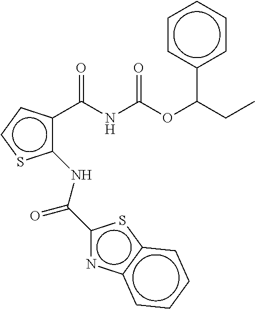 | C | | B | C | | ES-MS M/Z = 488.9 (M + 1) ¹H NMR (400 MHz, DMSO-d₆) 13.05 (s, 1H), 10.95 (s, 1H), 8.39-8.14 (m, 2H), 7.79 (d, J = 5.9 Hz, 1H), 7.73-7.60 (m, 2H), 7.47-7.38 (m, 4H), 7.36-7.31 (m, 1H), 7.23 (dd, J = 8.3, 6.7 Hz, 2H), 7.15 (dd, J = 15.1, 7.1 Hz, 1H), 5.69 (dd, J = 7.5, 6.1 Hz, 1H), 2.01-1.82 (m, 2H), 0.92 (t, J = 7.3 Hz, 3H). |
| 308 | 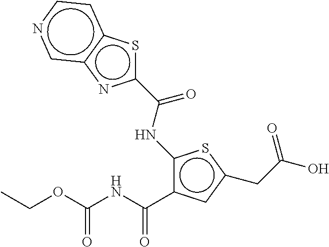 | C | | C | C | | ES-MS M/Z = 434.9 (M + 1) ¹H NMR (400 MHz, DMSO-d₆) 13.06 (s, 1H), 10.84 (s, 1H), 9.61 (s, 1H), 8.70 (d, J = 5.5 Hz, 1H), 8.39 (d, J = 5.5 Hz, 1H), 7.62 (s, 1H), 4.22 (q, J = 7.1 Hz, 2H), 1.29 (t, J = 7.1 Hz, 3H). |
| 309 | 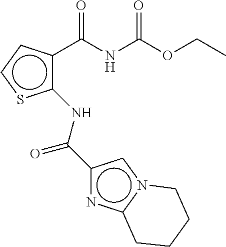 | B | | B | C | | ES-MS M/Z = 363 (M + 1) ¹H NMR (400 MHz, CD₃OD) 1.35 (s, 3H) 1.94-2.29 (m, 4H) 2.98-3.19 (m, 2 H) 4.19-4.38 (m, 4H) 6.91-7.18 (m, 1H) 7.30-7.68 (m, 1H) 8.25 (s, 1H) |
| 310 | 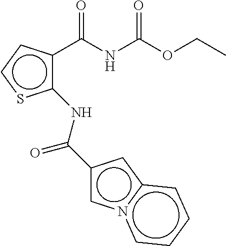 | C | | C | A | | ES-MS M/Z = 358 (M + 1) ¹H NMR (400 MHz, DMSO-d₆) 1.28-1.24 (t, 3H, J = 7.2Hz), 4.21- 4.16 (q, 2H, J = 7.2 Hz), 6.68- 6.67 (m, 1H), 6.81-6.78 (m, 2H), 7.00- 6.99 (d, 1H, J = 6.0 Hz), 7.55- 7.53 (d, 1H, J = 9.2 Hz), 7.69- 7.67 (d, 1H, J = 6.0 Hz), 8.17 (s, 1H), 8.38-8.36 (d, 1H, J = 6.8 Hz), 10.74 (s, 1H), 12.35 (s, 1H). |
| 311 | 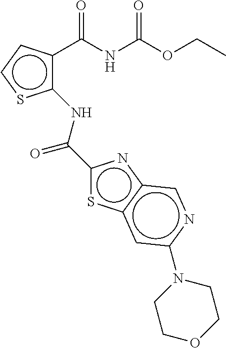 | A | | C | A | | ES-MS M/Z = 462.1 (M + 1) ¹H NMR (400 MHz, DMSO-d₆) 1.36 (t, J = 7.06 Hz, 3H) 3.67 (br. s., 4H) 3.80 (d, J = 4.85 Hz, 4H) 4.30 (q, J = 7.06 Hz, 2H) 7.25 (d, J = 5.73 Hz, 1H) 7.65 (s, 1H) 7.79-7.89 (m, 1H) 9.22 (s, 1H) 10.88- 10.97 (m, 1H) 12.99 -13.10 (m, 1H) |
| 312 | 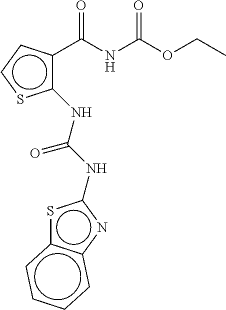 | C | | C | B | | ES-MS M/Z = 391 (M + 1) ¹H NMR (400 MHz, DMSO-d₆) 12.38 (s, 1H), 11.60 (s, 1H), 10.67 (s, 1H), 7.94 (d, J = 7.8 Hz, 1H), 7.69 (s, 1H), 7.63 (d, J = 6.0 Hz, 1H), 7.42 (ddd, J = 8.3, 7.3, 1.3 Hz, 1H), 7.28 (td, J = 7.6, 1.2 Hz, 1H), 6.99 (d, J = 6.0 Hz, 1H), 4.21 (q, J = 7.1 Hz, 2H), 1.29 (t, J = 7.1 Hz, 3H). |
| 313 | 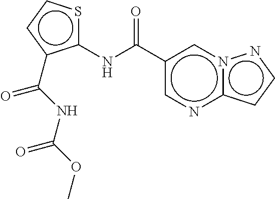 | C | | C | B | | ES-MS M/Z = 346 (M + 1) ¹H NMR (400 MHz, DMSO-d₆) 13.00 (s, 1H), 10.79 (s, 1H), 8.53 (s, 1H), 8.35 (d, J = 9.6 Hz, 1H), 8.01 (s, 1H), 7.84 (d, J = 9.6 Hz, 1H), 7.72 (d, J = 6.0 Hz, 1H), 7.13 (d, J = 6.0 Hz, 1H), 3.98 (s, 3H). |
| 314 | 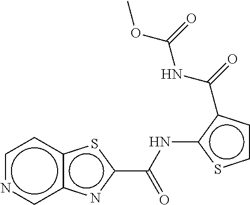 | C | | C | B | | ES-MS M/Z = 363 (M + 1) ¹H NMR (400 MHz, CD₃OD) 9.59 (s, 1H), 8.70 (d, J = 5.8 Hz, 1H), 8.44 (d, J = 5.8 Hz, 1H), 7.57 (d, J = 5.9 Hz, 1H), 7.14 (d, J = 5.9 Hz, 1H), 3.87 (s, 3H). |
| 315 | 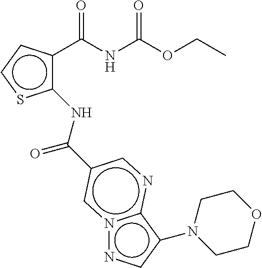 | A | | C | B | | ES-MS M/Z = 445 (M + 1) |
| 316 | 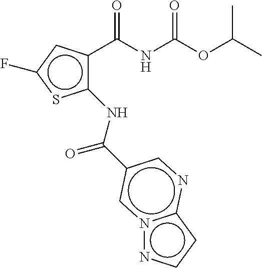 | B | | C | B | | ES-MS M/Z = 392 (M + 1) ¹H NMR (400 MHz, CD₃OD) 9.46 (s, 1H), 8.98 (d, J = 1.8 Hz, 1H), 8.36 (d, J = 3.4 Hz, 1H), 7.54 (d, J = 6.0 Hz, 1H), 7.04 (d, J = 6.1 Hz, 1H), 6.13 (s, 1H), 5.09 (m, 1H), 1.39 (d, J = 6.2 Hz, 6H). |
| 317 | 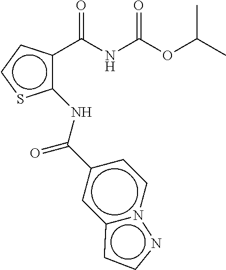 | A | | C | C | | ES-MS M/Z = 373 (M + 1) ¹H NMR (400 MHz, CDCl₃) 10.80 (s, 1H), 8.92 (dd, J = 7.3, 1.0 Hz, 1H), 8.48- 8.31 (m, 1H), 8.18 (d, J = 2.3 Hz, 1H), 7.74 (d, J = 5.9 Hz, 1H), 7.27 (dd, J = 7.3, 2.1 Hz, 1H), 7.11 (d, J = 5.9 Hz, 1H), 7.02 (dd, J = 2.3, 0.9 Hz, 1H), 4.94 (m, 1H), 1.30 (d, J = 6.3 Hz, 6H). |
| 318 | 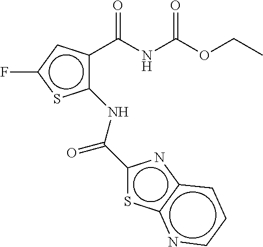 | A | | C | B | | ES-MS M/Z = 395 (M + 1) ¹H NMR (400 MHz, CDCl₃) 13.05 (s, 1H), 8.77 (dd, J = 4.6, 1.5 Hz, 1H), 8.55 (dd, J = 8.3, 1.6 Hz, 1H), 7.74 (s, 1H), 7.61 (dd, J = 8.4, 4.6 Hz, 1H), 6.57 (d, J = 2.7 Hz, 1H), 4.37 (q, J = 7.2 Hz, 2H), 1.40 (t, J = 7.1 Hz, 3H). |
| 319 | 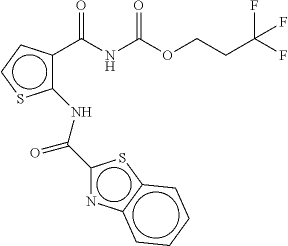 | A | | C | C | | ES-MS M/Z = 443.9 (M + 1) ¹H NMR: 13.06 (s, 1H), 11.03 (s, 1H), 8.32 (dt, J = 7.6, 1.6 Hz, 2H), 7.71 (m, 3H), 7.22 (d, J = 6 Hz, 1H), 4.43 (t, J = 6 Hz, 2H), 2.78 (m, 2H) |
| 320 | 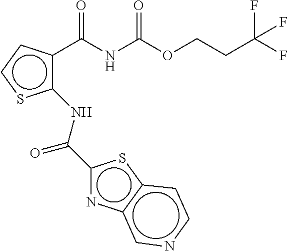 | A | | C | A | | ES-MS M/Z = 444.9 (M + 1) |
| 321 | 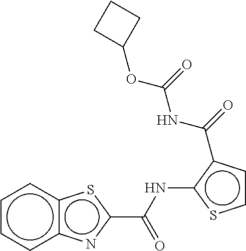 | A | | C | A | | ES-MS M/Z = 401.9 (M + 1) ¹H NMR: 13.09 (s, 1H), 10.89 (s, 1H), 8.32 (dd, J = 1.6, 0.8 Hz, 2H), 7.71 (m, 3H), 7.21 (d, J = 6 Hz, 1H), 5.01 (q, J = 7.2 Hz, 1H), 2.37 (m, 2H), 2.14 (m, 2H), 1.06 (m, 2H) |
| 322 | 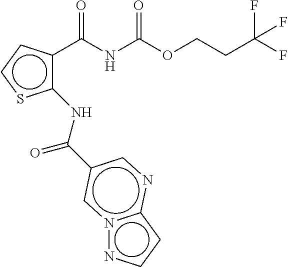 | C | | C | C | | ES-MS M/Z = 428.0 (M + 1) |
| 323 | 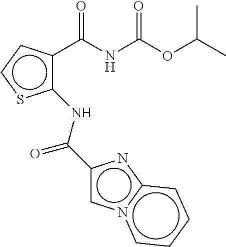 | B | | C | | | ES-MS M/Z = 373 (M + 1) ¹H NMR (400 MHz, CDCl₃) 12.88 (s, 1H), 8.33 (s, 1H), 8.27 (d, J = 7.0 Hz, 1H), 7.96 (s, 1H), 7.88 (d, J = 9.3 Hz, 1H), 7.40 (t, J = 8.0 Hz, 1H), 7.12 (dd, J = 5.9, 2.6 Hz, 1H), 6.99 (d, J = 6.8 Hz, 1H), 6.89 (d, J = 5.9 Hz, 1H), 5.13 (m, 1H), 1.37 (d, J = 6.3 Hz, 6H). |
| 324 | 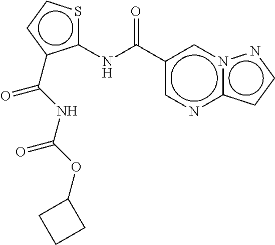 | A | | C | | | ES-MS M/Z = 386.0 (M + 1) ¹H NMR: 12.47 (s, 1H), 10.87 (s, 1H), 9.64 (d, J = 2.4 Hz, 1H), 8.93 (d, J = 2 Hz, 1H), 8.48 (d, J = 2Hz, 1H), 7.70 (d, J = 6 Hz, 1H), 7.14 (d, J = 5.6 Hz, 1H), 6.94 (d, J = 2 Hz, 1H), 5.00 (p, J = 7.2 Hz, 1H), 2.35 (m, 2H, 2.12 (m, 2H), 1.70 (m, 2H) |
| 325 | 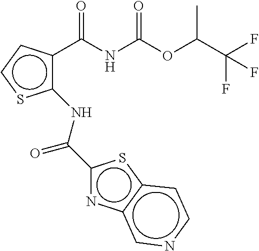 | A | | C | | | ES-MS M/Z = 445.0 (M + 1) ¹H NMR: 13.03 (s, 1H), 11.29 (s, 1H), 9.66 (d, J = 0.8 Hz, 1H), 8.72 (d, J = 5.6 Hz, 1H), 8.44 (dd, J = 5.6, 0.8 Hz, 1H), 7.81 (d, J = 6 Hz, 1H), 7.25 (d, J = 5.6 Hz, 1H) 5.57 (hept, J = 6.8 Hz, 1H), 1.48 (d, J = 6.8 Hz, 3H) |
| 326 | 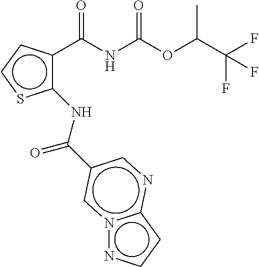 | C | | C | | | ES-MS M/Z = 427.9 (M + 1) |
| 327 | 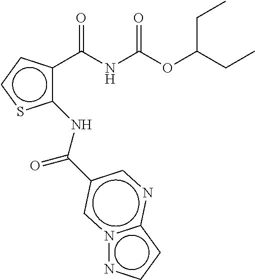 | B | | C | C | | ES-MS M/Z = 802.8 (2M + 1) |
| 328 | 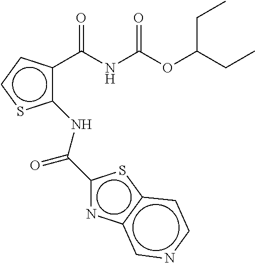 | A | | C | C | | ES-MS M/Z = 419.0 (M + 1) ¹H NMR: 13.14 (s, 1H), 10.86 (s, 1H), 9.64 (d, J = 0.8 Hz, 1H), 8.72 (d, J = 5.6 Hz, 1H), 8.43 (dd, J = 6, 1.2 Hz, 1H), 7.81 (d, J = 6 Hz, 1H), 7.24 (d, J = 5.6 Hz, 1H), 4.74 (m, 1H), 1.65 (m, 4H), 0.93 (t, J = 7.2 Hz, 6H) |
| 329 | 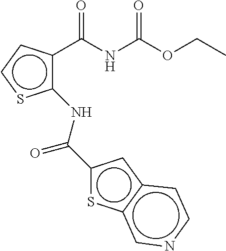 | A | | C | C | | ES-MS M/Z = 376 (M + 1) ¹H NMR (400 MHz, CD₃OD) 1.36 (s, 3H) 4.21-4.37 (m, 2H) 7.03-7.13 (m, 1H) 7.53-7.62 (m, 1H) 8.43-8.50 (m, 1H) 8.51-8.58 (m, 1H) 8.66 -8.76 (m, 1H) 9.64-9.75 (m, 1H) |
| 330 | 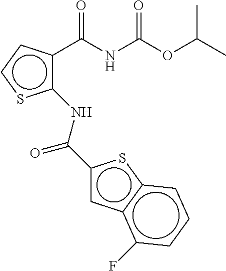 | A | | B | C | | ES-MS M/Z = 428.9 (M + 23) ¹H NMR (400 MHz, DMSO-d₆) 1.17- 1.32 (m, 6H) 4.89-5.00 (m, 1H) 7.06- 7.14 (m, 1H) 7.28-7.38 (m, 1H) 7.51-7.60 (m, 1H) 7.65-7.72 (m, 1H) 7.92-8.00 (m, 1H) 8.09-8.15 (m, 1H) 10.75-10.83 (m, 1H) 12.60- 12.63 (m, 1H) |
| 331 | 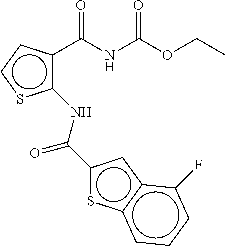 | A | | B | C | | ES-MS M/Z = 414.8 (M + 23) ¹H NMR (400 MHz, DMSO-d₆) 1.22- 1.30 (m, 3H) 4.15-4.24 (m, 2H) 7.08- 7.13 (m, 1H) 7.28-7.36 (m, 1H) 7.53-7.60 (m, 1H) 7.67-7.71 (m, 1H) 7.93-7.99 (m, 1H) 8.11 (s, 1H) 10.82-10.86 (m, 1H) 12.59-12.63 (m, 1H) |
| 332 | 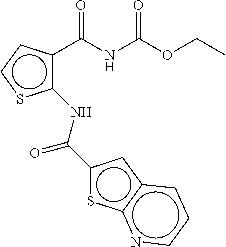 | A | | C | A | | ES-MS M/Z = 417.1 (M + 41) ¹H NMR (400 MHz, DMSO-d₆) 1.26 (t, J = 7.04 Hz, 3H) 4.14-4.25 (m, 2H) 7.11 (d, J = 5.67 Hz, 1H) 7.50-7.60 (m, 1H) 7.67-7.76 (m, 1H) 8.19 (s, 1H) 8.50-8.59 (m, 1H) 8.67- 8.75 (m, 1H) 10.79-10.89 (m, 1H) 12.61-12.68 (m, 1H) |
| 333 | 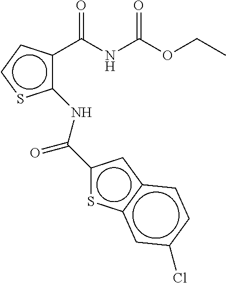 | A | | B | B | | ES-MS M/Z = 409 (M + 1) ¹H NMR (400 MHz, DMSO-d₆) 1.24- 1.29 (m, 3H) 4.14-4.26 (m, 2H) 7.10 (d, J = 5.87 Hz, 1H) 7.53 (s, 1H) 7.71 (d, J = 5.87 Hz, 1H) 8.11-8.16 (m, 1H) 8.17-8.20 (m, 1H) 8.26-8.31 (m, 1H) 10.77-10.87 (m, 1H) 12.56-12.64 (m, 1H) |
| 334 | 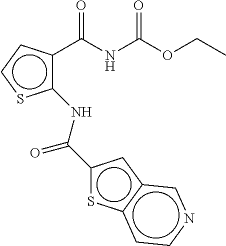 | A | | C | A | | ES-MS M/Z = 376.1 (M + 1) ¹H NMR (400 MHz, DMSO-d₆) 1.26 (s, 3H) 4.15-4.25 (m, 2H) 7.11-7.19 (m, 1H) 7.70-7.77 (m, 1H) 8.48- 8.55 (m, 1H) 8.59-8.66 (m, 1H) 8.68- 8.75 (m, 1H) 9.61-9.69 (m, 1H) 10.84-0.93 (m, 1H) 12.69-2.78 (m, 1H) |
| 335 | 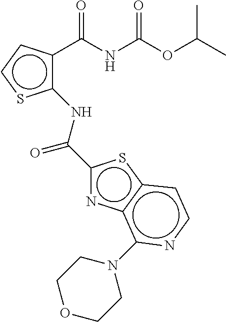 | | | | | | ES-MS M/Z = 476.1 (M + 1) ¹H NMR (400 MHz, DMSO-d₆) 1.28 (d, J = 5.87 Hz, 6H) 3.88 (br. s., 4H) 4.18 (br. s., 4H) 4.92-5.04 (m, 1H) 7.10-7.19 (m, 1H) 7.54-7.63 (m, 1H) 7.73-7.81 (m, 1H) 8.05-8.15 (m, 1H) 10.70-10.80 (m, 1H) 13.33-13.43 (m, 1H) |
| 336 | 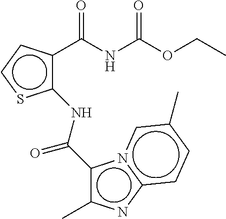 | C | | C | B | | ES-MS M/Z = 387 (M + 1) |
| 337 | 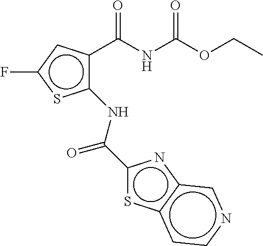 | A | | C | B | | ES-MS M/Z = 395 (M + 1) ¹H NMR (400 MHz, CDCl₃) 13.13 (s, 1H), 9.62 (d, J = 0.9 Hz, 1H), 8.76 (d, J = 5.8 Hz, 1H), 8.13 (d, J = 5.8 Hz, 1H), 7.76 (s, 1H), 6.60 (d, J = 2.7 Hz, 1H), 4.38 (q, J = 7.1 Hz, 2H), 1.41 (t, J = 7.1 Hz, 3H). |
| 338 | 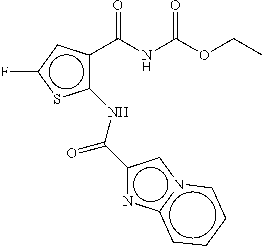 | A | | C | B | | ES-MS M/Z = 377 (M + 1) ¹H NMR (400 MHz, DMSO-d₆) 12.79 (s, 1H), 10.76 (d, J = 7.1 Hz, 1H), 8.41 (dt, J = 6.9, 1.2 Hz, 1H), 7.75-7.68 (m, 1H), 7.50-7.39 (m, 2H), 7.17-7.08 (m, 2H), 4.24-4.18 (m, 2H), 1.30 (t, J = 7.2 Hz, 3H). |
| 339 | 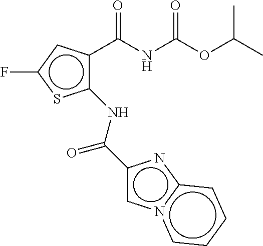 | A | | C | B | | ES-MS M/Z = 391 (M + 1) ¹H NMR (400 MHz, DMSO-d₆) 12.80 (s, 1H), 10.70 (d, J = 9.2 Hz, 1H), 8.41 (dt, J = 7.0,1.2 Hz, 1H), 7.82-7.72 (m, 1H), 7.51-7.39 (m, 2H), 7.19-7.05 (m, 2H), 4.90 (m, H), 1.31 d, J = 6.3 Hz, 6H). |
| 340 | 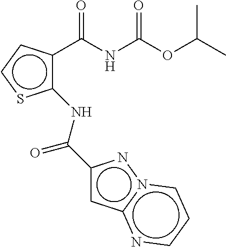 | A | | C | B | | ES-MS M/Z = 374 (M + 1) ¹H NMR (400 MHz, CD₃OD) 9.07 (ddd, J = 7.2, 1.7, 0.9 Hz, 1H), 8.65 (dd, J = 4.1, 1.7 Hz, 1H), 7.53 (d, J = 5.9 Hz, 1H), 7.27 (d, J = 0.9 Hz, 1H), 7.21 (dd, J = 7.1, 4.1 Hz, 1H), 7.03 (d, J = 5.9 Hz, 1H), 5.10 (m, 1H), 1.38 (d, J = 6.3 Hz, 6H). |
| 341 | 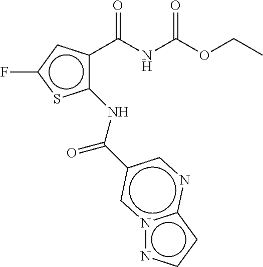 | A | | C | B | | ES-MS M/Z = 378 (M + 1) |
| 342 | 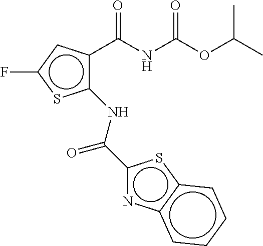 | A | | B | B | | ES-MS M/Z = 408 (M + 1) ¹H NMR (400 MHz, CDCl₃) 13.03 (s, 1H), 8.27 (ddd, J = 8.3, 1.3, 0.7 Hz, 1H), 8.01 (ddd, J = 8.0, 1.3, 0.6 Hz, 1H), 7.75 (s, 1H), 7.59 (dddd, J = 26.5, 8.3, 7.2, 1.3 Hz, 2H), 6.60 (dd, J = 2.8, 0.8 Hz, 1H), 5.14 (hept, J = 6.2 Hz, 1H), 1.37 (d, J = 6.3 Hz, 6H). |
| 343 | 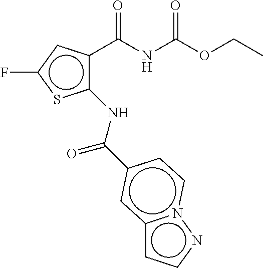 | A | | C | C | | ES-MS M/Z = 377 (M + 1) ¹H NMR (400 MHz, DMSO-d₆) δ 12.64 (s, 1H), 10.87 (s, 1H), 8.82 (dt, J = 7.4, 1.2 Hz, 1H), 8.55-8.27 (m, 2H), 7.74 (d, J = 5.9 Hz, 1H), 7.27 (dd, J = 7.4, 2.0 Hz, 1H), 7.14 (d, J = 5.9 Hz, 1H), 4.23 (q, J = 7.1 Hz, 2H), 1.30 (t, J = 7.1 Hz, 3H). |
| 344 | 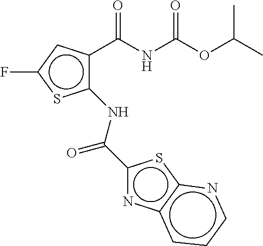 | A | | C | B | | ES-MS M/Z = 409 (M + 1) ¹H NMR (400 MHz, CDCl₃) 13.07 (s, 1H), 8.77 (dd, J = 4.6, 1.6 Hz, 1H), 8.54 (dd, J = 8.4, 1.6 Hz, 1H), 7.68 (s, 1H), 7.61 (dd, J = 8.3, 4.6 Hz, 1H), 6.58 (d, J = 2.7 Hz, 1H), 5.15 (p, J = 6.2 Hz, 1H), 1.38 (d, J = 6.3 Hz, 6H). |
| 345 | 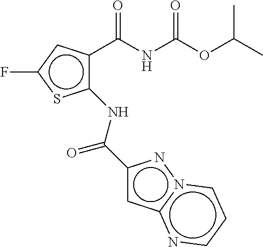 | A | | C | B | | ES-MS M/Z = 392 (M + 1) ¹H NMR (400 MHz, CDCl₃) 12.86 (s, 1H), 9.05-8.77 (m, 1H), 8.77-8.50 (m, 1H), 7.74 (s, 1H), 7.36 (d, J = 0.8 Hz, 1H), 7.03 (dd, J = 7.1, 4.0 Hz, 1H), 6.57 (d, J = 2.8 Hz, 1H), 5.13 (p, J = 6.2 Hz, 1H), 1.36 (d, J = 6.2 Hz, 6H). |
| 346 | 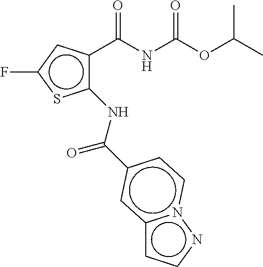 | B | | C | C | | ES-MS M/Z = 391 (M + 1) ¹H NMR (400 MHz, CDCl₃) 12.72 (s, 1H), 8.47-8.39 (m, 1H), 8.33 (dd, J = 2.0, 1.0 Hz, 1H), 7.94 (d, J = 3.7 Hz, 1H), 7.90 (s, 1H), 7.35 (dd, J = 7.4, 2.1 Hz, 1H), 7.11 (d, J = 5.9 Hz, 1H), 6.96 (d, J = 5.9 Hz, 1H), 5.16 (p, J = 6.2 Hz, 1H), 1.39 (d, J = 6.2 Hz, 6H). |
| 347 | 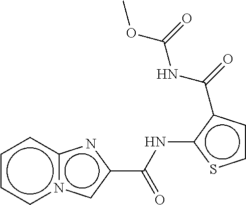 | C | | C | B | | ES-MS M/Z = 345 (M + 1) ¹H NMR (400 MHz, DMSO-d₆) 12.85 (s, 1H), 10.66 (s, 1H), 8.75-8.50 (m, 2H), 7.78 (d, J = 9.2 Hz, 1H), 7.69 (d, J = 5.9 Hz, 1H), 7.41 (dd, J = 9.3, 6.8 Hz, 1H), 7.10-7.05 (m, 2H), 3.82 (s, 3H). |
| 348 | 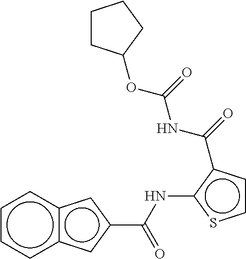 | C | | C | B | | ES-MS M/Z = 399 (M + 1) ¹H NMR (400 MHz, DMSO-d₆) 12.87 (s, 1H), 10.67 (s, 1H), 8.76-8.50 (m, 2H), 7.80 (dd, J = 9.2 Hz, 1H), 7.71 (d, J = 5.9 Hz, 1H), 7.43 (dd, J = 9.3, 6.8 Hz, 1H), 7.11-7.04 (m, 2H), 5.23-5.13 (m, 1H), 1.97-1.88 (m, 2H), 1.81-1.69 (m, 4H), 1.60 (m, 2H). |
| 349 | 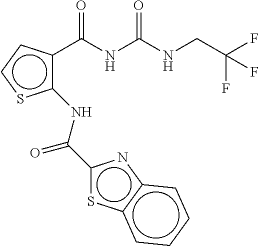 | C | | C | B | | ES-MS M/Z = 429 (M + 1) ¹H NMR (400 MHz, CDCl₃) 12.89 (s, 1H), 9.10 (s, 1H), 8.43 (s, 1H), 8.32 (d, J = 8.2 Hz, 1H), 8.05 (d, J = 8.0 Hz, 1H), 7.69-7.52 (m, 2H), 7.23 (d, J = 6.0 Hz, 1H), 7.02 (d, J = 5.9 Hz, 1H), 4.18- 4.07 (m, 2H). |
| 350 | 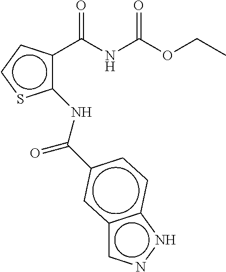 | C | | C | B | | ES-MS M/Z = 359.0 (M + 1) |
| 351 | 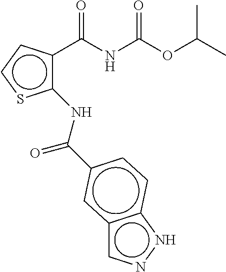 | C | | C | B | | ES-MS M/Z = 372.9 (M + 1) |
| 352 | 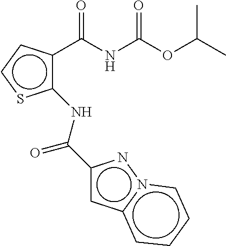 | A | | C | B | | ES-MS M/Z = 395.0 (M + 23) |
| 353 | 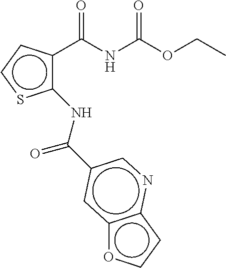 | B | | C | B | | ES-MS M/Z = 359.9 (M + 1) |
| 354 | 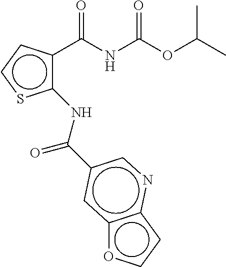 | C | | C | B | | ES-MS M/Z = 374.0 (M + 1) |
| 355 | 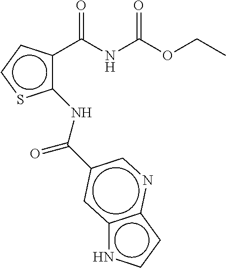 | C | | C | B | | ES-MS M/Z = 359.0 (M + 1) |
| 356 | 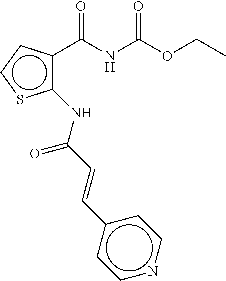 | C | | C | B | | ES-MS M/Z = 346.0 (M + 1) |
| 357 | 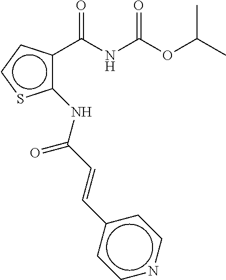 | | | | | | ES-MS M/Z = 360.0 (M + 1) |
| 358 | 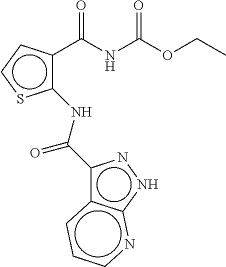 | C | | C | B | | ES-MS M/Z = 360.0 (M + 1) |
| 359 | 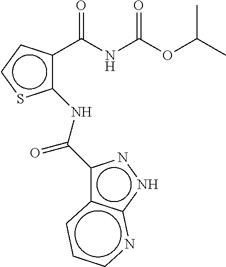 | C | | C | B | | ES-MS M/Z = 374.0 (M + 1) |
| 360 | 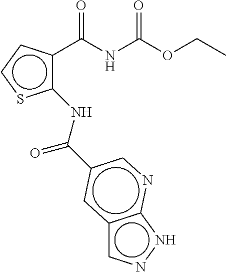 | | | | | | ES-MS M/Z = 360.0 (M + 1) |
| 361 | 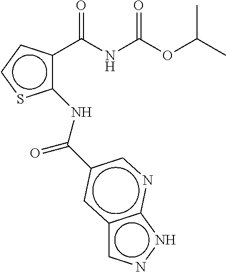 | | | | | | ES-MS M/Z = 374.0 (M + 1) |
| 362 | 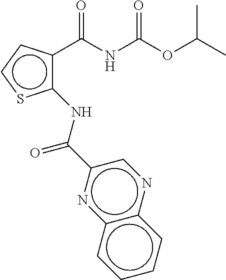 | | | | | | ES-MS M/Z = 385.0 (M + 1) |
| 363 | 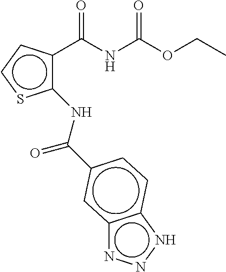 | C | | C | B | | ES-MS M/Z = 360.0 (M + 1) |
| 364 | 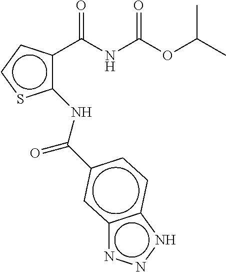 | B | | C | B | | ES-MS M/Z = 374.0 (M + 1) |
| 365 | 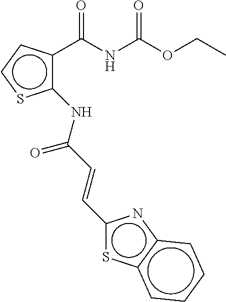 | A | | C | C | | ES-MS M/Z = 401.9 (M + 1) |
| 366 | 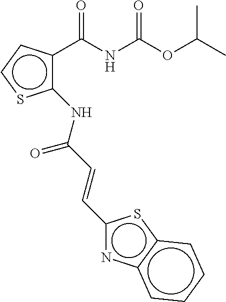 | A | | B | C | | ES-MS M/Z = 416.0 (M + 1) |
| 367 | 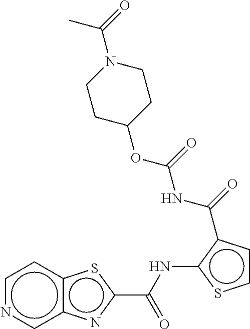 | C | | C | B | | ES-MS M/Z = 474 (M + 1) |
| 368 | 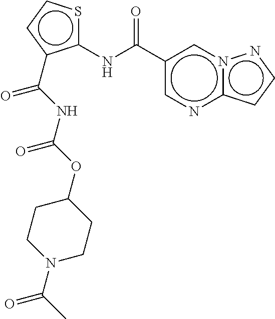 | C | | C | B | | ES-MS M/Z = 457 (M + 1) |
| 369 | 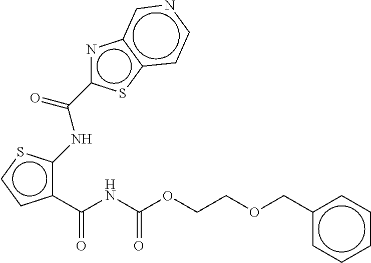 | A | | C | B | | ES-MS M/Z = 482.9 (M + 1) ¹H NMR (400 MHz, DMSO-d₆) 13.12 (s, 1H), 11.02 (s, 1H), 9.59 (d, J = 1.0 Hz, 1H), 8.70 (d, J = 5.6 Hz, 1H), 8.40 (dd, J = 5.6, 1.0 Hz, 1H), 7.81 (d, J = 5.9 Hz, 1H), 7.36 (s, 2H), 7.35 (d, J = 3.5 Hz, 2H), 7.34-7.25 (m, 2H), 7.23 (d, J = 5.9 Hz, 1H), 4.56 (s, 2H), 4.40-4.31 (m, 2H), 3.76-3.71 (m, 2H). |
| 370 | 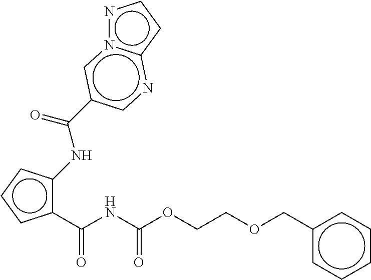 | A | | C | B | | ES-MS M/Z = 466 (M + 1) |
| 371 | 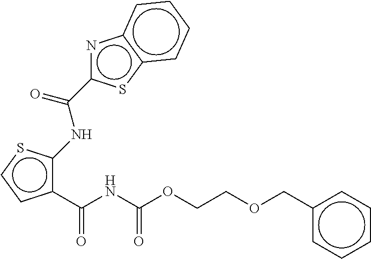 | A | | B | C | | ES-MS M/Z = 482 (M + 1) ¹H NMR (400 MHz, DMSO-d₆) 13.10 (s, 1H), 11.00 (s, 1H), 8.44-8.20 (m, 2H), 7.81 (d, J = 5.9 Hz, 1H), 7.74-7.62 (m, 2H), 7.40-7.33 (m, 4H), 7.32-7.25 (m, 1H), 7.21 (dd, J = 5.9, 0.8 Hz, 1H), 4.57 (s, 2H), 4.41-4.30 (m, 2H), 3.81- 3.66 (m, 2H). |
| 372 | 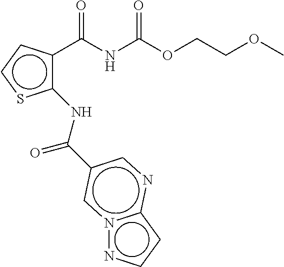 | A | | C | B | | ES-MS M/Z = 390 (M + 1) |
| 373 | 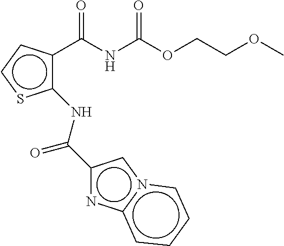 | C | | C | B | | ES-MS M/Z = 389 (M + 1) |
| 374 | 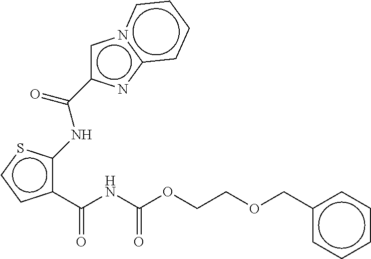 | A | | C | B | | ES-MS M/Z = 465 (M + 1) |
| 375 | 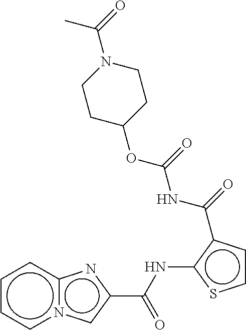 | C | | C | B | | ES-MS M/Z = 456 (M + 1) |
| 376 | 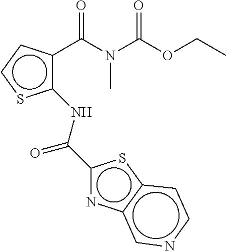 | C | | C | C | | ES-MS M/Z = 391 (M + 1) ¹H NMR (400 MHz, DMSO-d₆) 11.46 (s, 1H), 9.50 (s, 1H), 8.67 (s, 1H), 8.35 (d, J = 4.7 Hz, 1H), 8.32 (d, J = 5.5 Hz, 1H), 7.35 (d, J = 5.9 Hz, 1H), 6.96 (dd, J = 5.8, 0.8 Hz, 1H), 4.20 (q, J = 7.1 Hz, 2H), 2.76 (d, J = 4.5 Hz, 3H), 1.26 (t, J = 7.1 Hz, 3H). |
| 377 | 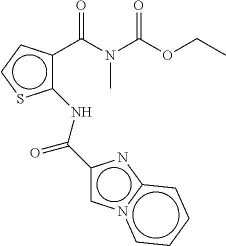 | C | | C | B | | ES-MS M/Z = 373 (M + 1) |
| 378 | 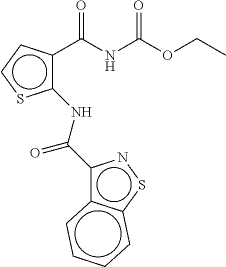 | C | | C | B | | ES-MS M/Z = 375.9 (M + 1) |
| 379 | 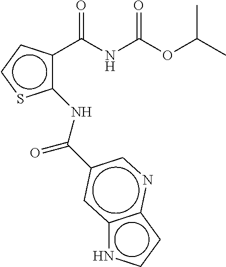 | C | | C | B | | ES-MS M/Z = 398.8 (M + 1) |
| 380 | 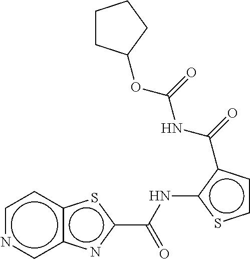 | A | | C | C | | ES-MS M/Z = 417 (M + 1) ¹H NMR (400 MHz, CD₃OD) 9.52 (s, 1H), 8.66 (d, J = 5.7 Hz, 1H), 8.35 (d, J = 5.7 Hz, 1H), 7.59 (d, J = 5.9 Hz, 1H), 7.12 (d, J = 5.9 Hz, 1H), 5.34-5.23 (m, 1H), 2.05-1.94 (m, 2H), 1.93-1.78 (m, 4H), 1.70 (m, 2H). |
| 381 | 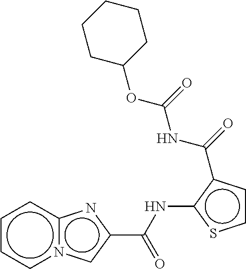 | C | | C | C | | ES-MS M/Z = 413 (M + 1) ¹H NMR (400 MHz, DMSO-d₆) 12.87 (s, 1H), 10.68 (s, 1H), 8.76-8.54 (m, 2H), 7.80 (dd, J = 9.2, 1.1 Hz, 1H), 7.72 (d, J = 5.9 Hz, 1H), 7.43 (ddd, J = 9.3, 6.8, 1.3 Hz, 1H), 7.12-7.03 (m, 2H), 4.81- 4.64 (m, 1H), 1.93 (m, 1H), 1.75 (d, J = 10.5 Hz, 2H), 1.60-1.18 (m, 7H). |
| 382 | 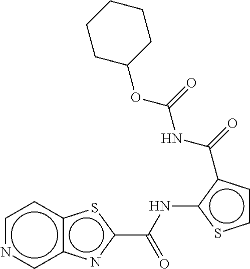 | A | | C | C | | ES-MS M/Z = 413 (M + 1) ¹H NMR (400 MHz, DMSO-d₆) 12.87 (s, 1H), 10.68 (s, 1H), 8.76-8.54 (m, 2H), 7.80 (dd, J = 9.2, 1.1 Hz, 1H), 7.72 (d, J = 5.9 Hz, 1H), 7.43 (ddd, J = 9.3, 6.8, 1.3 Hz, 1H), 7.12-7.03 (m, 2H), 4.81- 4.64 (m, 1H), 1.93 (m, 1H), 1.75 (d, J = 10.5 Hz, 2H), 1.60-1.18 (m, 7H). |
| 383 | 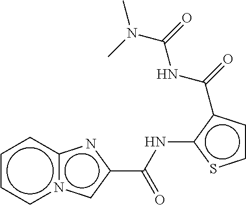 | C | | C | C | | ES-MS M/Z = 358 (M + 1) ¹H NMR (400 MHz, DMSO-d₆) 12.88 (s, 1H), 10.02 (s, 1H), 8.65 (m, 2H), 7.76 (d, J = 9.2 Hz, 1H), 7.55 (d, J = 6.0 Hz, 1H), 7.43 (m, 1H), 7.10 (d, J = 5.7 Hz, 1H), 7.06 (m, 1H), 2.99 (s, 6H). |
| 384 | 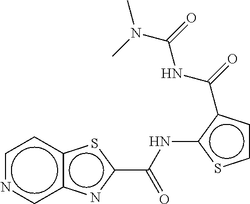 | C | | C | C | | ES-MS M/Z = 376 (M + 1) ¹H NMR (400 MHz, DMSO-d₆) 13.21 (s, 1H), 10.19 (s, 1H), 9.57 (s, 1H), 8.69 (d, J = 5.5 Hz, 1H), 8.37 (d, J = 5.6 Hz, 1H), 7.63 (d, J = 6.2 Hz, 1H), 7.26 (d, J = 6.0 Hz, 1H), 3.00 (s, 6H). |
| 385 | 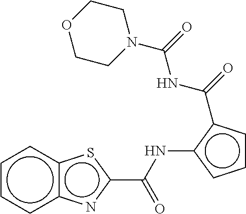 | C | | C | C | | ES-MS M/Z = 417 (M + 1) ¹H NMR (400 MHz, DMSO-d₆) 13.15 (s, 1H), 10.23 (s, 1H), 8.38-8.24 (m, 2H), 7.74-7.65 (m, 2H), 7.63 (d, J = 5.9 Hz, 1H), 7.24 (d, J = 5.8 Hz, 1H), 3.68-3.62 (m, 4H), 3.54-3.47 (m, 4H). |
| 386 | 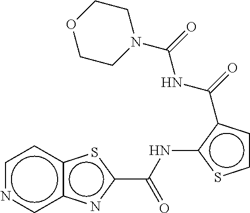 | C | | C | C | | ES-MS M/Z = 418 (M + 1) ¹H NMR (400 MHz, DMSO-d₆) 13.18 (s, 1H), 10.26 (s, 1H), 9.57 (s, 1H), 8.70 (d, J = 5.5 Hz, 1H), 8.37 (d, J = 5.5 Hz, 1H), 7.64 (d, J = 5.9 Hz, 1H), 7.27 (d, J = 5.8 Hz, 1H), 3.69-3.60 (m, 4H), 3.51 (m, 4H). |
| 387 | 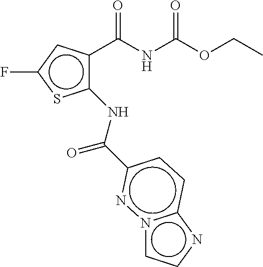 | B | | C | C | | ES-MS M/Z = 378 (M + 1) ¹H NMR (400 MHz, CDCl₃) 13.20 (s, 1H), 8.25 (d, J = 9.5 Hz, 1H), 8.02 (d, J = 9.7 Hz, 1H), 7.91 (s, 1H), 7.64 (d, J = 6.7 Hz, 1H), 7.13 (s, 1H), 7.01 (d, J = 6.1 Hz, 1H), 4.38 (q, J = 7.1 Hz, 2H), 1.40 (t, J = 7.2 Hz, 3H). |
| 388 | 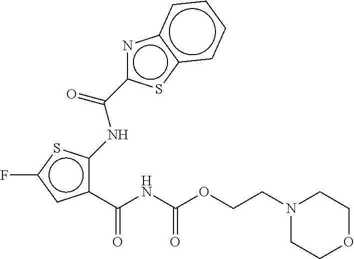 | A | | C | C | | ES-MS M/Z = 479 (M + 1) |
| 389 | 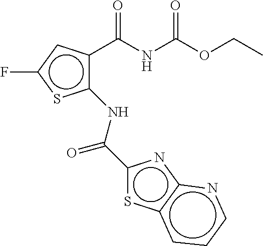 | B | | C | C | | ES-MS M/Z = 395 (M + 1) ¹H NMR (400 MHz, DMSO-d₆) 13.17 (s, 1H), 10.80 (s, 1H), 8.91 (dd, J = 4.5, 1.7 Hz, 1H), 8.82 (d, J = 8.2 Hz, 1H), 7.67 (dd, J = 8.2, 4.5 Hz, 1H), 7.52 (s, 1H), 4.24 (q, J = 7.1 Hz, 2H), 1.41 (t, J = 7.2 Hz, 3H). |
| 390 | 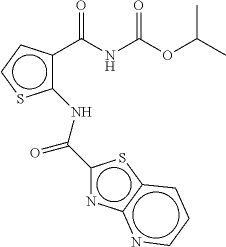 | A | | C | C | | ES-MS M/Z = 391 (M + 1) ¹H NMR (400 MHz, CDCl₃) 13.11 (s, 1H), 8.93 (dd, J = 4.5, 1.7 Hz, 1H), 8.41 (dd, J = 8.1, 1.7 Hz, 1H), 7.81 (s, 1H), 7.50 (dd, J = 8.2, 4.5 Hz, 1H), 7.13 (dd, J = 5.9, 0.8 Hz, 1H), 7.01 (dd, J = 5.9, 0.8 Hz, 1H), 5.16 (h, J = 6.2 Hz, 1H), 1.39 (d, J = 6.3 Hz, 6H). |
| 391 | 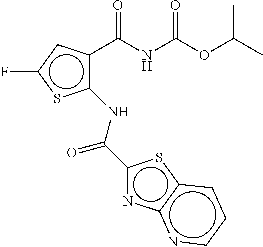 | A | | C | C | | ES-MS M/Z = 409 (M + 1) ¹H NMR (400 MHz, DMSO-d₆) 13.17 (s, 1H), 10.75 (s, 1H), 8.92 (dd, J = 4.4, 1.6 Hz, 1H), 8.84 (s, 1H), 7.68 (dd, J = 8.2, 4.5 Hz, 1H), 7.53 (s, 1H), 5.00 (p, J = 6.3 Hz, 1H), 1.32 (d, J = 6.2 Hz, 6H). |
| 392 | 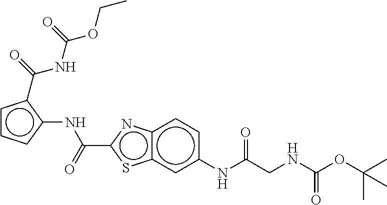 | B | | C | B | | ES-MS M/Z = 548 (M + 1) ¹H NMR (400 MHz, CDCl₃) 12.94 (s, 1H), 8.78 (s, 1H), 8.67-8.51 (m, 1H), 8.18-8.08 (m, 2H), 7.42 (dd, J = 8.9, 2.1 Hz, 1H), 7.15 (d, J = 5.9 Hz, 1H), 6.95 (d, J = 5.8 Hz, 1H), 5.37 {s, 1H), 4.36 (q, J = 7.1 Hz, 2H), 4.01 (d, J = 5.3 Hz, 2H), 1.52 (s, 9H), 1.39 (t, J = 7.1 Hz, 3H). |
| 393 | 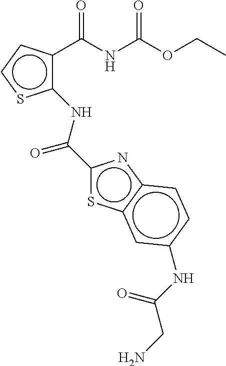 | C | | C | C | | ES-MS M/Z = 448 (M + 1) ¹H NMR (400 MHz, CD₃OD) 8.65 (d, J = 2.1 Hz, 1H), 8.15 (d, J = 8.9 Hz, 1H), 7.61 (dd, J = 8.8, 2.0 Hz, 1H), 7.56 (d, J = 5.9 Hz, 1H), 7.09 (d, J = 5.9 Hz, 1H), 4.33 (q, J = 7.1 Hz, 2H), 3.96 (s, 2H), 1.38 (t, J = 7.1 Hz, 3H). |
| 394 | 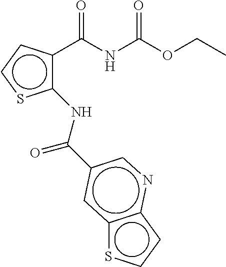 | A | | C | C | | ES-MS M/Z = 376.0 (M + 1) |
| 395 | 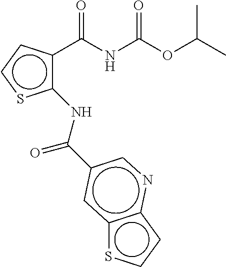 | A | | C | C | | ES-MS M/Z = 389.9 (M + 1) ¹H NMR: 12.68 (s, 1H), 10.81 (s, 1H), 9.17 (d, J = 2 Hz, 1H), 9.09 (d, J = 3.6 Hz, 1H), 8.46 (d, J= 5.46 Hz, 1H), 7.74 (m, 2H), 7.12 (d, J = 5.6 hz, 1H), 4.99 (hept, J = 6.4 Hz, 1H), 1.31 (d, J = 6.4 Hz, 6H) |
| 396 | 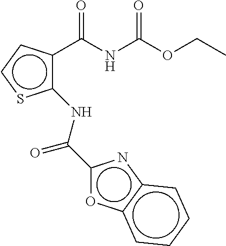 | A | | C | C | | ES-MS M/Z = 360.0 (M + 1) ¹H NMR: 13.10 (s, 1H), 10.89 (s, 1H), 8.07 (d, J = 7.6 Hz, 1H), 7.97 (d, J = 8 Hz, 1H), 7.80 (d, J = 6 Hz, 1H), 7.61 (m, 2H), 7.22 (d, J = 6 Hz, 1H), 4.24 (q, J = 6.8 Hz, 2H), 1.31 (t, J = 6.8 Hz, 3H) |
| 397 | 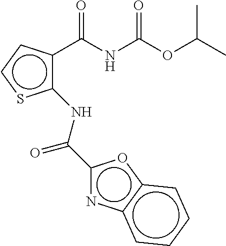 | A | | C | C | | ES-MS M/Z = 373.9 (M + 1) ¹H NMR: 13.10 (s, 1H), 10.84 (s, 1H), 8.07 (d, J = 7.6 Hz, 1H), 7.97 (d, J = 8 Hz, 1H), 7.80 (d, J = 5.6 Hz, 1H), 7.61 (dtd, J = 28,8.4, 1.2 Hz, 2H), 7.21 (d, J = 6 Hz, 1H), 5.00 (hept, J = 6.4,1H), 6.12 (d, J = 8.4, 6.4, 1.2 Hz) |
| 398 | 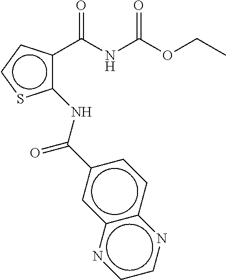 | A | | B | C | | ES-MS M/Z = 371.0 (M + 1) ¹H NMR: 12.87 (s, 1H), 10.88 (s, 1H), 9.11 (q, J = 1.6 Hz, 2H), 8.66 (t, J = 4 Hz, 1H), 8.35 (d, J = 1.2, 2H), 7.77 (d, J = 6 Hz, 1H), 7.16 (d, J = 5.6 Hz, 1H), 4.24 (q, J = 7.2 Hz, 2H), 1.30 (t, J = 6.8 Hz, 3H) |
| 399 | 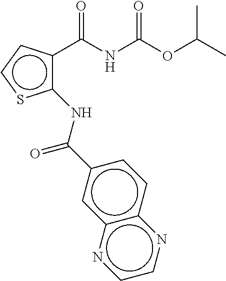 | A | | C | C | | ES-MS M/Z = 385.0 (M + 1) ¹H NMR: 12.88 (s, 1H), 10.83 (s, 1H), 9.12 (q, J = 1.6 Hz, 2H), 8.67 (t, J = 1.2 Hz, 1H), 8.35 (d, J = 0.8 Hz, 2H), 7.77 (d, J = 6 Hz, 1H), 7.15 (d, J = 6 Hz, 1H), 4.99 (hept, J = 6.4 Hz, 1H), 1.31 (d, J = 6.4 Hz, 6H) |
| 400 | 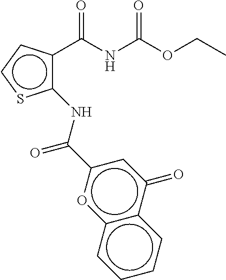 | A | | C | C | | ES-MS M/Z = 386.9 (M + 1) |
| 401 | 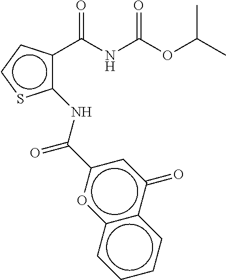 | A | | C | C | | ES-MS M/Z = 401.0 (M + 1) ¹H NMR: 13.05 (s, 1H), 10.86 (s, 1H), 8.11 (dd, J = 7.6, 1.6 Hz, 1H), 7.98 (ddd, J = 7.6, 2, 1.6 Hz, 1H), 7.64 (m, 2H), 7.22 (d, J = 5.6 Hz, 1H), 7.01 (s, 1H), 5.00 (hept, J = 6 Hz, 1H), 1.32 (d, J = 6 Hz, 6H) |
| 402 | 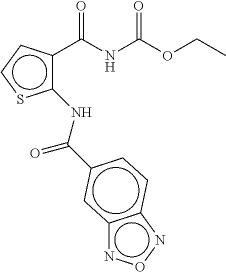 | A | | C | C | | ES-MS M/Z = 360.9 (M + 1) |
| 403 | 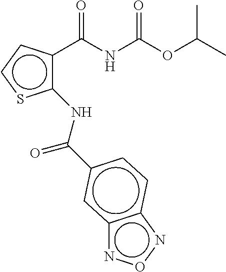 | A | | C | C | | ES-MS M/Z = 375.0 (M + 1) ¹H NMR: 12.65 (s, 1H), 10.84 (s, 1H), 8.71 (t, J = 0.8 Hz, 1H), 8.31 (d, J = 10 Hz, 1H), 7.99 (d, J = 10 Hz, 1H), 7.74 (d, J = 6 Hz, 1H), 7.17 (d, J = 6 Hz, 1H), 4.99 (hept, J = 6.4 Hz, 1H), 1.31 (d, J = 6.4 Hz, 6H) |
| 404 | 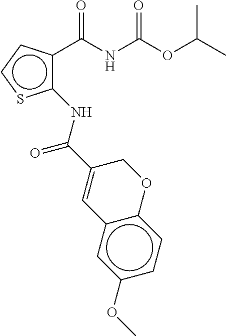 | A | | B | C | | ES-MS M/Z = 417.0 (M + 1) |
| 405 | 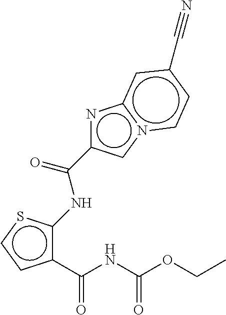 | B | | C | C | | ES-MS M/Z = 383.9 (M + 1) ¹H NMR (400 MHz, DMSO-d₆) 12.92 (s, 1H), 10.78 (s, 1H), 8.83 (s, 1H), 8.79 (dd, J = 7.2, 1.0 Hz, 1H), 8.67-8.63 (m, 1H), 7.73 (d, J = 6.0 Hz, 1H), 7.36 (dd, J = 7.1, 1.6 Hz, 1H), 7.11 (d, J = 5.8 Hz, 1H), 4.23 (q, J = 7.1 Hz, 2H), 1.30 (t, J = 7.1 Hz, 3H). |
| 406 | 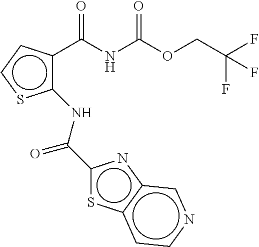 | A | | C | C | | ES-MS M/Z = 430.8 (M + 1) ¹H NMR (400 MHz, DMSO-d₆) 13.03 (s, 1H), 11.33 (s, 1H), 9.60 (s, 1H), 8.71 (d, J = 5.5 Hz, 1H), 8.39 (d, J = 5.5 Hz, 1H), 7.81 (d, J = 5.8 Hz, 1H), 7.26 (d, J = 5.9 Hz, 1H), 4.92 (q, J = 9.1 Hz, 2H). |
| 407 | 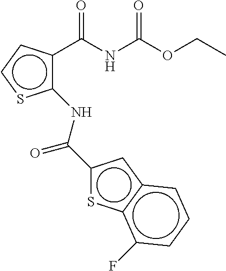 | A | | B | C | | ES-MS M/Z = 393.1 (M + 1) ¹H NMR (400 MHz, DMSO-d₆) 1.26 (br. s., 3 H) 4.12-4.26 (m, 2H) 7.06-7.15 (m, 1H) 7.39-7.47 (m, 1H) 7.48- 7.58 (m, 1H) 7.67-7.74 (m, 1H) 7.95- 8.05 (m, 1H) 8.22-8.30 (m, 1H) 10.76-10.92 (m, 1H) 12.55-12.72 (m, 1H) |
| 408 | 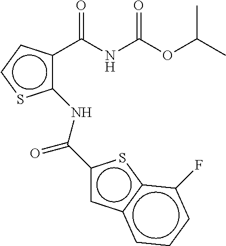 | A | | B | C | | ES-MS M/Z = 407.1 (M + 1) ¹H NMR (400 MHz, DMSO-d₆) 1.27 (d, J = 5.87 Hz, 6 H) 4.95 (br. s., 1H) 7.05-7.16 (m, 1H) 7.38-7.57 (m, 2H) 7.70 (d, J = 5.48 Hz, 1H) 7.95-8.06 (m, 1H) 8.22-8.31 (m, 1H) 10.73- 10.85 (m, 1H) 12.55-12.69 (m, 1H) |
| 409 | 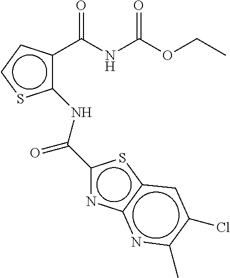 | A | | A | B | | ES-MS M/Z = 466.1 (M + 41) ¹H NMR (400 MHz, DMSO-d₆) 1.28- 1.36 (m, 3H) 2.65 (s, 3H) 4.09-4.21 (m, 2H) 6.63-6.73 (m, 1H) 7.13- 7.21 (m, 1H) 8.61-8.70 (m, 1H) 13.04-13.13 (m, 1H) |
| 410 | 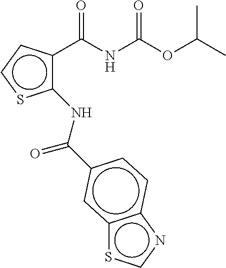 | A | | B | C | | ES-MS M/Z = 389.9 (M + 1) ¹H NMR (400 MHz, DMSO-d₆) 1.29 (d, J = 6.26 Hz, 6H) 4.92-5.01 (m, 1H) 7.14-7.21 (m, 1H) 7.72-7.78 (m, 1H) 8.39-8.43 (m, 1H) 8.44-8.48 (m, 1H) 8.70-8.76 (m, 1H) 9.70 (s, 1H) 10.83-10.90 (m, 1H) 12.76-12.84 (m, 1H) |
| 411 | 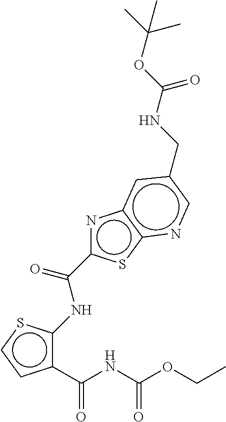 | A | | B | B | | ES-MS M/Z = 528 (M + 23) ¹H NMR (400 MHz, DMSO-d₆) 13.09 (s, 1H), 10.85 (s, 1H), 8.70 (s, 1H), 8.52 (s, 1H), 7.75 (d, J = 5.87 Hz, 1H), 7.56- 7.63 (m, 1H), 7.19 (d, J = 5.48 Hz, 1H), 4.35 (d, J = 5.48 Hz, 2H), 4.16-4.25 (m, 2H), 1.32-1.45 (m, 6H), 1.27 (t, J = 7.04 Hz, 3H). |
| 412 | 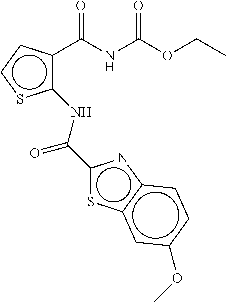 | A | | C | A | | ES-MS M/Z = 406.1 (M + 1) 1H NMR (400 MHz, DMSO-d6) 1.29- 1.38 (m, 3H) 3.96 (s, 3H) 4.30 (m, 2H) 7.24-7.26 (m, 1H) 7.35 (d, J = 8.40 Hz, 1H) 7.83 (d, J = 6.0 Hz, 1H) 7.93 (s, 1H) 8.26 (d, J = 8.8 Hz, 1H) 10.92 (s, 1H) 13.07 (s, 1H) |
| 413 | 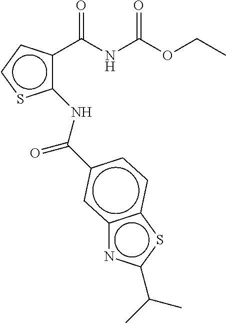 | A | | C | A | | ES-MS M/Z = 439.9 (M + 23) ¹H NMR (400 MHz, DMSO-d₆) 1.29 (t, J = 7.04 Hz, 3H) 1.44 (d, J = 7.04 Hz, 6H) 3.44-3.53 (m, 1H) 4.15-4.28 (m, 2H) 7.11 (d, J = 5.87 Hz, 1H) 7.69-7.80 (m, 1H) 7.89-8.03 (m, 1H) 8.28- 8.37 (m, 1H) 8.42-8.52 (m, 1H) 10.86 (s, 1H) 12.69-12.84 (m, 1H). |
| 414 | 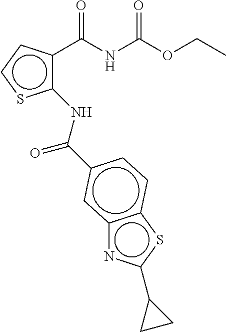 | A | | C | A | | ES-MS M/Z = 437.8 (M + 23) ¹H NMR (400 MHz, DMSO-d₆) 1.17- 1.41 (m, 7H) 2.55-2.64 (m, 1H) 4.22 (d, J = 7.04 Hz, 2H) 7.10 (d, J = 5.48 Hz, 1H) 7.74 (d, J = 5.87 Hz, 1H) 7.92 (s, 1H) 8.22-8.30 (m, 1H) 8.34 (s, 1H) 10.86 (s, 1H) 12.62-12.81 (m, 1H). |
| 415 | 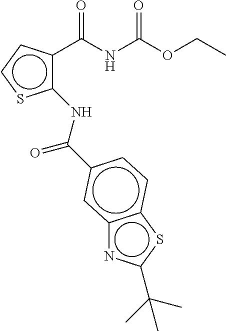 | C | | C | A | | ES-MS M/Z = 431.9 (M + 1) 1H NMR (400 MHz, DMSO-d6) 1.26 (t, J = 7.04 Hz, 3H) 1.47 (s, 9H) 4.11-4.30 (m, 2H) 7.08 (d, J = 5.48 Hz, 1H) 7.71 (d, J = 5.87 Hz, 1H) 7.94 (d, J = 7.43 Hz, 1H) 8.30 (d, J = 8.22 Hz, 1H) 8.43 (s, 1H) 10.73-10.93 (m, 1H) 12.70 -12.79 (m, 1H). |
| 416 | 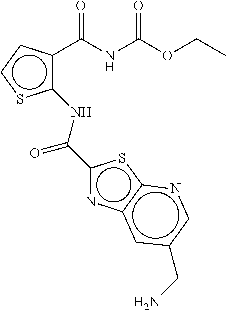 | B | | C | A | | ES-MS M/Z = 427.9 (M + 23) ¹H NMR (400 MHz, DMSO-d₆) 13.16 (s, 1H), 10.89 (s, 1H), 8.93 (d, J = 5.2 Hz, 2H), 8.67 (s, 3H), 7.79 (d, J = 6 Hz, 1H), 7.22 (d, J = 6 Hz, 1H), 4.31 (d, J = 5.6 Hz, 2H), 4.22 (m, 2H), 1.30 (t, J = 7.2 Hz, 3H). |
| 417 | 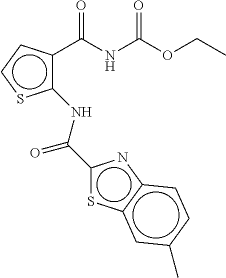 | | | | | | ES-MS M/Z = 390 (M + 1) ¹H NMR (400 MHz, DMSO-d₆) 1.18- 1.34 (m, 3 H) 2.30-2.46 (m, 3H) 4.20 (q, J = 7.04 Hz, 2H) 7.12-7.19 (m, 1H) 7.49 (d, J = 8.61 Hz, 1H) 7.74 (d, J = 5.87 Hz, 1H) 8.06 (s, 1H) 8.16 (d, J = 8.22 Hz, 1H) 10.84 (s, 1H) 13.02 (s, 1H) |
| 418 | 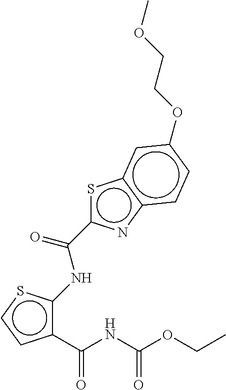 | | | | | | ES-MS M/Z = 450.1 (M + 1) ¹H NMR (400 MHz, DMSO-d₆) 1.14- 1.40 (m, 3H) 3.29 (s, 3H) 3.60-3.76 (m, 2H) 4.05-4.44 (m, 4H) 7.06- 7.35 (m, 2H) 7.65-7.90 (m, 2 H) 8.15 (d, J = 9.00 Hz, 1H) 10.83 (s, 1H) 12.87- 13.15 (m, 1H) |
| 419 | 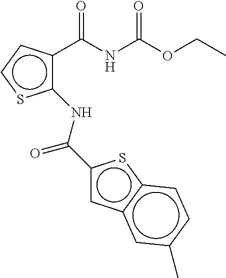 | | | | | | ES-MS M/Z = 389.1 (M + 1) ¹H NMR (400 MHz, DMSO-d₆) 12.59 (s, 1H), 10.83 (s, 1H), 7.90-8.09 (m, 3H), 7.71 (d, J = 4 Hz, 1H), 7.36 (d, J = 8 Hz, 1H), 7.09 (d, J = 4 Hz, 1H), 4.17-4.21(m, 2H), 2.42 (s, 3H), 1.26 (t, J = 6 Hz, 3H). |
| 420 | 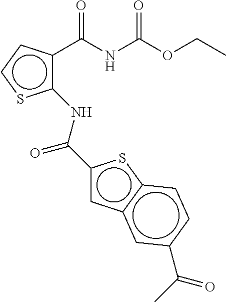 | | | | | | ES-MS M/Z = 417.2 (M + 1) ¹H NMR (400 MHz, DMSO-d₆) 12.67 (s, 1H), 10.86 (s, 1H), 8.83 (s, 1H), 8.22- 8.33 (m, 2H), 8.02 (d, J = 8 Hz, 1H), 7.72 (d, J = 8 Hz, 1H), 7.11 (d, J = 8 Hz, 1H), 4.17-4.23 (dd, J = 8, 16 Hz, 2H), 2.65 (s, 3H), 1.27 (t, J = 6 Hz, 3H). |
| 421 | 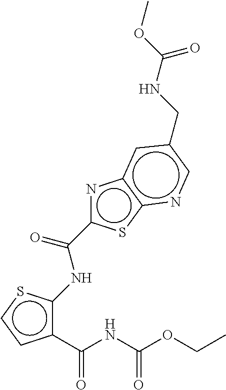 | | | | | | ES-MS M/Z = 485.9 (M + 23) ¹H NMR (400 MHz, DMSO-d₆) 13.08 (s, 1H), 10.85 (s, 1H), 8.71 (s, 1H), 8.54 (s, 1H), 7.82-7.90 (m, 1H), 7.75 (d, J = 6.0 Hz, 1H), 7.17 (d, J = 5.6 Hz, 1H), 4.40 (d, J = 5.6 Hz, 2H), 4.20 (dd, J = 6.8 Hz, 2H), 3.55 (s, 3H), 1.27 (t, J = 8.8 Hz, 3H). |
| 422 | 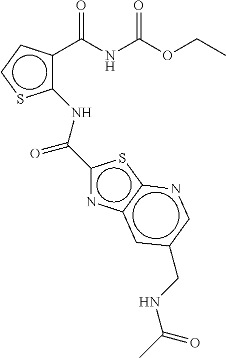 | | | | | | ES-MS M/Z = 447.9 (M + 1) ¹H NMR (400 MHz, DMSO-d₆) 13.09 (s, 1H), 10.86 (s, 1H), 8.71 (s, 1H), 8.54 (d, J = 6.4 Hz, 2 H), 7.74-7.78 (m, 1H), 7.17-7.21 (m, 1H), 4.45-4.48 (d, J = 5.6 Hz, 2H), 4.17-4.23 (m, 2H), 1.89 (s, 3H) 1.25-1.29 (t, J = 6.8 Hz, 3H) |
| 423 | 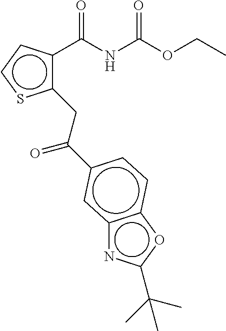 | | | | | | ES-MS M/Z = 416 (M + 1) ¹H NMR (400 MHz, CDCl₃) 1.31-1.41 (m, 3H) 1.50 (s, 9H) 4.23-4.42 (m, 2H) 6.72- 6.92 (m, 1H) 6.99-7.09 (m, 1H) 7.51-7.66 (m, 1H) 7.76-7.88 (m, 1H) 7.98-8.12 (m, 1H) 8.36-8.54 (m, 1H) 12.51-12.83 (m, 1H). |
| 424 | 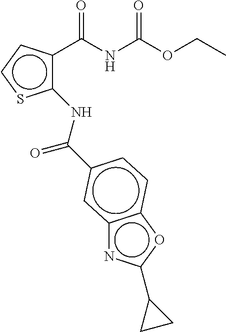 | | | | | | ES-MS M/Z = 399.9 (M + 1) ¹H NMR (400 MHz, CDCl₃) 1.16 (d, J = 5.48 Hz, 2H) 1.21-1.38 (m, 5H) 2.09-2.24 (m, 1H) 4.28 (d, J = 7.04 Hz, 2H) 6.82 (d, J = 5.87 Hz, 1H) 6.99 (d, J = 5.87 Hz, 1H) 7.38-7.53 (m, 1H) 7.74-7.87 (m, 1H) 7.87-7.99 (m, 1H) 8.23-8.39 (m, 1H) 12.13-12.88 (m, 1H). |
| 425 | 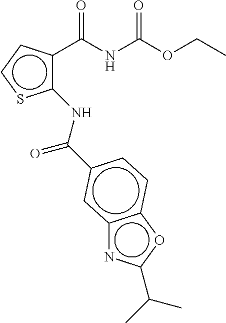 | | | | | | ES-MS M/Z = 401.9 (M + 1) ¹H NMR (400 MHz, CDCl₃) 1.36 (s, 2H) 1.47 (d, J = 6.65 Hz, 6H) 3.15-3.38 (m, 1H) 4.15-4.44 (m, 1H) 6.88 (s, 1H) 7.04 (d, J = 5.87 Hz, 1H) 7.58 (s, 1H) 7.83-7.92 (m, 1H) 7.95-8.08 (m, 1H) 8.33-8.50 (m, 1H) 12.54-12.77 (m, 1H). |
| 426 | 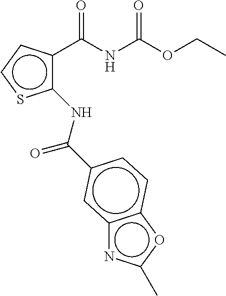 | | | | | | ES-MS M/Z = 395.9 (M + 23) ¹H NMR (400 MHz, CDCl₃) 1.31 (s, 3H) 2.63 (s, 3H) 4.28 (d, J = 7.04 Hz, 2H) 6.80-6.85 (m, 1H) 6.97-7.01 (m, 1H) 7.48-7.55 (m, 1H) 7.79-7.84 (m, 1H) 7.93-8.01 (m, 1H) 8.32-8.38 (m, 1H) 12.53-12.60 (m, 1H). |
| 427 | 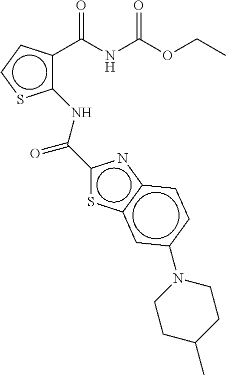 | | | | | | ES-MS M/Z = 473.2 (M + 1) ¹H NMR (400 MHz, DMSO-d₆) 0.91 (d, J = 6.65 Hz, 3H) 1.26 (t, J = 7.04 Hz, 5H) 1.56 (br. s., 1H) 1.68 (d, J = 12.52 Hz, 2H) 2.82 (t, J = 11.54 Hz, 2H) 3.87 (d, J = 12.52 Hz, 2H) 4.19 (q, J = 7.04 Hz, 2H) 7.13 (d, J = 5.48 Hz, 1H) 7.36 (d, J = 7.43 Hz, 1H) 7.63 (s, 1H) 7.72 (d, J = 5.87 Hz, 1H) 8.03 (d, J = 9.39 Hz, 1H) 10.82 (s, 1H) 12.83-12.98 (m, 1H) |
| 428 | 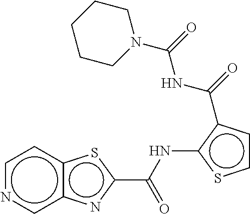 | B | | C | A | | ES-MS M/Z = 416 (M + 1) ¹H NMR (400 MHz, DMSO-d₆) 13.23 (s, 1H), 10.18 (s, 1H), 9.59 (s, 1H), 8.70 (d, J = 5.5 Hz, 1H), 8.40 (d, J = 5.6 Hz, 1H), 7.64 (d, J = 5.9 Hz, 1H), 7.26 (d, J = 5.8 Hz, 1H), 3.68 (m, 4H), 1.70-1.47 (m, 6H). |
| 429 | 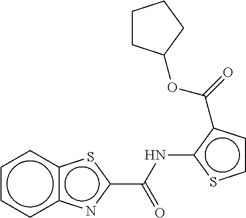 | C | | C | A | | ES-MS M/Z = 373 (M + 1) ¹H NMR(400 MHz, DMSO-d₆) 12.28 (s, 1H), 8.33 (d, J = 7.8 Hz, 1H), 8.26 (d, J = 8.5 Hz, OH), 7.70 (dddd, J = 20.5, 8.3, 7.2, 1.3 Hz, 2H), 7.29 (d, J = 5.7 Hz, 1H), 7.22 (d, J= 5.7 Hz, 1H), 5.49- 5.36 (m, 1H), 1.99 (m, 1H), 1.95-1.77 (m, 3H), 1.67 (m, 1H). |
| 430 | 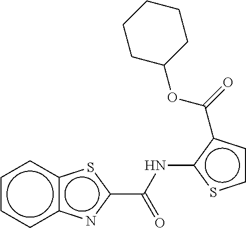 | C | | C | A | | ES-MS M/Z = 387 (M + 1) ¹H NMR (400 MHz, DMSO-d₆) 12.37 (s, 1H), 8.33 (d, J = 7.4 Hz, 1H), 8.26 (d, J = 7.5 Hz, 1H), 7.71 (dt, J = 13.8, 7.5 Hz, 2H), 7.31 (d, J = 5.7 Hz, 1H), 7.23 (d, J = 5.7 Hz, 1H), 5.06 (tt, J = 8.7, 3.8 Hz, 1H), 2.04-1.88 (m, 2H), 1.86-1.71 (m, 2H), 1.62 (m, 3H), 1.49-1.31 (m, 3H). |
| 431 | 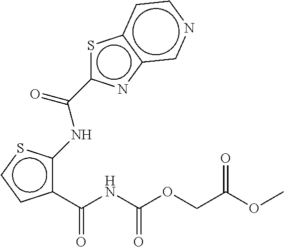 | A | | C | B | | ES-MS M/Z = 421 (M + 1) ¹H NMR (400 MHz, DMSO-d₆) 13.08 (s, 1H), 11.24 (s, 1H), 9.60 (s, 2H), 8.71 (d, J = 5.8 Hz, 1H), 8.39 (d, J = 5.7 Hz, 1H), 7.82 (d, J = 6.0 Hz, 1H), 7.26 (d, J = 5.8 Hz, 1H), 4.85 (s, 2H), 3.74 (s, 3H). |
| 432 | 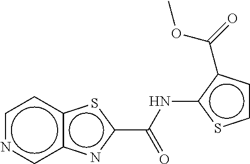 | C | | C | B | | ES-MS M/Z = 320 (M + 1) ¹H NMR (400 MHz, DMSO-d6) 12.48 (s, 1H), 9.60 (s, 1H), 8.71 (d, J = 5.6 Hz, 1H), 8.41 (d, J = 5.6, 1H), 7.33 (d, J = 5.7 Hz, 1H), 7.26 (d, J = 5.7 Hz, 1H), 3.94 (s, 3H). |
| 433 | 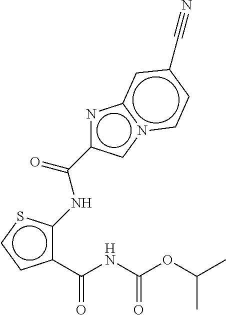 | B | | C | B | | ES-MS M/Z = 398 (M + 1) 1H NMR (400 MHz, DMSO-d6) 12.91 (s, 1H), 10.72 (s, 1H), 9.41 (d, J = 1.6 Hz, 1H), 8.70 (s, 1H), 7.99 (d, J = 9.5 Hz, 1H), 7.73 (d, J = 6.0 Hz, 1H), 7.70 (dd, J = 9.5, 1.7 Hz, 1H), 7.10 (d, J = 5.9 Hz, 1H), 6.54 (s, 0H), 4.98 (p, J = 6.3 Hz, 1H), 1.30 (d, J = 6.2 Hz, 6H). |
| 434 | 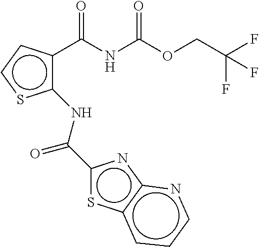 | C | | C | B | | ES-MS M/Z = 430.9 (M + 1) ¹H NMR (400 MHz, DMSO-d₆) 13.09 (s, 1H), 11.31 (s, 1H), 8.92 (dd, J = 4.5, 1.7 Hz, 1H), 8.84 (dd, J = 8.2, 1.7 Hz, 1H), 7.82 (d, J = 5.9 Hz, 1H), 7.69 (dd, J = 8.2, 4.5 Hz, 1H), 7.26 (d, J = 5.8 Hz, 1H), 4.93 (q, J = 9.1 Hz, 2H). |
| 435 | 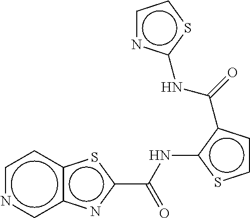 | C | | C | B | | ES-MS M/Z = 387.9 (M + 1) ¹H NMR (400 MHz, DMSO-d₆) 13.18 (s, 1H), 9.66 (s, 1H), 8.73 (d, J = 5.6 Hz, 1H), 8.47 (s, 1H), 7.98 (d, J = 5.7 Hz, 1H), 7.62 (d, J = 3.6 Hz, 1H), 7.38 (d, J = 3.6 Hz, 1H), 7.30 (d, J = 5.8 Hz, 1H). |
| 436 | 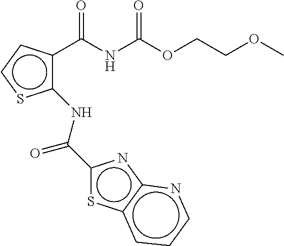 | C | | C | B | | ES-MS M/Z = 407 (M + 1) ¹H NMR (400 MHz, DMSO-d₆) 13.18 (s, 1H), 11.00 (s, 1H), 8.92 (dd, J = 4.6, 1.7 Hz, 1H), 8.83 (dd, J = 8.2, 1.7 Hz, 1H), 7.82 (d, J = 5.9 Hz, 1H), 7.68 (dd, J = 8.2, 4.6 Hz, 1H), 7.23 (d, J = 5.9 Hz, 1H), 4.37-4.22 (m, 2H), 3.70-3.52 (m, 2H), 3.30 (s, 3H). |
| 437 | 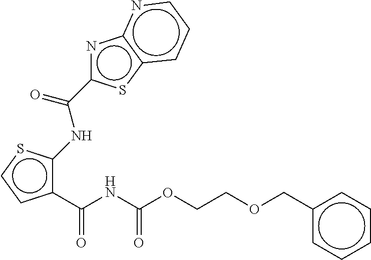 | A | | B | C | | ES-MS M/Z = 482.9 (M + 1) ¹H NMR (400 MHz, DMSO-d₆) 13.18 (s, 1H), 11.00 (s, 1H), 8.92 (dd, J = 4.6, 1.7 Hz, 1H), 8.83 (dd, J = 8.2, 1.7 Hz, 1H), 7.81 (d, J = 5.9 Hz, 1H), 7.68 (dd, J = 8.2, 4.5 Hz, 1H), 7.36 (s, 2H), 7.35 (d, J = 1.9 Hz, 2H), 7.34-7.25 (m, 1H), 7.23 (d, J = 5.8 Hz, 1H), 4.55 (s, 2H), 4.40-4.31 (m, 2H), 3.76-3.70 (m, 2H). |
| 438 | 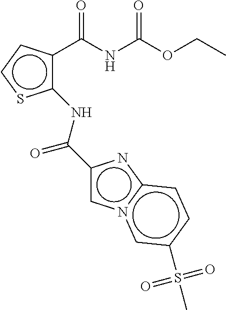 | A | | C | B | | ES-MS M/Z = 436.9 (M + 1) ¹H NMR (400 MHz, DMSO-d₆) 12.93 (s, 1H), 10.79 (s, 1H), 9.39 (t, J = 1.4 Hz, 1H), 8.88 (s, 1H), 8.04 (d, J = 9.6 Hz, 1H), 7.82 (dd, J = 9.6, 1.9 Hz, 1H), 7.74 (d, J = 5.9 Hz, 1H), 7.11 (d, J = 5.8 Hz, 1H), 4.23 (q, J = 7.1 Hz, 2H), 1.30 (t, J = 7.1 Hz, 3H). |
| 439 | 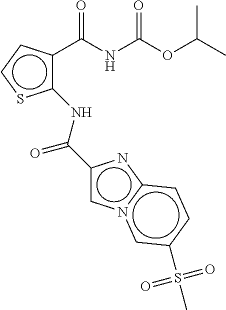 | C | | C | B | | ES-MS M/Z = 450.9 (M + 1) ¹H NMR (400 MHz, DMSO-d₆) 12.93 (s, 1H), 10.72 (s, 1H), 9.48-9.28 (m, 1H), 8.87 (s, 1H), 8.03 (d, J = 9.5 Hz, 1H), 7.81 (dd, J = 9.6, 1.9 Hz, 1H), 7.73 (d, J = 6.0 Hz, 1H), 7.10 (d, J = 5.8 Hz, 1H), 4.98 (p, J = 6.4 Hz, 1H), 3.36 (s, 3H), 1.31 (d, J = 6.3 Hz, 6H). |
| 440 | 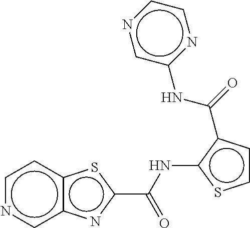 | C | | C | B | | ES-MS M/Z = 382.9 (M + 1) ¹H NMR (400 MHz, DMSO-d₆) 13.30 (s, 1H), 11.10 (s, 1H), 9.64 (s, 1H), 9.45 (d, J = 1.5 Hz, 1H), 8.71 (d, J = 5.6 Hz, 1H), 8.57-8.52 (m, 1H), 8.49 (d, J = 2.6 Hz, 1H), 8.42 (d, J = 5.6 Hz, 1H), 8.00 (d, J = 5.9 Hz, 1H), 7.30 (d, J = 5.8 Hz, 1H). |
| 441 | 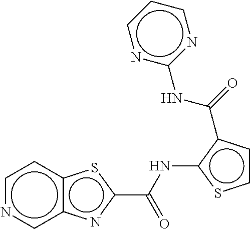 | A | | C | B | | ES-MS M/Z = 382.9 (M + 1) ¹H NMR (400 MHz, DMSO-d₆) 13.37 (s, 1H), 11.06 (s, 1H), 9.57 (s, 1H), 8.82 (d, J = 4.8 Hz, 2H), 8.69 (d, J = 5.5 Hz, 1H), 8.37 (d, J = 5.3 Hz, 1H), 8.16 (s, 2H), 7.95 (s, 1H), 7.91 (d, J = 6.0 Hz, 1H), 7.36 (t, J = 4.9 Hz, 1H), 7.27 (d, J = 6.0 Hz, 1H). |
| 442 | 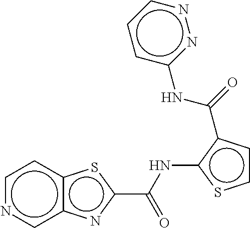 | B | | C | B | | ES-MS M/Z = 382.9 (M + 1) ¹H NMR (400 MHz, DMSO-d₆) 13.26 (s, 1H), 11.42 (s, 1H), 9.56 (s, 1H), 9.09 (dd, J = 4.7, 1.5 Hz, 1H), 8.69 (d, J = 5.6 Hz, 1H), 8.42 (dd, J = 9.0, 1.4 Hz, 1H), 8.40-8.37 (m, 1H), 8.05 (d, J = 5.9 Hz, 1H), 7.83 (dd, J = 8.9, 4.7 Hz, 1H), 7.31 (d, J = 5.9 Hz, 1H). |
| 443 | 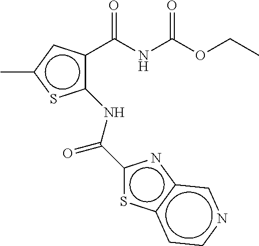 | A | | C | | | ES MS M/Z = 391 (M + 1) ¹H NMR (400 MHz, CD₃OD) 9.59 (d, J = 0.9 Hz, 1H), 8.69 (d, J = 5.9 Hz, 1H), 8.46 (dd, J = 5.8, 0.9 Hz, 1H), 7.21 (d, J = 1.2 Hz, 1H), 4.32 (q, J = 7.2 Hz, 2H), 2.48 (d, J = 1.2 Hz, 3H), 1.39 (t, J = 7.1 Hz, 3H). |
| 444 | 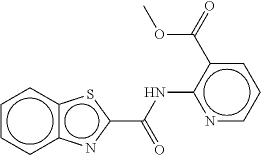 | B | | C | | | ES MS M/Z = 314 (M + 1) ¹H NMR (400 MHz, CD₃OD) 8.63 (dd, J = 4.9, 1.9 Hz, 1H), 8.54 (dd, J = 7.9, 1.9 Hz, 1H), 8.29-8.25 (m, 1H), 8.17 (dd, J = 8.0, 1.3Hz, 1H), 7.66 (ddd, J = 8.3, 7.2, 1.3 Hz, 2H), 7.36 (d, J = 7.9 Hz, 1H), 4.07 (s, 3H). |
| 445 | 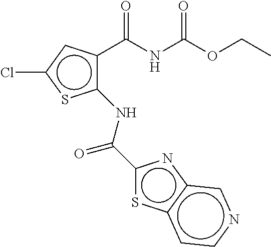 | A | | C | | | ES-MS M/Z = 411 (M + 1) ¹H NMR (400 MHz, CD₃OD) 9.43 (d, J = 0.9 Hz, 1H), 8.56 (d, J = 5.7 Hz, 1H), 8.26 (dd, J = 5.7, 1.0 Hz, 1H), 7.44 (s, 1H), 4.20 (q, J = 7.1 Hz, 2H), 1.27 (t, J = 7.1 Hz, 3H). |
| 446 | 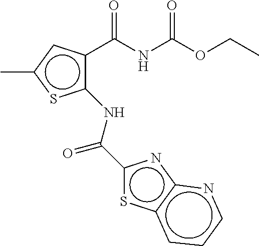 | A | | B | | | ES MS M/Z = 391 (M + 1) ¹H NMR (400 MHz, CD₃OD) 8.86 (dd, J = 4.6, 1.6 Hz, 1H), 8.71 (dd, J = 8.2, 1.6 Hz, 1H), 7.64 (dd, J = 8.2, 4.6 Hz, 1H), 7.19 (s, 1H), 4.31 (q, J = 7.1 Hz, 2H), 2.48 (s, 3H), 1.38 (t, J = 7.1 Hz, 3H). |
| 447 | 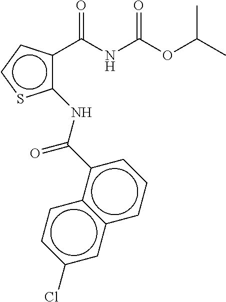 | B | | C | | | ES-MS M/Z = 417.9 (M + 1) |
| 448 | 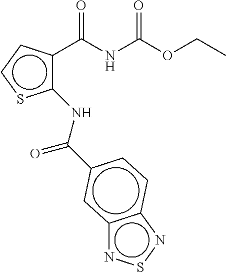 | A | | B | | | ES-MS M/Z = 376.9 (M + 1) |
| 449 | 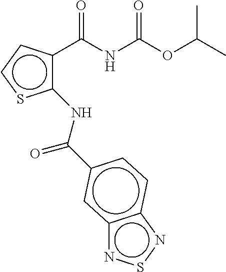 | A | | B | | | ES-MS M/Z = 390.9 (M + 1) |
| 450 | 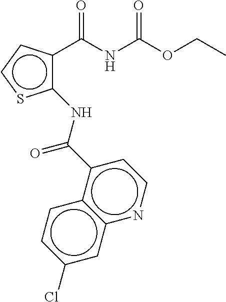 | C | | C | | | ES-MS M/Z = 404 (M + 1) |
| 451 | 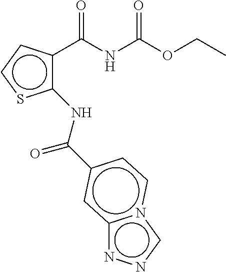 | A | | B | | | ES-MS M/Z = 360 (M + 1) |
| 452 | 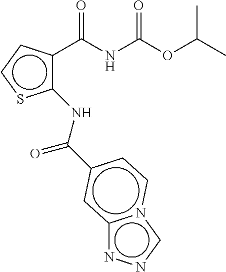 | A | | C | | | ES-MS M/Z = 374 (M + 1) |
| 453 | 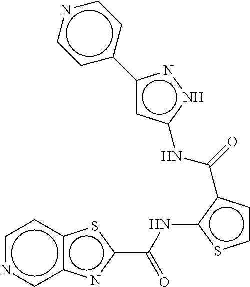 | C | | C | | | ES-MS M/Z = 447.9 (M + 1) ¹H NMR (400 MHz, DMSO-d₆) 13.44 (s, 1H), 11.08 (s, 1H), 9.57 (s, 1H), 8.84 (d, J = 6.6 Hz, 1H), 8.70 (d, J = 5.5 Hz, 1H), 8.39 (dd, J = 5.6, 1.0 Hz, 1H), 8.19 (s, 2H), 7.89 (s, 1H), 7.38 (s, 1H), 7.31 (d, J = 5.8 Hz, 1H). |
| 454 | 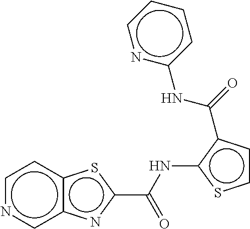 | C | | C | | | ES-MS M/Z = 381.9 (M + 1) ¹H NMR (400 MHz, DMSO-d₆) 13.38 (s, 1H), 10.79 (s, 1H), 9.59 (s, 1H), 8.70 (d, J = 5.5 Hz, 1H), 8.48-8.43 (m, 1H), 8.39 (d, J = 5.5 Hz, 1H), 8.20 (d, J = 8.4 Hz, 1H), 8.01 (d, J = 5.9 Hz, 1H), 7.97- 7.88 (m, 1H), 7.28 (d, J = 6.2 Hz, 1H), 7.26-7.23 (m, 1H). |
| 455 | 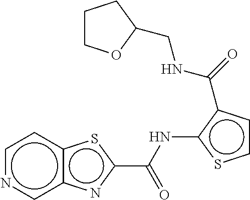 | C | | C | | | ES-MS M/Z = 388.9 (M + 1) ¹H NMR (400 MHz, DMSO-d₆) 13.75 (s, 1H), 9.71 (s, 1H), 8.74 (d, J = 5.7 Hz, 1H), 8.71 (d, J = 5.9 Hz, 1H), 8.51 (d, J = 5.7 Hz, 1H), 7.65 (d, J = 5.8 Hz, 1H), 7.24 (d, J = 5.8 Hz, 1H), 4.06-4.00 (m, 1H), 3.81 (dt, J = 8.1, 6.5 Hz, 1H), 3.71- 3.60 (m, 1H), 3.39 (dd, J = 6.2, 4.7 Hz, 2H), 2.03-1.90 (m, 1H), 1.90- 1.77 (m, 2H), 1.61 (ddt, J = 11.6, 8.2, 6.6 Hz, 1H). |
| 456 | 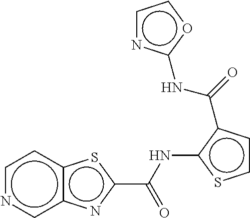 | B | | C | | | ES-MS M/Z = 371.9 (M + 1) ¹H NMR (400 MHz, DMSO-d₆) 13.79 (s, 1H), 9.61 (s, 1H), 8.71 (d, J = 5.6 Hz, 1H), 8.43 (dd, J = 5.5, 1.0 Hz, 1H), 8.04 (s, 1H), 7.68 (s, 1H), 7.31 (d, J = 1.2 Hz, 1H), 7.26 (d, J = 5.8 Hz, 1H). |
| 457 | 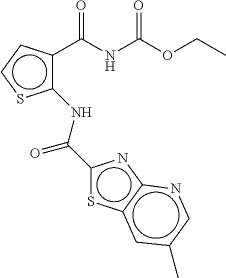 | A | | B | | | ES-MS M/Z = 391 (M + 1) |
| 458 | 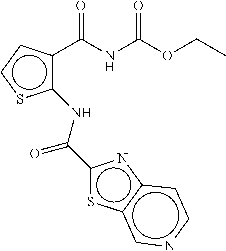 | C | | C | | | ES-MS M/Z = 377 (M + 1) ¹H NMR (400 MHz, DMSO-d₆) 13.14 (s, 1H), 9.32 (s, 1H), 8.64 (d, J = 5.6 Hz, 1H), 8.02 (d, J = 5.5 Hz, 1H), 7.20 (d, J = 5.8 Hz, 1H), 6.72 (d, J = 5.9 Hz, 1H), 4.20 (q, J = 7.1 Hz, 2H), 1.36 (t, J = 7.1 Hz, 3H). |
| 459 | 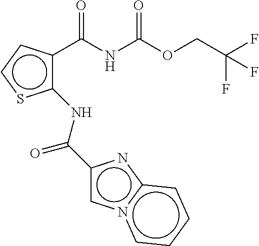 | | | | | | ES-MS M/Z = 413 (M + 1) |
| 460 | 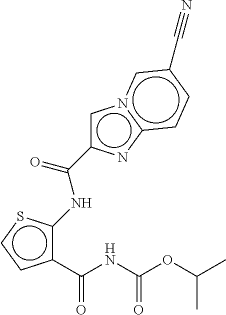 | | | | | | ES-MS M/Z = 398 (M + 1) |

1.-8. (canceled)

9. A compound of Formula (Ia), a pharmaceutically acceptable salt, solvate, polymorph, prodrug, metabolite, N-oxide, stereoisomer, or isomer thereof: 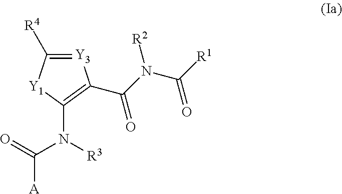
wherein:
Y₁ is S or O or NR²;
Y₃ is CR⁵ or N;
R¹ is —O-(optionally substituted alkyl), —O-(alkenyl), —O-(alkynyl), —O-(cycloalkyl), —O-(heterocyclyl), —O-(optionally substituted aralkyl), —O-(optionally substituted heteroaralkyl), —O-(alkyl)-(alkoxy), —O-(alkyl)-(aralkoxy), —O-(alkyl)-(heterocyclyl), —O-(alkyl)-(COOR^{a}), —O-(alkyl)-(NR⁶R⁷), —NR⁶R⁷ or R⁸;
R² and R³ are each independently selected from H, optionally substituted alkyl, and optionally substituted aryl; or R¹ and R² taken together form a heterocycle;
R⁴ is H, halogen, —CN, alkyl, aryl, —R^{b}COOR^{a} or —R^{b}CH(COOR^{a})₂;
R⁵ is H, halogen, optionally substituted alkyl, or cycloalkyl; or R⁴ and R⁵ taken together form a carbocycle or an optionally substituted heterocycle;
R⁶ and R⁷ are each independently selected from H and optionally substituted alkyl;
wherein the optional substituent is halogen; or R⁶ and R⁷ taken together form an optionally substituted heterocycle with the nitrogen to which they are attached;
wherein the optional substituent is halogen;
R⁸ is optionally substituted alkyl;
each R^{a} is independently selected from H and alkyl;
R^{b} is a bond or alkylenyl;
R^{c} is a bond or alkenylenyl; and
A is optionally substituted aryl, optionally substituted heterocyclyl, optionally substituted heteroaryl, optionally substituted carbocyclyl, optionally substituted aralkyl, optionally substituted heteroaralkyl or —R^{c}-(optionally substituted heteroaryl).

10.-16. (canceled)

17. The compound of claim 9, wherein Y₃ is CR⁵.

18. The compound of claim 9, wherein Y₁ is S.

19. The compound of claim 9, wherein Y₁ is S and Y3 is CH.

20. The compound of claim 9, wherein R⁴ is H.

21. The compound of claim 9, wherein R¹ is —O-(optionally substituted alkyl); and R2 and R3 are both H.

22. The compound of claim 9, wherein A is optionally substituted aryl, optionally substituted heterocyclyl, optionally substituted heteroaryl, optionally substituted carbocyclyl, optionally substituted aralkyl, optionally substituted heteroaralkyl or —Rc-(optionally substituted heteroaryl); and the optionally substituted aryl, the optionally substituted heterocyclyl, the optionally substituted heteroaryl, the optionally substituted carbocyclyl, the optionally substituted aralkyl and the optionally substituted heteroaralkyl are substituted with 1-6 R10; wherein
each R¹⁰ is independently selected from H, halogen, —CN, —NO₂, —CF₃, alkyl, —SR⁶, —OR⁶,
—NR⁶R⁷, —NR⁶C(═O)(alkyl), —NR⁶C(═O)(cycloalkyl), —NR⁶C(═O)(heterocyclyl),
—NR⁶C(═O)(aryl), —NR⁶C(═O)(heteroaryl), —C(═O)NR⁶R⁷,
—C(═O)NR⁶(cycloalkyl),
—C(═O)NR⁶(heterocycloalkyl), —C(═O)NR⁶(aryl), —C(═O)NR⁶(heteroaryl),
—NR⁶C(═O)NR⁶R⁷, —NR⁶C(═O)NR⁷(cycloalkyl),
—NR⁶C(═O)NR⁷(heterocycloalkyl),
—NR⁶C(═O)NR⁷(aryl), —NR⁶C(═O)NR⁷(heteroaryl), —NR⁶C(═O)O(alkyl),
—NR⁶C(═O)O(cycloalkyl), —NR⁶C(═O)O(heterocycloalkyl), —NR⁶C(═O)O(aryl),
—NR⁶C(═O)O(heteroaryl), —NR⁶SO₂(alkyl), —NR⁶SO₂(cycloalkyl),
—NR⁶SO₂(heterocycloalkyl), —NR⁶SO₂(aryl), —NR⁶SO₂(heteroaryl), —SO₂NR⁶R⁷,
—SO₂NR⁶(cycloalkyl), —SO₂NR⁶(heterocycloalkyl), —SR⁶, —SO₂R⁶, —SO₂NR⁶(aryl),
—SO₂NR⁶(heteroaryl), haloalkyl, aryl, heteroaryl, heterocyclyl and tetrazoyl.

23. (canceled)

24. The compound of claim 22, wherein A is optionally substituted heteroaryl.

25. The compound of claim 24, wherein A is selected from:

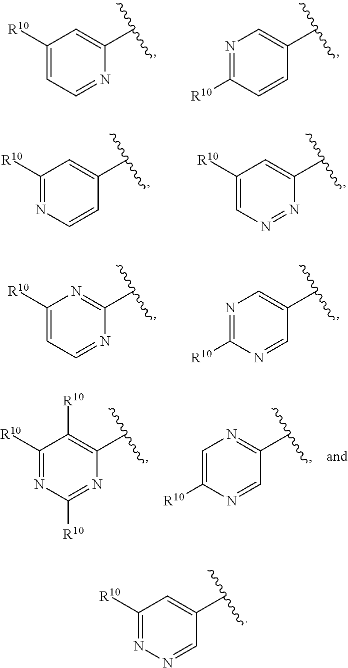

26. (canceled)

27. The compound of claim 24, wherein A is selected from: 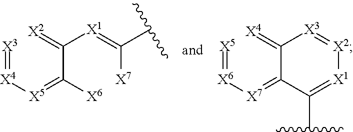
wherein:
X¹, X², X³, X⁴, X⁵, X⁶, and X⁷ are independently selected from N and CR¹⁰; and at least one of X¹-X⁷ is N.

28. The compound of claim 27 wherein A is selected from:

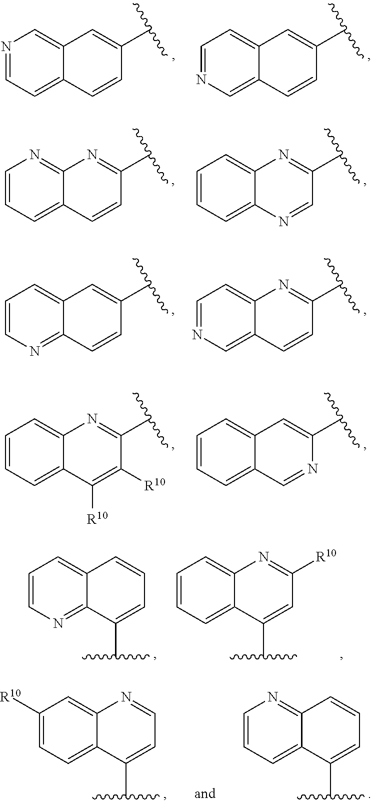

29. The compound of claim 24, wherein A is selected from: 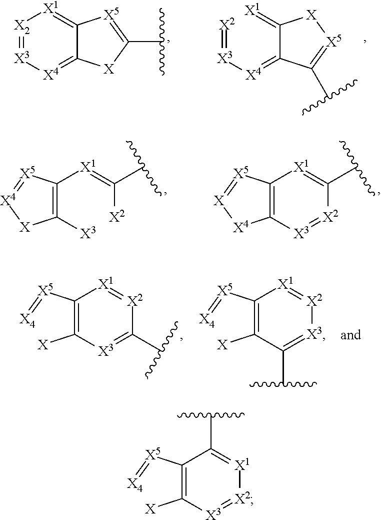
wherein:
X¹, X², X³, X⁴, X⁵, X⁶, and X⁷ are independently selected from N and CR¹⁰; and
X is O, S, or NR².

30. The compound of claim 29, wherein A is selected from:

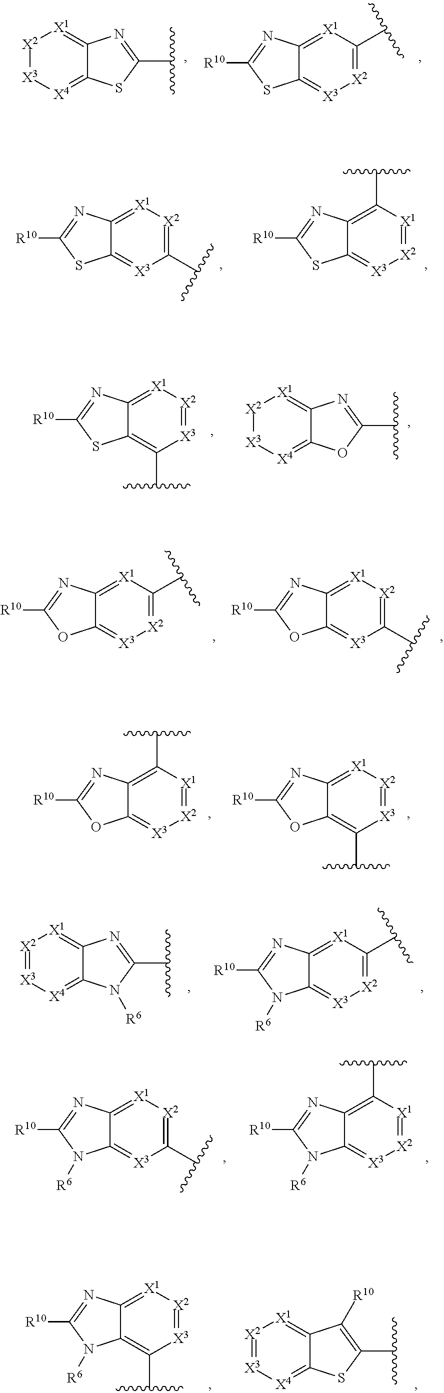

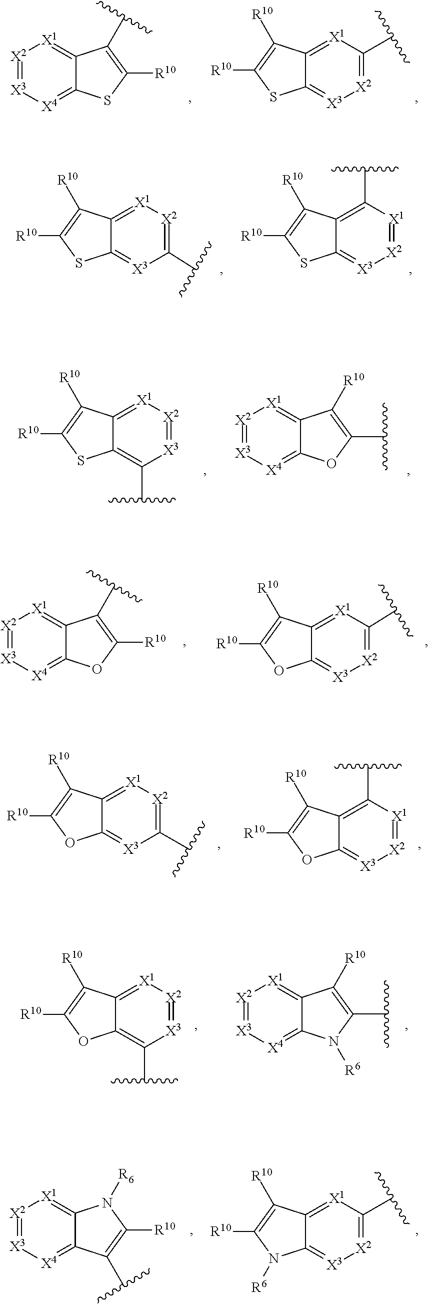

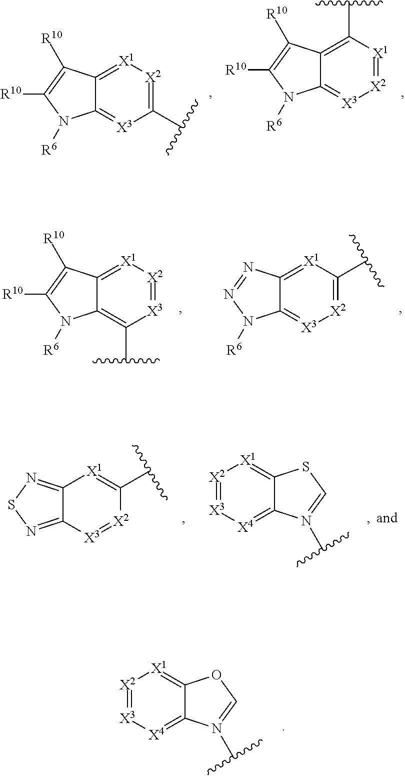

31. The compound of claim 30, wherein A is selected from:

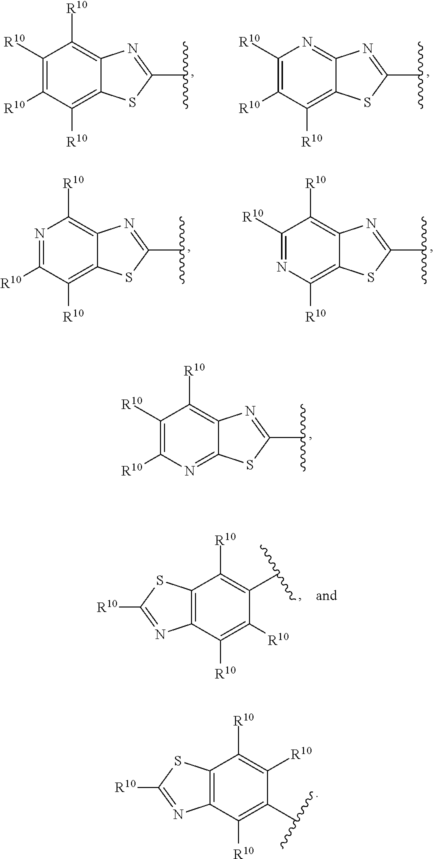

32.-35. (canceled)

36. The compound of claim 24, wherein A is selected from: 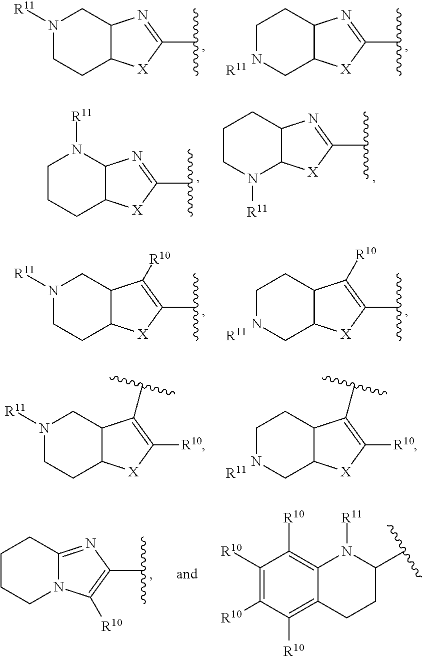
wherein:
X is O, S, or NR²; and
R¹¹ is H, alkyl, aryl, heteroaryl, —SO₂-(alkyl), —SO₂-(cycloalkyl), —SO₂-(aryl),
—SO₂-(heteroaryl), —SO₂-(heterocycloalkyl), —C(═O)O(alkyl), —C(═O)O(cycloalkyl),
—C(═O)O(heterocycloalkyl), —C(═O)O(aryl), —C(═O)O(heteroaryl), —C(═O)NR⁶R⁷,
—C(═O)NR⁶(cycloalkyl), —C(═O)NR⁶(heterocycloalkyl), —C(═O)NR⁶(aryl),
—C(═O)NR⁶(heteroaryl), —C(═O)(alkyl), —C(═O)(cycloalkyl),
—C(═O)(heterocycloalkyl),
—C(═O)(aryl), or —C(═O)(heteroaryl).

37. The compound of claim 24, wherein A is selected from:

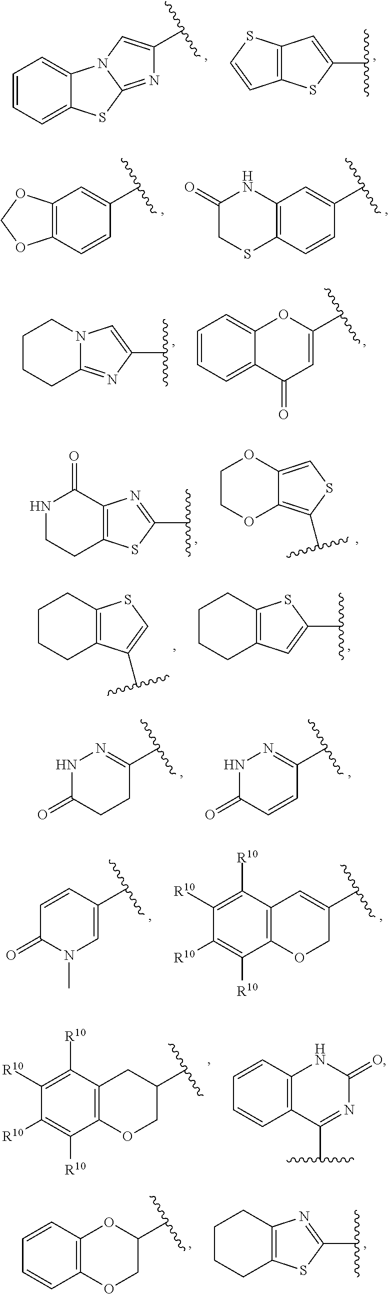

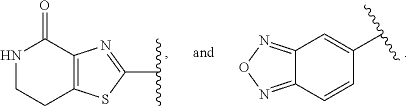 [SMILES: *c1nc2c(s1)CCNC2=O ; *c1ccc2nonc2c1]

38.-52. (canceled)

53. A compound selected from: 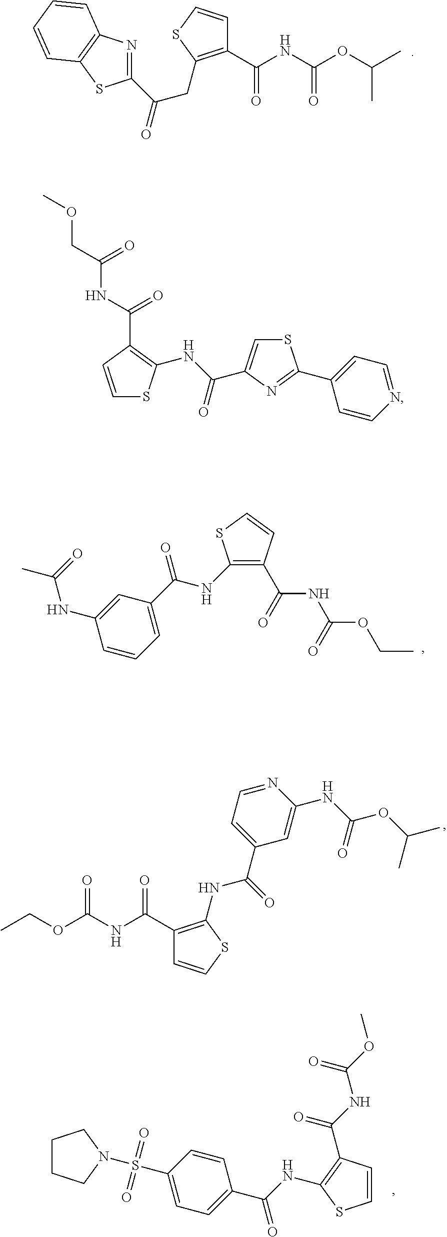 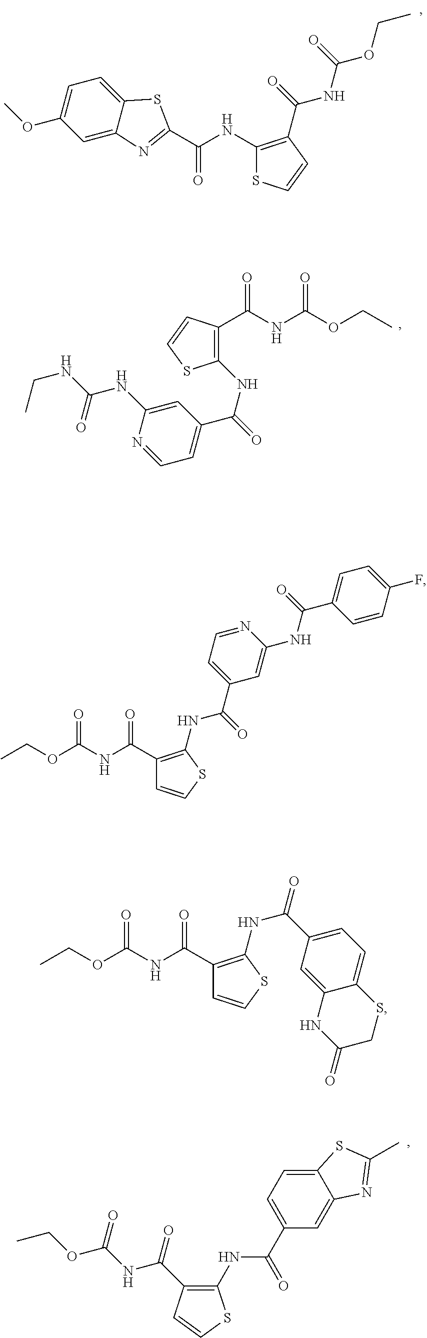 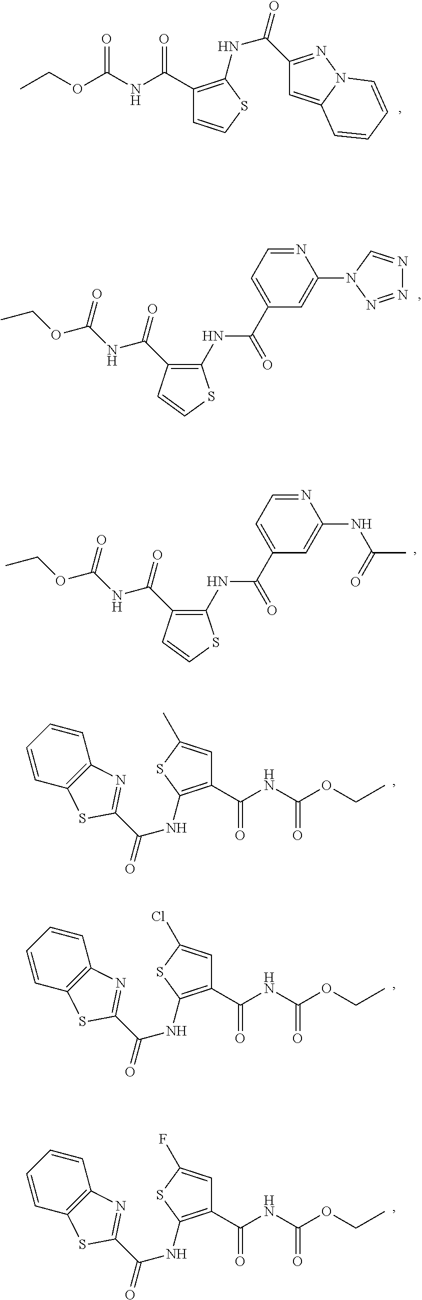 [SMILES: CCOC(=O)NC(=O)c1ccsc1NC(=O)c1cc2ccccn2n1 ; CCOC(=O)NC(=O)c1ccsc1NC(=O)c1ccnc(-n2cnnn2)c1 ; CCOC(=O)NC(=O)c1ccsc1NC(=O)c1ccnc(NC(C)=O)c1 ; CCOC(=O)NC(=O)c1cc(C)sc1NC(=O)c1nc2ccccc2s1 ; CCOC(=O)NC(=O)c1cc(Cl)sc1NC(=O)c1nc2ccccc2s1 ; CCOC(=O)NC(=O)c1cc(F)sc1NC(=O)c1nc2ccccc2s1] 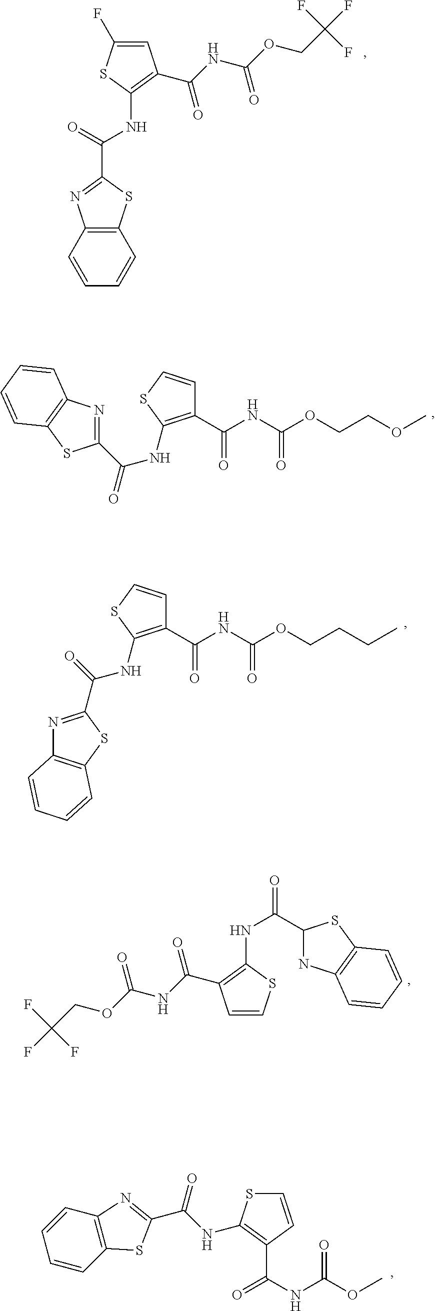 [SMILES: O=C(NC(=O)c1cc(F)sc1NC(=O)c1nc2ccccc2s1)OCC(F)(F)F ; COCCOC(=O)NC(=O)c1ccsc1NC(=O)c1nc2ccccc2s1 ; CCCCOC(=O)NC(=O)c1ccsc1NC(=O)c1nc2ccccc2s1 ; O=C(NC(=O)c1ccsc1NC(=O)C1Nc2ccccc2S1)OCC(F)(F)F ; COC(=O)NC(=O)c1ccsc1NC(=O)c1nc2ccccc2s1] 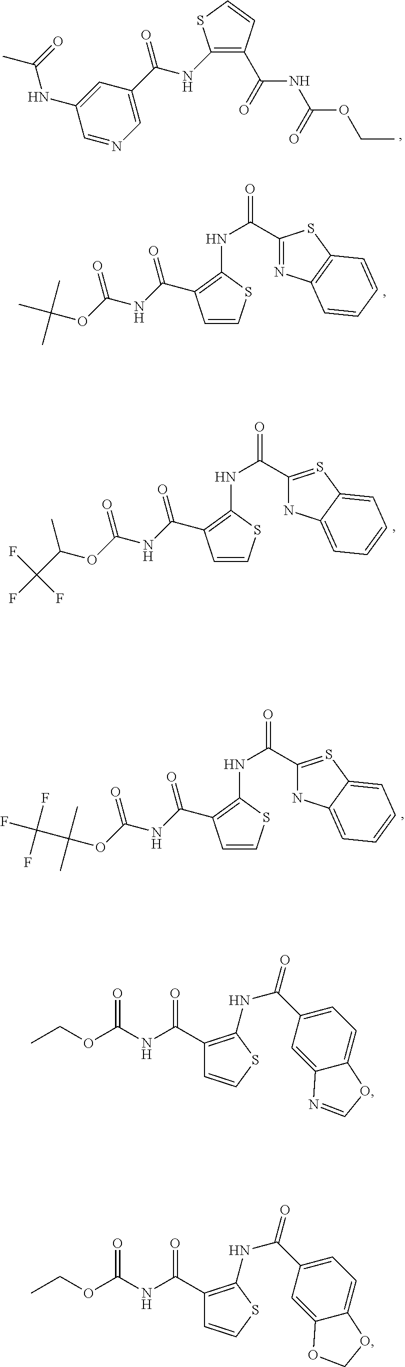 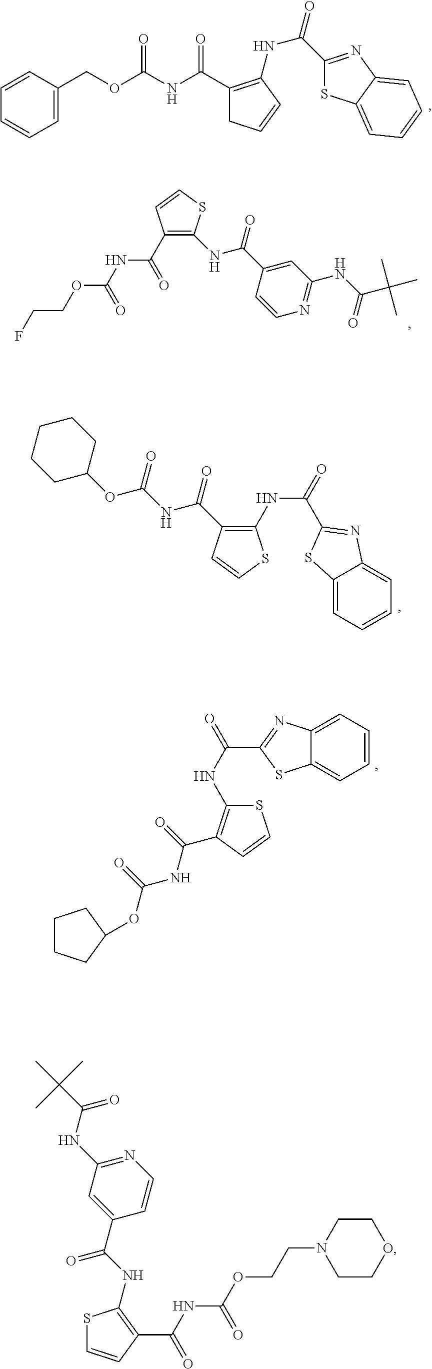 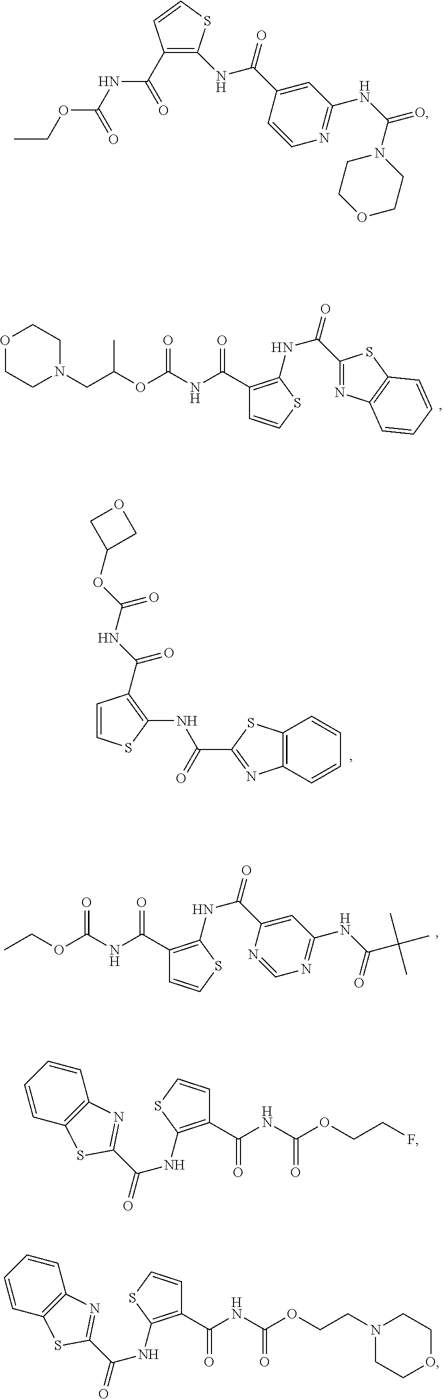 [SMILES: CCOC(=O)NC(=O)c1ccsc1NC(=O)c1ccnc(NC(=O)N2CCOCC2)c1 ; CC(CN1CCOCC1)OC(=O)NC(=O)c1ccsc1NC(=O)c1nc2ccccc2s1 ; O=C(NC(=O)c1ccsc1NC(=O)c1nc2ccccc2s1)OC1COC1 ; CCOC(=O)NC(=O)c1ccsc1NC(=O)c1cc(NC(=O)C(C)(C)C)ncn1 ; O=C(NC(=O)c1ccsc1NC(=O)c1nc2ccccc2s1)OCCF ; O=C(NC(=O)c1ccsc1NC(=O)c1nc2ccccc2s1)OCCN1CCOCC1] 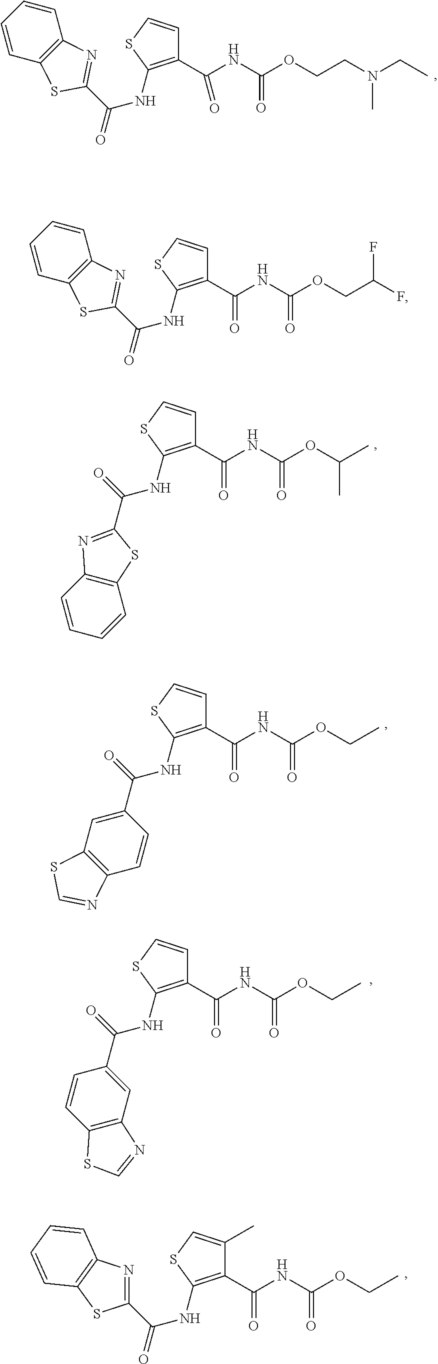 [SMILES: CCN(C)CCOC(=O)NC(=O)c1ccsc1NC(=O)c1nc2ccccc2s1 ; O=C(NC(=O)c1ccsc1NC(=O)c1nc2ccccc2s1)OCC(F)F ; CC(C)OC(=O)NC(=O)c1ccsc1NC(=O)c1nc2ccccc2s1 ; CCOC(=O)NC(=O)c1ccsc1NC(=O)c1ccc2ncsc2c1 ; CCOC(=O)NC(=O)c1ccsc1NC(=O)c1ccc2scnc2c1 ; CCOC(=O)NC(=O)c1c(C)csc1NC(=O)c1nc2ccccc2s1] 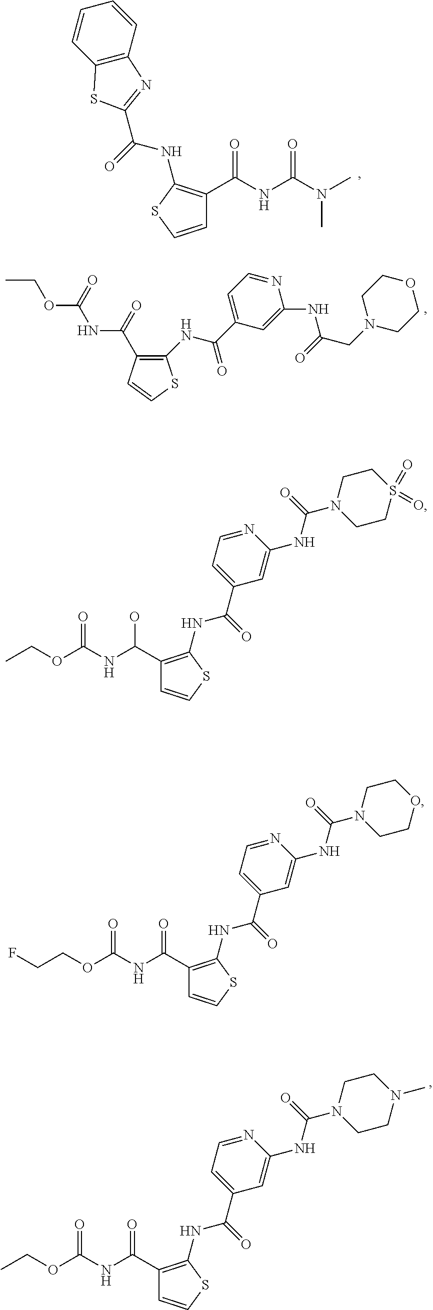 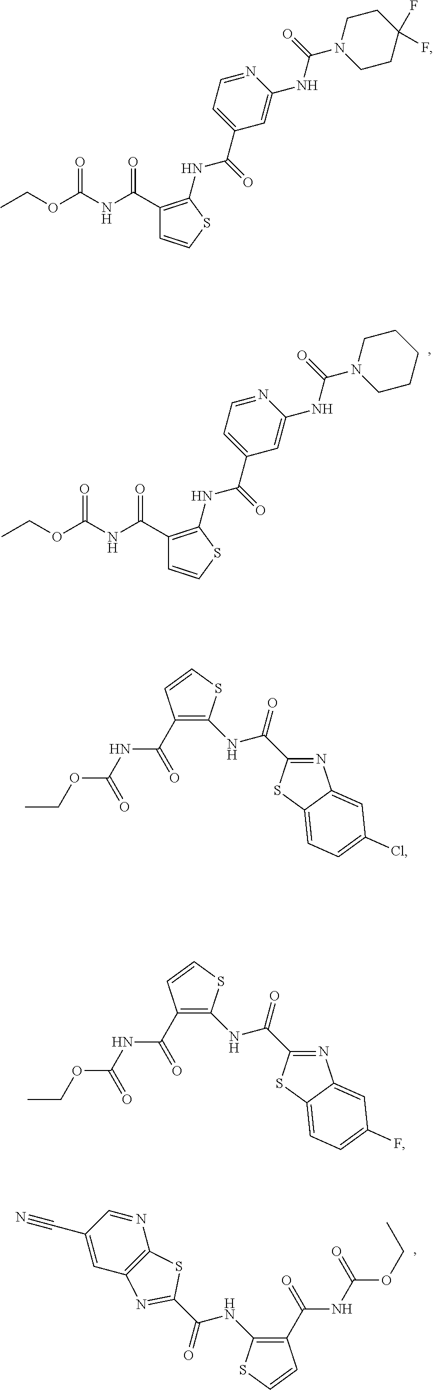 [SMILES: CCOC(=O)NC(=O)c1ccsc1NC(=O)c1ccnc(NC(=O)N2CCC(F)(F)CC2)c1 ; CCOC(=O)NC(=O)c1ccsc1NC(=O)c1ccnc(NC(=O)N2CCCCC2)c1 ; CCOC(=O)NC(=O)c1ccsc1NC(=O)c1nc2cc(Cl)ccc2s1 ; CCOC(=O)NC(=O)c1ccsc1NC(=O)c1nc2cc(F)ccc2s1 ; CCOC(=O)NC(=O)c1ccsc1NC(=O)c1nc2cc(C#N)cnc2s1] 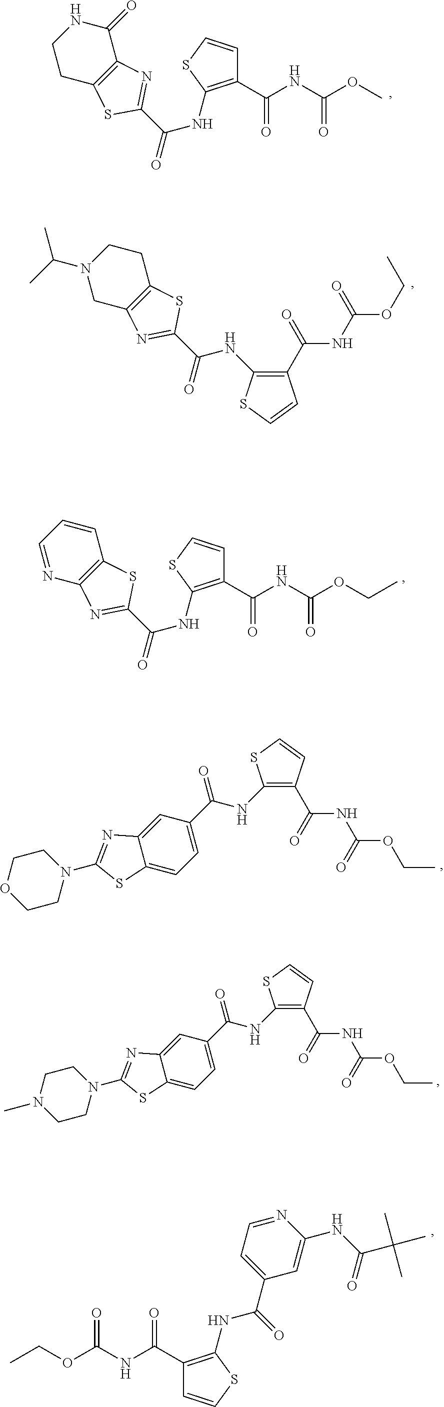 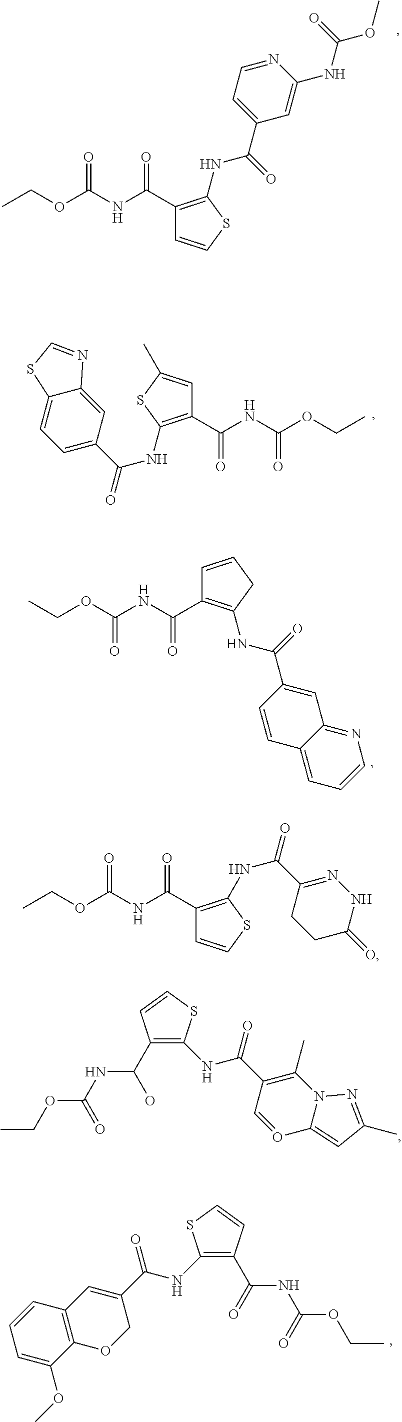 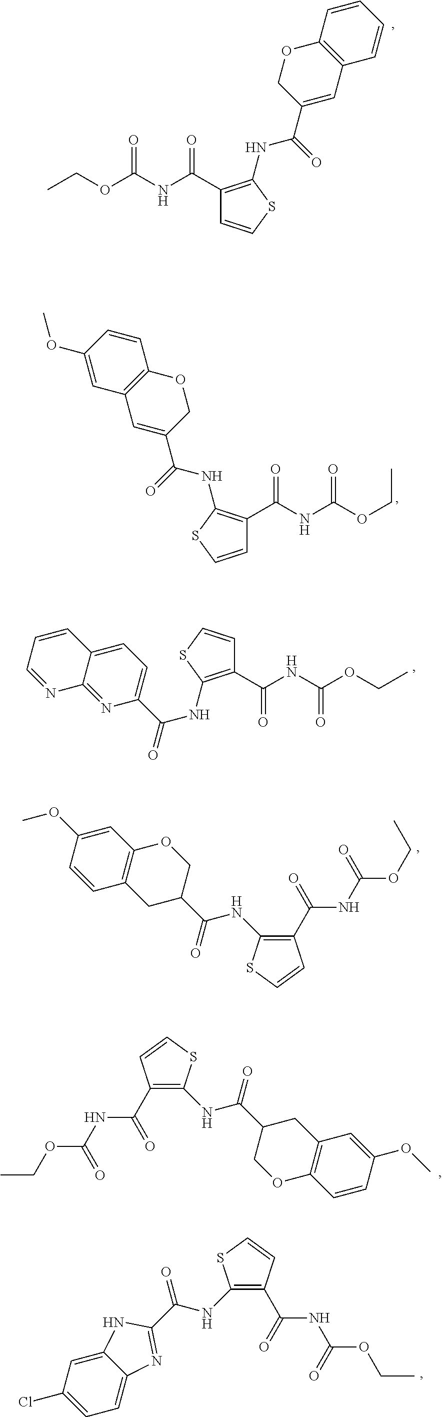 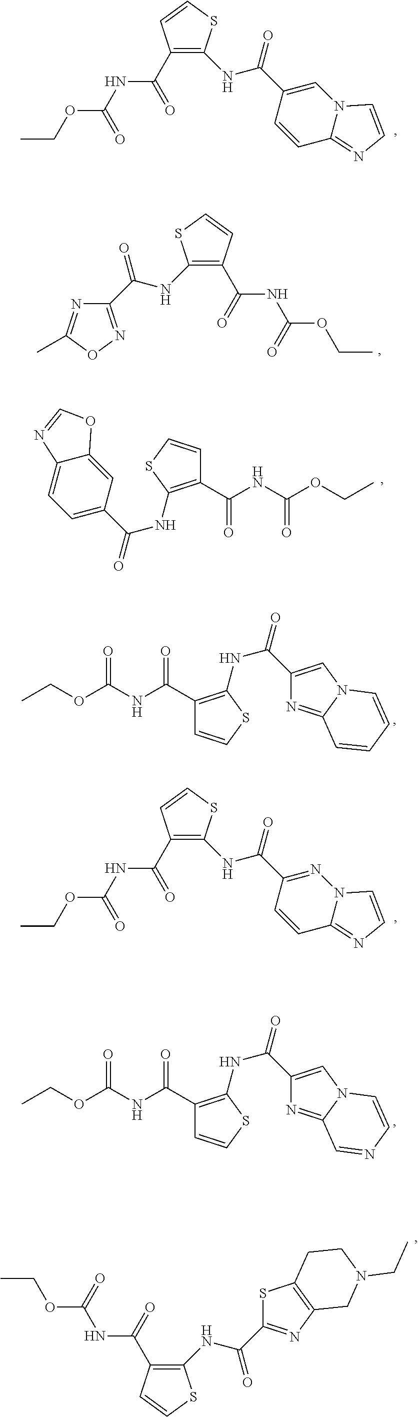 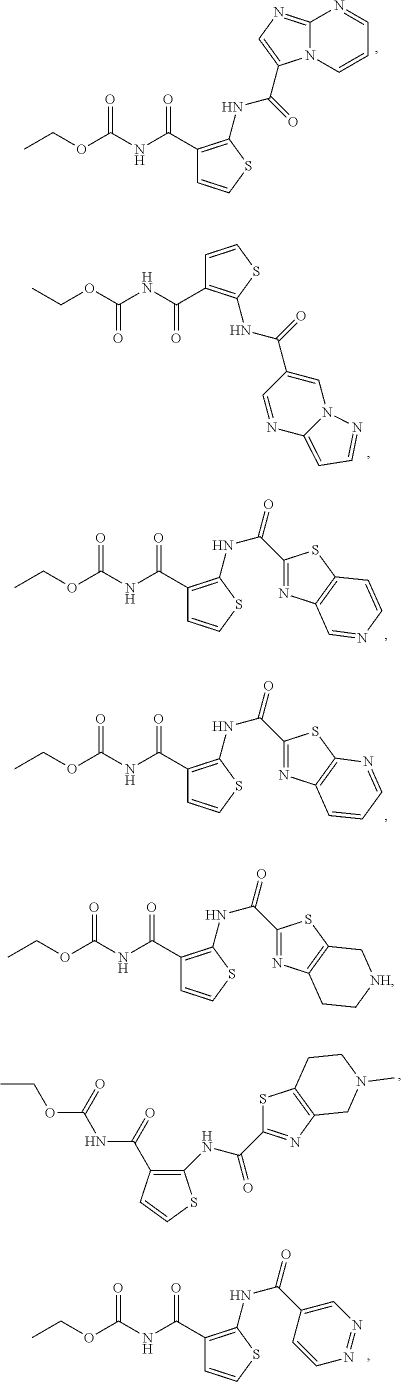 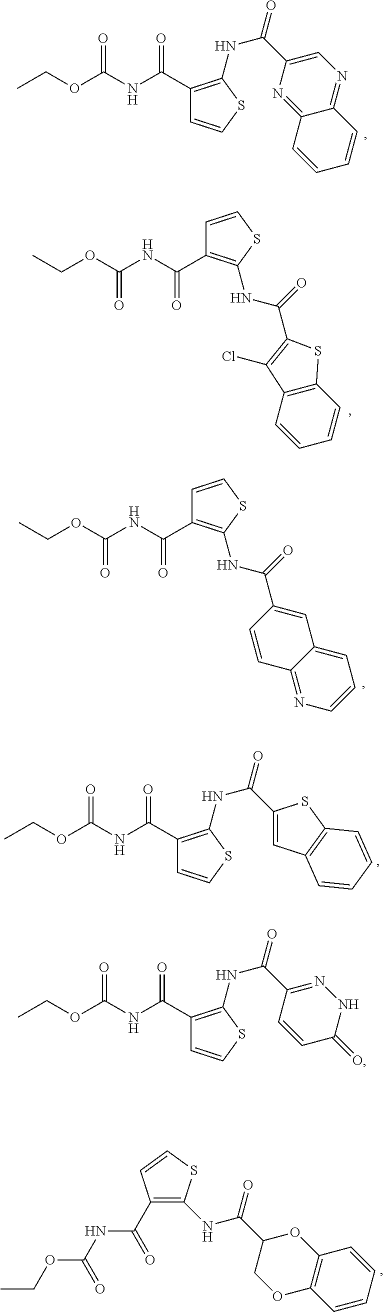 [SMILES: CCOC(=O)NC(=O)c1ccsc1NC(=O)c1cnc2ccccc2n1 ; CCOC(=O)NC(=O)c1ccsc1NC(=O)c1sc2ccccc2c1Cl ; CCOC(=O)NC(=O)c1ccsc1NC(=O)c1ccc2ncccc2c1 ; CCOC(=O)NC(=O)c1ccsc1NC(=O)c1cc2ccccc2s1 ; CCOC(=O)NC(=O)c1ccsc1NC(=O)c1ccc(=O)[nH]n1 ; CCOC(=O)NC(=O)c1ccsc1NC(=O)C1COc2ccccc2O1] 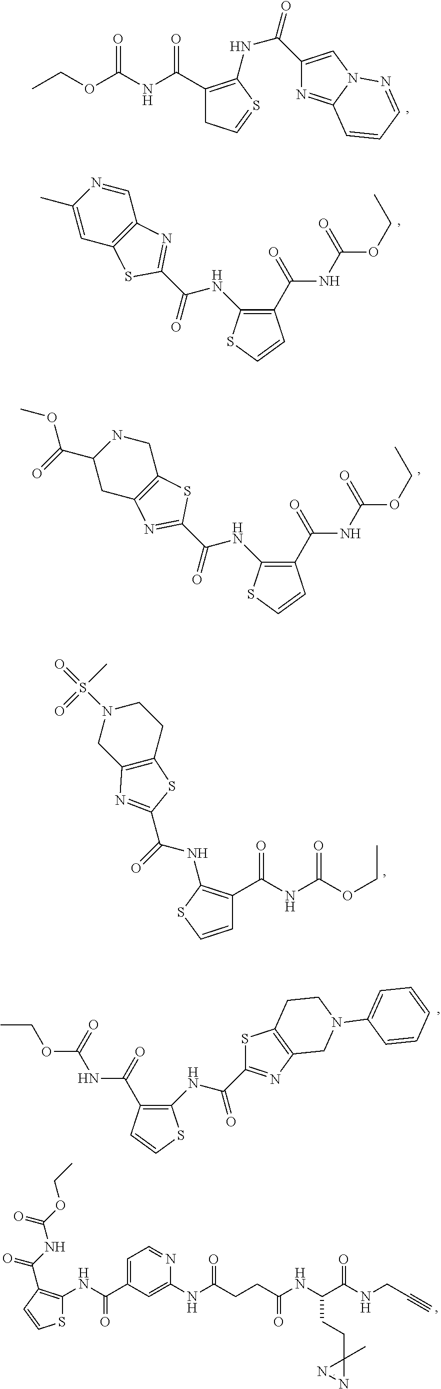 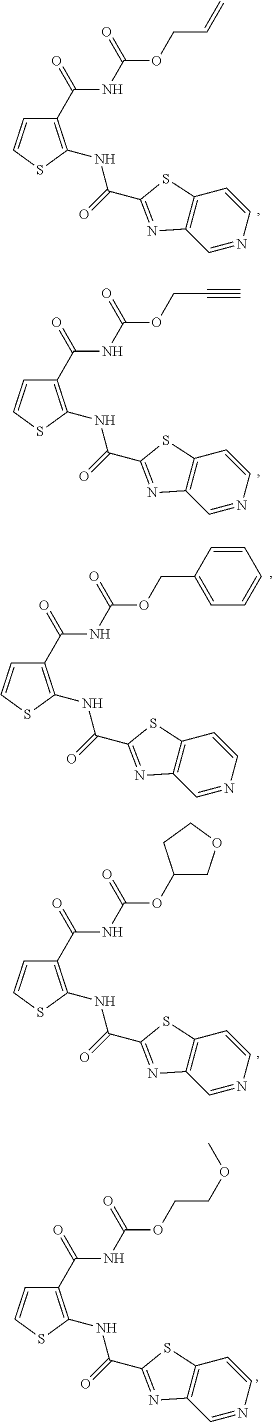 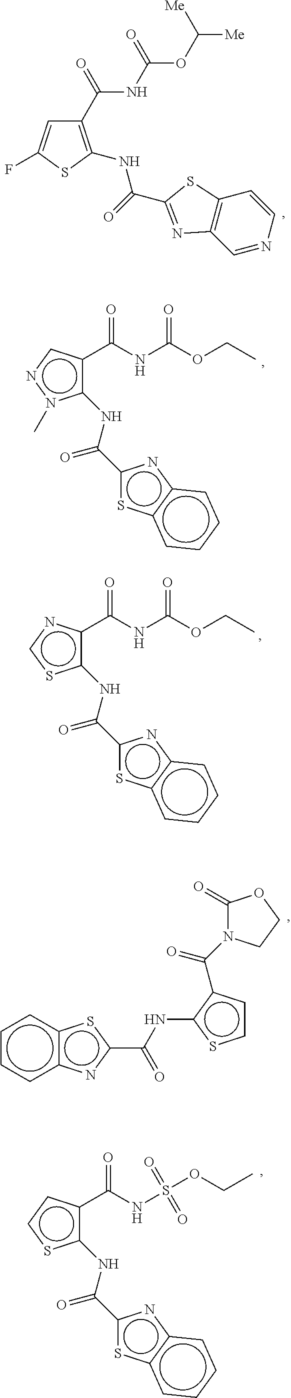 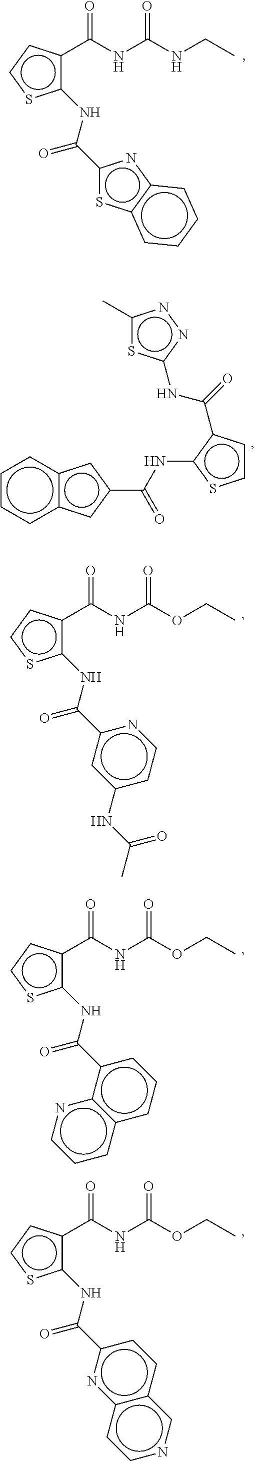 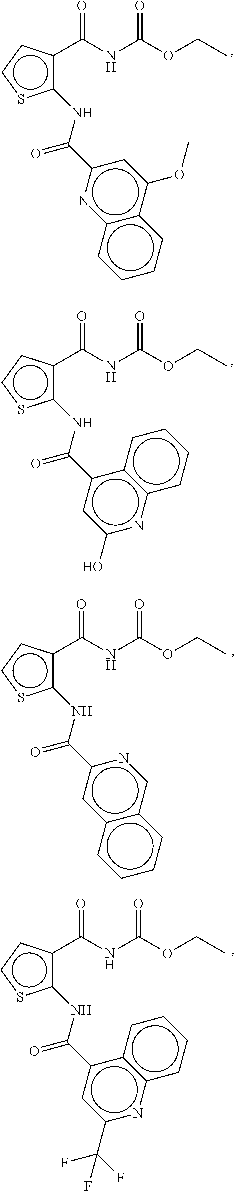 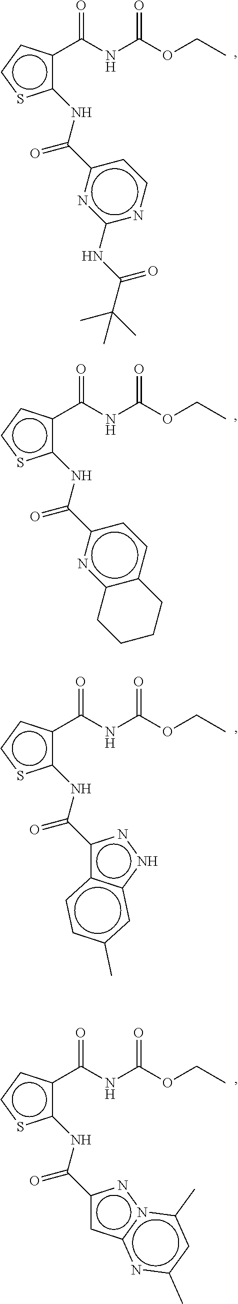 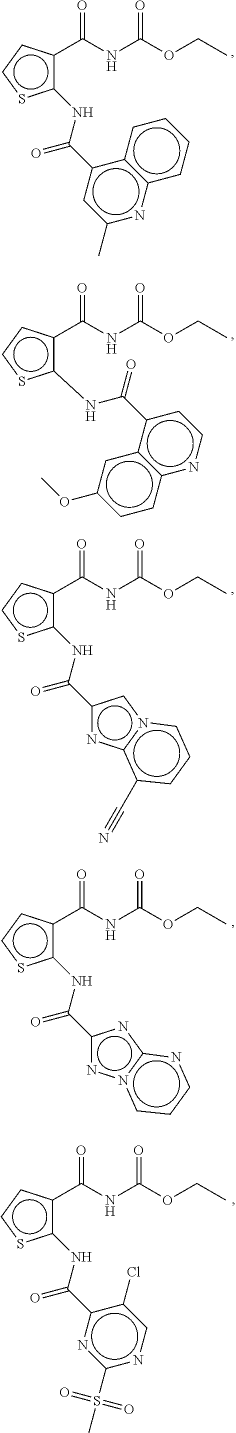 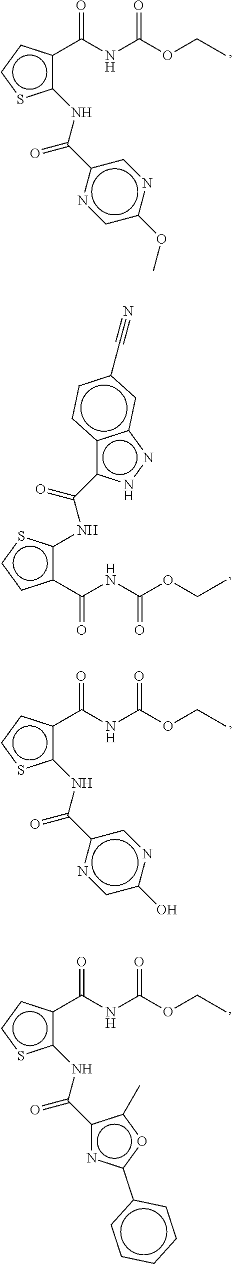 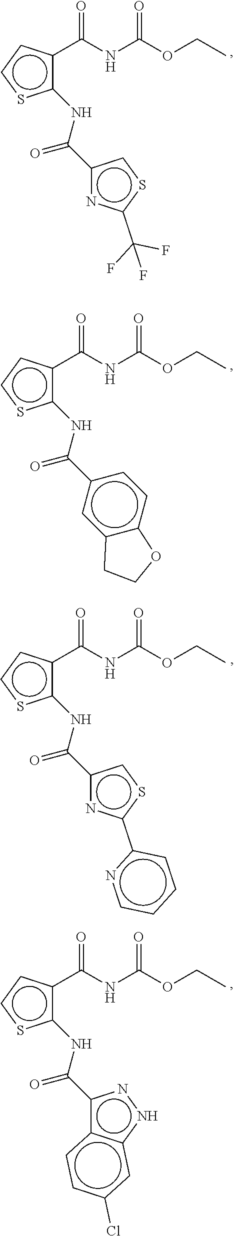 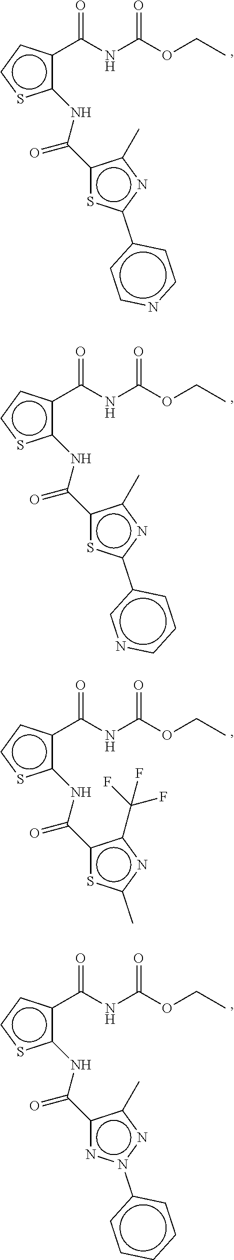 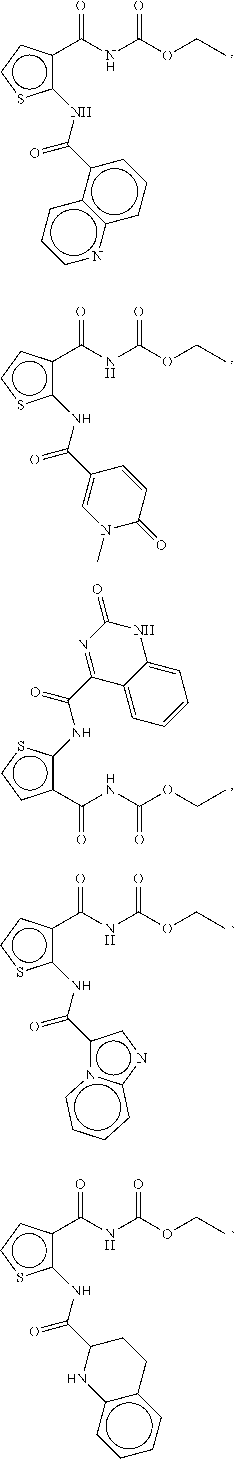 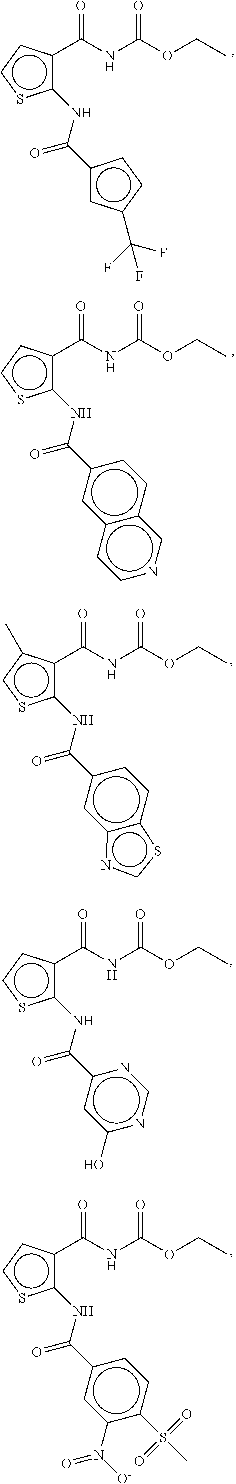 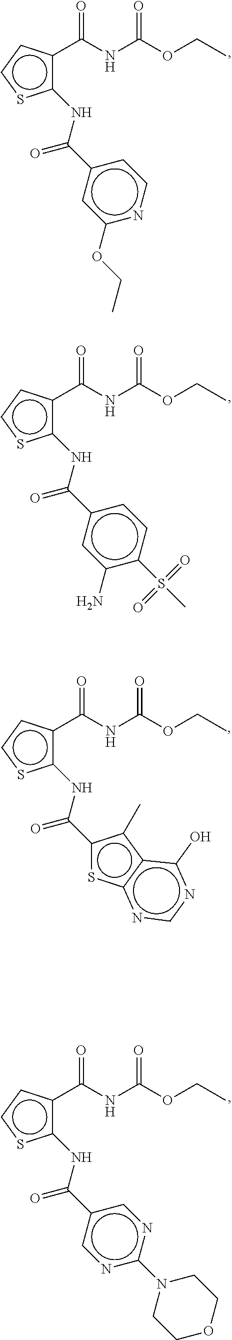 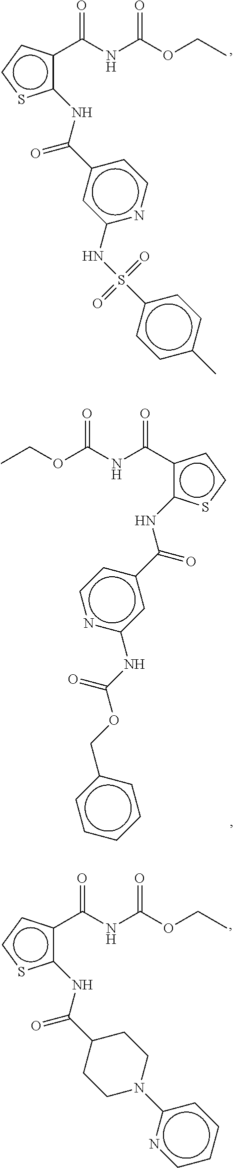 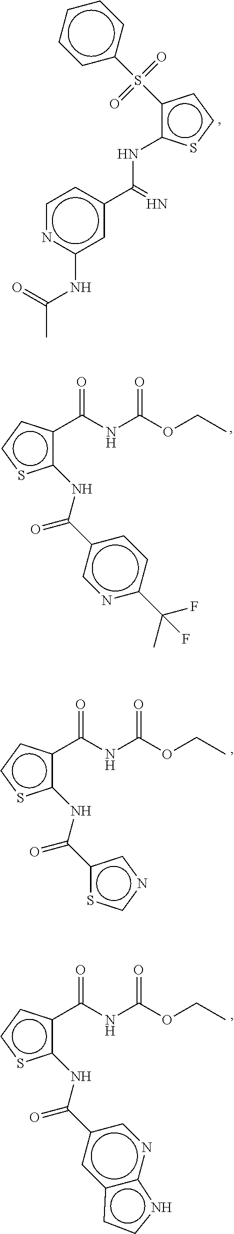 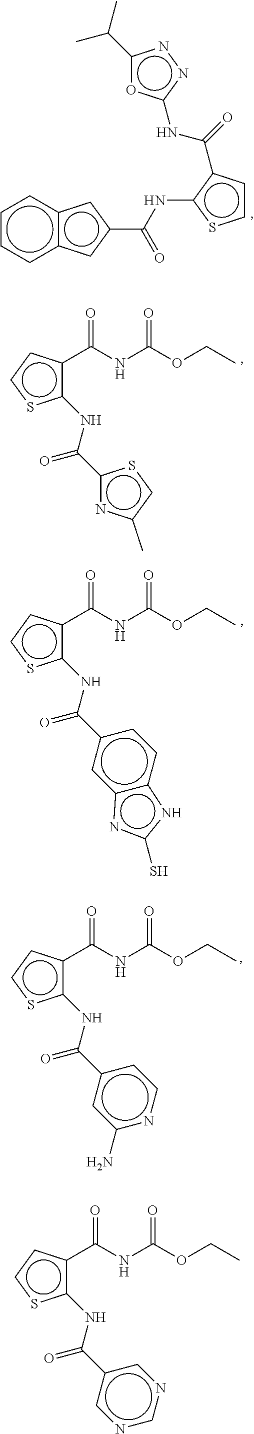 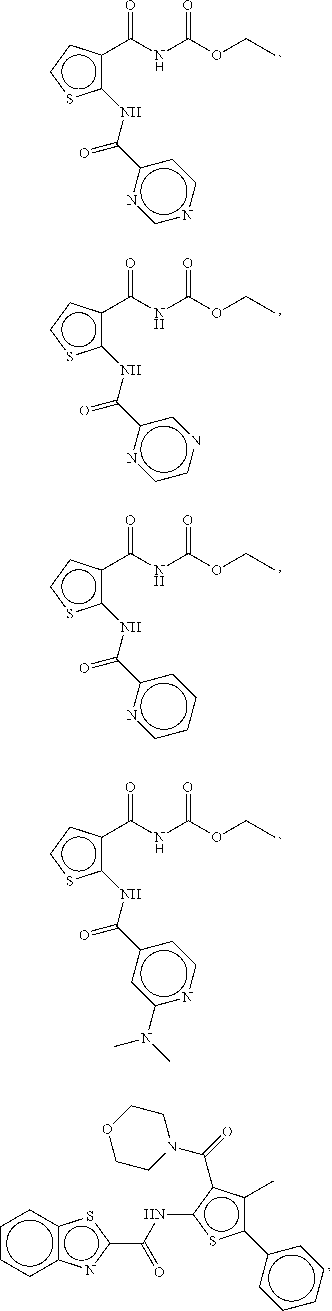 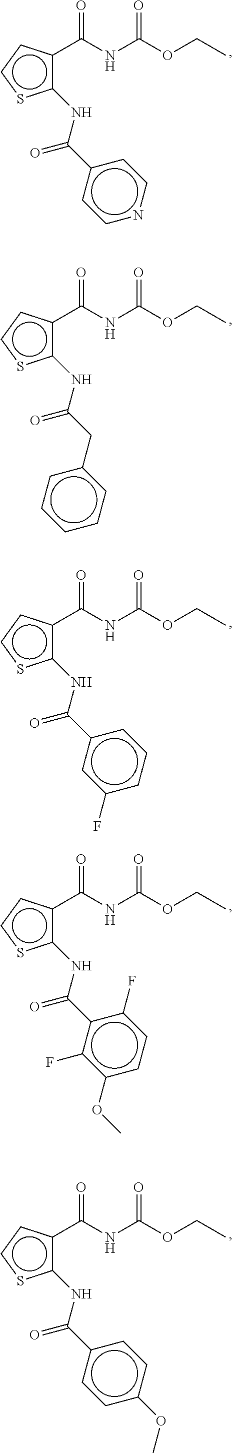 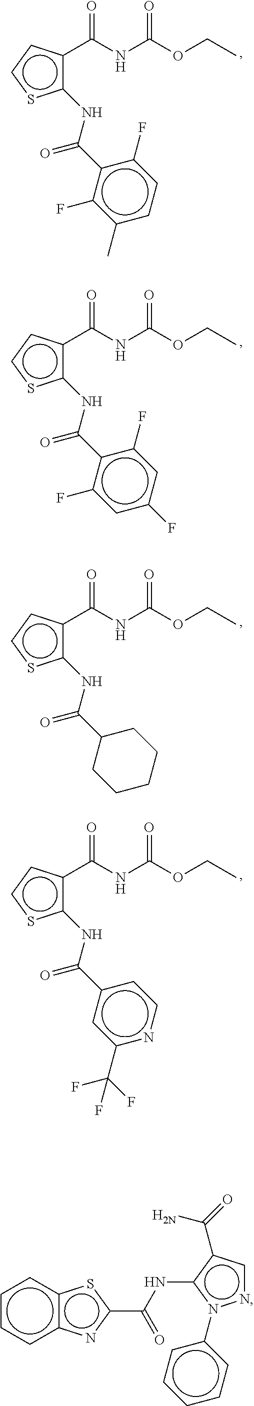 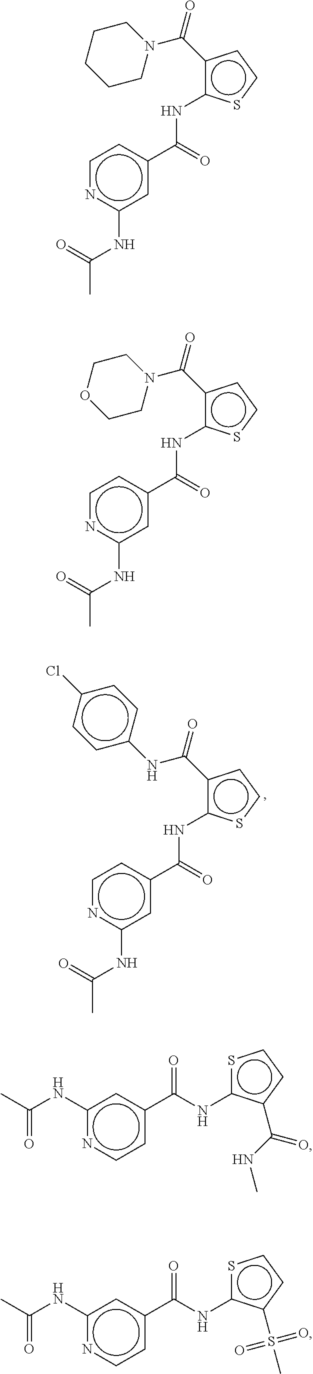 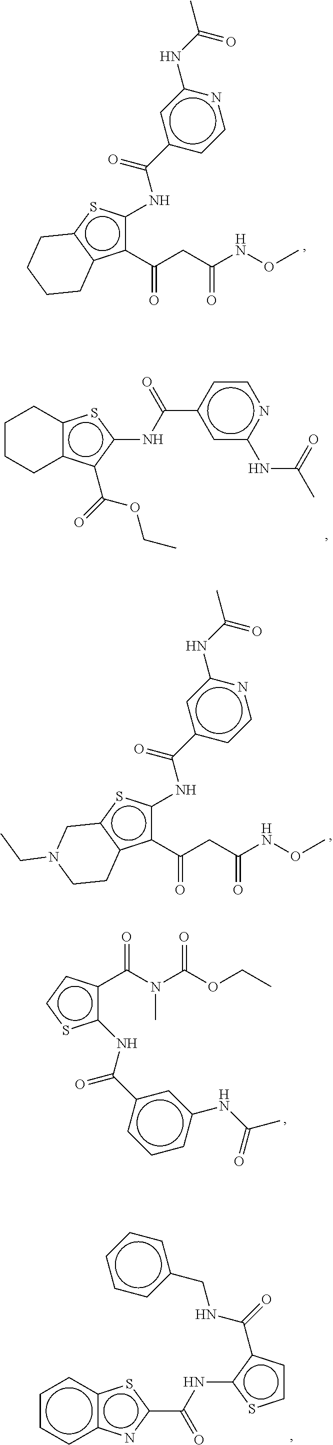 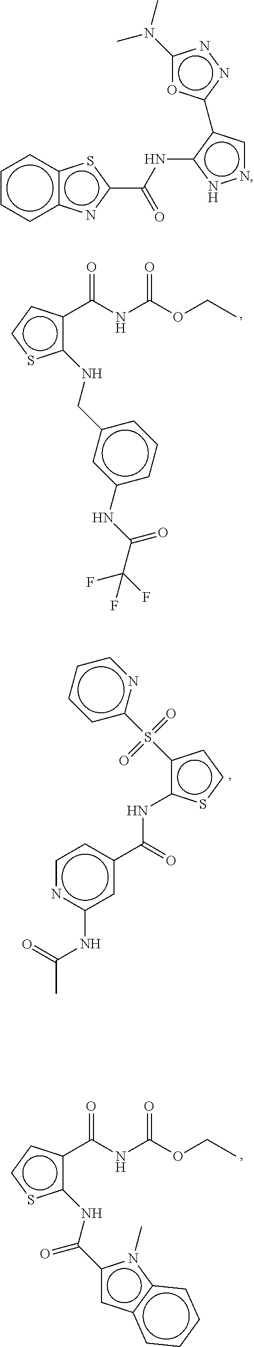 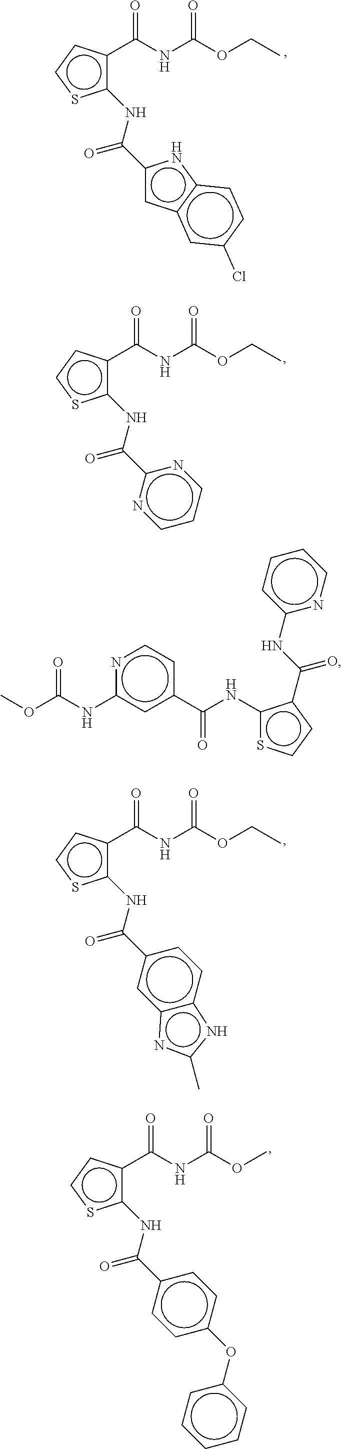 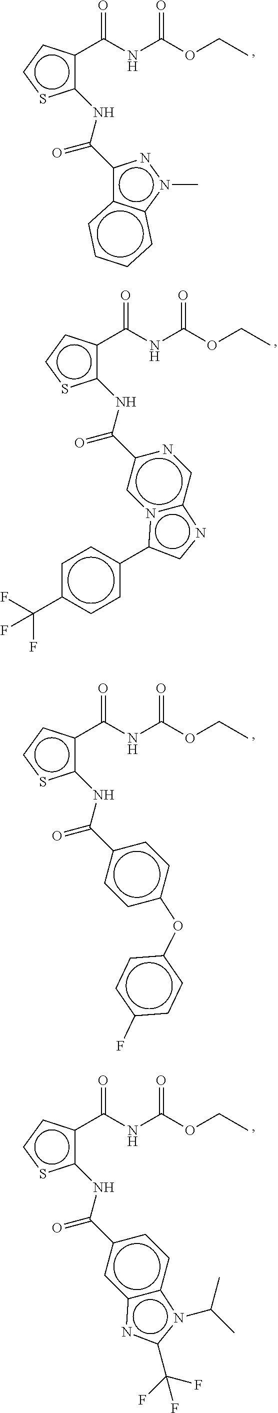 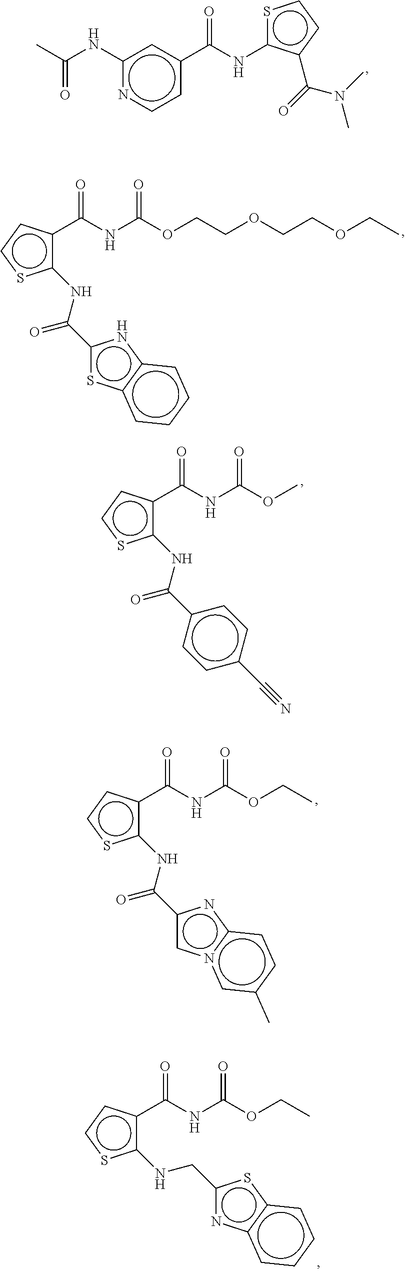 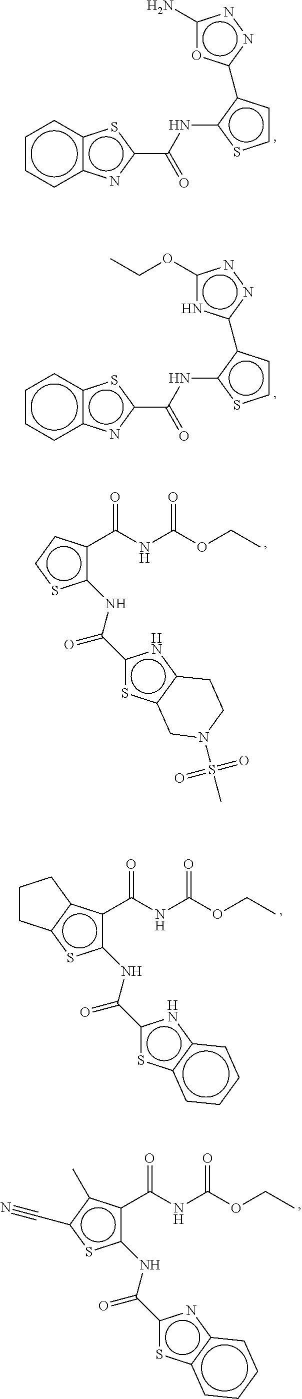 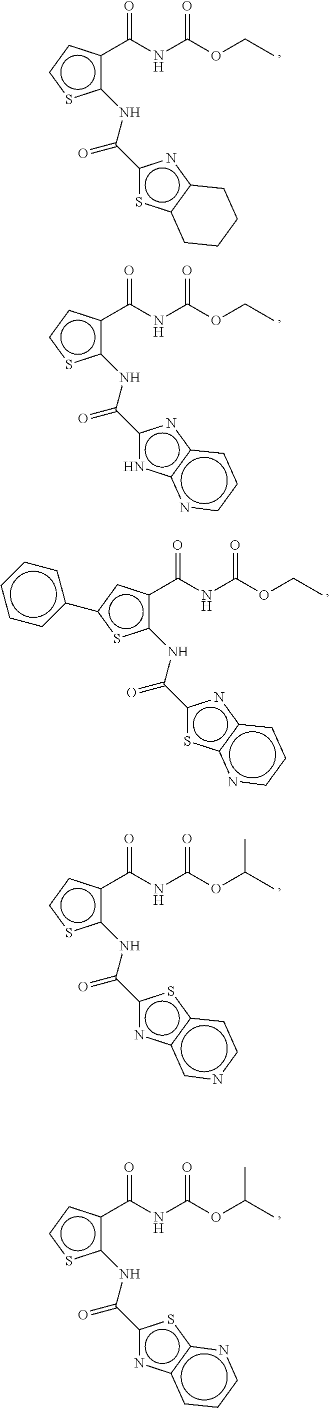 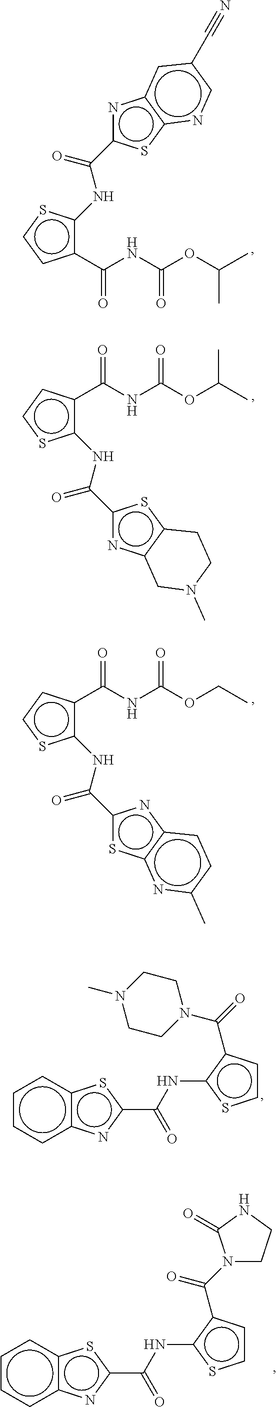 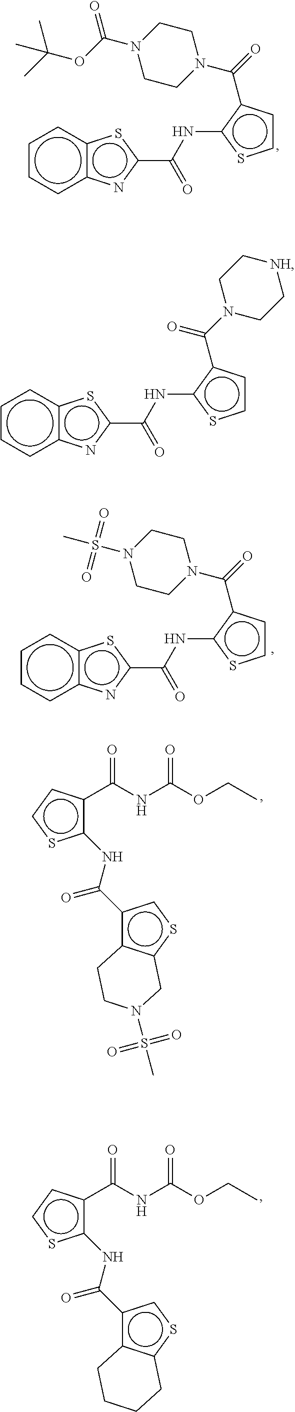 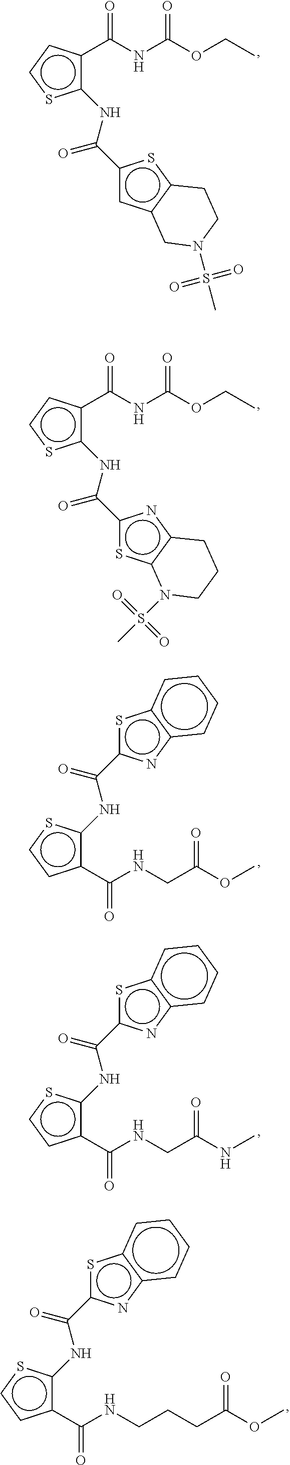 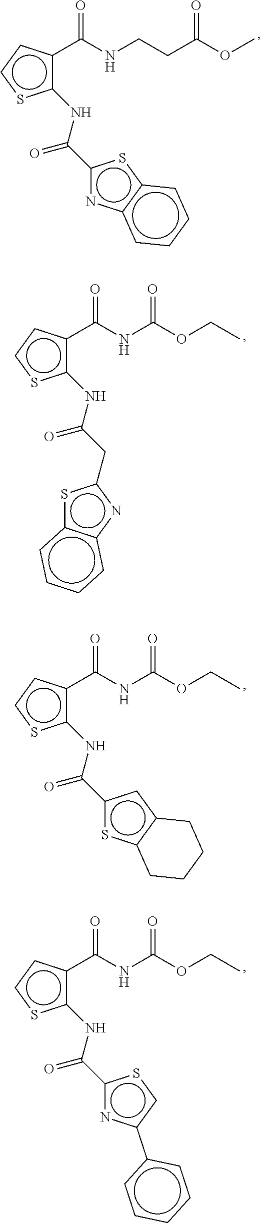 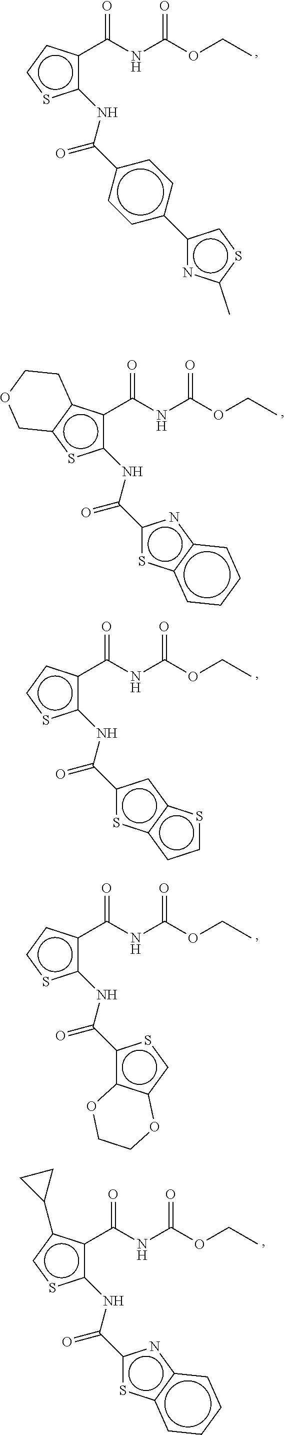 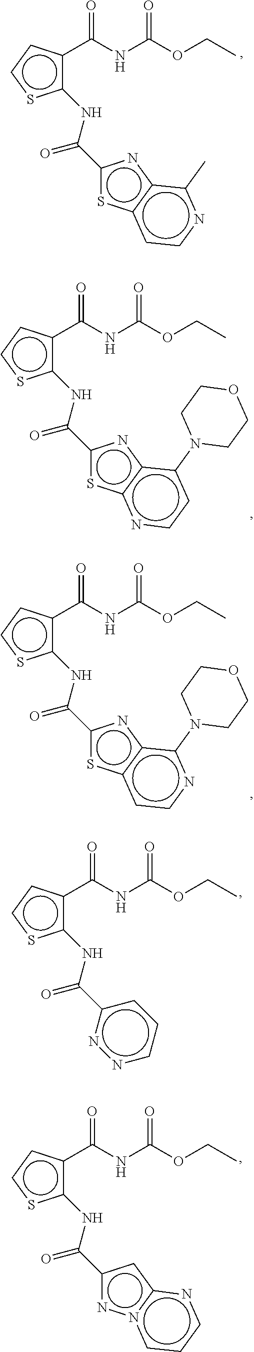 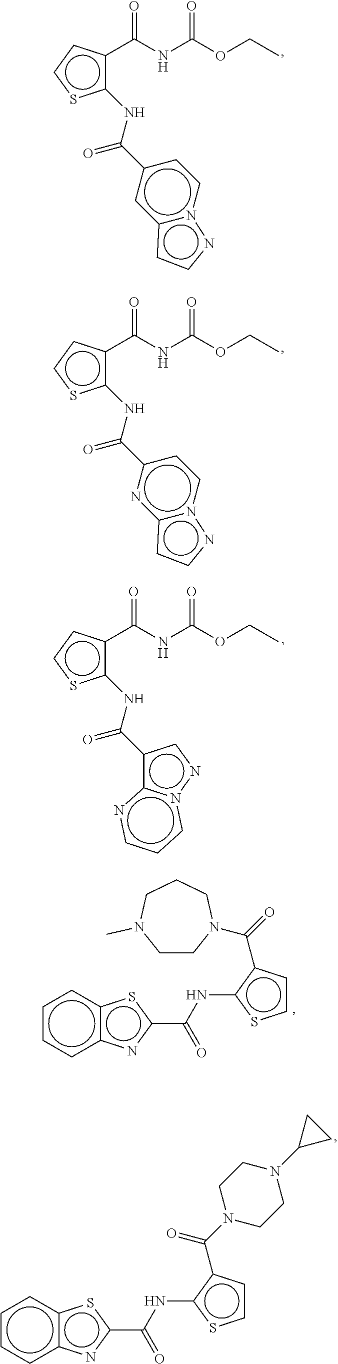 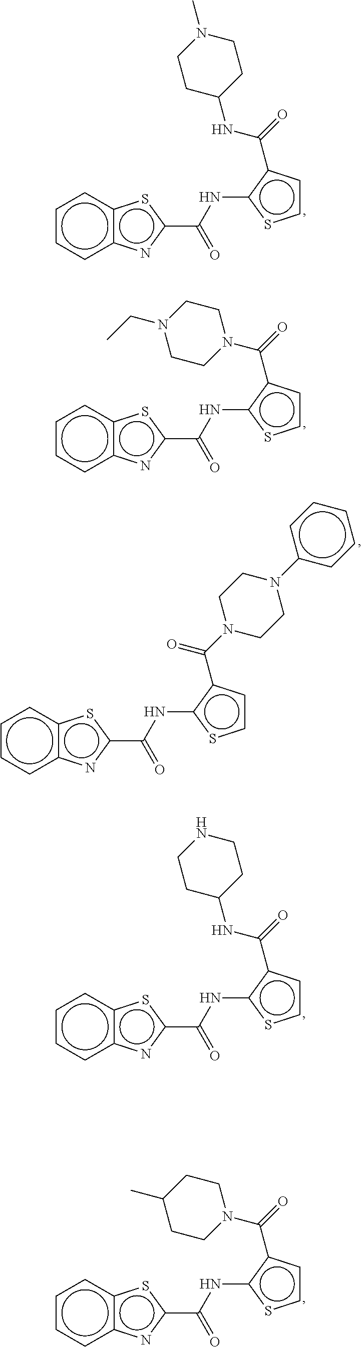 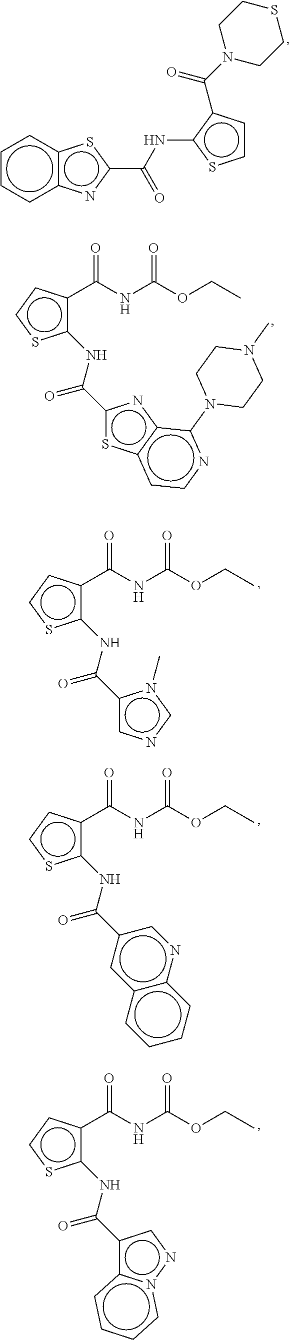 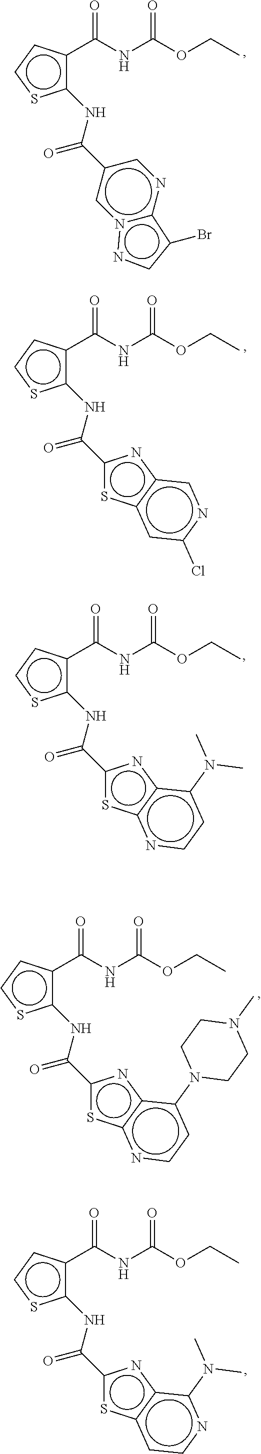 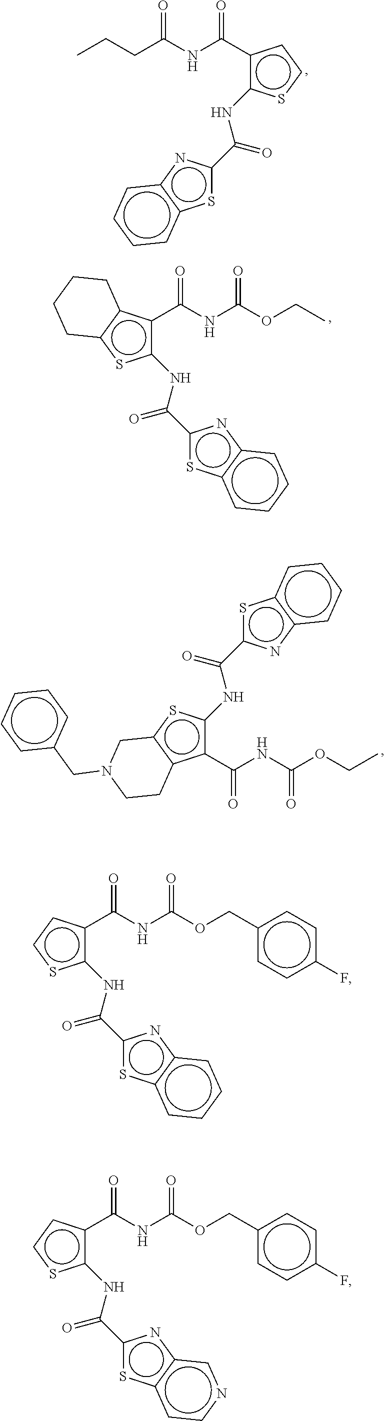 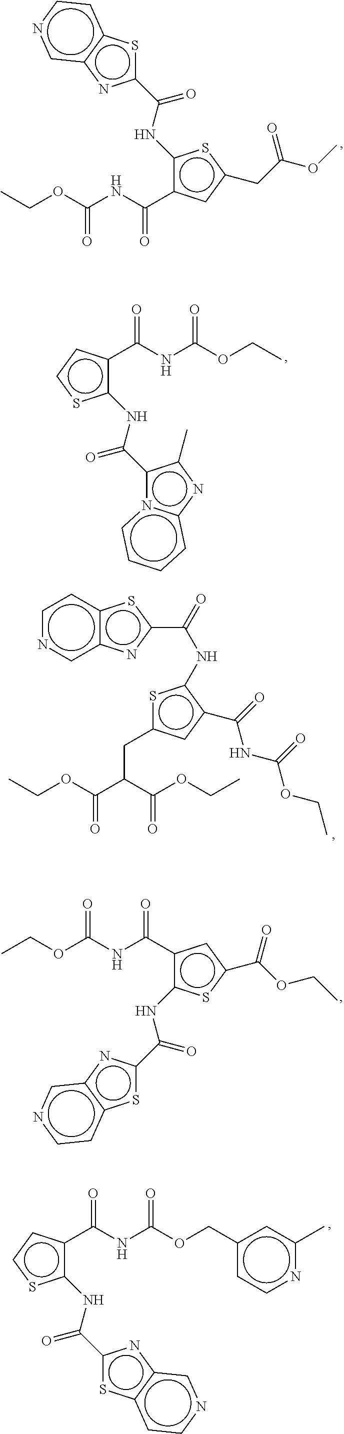 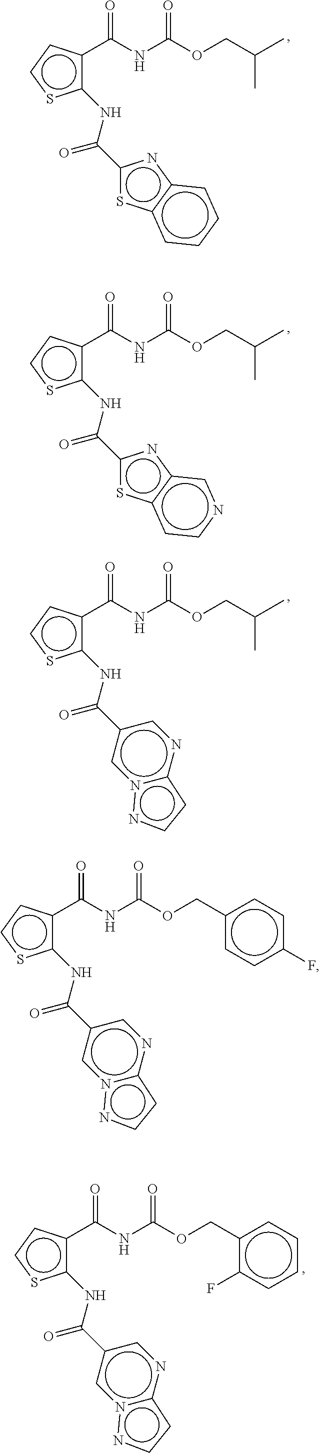 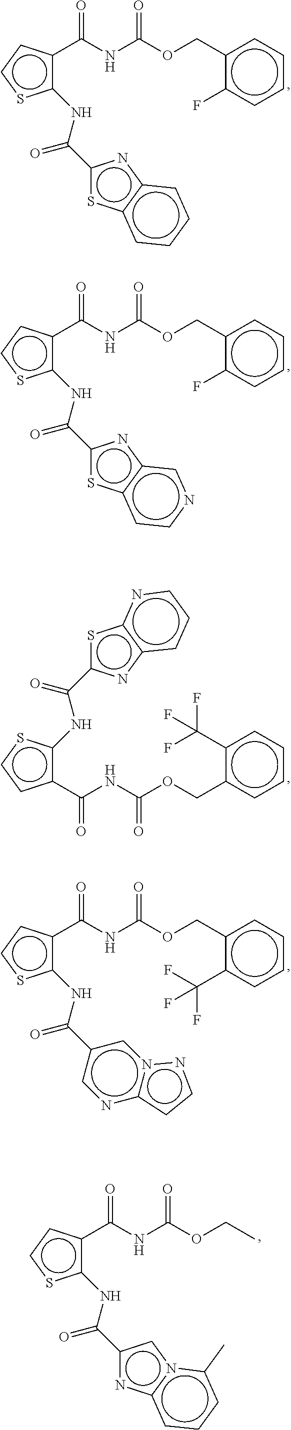 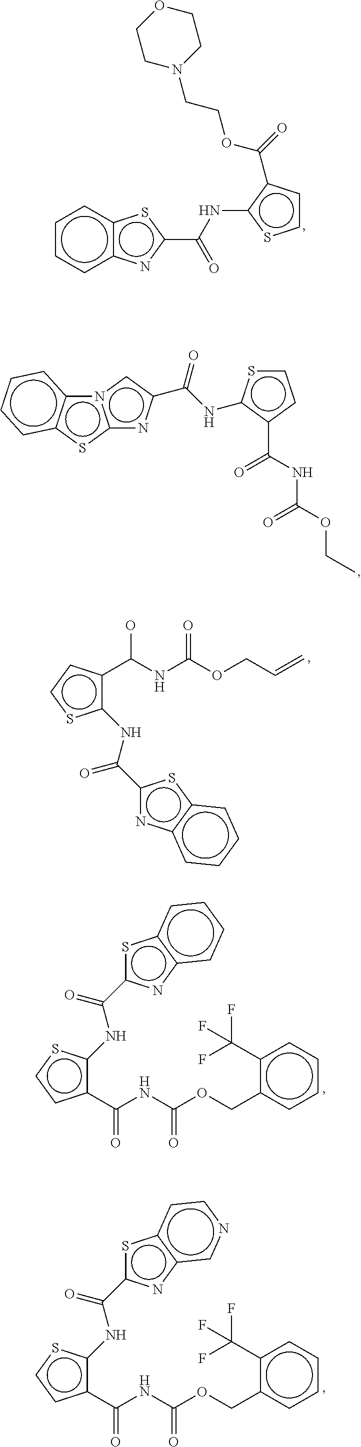 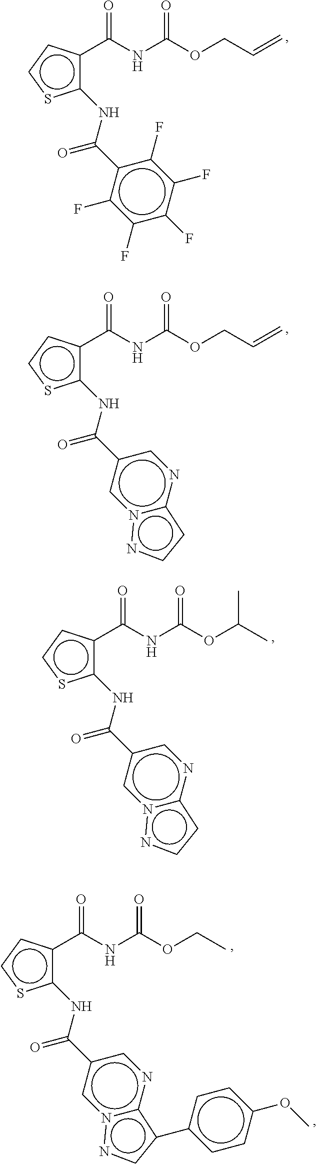 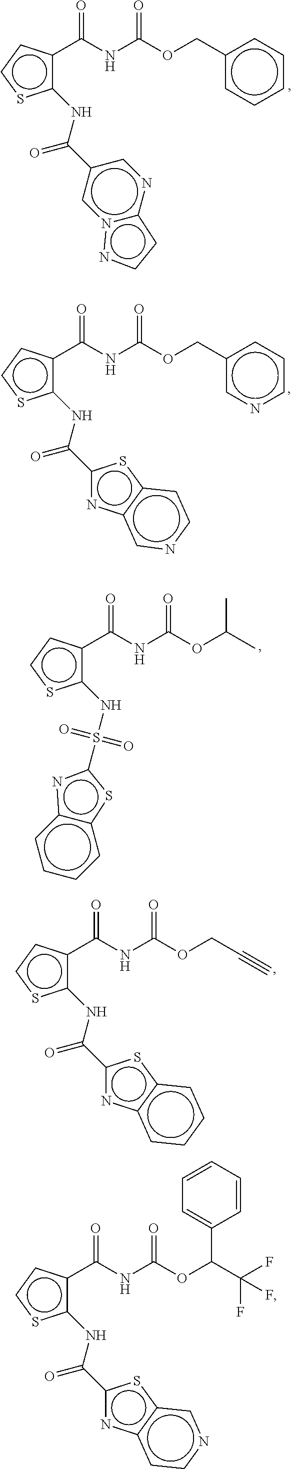 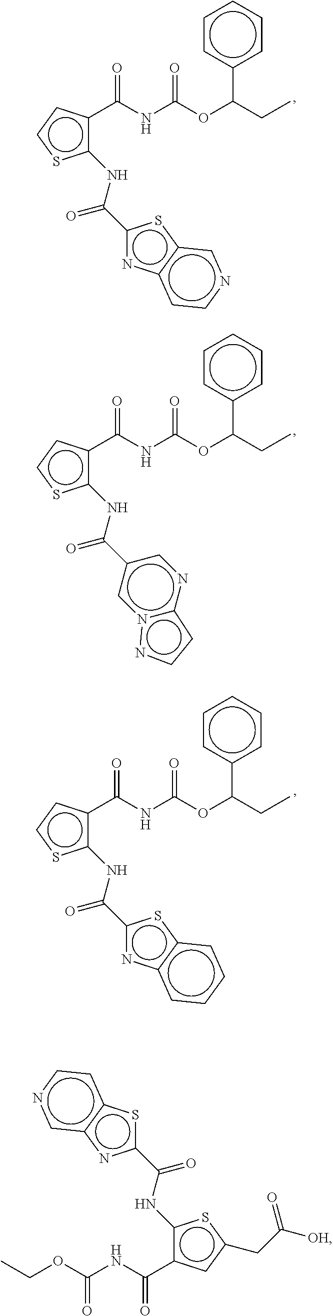 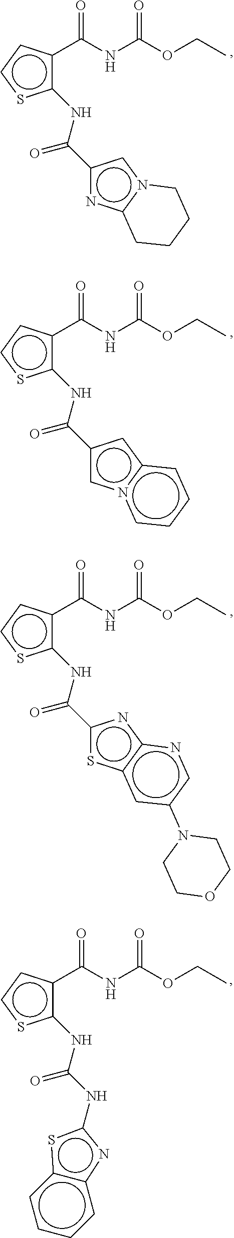 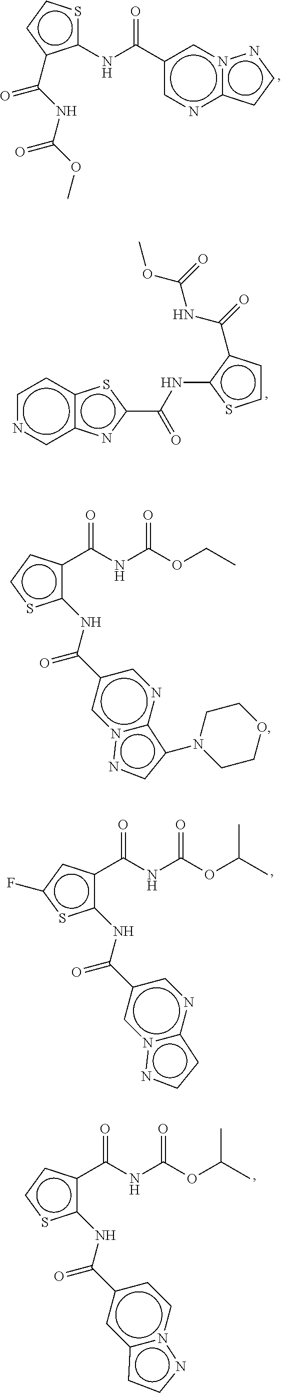 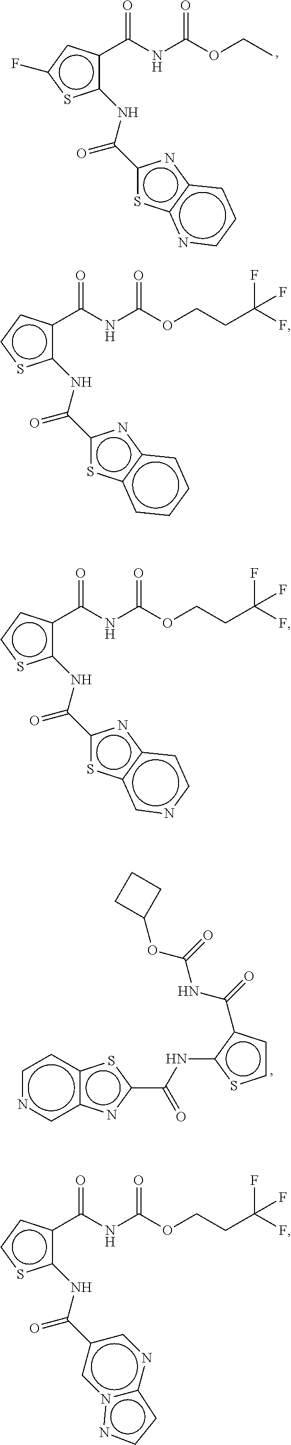 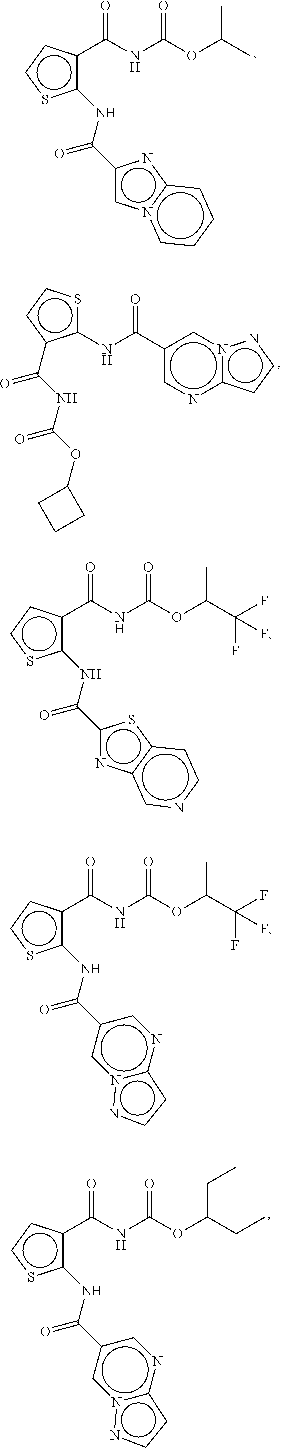 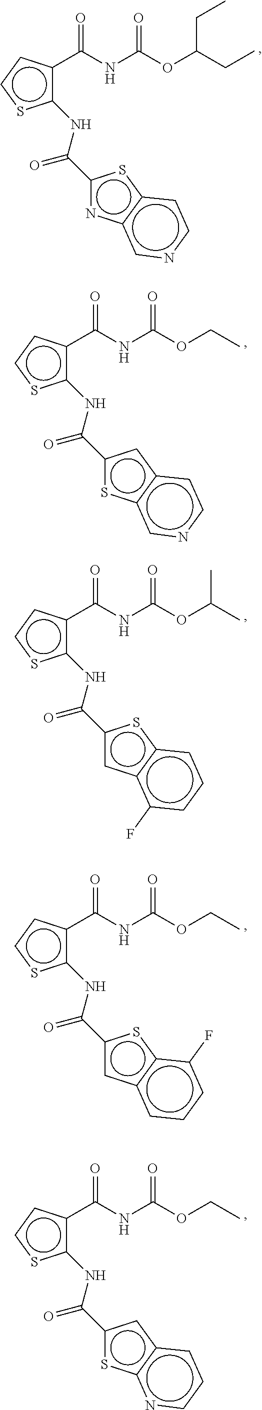 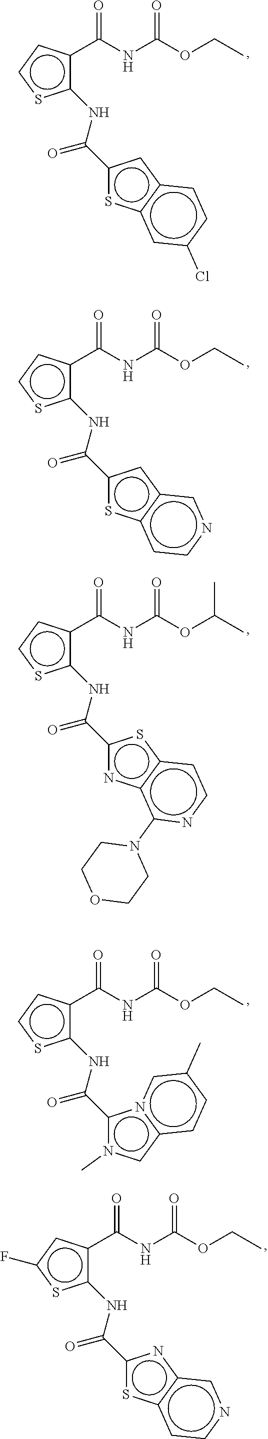 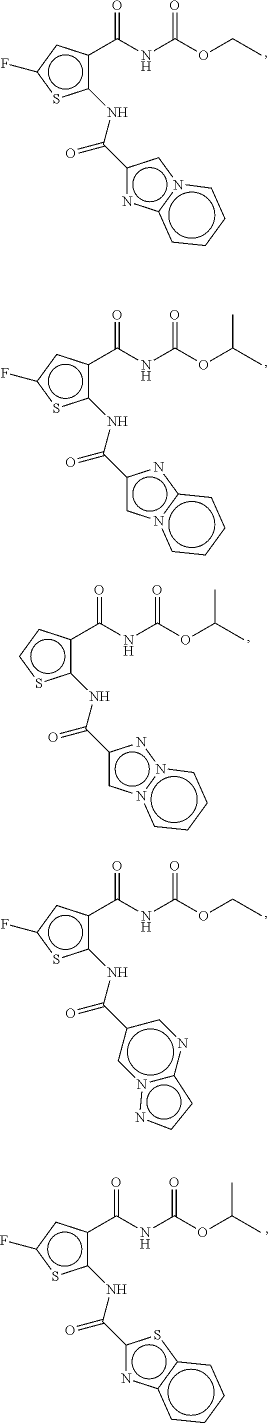 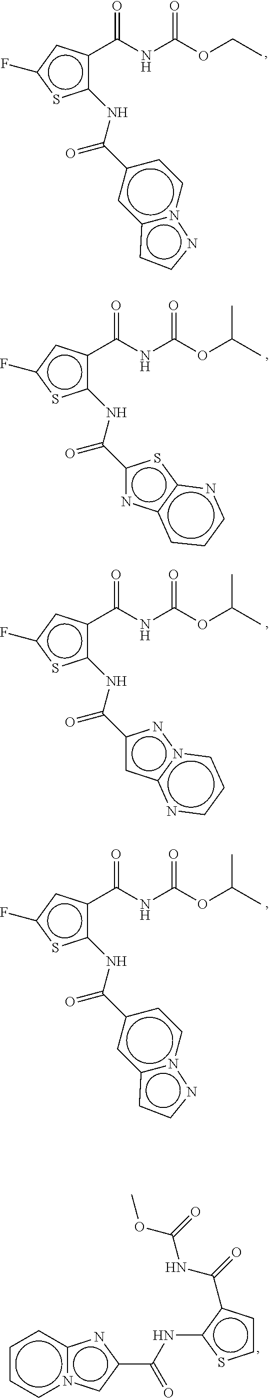 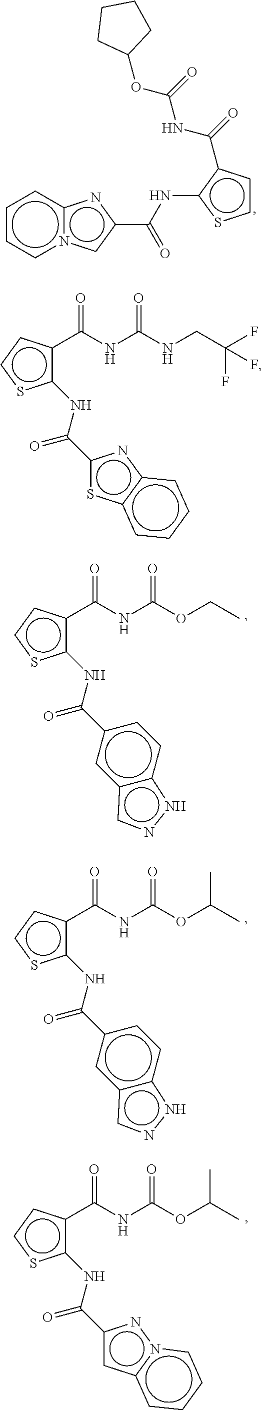 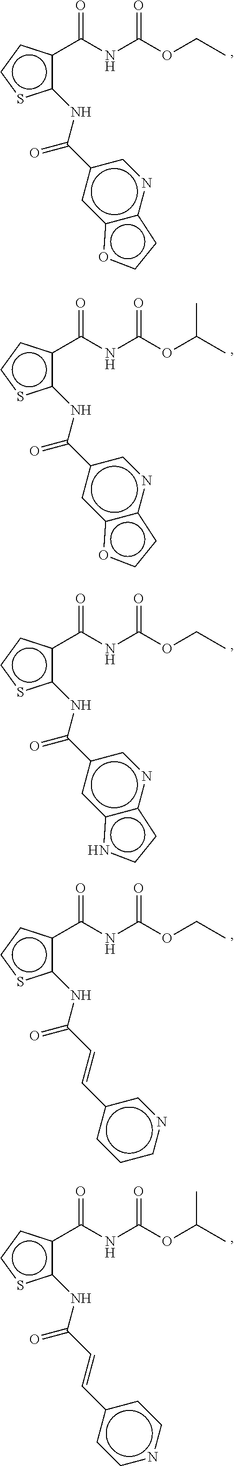 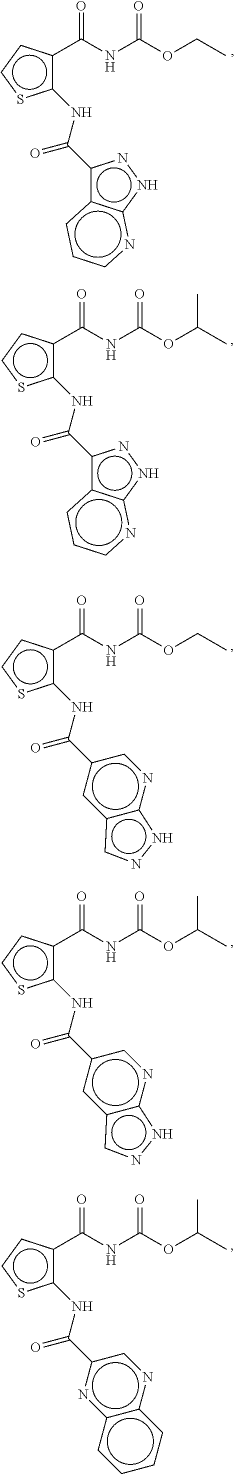 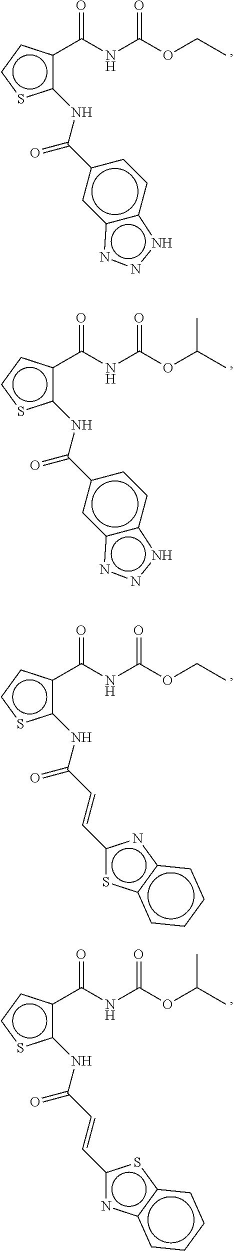 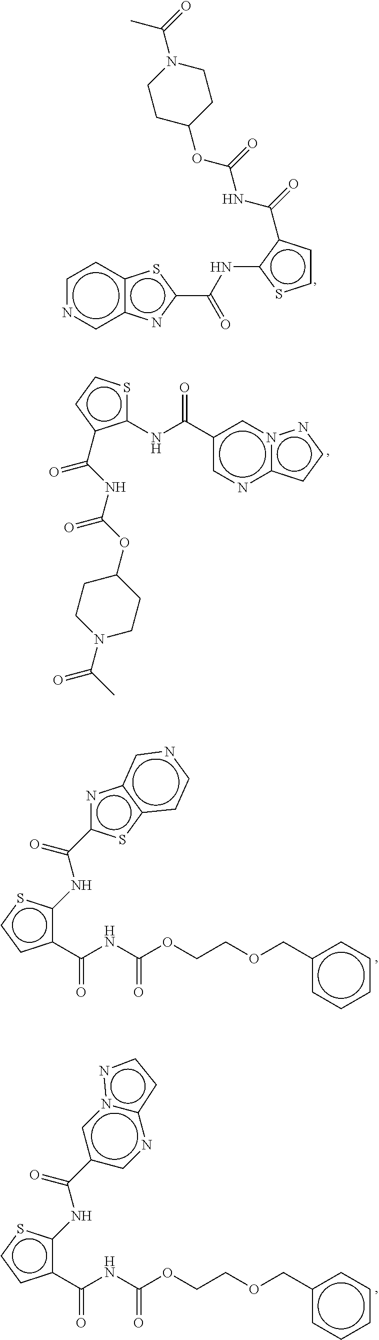 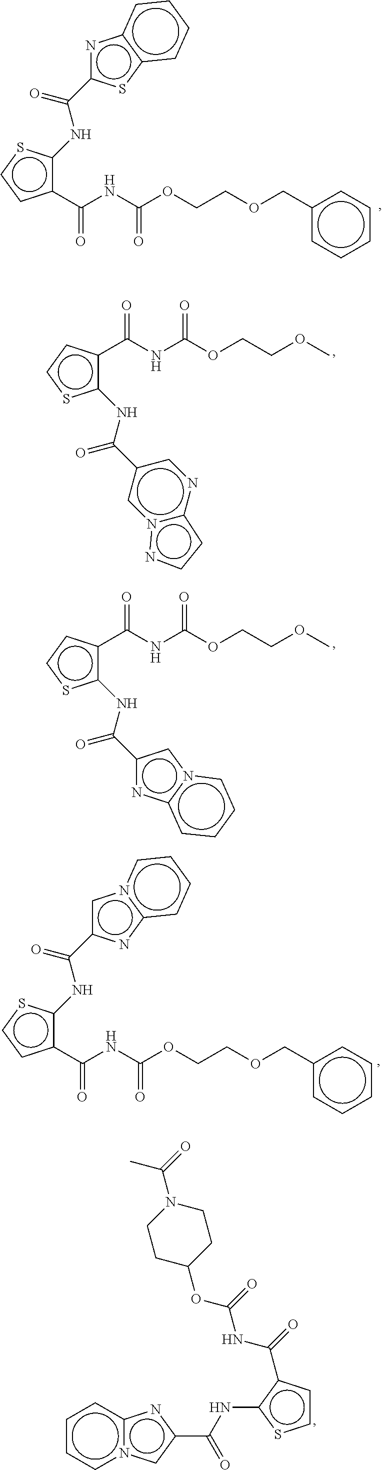 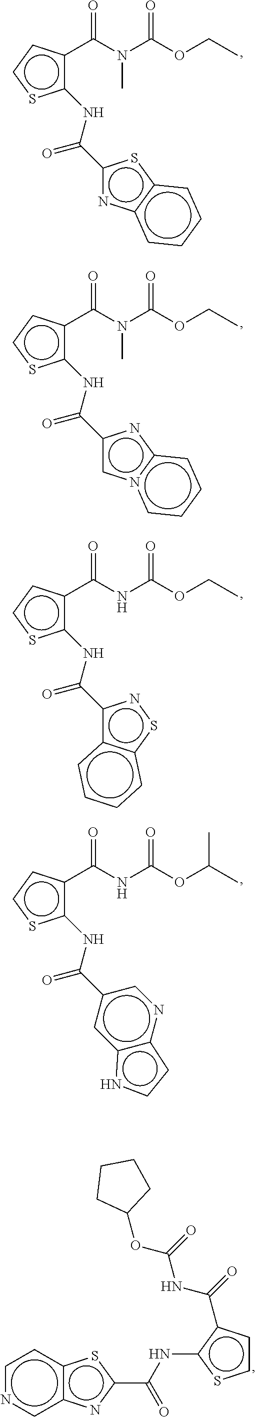 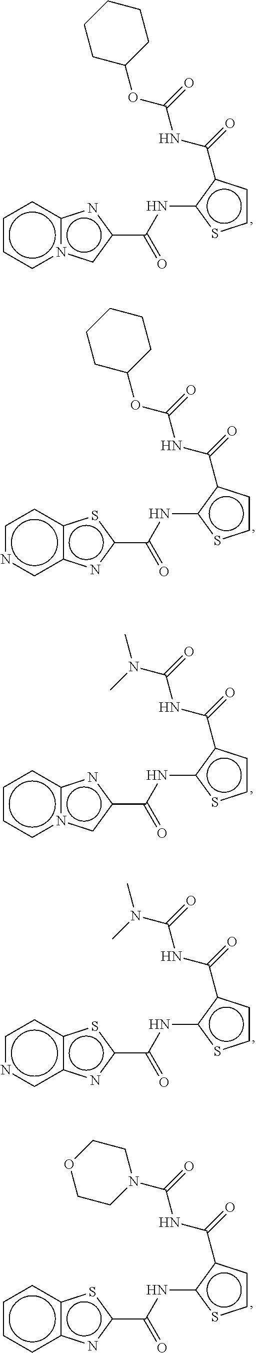 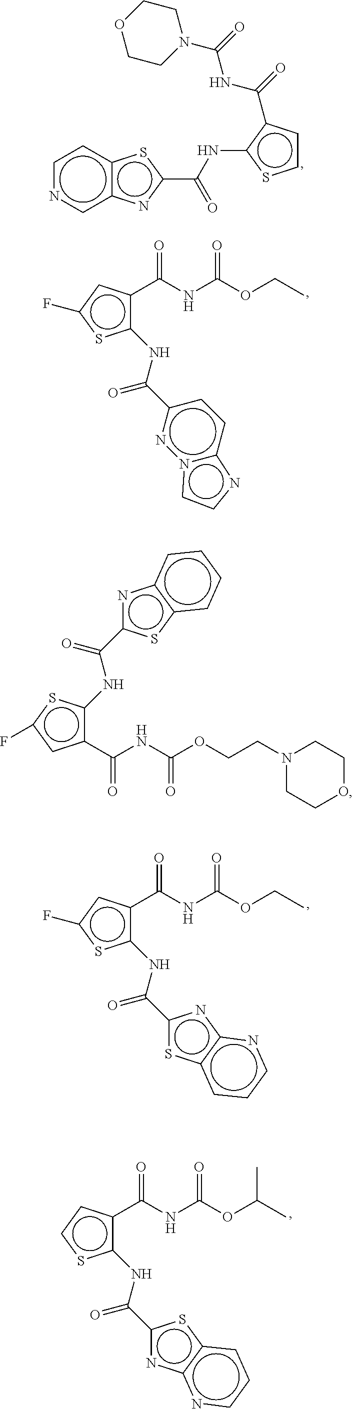 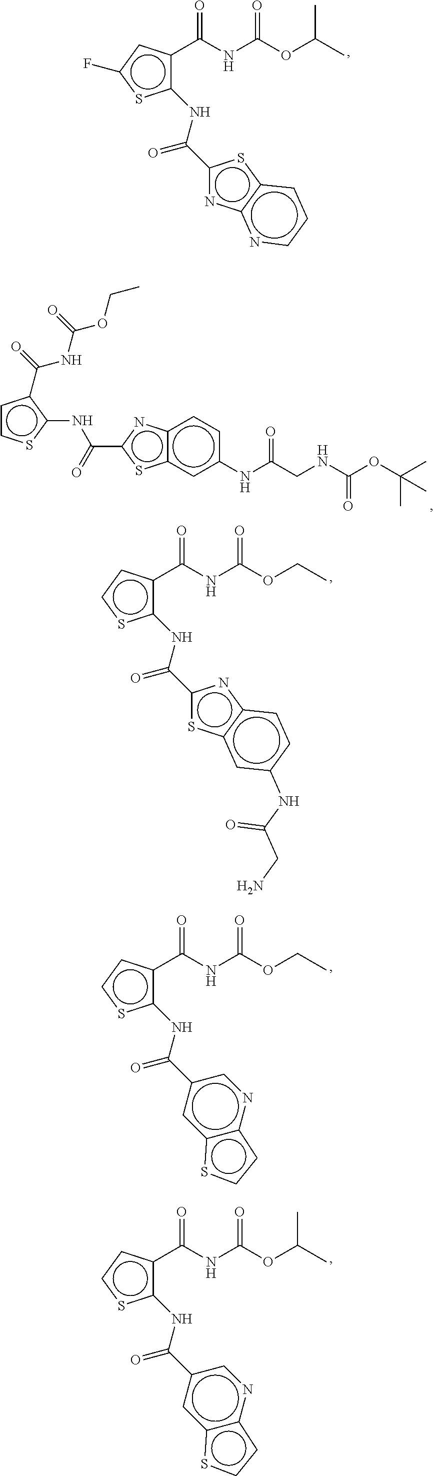 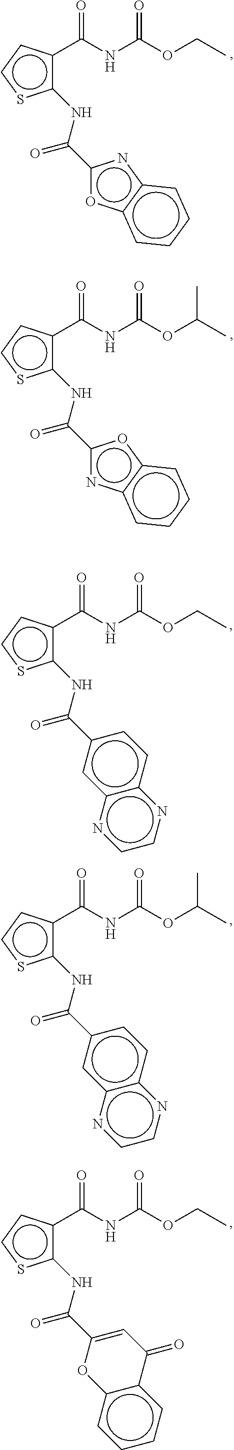 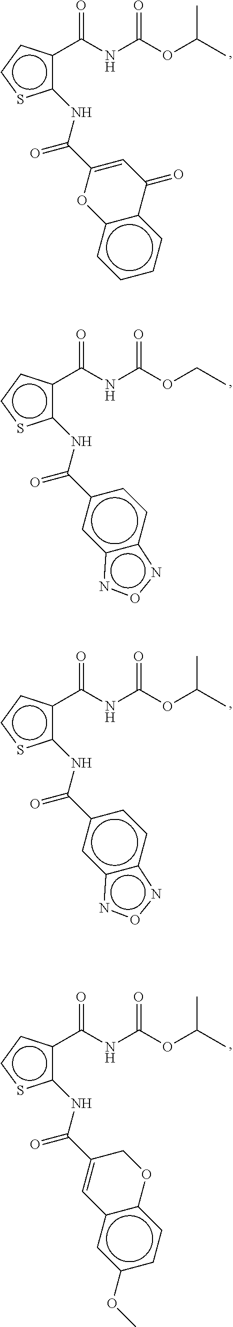 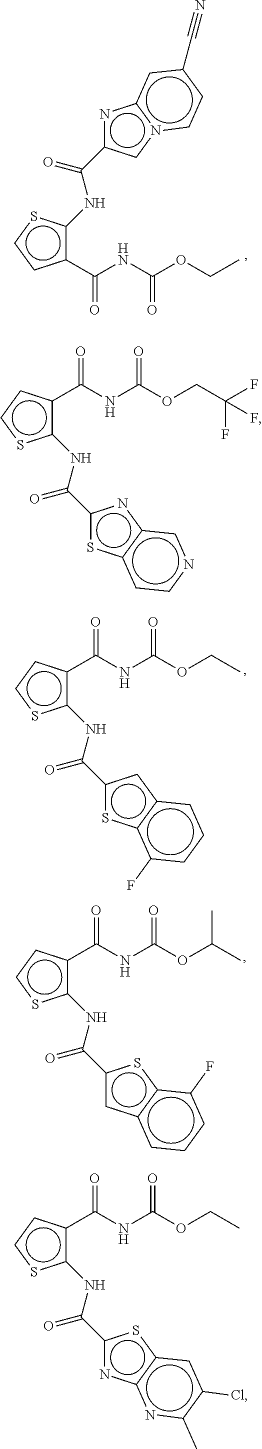 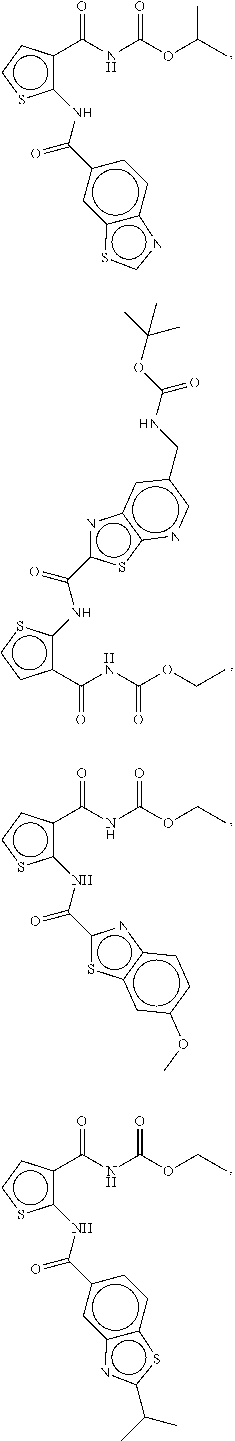 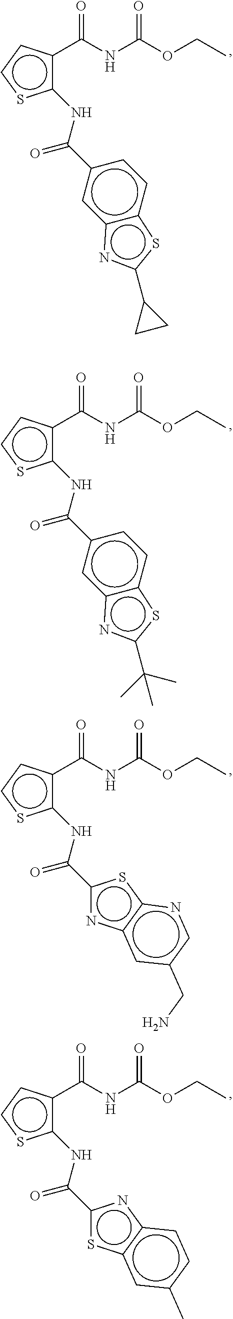 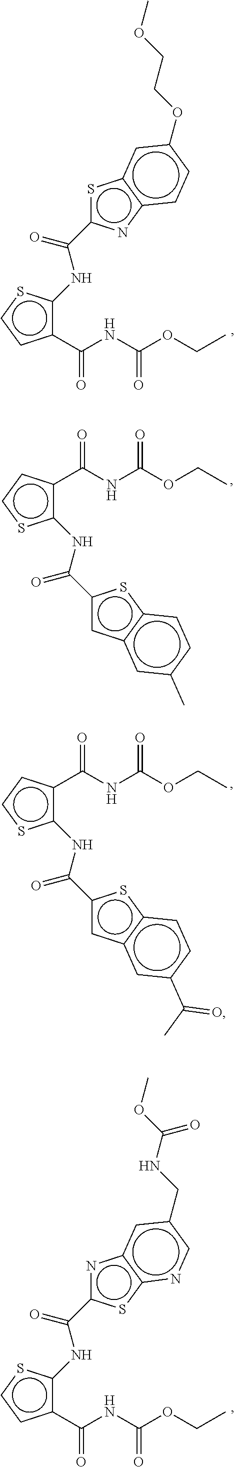 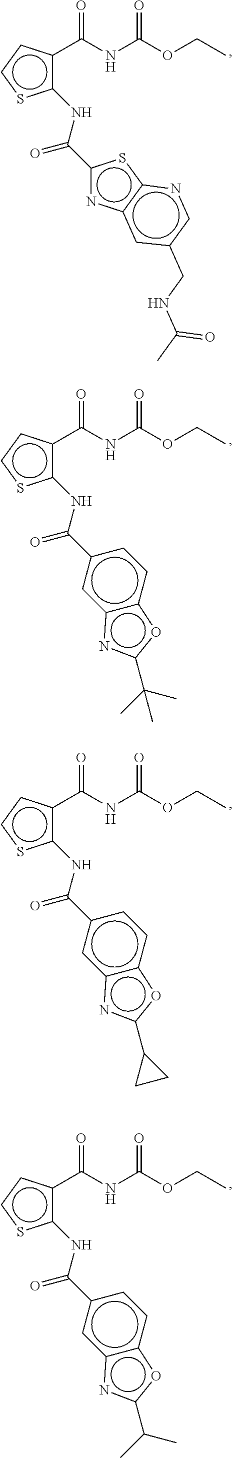 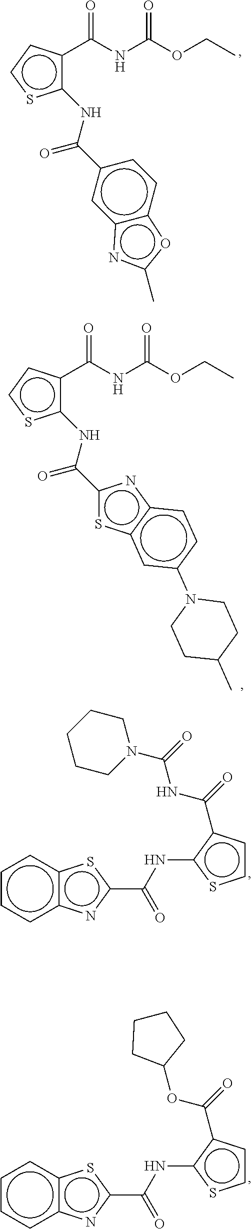 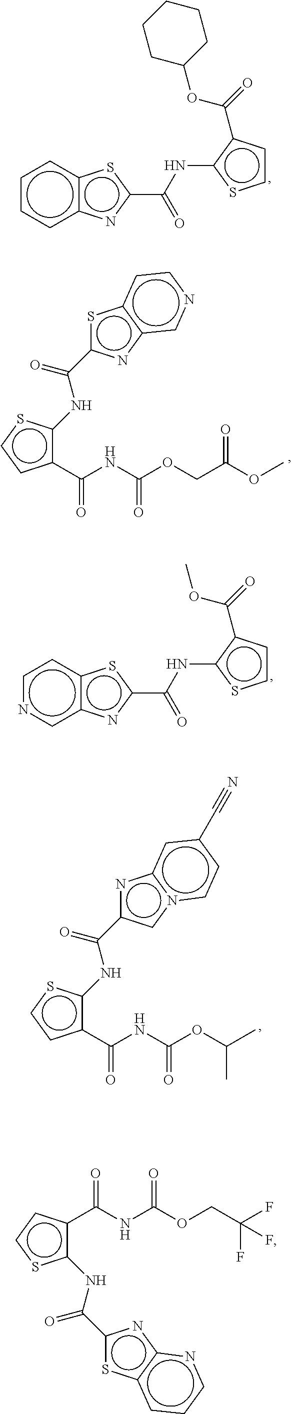 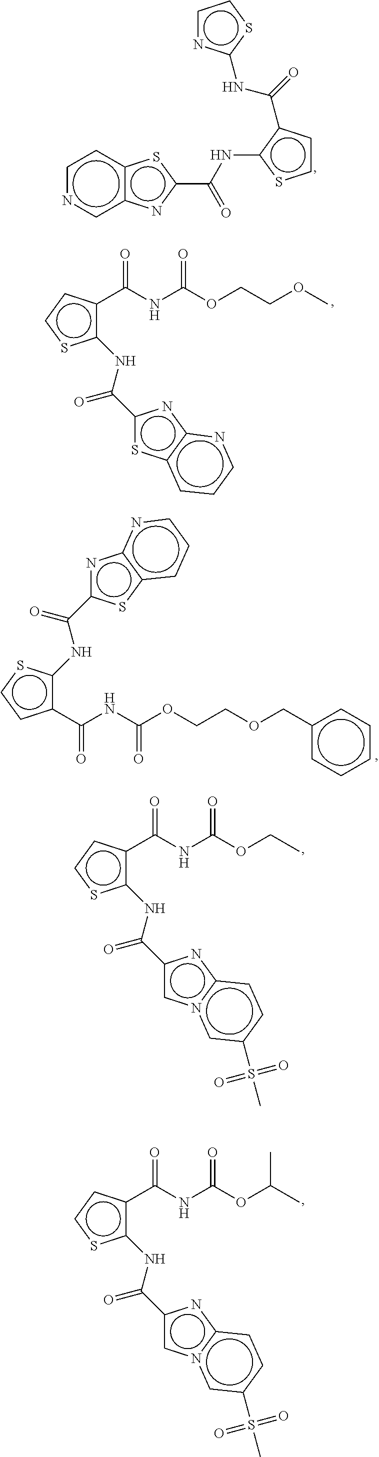 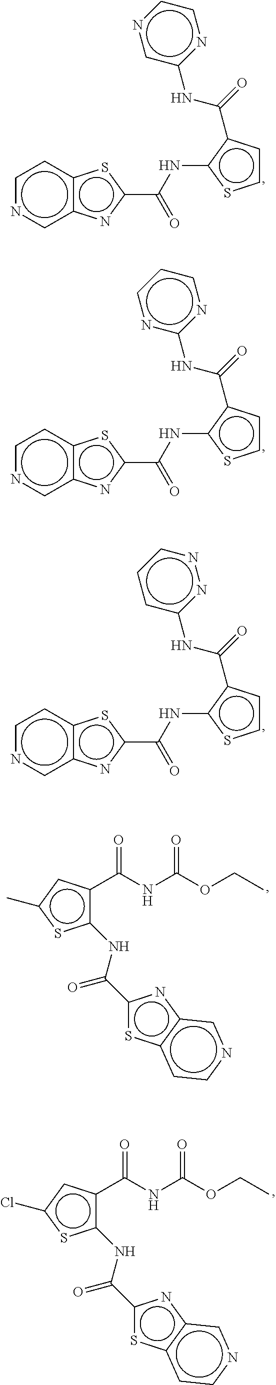 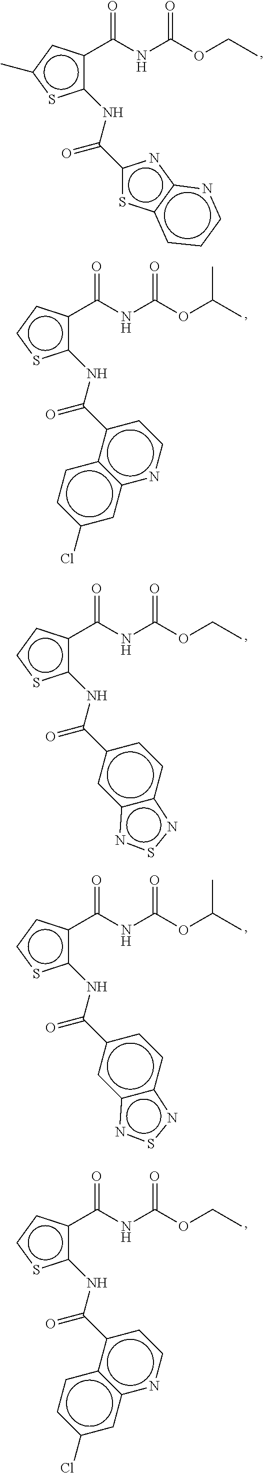 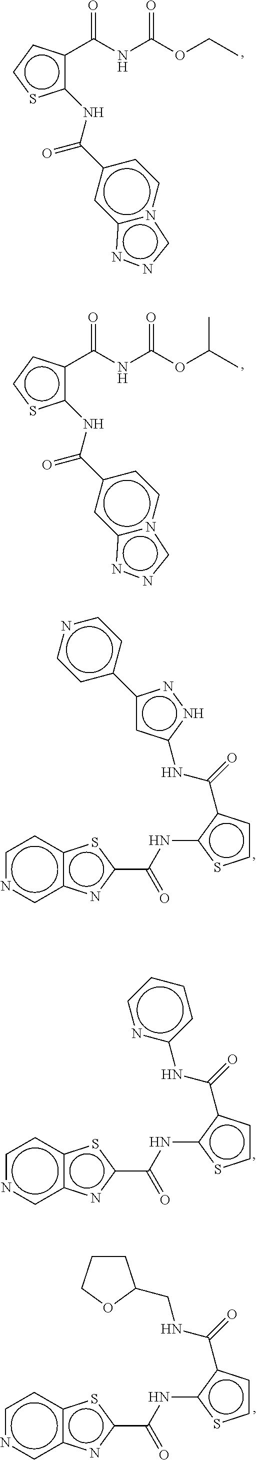 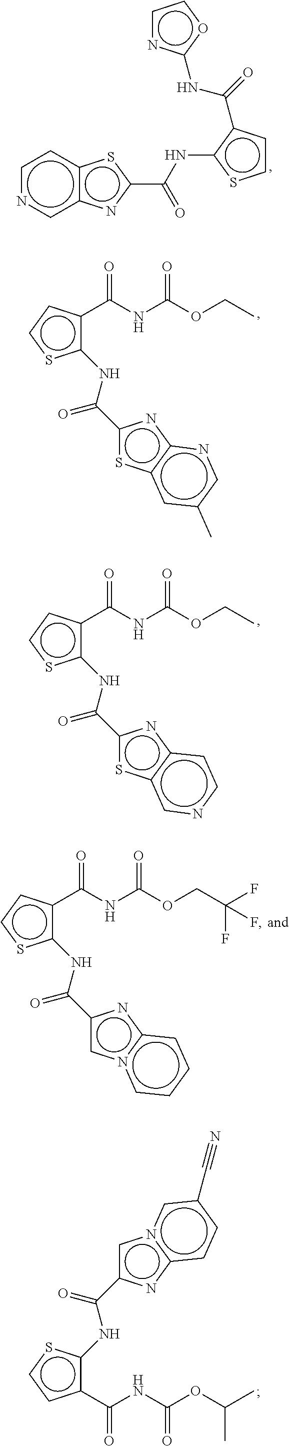
or pharmaceutically acceptable salt, solvate, polymorph, prodrug, metabolite, N-oxide, stereoisomer, or isomer thereof.

54. A compound selected from: 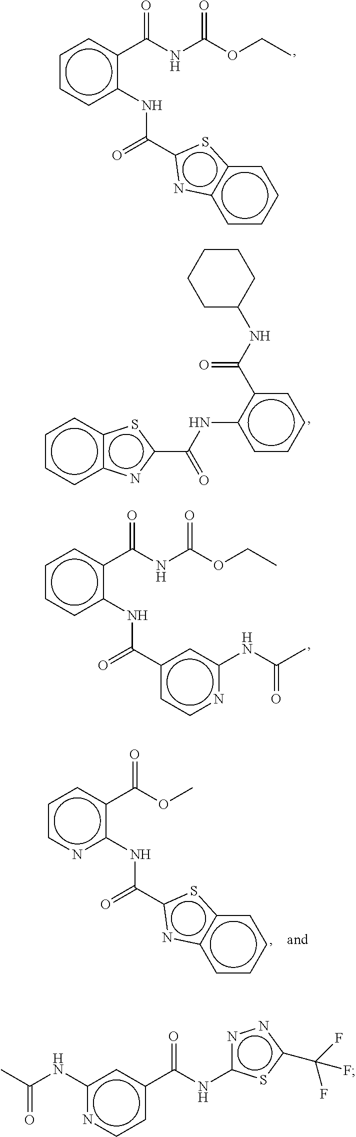
or pharmaceutically acceptable salt, solvate, polymorph, prodrug, metabolite, N-oxide, stereoisomer, or isomer thereof.

55.-64. (canceled)

65. A pharmaceutical composition comprising a compound of claim 9, or a pharmaceutically acceptable salt, solvate, polymorph, prodrug, metabolite, N-oxide, stereoisomer, or isomer thereof, and a pharmaceutically acceptable excipient.

66. A method to treat drug resistant and persistent tuberculosis in a mammal, the method comprising administering a composition comprising a therapeutically effective amount of a compound of claim 9.
